(19) United States
(12) Patent Application Publication
(10) Pub. No.: US 2024/0382494 A1
(43) Pub. Date: Nov. 21, 2024

(54) THERAPEUTIC COMPOUNDS

(71) Applicant: RUTGERS, THE STATE UNIVERSITY OF NEW JERSEY, New Brunswick, NJ (US)

(72) Inventor: Jun WANG, New Brunswick, NJ (US)

(21) Appl. No.: 18/648,062

(22) Filed: Apr. 26, 2024

Related U.S. Application Data
(60) Provisional application No. 63/462,847, filed on Apr. 28, 2023.

(51) Int. Cl. A61K 31/5355; A61K 9/12; A61K 9/20; A61K 31/4709; A61K 31/496; A61K 31/506; A61P 31/14; C07D 215/06; C07D 401/04; C07D 401/14; C07D 405/04; C07D 409/04; C07D 413/04; C07D 413/14; C07D 417/04; C07D 471/04; C07D 471/08; C07D 487/06; C12N 9/99

(52) U.S. Cl. CPC A61K 31/5355; A61K 9/12; A61K 9/2018; A61K 9/2027; A61K 9/2054; A61K 31/4709; A61K 31/496; A61K 31/506; A61P 31/14; C07D 215/06; C07D 401/04; C07D 401/14; C07D 405/04; C07D 409/04; C07D 413/04; C07D 413/14; C07D 417/04; C07D 471/04; C07D 471/08; C07D 487/06; C12N 9/99

(57) ABSTRACT

The invention provides a compound of formula (I):

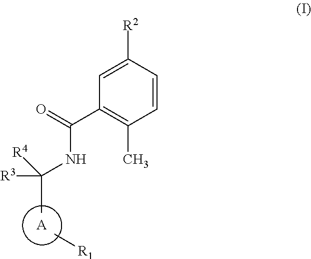

or a salt thereof, R¹-R⁴, and A have any of the values described in the specification, as well as compositions comprising a compound of formula I. The compounds are useful as inhibitors of papain-like protease and as antiviral agents.

### CROSS-REFERENCE TO RELATED APPLICATIONS

This application claims priority from U.S. Provisional Patent Application No. 63/462,847 that was filed on 28 Apr. 2023. The entire content of U.S. Provisional Patent Application No. 63/462,847 is hereby incorporated herein by reference.

### BACKGROUND

The COVID-19 pandemic is by far the worst pandemic since the 1918 Spanish flu. The etiological agent of COVID-19 is SARS-CoV-2, a single-stranded positive sense RNA virus that belongs to the beta coronavirus genus (Hu, B., et al., Nat Rev Microbiol 2021, 19, 141-154). Two additional coronaviruses within the same genus, SARS-CoV, and MERS-CoV, have caused epidemics in humans with mortality rates of 9.6% and 34.3%, respectively. Although SARS-CoV-2 has a lower mortality rate of 2.1% compared to SARS-CoV and MERS-CoV, it has led to a far greater death toll due to its higher transmission (Piroth, L, et al., The Lancet Respiratory Medicine 2021, 9, 251-259). SARS-CoV-2 differs from SARS-CoV and MERS-CoV in that it has a long incubation time after the initial infection (i to 2 weeks), and a large percentage of infected patients continue to shed the virus while being asymptomatic, presenting a daunting task for surveillance and containment (Bar-On, Y. M., et al., Elife 2020, 9, e57309).

Three mRNA vaccines developed by Pfizer/BioNtech, Moderna, and Johnson and Johnson have been approved by FDA in the United States (Li, Y., et al., A Comprehensive Review of the Global Efforts on COVID-19 Vaccine Development. ACS Central Science 2021). For small molecule antivirals, remdesivir received FDA approval on Oct. 22, 2020 (Eastman, R. T., et al., ACS Central Science 2020, 6, 672-683). Although the polymerase of SARS-CoV-2 has proof-reading function, it continues to mutate at a rate about 10⁻⁶ per site per cycle (Bar-On, Y. M., et al., Elife 2020, 9, e57309). Several variants have already emerged and widely circulated among humans since the beginning of the pandemic (Lauring, A. S.; Hodcroft, E. B., JAMA 2021, 325, 529-531). Therefore, there is a dire need for additional antivirals with a novel mechanism of action.

Antivirals are not substituents of vaccines, but rather an important complement that can be used for the treatment of infection from both wild-type (WT) and variant viruses. Among the viral proteins that have been actively pursued as SARS-CoV-2 antiviral drug targets, the main protease (M^{pro}) and papain-like protease (PL^{pro}) are the most promising ones (Ma, C., et al., Cell Res 2020, 30, 678-692; and Sacco, M. D, et al., Sci Adv 2020, 6, eabe0751). M^{pro} and PL^{pro} are involved in the proteolytic digestion of the viral polyproteins pp1a and pp1ab, yielding individual functional viral proteins for the replication complex formation. PL^{pro} cleaves at three sites with the recognition sequence “LXGG↓XX” (Rut, W., et al., Sci Adv 2020, 6). PL^{pro} has been shown to play additional roles in dysregulating host immune response and impairing the host type I interferon antiviral effect through its deubiquitinating and deISG15ylating (interferon-induced gene 15) activities, respectively (Freitas, B. T., et al., ACS Infect Dis 2020, 6, 2099-2109; Shin, D., et al., Nature 2020, 587, 657-662; and Klemm, T., et al., Embo J 2020, 39, e106275). SARS-CoV-2 PL^{pro} cleaves ISG15 and polyubiquitin modifications from cellular proteins, and inhibition of PL^{P} led to the accumulation of ISG15-conjugates and poly-ubiquitin-conjugates (Fu, Z., et al., Nat Commun 2021, 12, 488). While SARS-CoV PL^{pro} prefers ubiquitinated substrates, SARS-CoV-2 PL^{pro} prefers the ISGlyated proteins as substrates (Freitas, B. T., et al., ACS Infect Dis 2020, 6, 2099-2109; Shin, D., et al., Nature 2020, 587, 657-662; and Klemm, T., et al., Embo J 2020, 39, e106275). PL^{pro} is part of a membrane anchored multi-domain protein named non-structural protein 3 (nsp-3), an essential component of the replicase-transcriptase complex. The pleiotropic roles of SARS-CoV-2 PL^{pro} make it a promising antiviral drug target. Substantial morbidity and mortality associated with COVID-19 infection is caused by cytokine storm (Berlin, D. A.; Gulick, R. M.; Martinez, F. J., N Engl J Med 2020, 383, 2451-2460), and suppressing host immune response using dexamethasone and baricitinib has been shown to provide therapeutic benefits in the treatment of severe infections (Kalil, A. C., et al., New England Journal of Medicine 2020, 384, 795-807; and New England Journal of Medicine 2020, 384, 693-704).

Significant progress has been made in developing SARS-CoV-2 M^{pro} inhibitors (Ma, C., et al., Cell Res 2020, 30, 678-692; Sacco, M. D, et al., Sci Adv 2020, 6, eabe0751; Qiao, J., et al., Science 2021, 371, 1374-1378; Zhang, L., et al., Science 2020, 368, 409-412) and the Pfizer's nirmatrelvir is approved by FDA. In comparison, PL^{pro} represents a more challenging drug target, and GRL0617 remains one of the most potent PL^{pro} inhibitors reported to date despite several high-throughput screening and medicinal chemistry optimization campaigns (Rut, W., et al., Sci Adv 2020, 6; Freitas, B. T., et al., ACS Infect Dis 2020, 6, 2099-2109; Shin, D., et al., Nature 2020, 587, 657-66; Klemm, T., et al., Embo J 2020, 39, e106275; Osipiuk, J., et al., Nat Commun 2021, 12, 743; and Ratia, K., et al., Proc. Natl. Acad. Sci. U.S.A 2008, 105, 16119-24). GRL0617 was originally developed as a deubiquitinase inhibitor and was later identified as a SARS-CoV PL^{pro} inhibitor through a high-throughput screening (Ratia, K., et al., Proc. Natl. Acad. Sci. U.S.A 2008, 105, 16119-24). As SARS-CoV-2 and SARS-CoV PL^{pro} share a sequence identity of 83% and similarity of 90%, GRL0617 was also repurposed for SARS-CoV-2 PL^{pro} and it was reported to inhibit SARS-CoV-2 PL^{pro} with IC₅₀ values of around 2 μM and SARS-CoV-2 viral replication with EC₅₀ values around 20 μM from multiple studies (Klemm, T., et al., Embo J 2020, 39, e106275; Fu, Z., et al., Nat Commui 2021, 12, 488; Osipiuk, J., et al., Nat (Commun 2021, 12, 743; and Gao, X., et al., Acta Pharm Sin B 2021, 11, 237-245). Currently there is a need for SARS-CoV-2 antiviral agents that can be used alone or in combination with other drugs (e.g., protease inhibitors).

### SUMMARY

The invention provides SARS-CoV-2 antiviral agents that can be used alone or in combination with other drugs (e.g., antiviral agents, such as, for example, protease inhibitors).

Accordingly, in one embodiment, the invention provides a compound of the invention, which is a compound of formula (I):

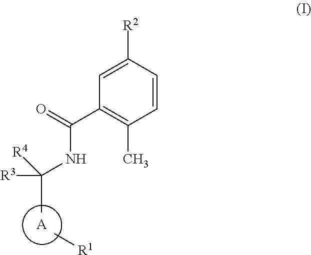

or a salt thereof, wherein:
R¹ is halo, NR^{a}R^{b}, phenyl, benzyloxy, a 3-10 membered heterocycle, or a 5-10 membered heteroaryl, which phenyl, benzyloxy, 3-10 membered heterocycle, and a 5-10 membered heteroaryl is optionally substituted with one or more groups R^{x} independently selected from the group consisting of halo, hydroxy, cyano, (C₁-C₆)alkyl, (C₁-C₆)alkoxy, (C₁-C₆)alkylthio, (C₃-C₆)cycloalkyl, NR^{a}R^{b}, oxo, 3-10 membered heterocycle, a 5-10 membered heteroaryl, —C(═O)NR^{a}R^{b}, —C(═O)3-10 membered heterocycle, —C(═O)5-10 membered heteroaryl, methylenedioxy, and (C₁-C₆)alkylsulfonyl, wherein any R^{x} is optionally substituted with one or more groups R^{y} independently selected from the group consisting of halo, hydroxy, cyano, (C₁-C₆)alkyl, (C₁-C₆)alkoxy, NR^{c}R^{d}—C(═O)NR^{c}R^{d} (C₃-C₆)cycloalkyl, carboxy, 3-10 membered heterocycle, —C(═O)3-10 membered heterocycle, and phenyl, wherein any R^{y} is optionally substituted with one or more groups independently selected from the group consisting of hydroxy, (C₁-C₆)alkyl, (C₁-C₆)alkoxy, cyano, and halo;
A is a 9-membered bicyclic heteroaryl or a 10-membered bicyclic heteroaryl, which 9-membered bicyclic heteroaryl and 10-membered bicyclic heteroaryl is optionally substituted with one or more groups independently selected from the group consisting of halo, cyano, (C₁-C₆)alkyl, (C₁-C₆)alkoxy, (C₁-C₆)cycloalkyl, (C₁-C₆)alkoxycarbonyl, and (C₁-C₆)alkanoyloxy, wherein any (C₁-C₆)alkyl, (C₁-C₆)alkoxy, (C₃-C₆)cycloalkyl, (C₁-C₆)alkoxycarbonyl, and (C₁-C₆)alkanoyloxy is optionally substituted with one or more groups independently selected from halo;
R² is R^{e}, —OR^{e}, —SR^{e}, or —NR^{g}R^{h}.
R³ is H or (C₁-C₃)alkyl and R⁴ is H or (C₁-C₃)alkyl; or R³ and R⁴ taken together with the carbon to which they are attached form a (C₃-C₆)cycloalkyl;
each R^{a} and R^{b} is independently selected from the group consisting of H, (C₁-C₆)alkyl, (C₃-C₆)cycloalkyl, (C₃-C₆)cycloalkyl(C₁-C₆)alkyl, and (C₁-C₆)alkanolyl;
each R^{c} and R^{d} is independently selected from the group consisting of H, (C₁-C₆)alkyl, (C₃-C₆)cycloalkyl, (C₃-C₆)cycloalkyl(C₁-C₆)alkyl, and (C₁-C₆)alkanolyl;
R^{e} is a 3-10 membered heterocycle that is optionally substituted with one or more groups independently selected from the group consisting of halo and (C₁-C₆)alkyl; or R^{e} is (C₁-C₆)alkyl, that is substituted with NRⁱR^{k} or with a 3-10 membered heterocycle that is optionally substituted with one or more groups independently selected from the group consisting of halo and (C₁-C₆)alkyl;
each R^{g} and R^{h} is independently selected from the group consisting of H, (C₁-C₆)alkyl, (C₃-C₆)cycloalkyl, (C₃-C₆)cycloalkyl(C₁-C₆)alkyl, (C₁-C₆)alkanolyl, a 3-10 membered heterocycle, —C(═NH)—NH₂, and (C₁-C₆)alkylsulfonyl, wherein any (C₁-C₆)alkyl, (C₃-C₆)cycloalkyl, (C₃-C₆)cycloalkyl(C₁-C₆)alkyl, (C₁-C₆)alkanolyl, 3-10 membered heterocycle, and (C₁-C₆)alkylsulfonyl is optionally substituted with one or more groups independently selected from halo, (C₁-C₆)alkyl, a 3-10 membered heterocycle and NRⁿR^{m}; and wherein any a 3-10 membered heterocycle is optionally substituted with one or more groups independently selected from the group consisting of halo and (C₁-C₆)alkyl; or R^{g} and R^{h} together with the nitrogen to which they are attached form a 3-10 membered heterocycle that is optionally substituted with one or more groups independently selected from the group consisting of halo, (C₁-C₆)alkyl, 3-10 membered heterocycle, and NRⁿR^{m};
each Rⁱ and R^{k} is independently selected from the group consisting of H, (C₁-C₆)alkyl, (C₃-C₆)cycloalkyl, (C₃-C₆)cycloalkyl(C₁-C₆)alkyl, —C(═NH)—NH₂, and (C₁-C₆)alkanolyl; and each R^{m} and Rⁿ is independently selected from the group consisting of H, (C₁-C₆)alkyl, (C₃-C₆)cycloalkyl, (C₃-C₆)cycloalkyl(C₁-C₆)alkyl, —C(═NH)—NH₂, and (C₁-C₆)alkanolyl.

The invention also provides a pharmaceutical composition comprising a compound of formula (I) or a pharmaceutically acceptable salt thereof and a pharmaceutically acceptable excipient.

The invention also provides a method for promoting an antiviral effect in an animal, comprising administering a compound of formula (I) or a pharmaceutically acceptable salt thereof to the animal.

The invention also provides a method for inhibiting a papain-like protease in an animal in need thereof, comprising administering a compound of formula (I) or a pharmaceutically acceptable salt thereof to the animal.

The invention also provides a compound of formula (I) or a pharmaceutically acceptable salt thereof for use in medical therapy.

The invention also provides a compound of formula (I) or a pharmaceutically acceptable salt thereof for the prophylactic or therapeutic treatment of a viral infection.

The invention also provides the use of a compound of formula (I) or a pharmaceutically acceptable salt thereof to prepare a medicament for treating a viral infection in an animal.

The invention also provides processes and intermediates disclosed herein that are useful for preparing a compound of formula I or a salt thereof.

### DETAILED DESCRIPTION

The following definitions are used, unless otherwise described: halo or halogen is fluoro, chloro, bromo, or iodo. Alkyl, alkoxy, etc. denote both straight and branched groups; but reference to an individual radical such as propyl embraces only the straight chain radical, a branched chain isomer such as isopropyl being specifically referred to.

The term “alkyl”, by itself or as part of another substituent, means, unless otherwise stated, a straight or branched chain hydrocarbon radical, having the number of carbon atoms designated (i.e., C₁₋₈ means one to eight carbons). Examples include (C₁-C₅)alkyl, (C₂-C₅)alkyl, C₁-C₆)alkyl, (C₂-C₆)alkyl and (C₃-C₆)alkyl. Examples of alkyl groups include methyl, ethyl, n-propyl, iso-propyl, n-butyl, t-butyl, iso-butyl, sec-butyl, n-pentyl, n-hexyl, n-heptyl, n-octyl, and and higher homologs and isomers.

The term “alkoxy” refers to an alkyl groups attached to the remainder of the molecule via an oxygen atom (“oxy”).

The term “cycloalkyl” refers to a saturated or partially unsaturated (non-aromatic) all carbon ring having 3 to 8 carbon atoms (i.e., (C₃-C₅)carbocycle). The term also includes multiple condensed, saturated all carbon ring systems (e.g., ring systems comprising 2, 3 or 4 carbocyclic rings). Accordingly, carbocycle includes multicyclic carbocyles such as a bicyclic carbocycles (e.g., bicyclic carbocycles having about 3 to 15 carbon atoms, about 6 to 15 carbon atoms, or 6 to 12 carbon atoms such as bicyclo[3.1.0]hexane and bicyclo[2.1.1]hexane), and polycyclic carbocycles (e.g tricyclic and tetracyclic carbocycles with up to about 20 carbon atoms). The rings of the multiple condensed ring system can be connected to each other via fused, spiro and bridged bonds when allowed by valency requirements. For example, multicyclic carbocyles can be connected to each other via a single carbon atom to form a spiro connection (e.g., spiropentane, spiro[4,5]decane, etc), via two adjacent carbon atoms to form a fused connection (e.g., carbocycles such as decahydronaphthalene, norsabinane, norcarane) or via two non-adjacent carbon atoms to form a bridged connection (e.g., norbornane, bicyclo[2.2.2]octane, etc). Non-limiting examples of cycloalkyls include cyclopropyl, cyclobutyl, cyclopentyl, cyclohexyl, bicyclo[2.2.1]heptane, pinane, and adamantane.

The term “aryl” as used herein refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, 6 to 12 carbon atoms, or 6 to 10 carbon atoms. Aryl includes a phenyl radical. Aryl also includes multiple condensed carbon ring systems (e.g., ring systems comprising 2, 3 or 4 rings) having about 9 to 20 carbon atoms in which at least one ring is aromatic and wherein the other rings may be aromatic or not aromatic (i.e., cycloalkyl. The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring. Non-limiting examples of aryl groups include, but are not limited to, phenyl, indenyl, indanyl, naphthyl, 1, 2, 3, 4-tetrahydronaphthyl, anthracenyl, and the like.

The term “heterocycle” refers to a single saturated or partially unsaturated ring that has at least one atom other than carbon in the ring, wherein the atom is selected from the group consisting of oxygen, nitrogen and sulfur; the term also includes multiple condensed ring systems that have at least one such saturated or partially unsaturated ring, which multiple condensed ring systems are further described below. Thus, the term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The sulfur and nitrogen atoms may also be present in their oxidized forms. Exemplary heterocycles include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more groups selected from cycloalkyl, aryl, and heterocycle to form the multiple condensed ring system. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heterocycle) can be at any position of the multiple condensed ring system including a heterocycle, aryl and carbocycle portion of the ring. In one embodiment the term heterocycle includes a 3-15 membered heterocycle. In one embodiment the term heterocycle includes a 3-10 membered heterocycle. In one embodiment the term heterocycle includes a 3-8 membered heterocycle. In one embodiment the term heterocycle includes a 3-7 membered heterocycle. In one embodiment the term heterocycle includes a 3-6 membered heterocycle. In one embodiment the term heterocycle includes a 4-6 membered heterocycle. In one embodiment the term heterocycle includes a 3-10 membered monocyclic or bicyclic heterocycle comprising 1 to 4 heteroatoms. In one embodiment the term heterocycle includes a 3-8 membered monocyclic or bicyclic heterocycle heterocycle comprising 1 to 3 heteroatoms. In one embodiment the term heterocycle includes a 3-6 membered monocyclic heterocycle comprising 1 to 2 heteroatoms. In one embodiment the term heterocycle includes a 4-6 membered monocyclic heterocycle comprising 1 to 2 heteroatoms. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, 1,4-benzodioxanyl, spiro[cyclopropane-1,1′-isoindolinyl]-3′-one, isoindolinyl-1-one, 2-oxa-6-azaspiro[3.3]heptanyl, imidazolidin-2-one imidazolidine, pyrazolidine, butyrolactam, valerolactam, imidazolidinone, hydantoin, dioxolane, phthalimide, and 1,4-dioxane. In one embodiment the term heterocycle includes a 3-8 membered monocyclic or bicyclic heterocycle comprising 1 to 3 nitrogen atoms. In one embodiment the term heterocycle includes a 3-8 membered monocyclic or bicyclic heterocycle comprising 1 to 2 nitrogen atoms. In one embodiment the term heterocycle includes a 3-8 membered monocyclic or bicyclic heterocycle comprising 1 nitrogen atom.

The term “heteroaryl” as used herein refers to a single aromatic ring that has at least one atom other than carbon in the ring, wherein the atom is selected from the group consisting of oxygen, nitrogen and sulfur, “heteroaryl” also includes multiple condensed ring systems that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl or furyl. “Heteroaryl” also includes multiple condensed ring systems (e.g., ring systems comprising 2, 3 or 4 rings) wherein a heteroaryl group, as defined above, is condensed with one or more rings selected from cycloalkyl, aryl, heterocycle, and heteroaryl. It is to be understood that the point of attachment for a heteroaryl or heteroaryl multiple condensed ring system can be at any suitable atom of the heteroaryl or heteroaryl multiple condensed ring system including a carbon atom and a heteroatom (e.g., a nitrogen). Exemplary heteroaryls include but are not limited to pyridyl, pyrrolyl, pyrazinyl, pyrimidinyl, pyridazinyl, pyrazolyl, thienyl, indolyl, imidazolyl, triazolyl, tetrazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, and quinazolyl. In one embodiment the heteroaryl is a 5-membered heteroaryl. In one embodiment the heteroaryl is a 6-membered heteroaryl. In one embodiment the heteroaryl is a 5-membered heteroaryl that comprises one or two heteroatoms selected from O, N, and S. In one embodiment the heteroaryl is a 6-membered heteroaryl that comprises one or two heteroatoms selected from O, N, and S.

As used herein, the term “heteroatom” includes oxygen (O), nitrogen (N), sulfur (S) and silicon (Si).

As used herein, the term “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include phenylsulfonylethyl, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see P. G. M. Wuts and T. W. Greene, Greene's Protective Groups in Organic Synthesis 4^{th} edition, Wiley-Interscience, New York, 2006.

As used herein a wavy line “ [symbol] ” that intersects a bond in a chemical structure indicates the point of attachment of the bond that the wavy bond intersects in the chemical structure to the remainder of a molecule.

The terms “treat”, “treatment”, or “treating” to the extent it relates to a disease or condition includes inhibiting the disease or condition, eliminating the disease or condition, and/or relieving one or more symptoms of the disease or condition. The terms “treat”, “treatment”, or “treating” also refer to both therapeutic treatment and/or prophylactic treatment or preventative measures, wherein the object is to prevent or slow down (lessen) an undesired physiological change or disorder, such as, for example, the development or spread of cancer. For example, beneficial or desired clinical results include, but are not limited to, alleviation of symptoms, diminishment of extent of disease or disorder, stabilized (i.e., not worsening) state of disease or disorder, delay or slowing of disease progression, amelioration or palliation of the disease state or disorder, and remission (whether partial or total), whether detectable or undetectable. “Treat”, “treatment”, or “treating,” can also mean prolonging survival as compared to expected survival if not receiving treatment. Those in need of treatment include those already with the disease or disorder as well as those prone to have the disease or disorder or those in which the disease or disorder is to be prevented. In one embodiment “treat”, “treatment”, or “treating” does not include preventing or prevention,

The phrase “therapeutically effective amount” or “effective amount” includes but is not limited to an amount of a compound of the that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “animal” includes mammals.

The term “mammal” as used herein refers to humans, higher non-human primates, rodents, domestic, cows, horses, pigs, sheep, dogs and cats. In one embodiment, the mammal is a human.

The term “patient” as used herein refers to any animal including mammals. In one embodiment, the patient is a mammalian patient In one embodiment, the patient is a human patient. In one embodiment, the mammal is a human.

The compounds disclosed herein can also exist as tautomeric isomers in certain cases. Although only one delocalized resonance structure may be depicted, all such forms are contemplated within the scope of the invention.

It is understood by one skilled in the art that this invention also includes any compound claimed that may be enriched at any or all atoms above naturally occurring isotopic ratios with one or more isotopes such as, but not limited to, deuterium (²H or D). As a non-limiting example, a —CH₃ group may be substituted with —CD3.

The pharmaceutical compositions of the invention can comprise one or more excipients. When used in combination with the pharmaceutical compositions of the invention the term “excipients” refers generally to an additional ingredient that is combined with the compound of formula (I) or the pharmaceutically acceptable salt thereof to provide a corresponding composition. For example, when used in combination with the pharmaceutical compositions of the invention the term “excipients” includes, but is not limited to: carriers, binders, disintegrating agents, lubricants, sweetening agents, flavoring agents, coatings, preservatives, and dyes.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted.

Specific values listed below for radicals, substituents, and ranges, are for illustration only; they do not exclude other defined values or other values within defined ranges for the radicals and substituents. It is to be understood that two or more values may be combined. It is also to be understood that the values listed herein below (or subsets thereof) can be excluded.

Specifically, (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₁-C₆)alkanoyl can be formyl, acetyl, propanoyl or butanoyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

A specific compound or salt of formula (I) is a compound of formula (Ia):

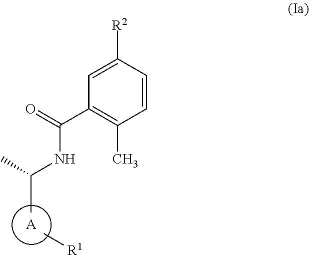

or a salt thereof A specific compound or salt of formula (I) is a compound of formula (Ib):

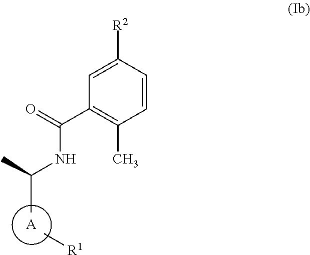

or a salt thereof.

In one embodiment, R¹ is halo.

In one embodiment, R¹ is NR^{a}R^{b}.

In one embodiment, R¹ is phenyl that is optionally substituted with one or more groups R^{x} independently selected from the group consisting of halo, hydroxy, cyano, (C₁-C₆)alkyl, (C₁-C₆)alkoxy, (C₃-C₆)cycloalkyl, NR^{a}R^{b}, oxo, 5-membered heteroaryl, 6-membered heterocycle, —C(═O)NR^{a}R^{b}, 4-membered heterocycle, 5-membered heterocycle, methylenedioxy, and (C₁-C₆)alkyl sulfonyl, wherein any R^{x} is optionally substituted with one or more groups R^{y} independently selected from the group consisting of halo, hydroxy, cyano, (C₁-C₆)alkyl, (C₁-C₆)alkoxy, NR^{c}R^{d}—C(═O)NR^{c}R^{d} (C₃-C₆)cycloalkyl, carboxy, 6-membered heterocycle, and phenyl, wherein any R^{y} is optionally substituted with one or more groups independently selected from the group consisting of hydroxy, (C₁-C₆)alkyl, (C₁-C₆)alkoxy, cyano, and halo.

In one embodiment, R¹ is a 5-membered heteroaryl that is optionally substituted with one or more groups R^{x} independently selected from the group consisting of halo, hydroxy, cyano, (C₁-C₆)alkyl, (C₁-C₆)alkoxy, (C₃-C₆)cycloalkyl, NR^{a}R^{b}, oxo, 5-membered heteroaryl, 6-membered heterocycle, —C(═O)NR^{a}R^{b}, 4-membered heterocycle, 5-membered heterocycle, methylenedioxy, and (C₁-C₆)alkyl sulfonyl, wherein any R^{x} is optionally substituted with one or more groups R^{y} independently selected from the group consisting of halo, hydroxy, cyano, (C₁-C₆)alkyl, (C₁-C₆)alkoxy, NR^{c}R^{d}—C(═O)NR^{c}R^{d} (C₃-C₆)cycloalkyl, carboxy, 6-membered heterocycle, and phenyl, wherein any R^{y} is optionally substituted with one or more groups independently selected from the group consisting of hydroxy, (C₁-C₆)alkyl, (C₁-C₆)alkoxy, cyano, and halo.

In one embodiment, R¹ is a 6-membered heteroaryl that is optionally substituted with one or more groups R^{x} independently selected from the group consisting of halo, hydroxy, cyano, (C₁-C₆)alkyl, (C₁-C₆)alkoxy, (C₃-C₆)cycloalkyl, NR^{a}R^{b}, oxo, 5-membered heteroaryl, 6-membered heterocycle, —C(═O)NR^{a}R^{b}, 4-membered heterocycle, 5-membered heterocycle, methylenedioxy, and (C₁-C₆)alkyl sulfonyl, wherein any R^{x} is optionally substituted with one or more groups R^{y} independently selected from the group consisting of halo, hydroxy, cyano, (C₁-C₆)alkyl, (C₁-C₆)alkoxy, NR^{c}R^{d}—C(═O)NR^{c}R^{d} (C₃-C₆)cycloalkyl, carboxy, 6-membered heterocycle, and phenyl, wherein any R^{y} is optionally substituted with one or more groups independently selected from the group consisting of hydroxy, (C₁-C₆)alkyl, (C₁-C₆)alkoxy, cyano, and halo.

In one embodiment, R¹ is a 5-membered heterocycle that is optionally substituted with one or more groups R^{x} independently selected from the group consisting of halo, hydroxy, cyano, (C₁-C₆)alkyl, (C₁-C₆)alkoxy, (C₃-C₆)cycloalkyl, NR^{a}R^{b}, oxo, 5-membered heteroaryl, 6-membered heterocycle, —C(═O)NR^{a}R^{b}, 4-membered heterocycle, 5-membered heterocycle, methylenedioxy, and (C₁-C₆)alkyl sulfonyl, wherein any R^{x} is optionally substituted with one or more groups R^{y} independently selected from the group consisting of halo, hydroxy, cyano, (C₁-C₆)alkyl, (C₁-C₆)alkoxy, NR^{c}R^{d}—C(═O)NR^{c}R^{d} (C₃-C₆)cycloalkyl, carboxy, 6-membered heterocycle, and phenyl, wherein any R^{y} is optionally substituted with one or more groups independently selected from the group consisting of hydroxy, (C₁-C₆)alkyl, (C₁-C₆)alkoxy, cyano, and halo.

In one embodiment, R¹ is a 6-membered heterocycle that is optionally substituted with one or more groups R^{x} independently selected from the group consisting of halo, hydroxy, cyano, (C₁-C₆)alkyl, (C₁-C₆)alkoxy, (C₃-C₆)cycloalkyl, NR^{a}R^{b}, oxo, 5-membered heteroaryl, 6-membered heterocycle, —C(═O)NR^{a}R^{b}, 4-membered heterocycle, 5-membered heterocycle, methylenedioxy, and (C₁-C₆)alkyl sulfonyl, wherein any R^{x} is optionally substituted with one or more groups R^{y} independently selected from the group consisting of halo, hydroxy, cyano, (C₁-C₆)alkyl, (C₁-C₆)alkoxy, NR^{c}R^{d}—C(═O)NR^{c}R^{d} (C₃-C₆)cycloalkyl, carboxy, 6-membered heterocycle, and phenyl, wherein any R^{y} is optionally substituted with one or more groups independently selected from the group consisting of hydroxy, (C₁-C₆)alkyl, (C₁-C₆)alkoxy, cyano, and halo.

In one embodiment, R¹ is a 9-membered bicyclic heterocycle that is optionally substituted with one or more groups R^{x} independently selected from the group consisting of halo, hydroxy, cyano, (C₁-C₆)alkyl, (C₁-C₆)alkoxy, (C₃-C₆)cycloalkyl, NR^{a}R^{b}, oxo, 5-membered heteroaryl, 6-membered heterocycle, —C(═O)NR^{a}R^{b}, 4-membered heterocycle, 5-membered heterocycle, methylenedioxy, and (C₁-C₆)alkyl sulfonyl, wherein any R^{x} is optionally substituted with one or more groups R^{y} independently selected from the group consisting of halo, hydroxy, cyano, (C₁-C₆)alkyl, (C₁-C₆)alkoxy, NR^{c}R^{d}—C(═O)NR^{c}R^{d} (C₁-C₆)cycloalkyl, carboxy, 6-membered heterocycle, and phenyl, wherein any R^{y} is optionally substituted with one or more groups independently selected from the group consisting of hydroxy, (C₁-C₆)alkyl, (C₁-C₆)alkoxy, cyano, and halo.

In one embodiment, R¹ is a 9-membered bicyclic heteroaryl that is optionally substituted with one or more groups R^{x} independently selected from the group consisting of halo, hydroxy, cyano, (C₁-C₆)alkyl, (C₁-C₆)alkoxy, (C₃-C₆)cycloalkyl, NR^{a}R^{b}, oxo, 5-membered heteroaryl, 6-membered heterocycle, —C(═O)NR^{a}R^{b}, 4-membered heterocycle, 5-membered heterocycle, methylenedioxy, and (C₁-C₆)alkyl sulfonyl, wherein any R^{x} is optionally substituted with one or more groups R^{y} independently selected from the group consisting of halo, hydroxy, cyano, (C₁-C₆)alkyl, (C₁-C₆)alkoxy, NR^{c}R^{d}—C(═O)NR^{c}R^{d} (C₃-C₆)cycloalkyl, carboxy, 6-membered heterocycle, and phenyl, wherein any R^{y} is optionally substituted with one or more groups independently selected from the group consisting of hydroxy, (C₁-C₆)alkyl, (C₁-C₆)alkoxy, cyano, and halo.

In one embodiment, R¹ is selected from the group consisting of chloro,

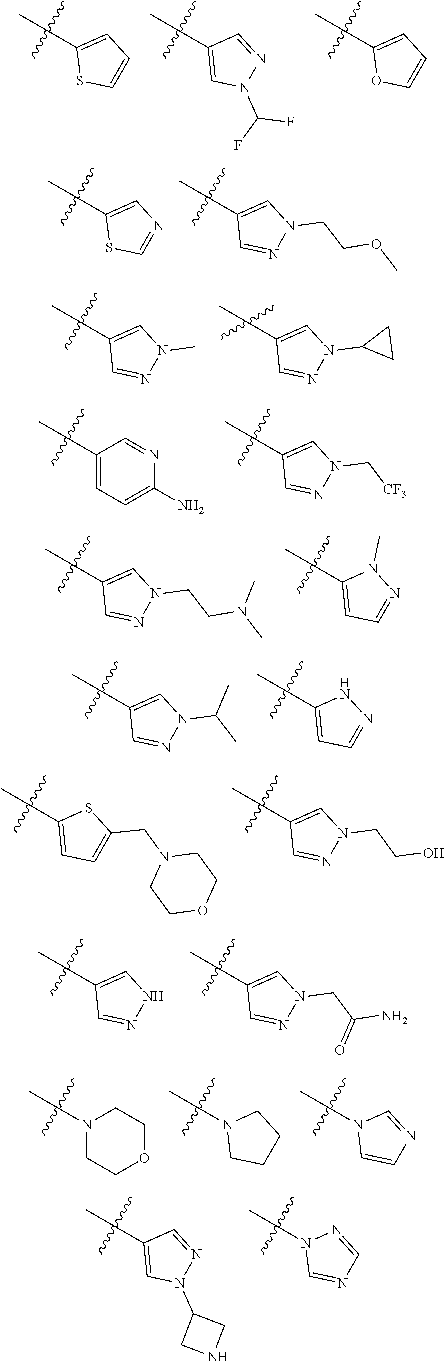

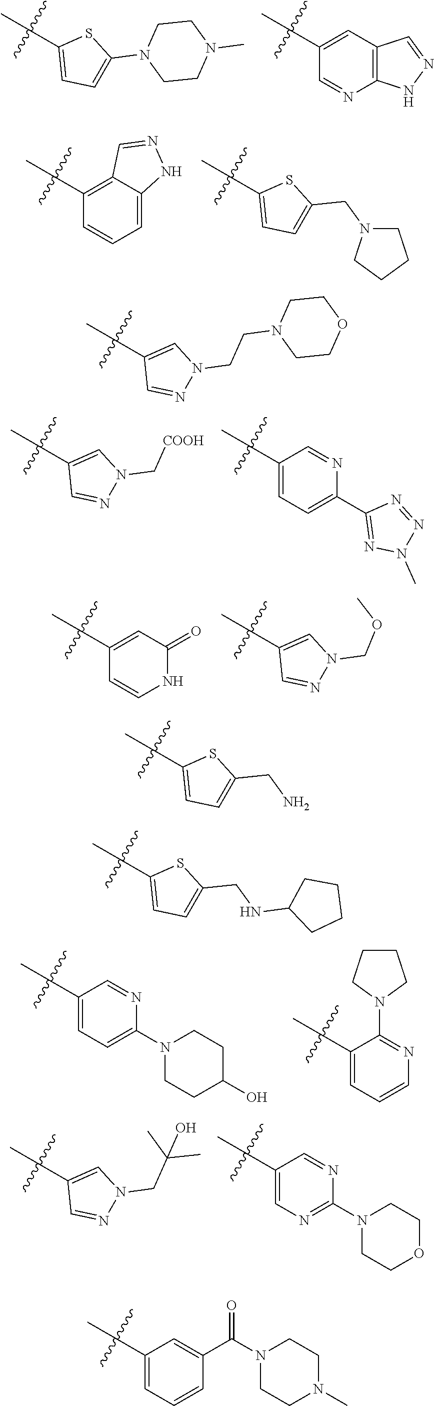

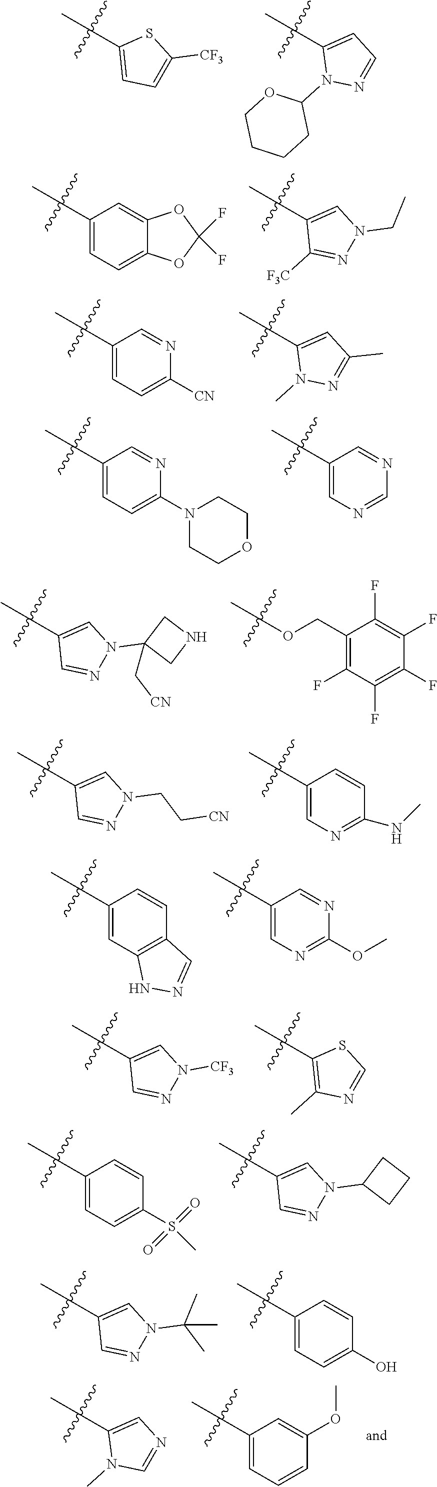

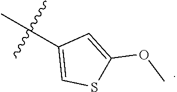 [SMILES: *c1csc(OC)c1]

In one embodiment, R¹ is selected from the group consisting of:

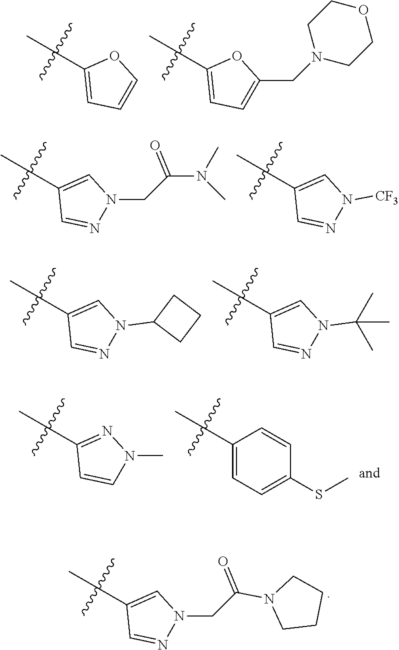

In one embodiment, R² is —OR^{e}.

In one embodiment, R² is —SR^{e}.

In one embodiment, R² is —NR^{g}R^{h}.

In one embodiment, R² is —OCH₂CH₂N(CH₃)₂.

In one embodiment, R² is selected from the group consisting of amino, methylamino, dimethylamino, 2-aminoethoxy,

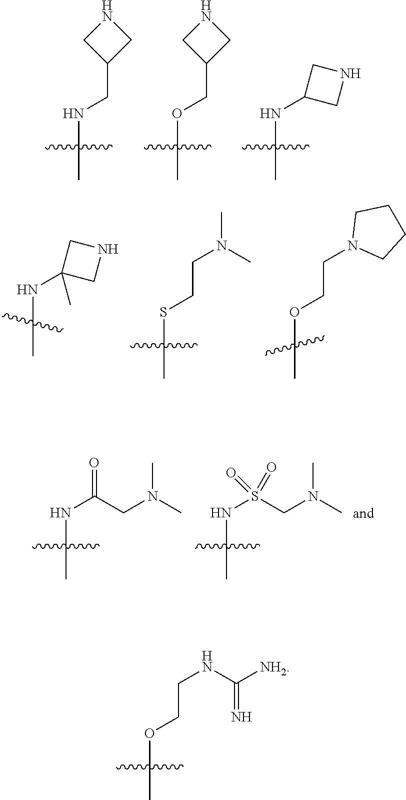

In one embodiment, R² is selected from the group consisting of

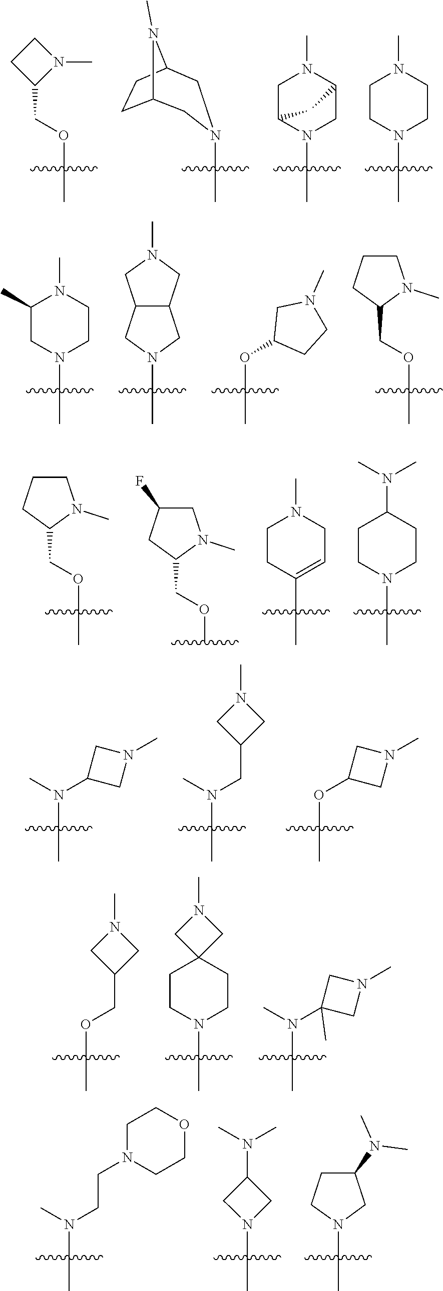

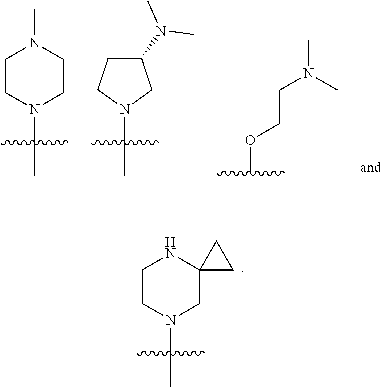

In one embodiment, A is a 9-membered bicyclic heteroaryl that is optionally substituted with one or more groups independently selected from the group consisting of halo, cyano, (C₁-C₆)alkyl, (C₁-C₆)alkoxy, (C₃-C₆)cycloalkyl, (C₁-C₆)alkoxycarbonyl, and (C₁-C₆)alkanoyloxy, wherein any (C₁-C₆)alkyl, (C₁-C₆)alkoxy, (C₃-C₆)cycloalkyl, (C₁-C₆)alkoxycarbonyl, and (C₁-C₆)alkanoyloxy is optionally substituted with one or more groups independently selected from halo.

In one embodiment, A is a 90-membered bicyclic heteroaryl that is optionally substituted with one or more groups independently selected from the group consisting of halo, cyano, (C₁-C₆)alkyl, (C₁-C₆)alkoxy, (C₃-C₆)cycloalkyl, (C₁-C₆)alkoxycarbonyl, and (C₁-C₆)alkanoyloxy, wherein any (C₁-C₆)alkyl, (C₁-C₆)alkoxy, (C₃-C₆)cycloalkyl, (C₁-C₆)alkoxycarbonyl, and (C₁-C₆)alkanoyloxy is optionally substituted with one or more groups independently selected from halo.

In one embodiment, A is a 9-membered bicyclic heteroaryl.

In one embodiment, A is a 10-membered bicyclic heteroaryl.

In one embodiment, A is:

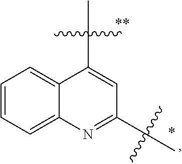 [SMILES: *c1cc(*)c2ccccc2n1]

wherein * designates the point of attachment to R¹ and ** designates the point of attachment to:

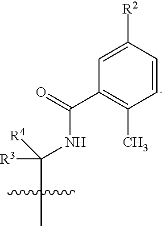

In one embodiment, A is selected from the group consisting of:

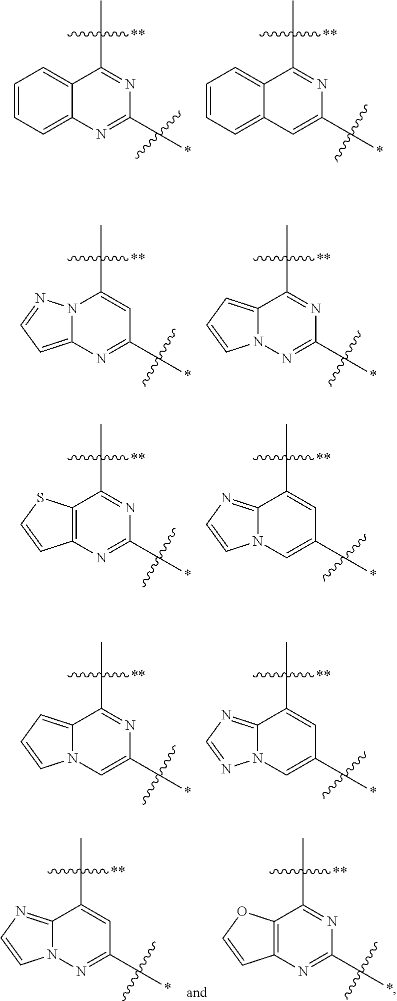

wherein * designates the point of attachment to R¹ and ** designates the point of attachment to:

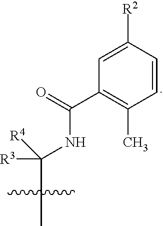

In one embodiment, A is:

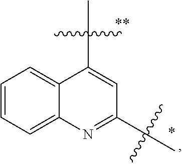 [SMILES: *c1cc(*)c2ccccc2n1]

wherein * designates the point of attachment to R¹ and ** designates the point of attachment to:

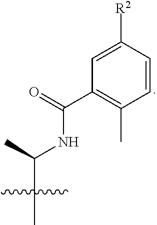

In one embodiment, A is selected from the group consisting of:

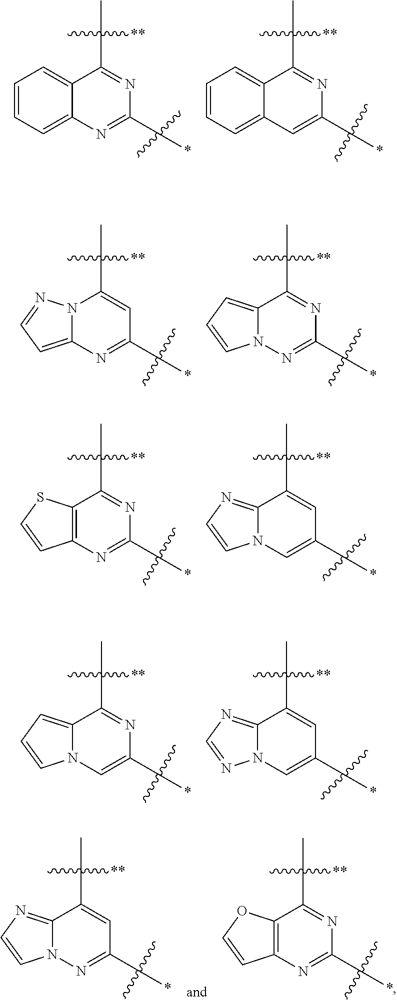

wherein * designates the point of attachment to R¹ and * * designates the point of attachment to:

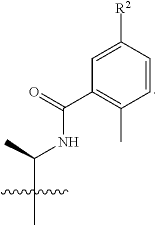

In one embodiment, a compound of formula (I) or salt thereof, wherein:
R¹ is halo, NR^{a}R^{b}, phenyl, benzyloxy, a 5-membered heteroaryl, a 6-membered heteroaryl, a 5-membered heterocycle, a 6-membered heterocycle, a 9-membered bicyclic heterocycle, or a 9-membered bicyclic heteroaryl, which phenyl, benzyloxy, 5-membered heteroaryl, 6-membered heteroaryl, 5-membered heterocycle, 6-membered heterocycle, 9-membered bicyclic heterocycle, and 9-membered bicyclic heteroaryl is optionally substituted with one or more groups R^{x} independently selected from the group consisting of halo, hydroxy, cyano, (C₁-C₆)alkyl, (C₁-C₆)alkoxy, (C₃-C₆)cycloalkyl, NR^{a}R^{b}, oxo, 5-membered heteroaryl, 6-membered heterocycle, —C(═O)NR^{a}R^{b}, 4-membered heterocycle, 5-membered heterocycle, methylenedioxy, and (C₁-C₆)alkyl sulfonyl, wherein any R^{x} is optionally substituted with one or more groups R^{y} independently selected from the group consisting of halo, hydroxy, cyano, (C₁-C₆)alkyl, (C₁-C₆)alkoxy, N^{c}R^{d}—C(═O)NR^{c}R^{d} (C₃-C₆)cycloalkyl, carboxy, 6-membered heterocycle, and phenyl, wherein any R^{y} is optionally substituted with one or more groups independently selected from the group consisting of hydroxy, (C₁-C₆)alkyl, (C₁-C₆)alkoxy, cyano, and halo;
A is a 9-membered bicyclic heteroaryl or a 10-membered bicyclic heteroaryl, which 9-membered bicyclic heteroaryl and 10-membered bicyclic heteroaryl is optionally substituted with one or more groups independently selected from the group consisting of halo, cyano, (C₁-C₆)alkyl, (C₁-C₆)alkoxy, (C₁-C₆)cycloalkyl, (C₁-C₆)alkoxycarbonyl, and (C₁-C₆)alkanoyloxy, wherein any (C₁-C₆)alkyl, (C₁-C₆)alkoxy, (C₃-C₆)cycloalkyl, (C₁-C₆)alkoxycarbonyl, and (C₁-C₆)alkanoyloxy is optionally substituted with one or more groups independently selected from halo;
R² is —OR^{e}, —SR^{e}, or —NR^{g}R^{h},
each R^{a} and R^{b} is independently selected from the group consisting of H, (C₁-C₆)alkyl, (C₃-C₆)cycloalkyl, (C₃-C₆)cycloalkyl(C₁-C₆)alkyl, and (C₁-C₆)alkanolyl; or R^{a} and R^{b} together with the nitrogen to which they are attached form a aziridino, azetidino, morpholino, piperazino, pyrrolidino or piperidino, which a aziridino, azetidino, morpholino, piperazino, pyrrolidino and piperidino, is optionally substituted with (C₁-C₆)alkyl;
each R^{c} and R^{d} is independently selected from the group consisting of H, (C₁-C₆)alkyl, (C₃-C₆)cycloalkyl, (C₃-C₆)cycloalkyl(C₁-C₆)alkyl, and (C₁-C₆)alkanolyl; or R^{c} and R^{d} together with the nitrogen to which they are attached form a aziridino, azetidino, morpholino, piperazino, pyrrolidino or piperidino, which a aziridino, azetidino, morpholino, piperazino, pyrrolidino and piperidino, is optionally substituted with (C₁-C₆)alkyl;
R^{e} is (C₁-C₆)alkyl, that is substituted with a 4-membered heterocycle or NRⁱR^{k};
each R^{g} and R^{h} is independently selected from the group consisting of H, (C₁-C₆)alkyl, (C₁-C₆)cycloalkyl, (C₁-C₆)cycloalkyl(C₁-C₆)alkyl, (C₁-C₆)alkanolyl, 4-membered heterocycle, —C(═NH)—NH₂, and (C₁-C₆)alkylsulfonyl, wherein any (C₁-C₆)alkyl, (C₃-C₆)cycloalkyl, (C₃-C₆)cycloalkyl(C₁-C₆)alkyl, (C₁-C₆)alkanolyl, and (C₁-C₆)alkylsulfonyl is optionally substituted with one or more groups independently selected from 4-membered heterocycle and NRⁿR^{m}; or R^{g} and R^{h} together with the nitrogen to which they are attached form a aziridino, azetidino, morpholino, piperazino, pyrrolidino or piperidino, which a aziridino, azetidino, morpholino, piperazino, pyrrolidino and piperidino, is optionally substituted with (C₁-C₆)alkyl;
each Rⁱ and R^{k} is independently selected from the group consisting of H, (C₁-C₆)alkyl, (C₃-C₆)cycloalkyl, (C₃-C₆)cycloalkyl(C₁-C₆)alkyl, —C(═NH)—NH₂, and (C₁-C₆)alkanolyl; or Rand R^{k} together with the nitrogen to which they are attached form a aziridino, azetidino, morpholino, piperazino, pyrrolidino or piperidino, which a aziridino, azetidino, morpholino, piperazino, pyrrolidino and piperidino, is optionally substituted with (C₁-C₆)alkyl; and
each R^{m} and Rⁿ is independently selected from the group consisting of H, (C₁-C₆)alkyl, (C₃-C₆)cycloalkyl, (C₃-C₆)cycloalkyl(C₁-C₆)alkyl, —C(═NH)—NH₂, and (C₁-C₆)alkanolyl; or R^{m} and Rⁿ together with the nitrogen to which they are attached form a aziridino, azetidino, morpholino, piperazino, pyrrolidino or piperidino, which a aziridino, azetidino, morpholino, piperazino, pyrrolidino and piperidino, is optionally substituted with (C₁-C₆)alkyl is provided.

In cases where compounds are sufficiently basic or acidic, a salt of a compound of formula I can be useful as an intermediate for isolating or purifying a compound of formula I. Additionally, administration of a compound of formula I as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds of formula I can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Compounds of the invention can also be administered in combination with other therapeutic agents, for example, other agents that are useful for the treatment of viral infections (e.g., protease inhibitors). Examples of such agents include nirmatrelvir, ensitrelvir, molnupiravir, VV116, and remdesivir. Accordingly, in one embodiment the invention also provides a composition comprising a compound of formula I, or a pharmaceutically acceptable salt thereof, at least one other therapeutic agent, and a pharmaceutically acceptable diluent or carrier. The invention also provides a kit comprising a compound of formula I, or a pharmaceutically acceptable salt thereof, at least one other therapeutic agent, packaging material, and instructions for administering the compound of formula I or the pharmaceutically acceptable salt thereof and the other therapeutic agent or agents to an animal to treat a viral infection.

The invention will now be illustrated by the following non-limiting Examples.

### EXAMPLES

### General Synthetic Procedure Scheme 1

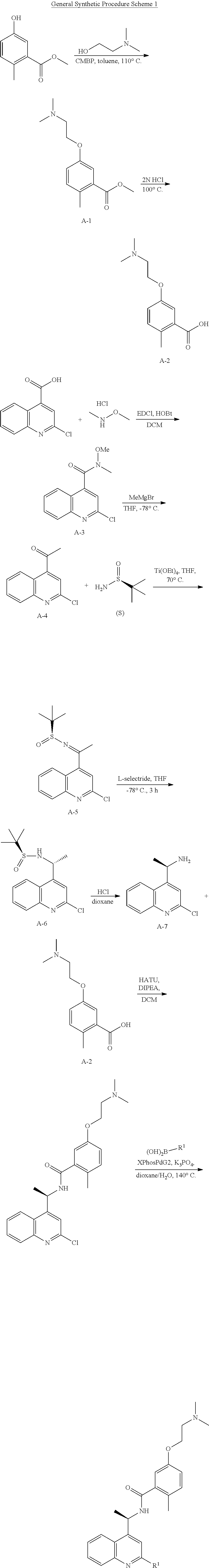

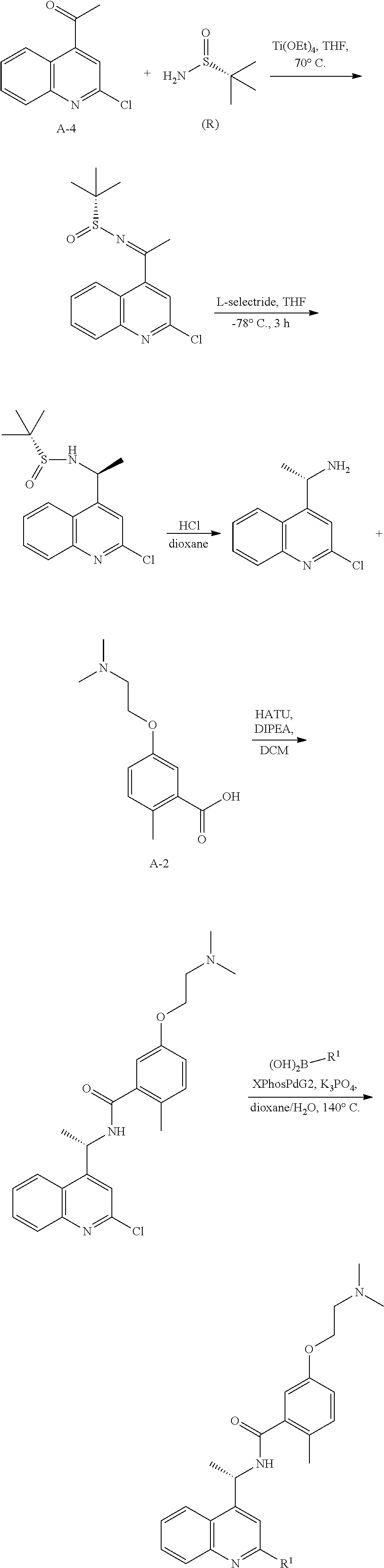

### Synthetic Procedures for Scheme 1

### General Procedures for Mitsonobu Reaction.

To a solution of compound methyl 5-hydroxy-2-methylbenzoate (1.0 equiv) in dry toluene in a sealed tube 2-(dimethylamino)ethan-1-ol (1.5 equiv) and cyanomethylene trimethyl-phosphorane (CMMP) (2.0 equiv) were added. The reaction mixture was degassed and purged with argon, and then the tube was sealed and heated to 110° C. overnight. When the starting material was consumed entirely as monitored by LCMS, the mixture was diluted with EtOAc and washed with water and brine. The organic layer was dried over Na₂SO₄, filtered, and concentrated. The residue was purified by silica gel column chromatography (DCM/MeOH=20:1) to provide the desired product A-1 for the next step without purification.

### General Procedures for Ester Hydrolysis.

A solution of methyl 5-(2-(dimethylamino)ethoxy)-2-methylbenzoate A-1 (4.00 g, 16.8 mmol, 1.0 equiv) in HCl (2 M aqueous, 30 mL) was stirred at 110° C. for 16 h. LCMS indicated that the starting material completely consumed, and the desired product was detected. The reaction mixture was allowed to cool to room temperature, basified to pH 6 using NaOH (2 M aqueous) the mixture was concentrated under vacuum to give the desired product 5-(2-(dimethylamino)ethoxy)-2-methylbenzoic acid (A-2, 3.8 g, 90%) as a white solid.

### Preparation of 5-(2-(dimethylamino)ethoxy)-2-methylbenzoic acid (A-2)

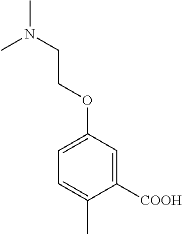 [SMILES: Cc1ccc(OCCN(C)C)cc1C(=O)O]

Prepared as described in the General procedure for ester hydrolysis above. White solid, 90% yield, ¹H NMR (400 MHz, CD3OD) δ 7.03 (d, J=2.8 Hz, 1H), 6.94 (d, J=8.4 Hz, 1H), 6.72 (dd, J=8.4, 2.8 Hz, 1H), 4.10 (t, J=4.9 Hz, 2H), 3.31 (t, J=5.0 Hz, 2H), 2.71 (s, 6H), 2.24 (s, 3H). ¹³C NMR (101 MHz, CD3OD) δ 175.99, 156.81, 132.84, 130.39, 116.50, 115.35, 63.65, 57.41, 43.86, 20.10 LCMS calcd for C₁₅H₁₈NO₃ (M+H⁺): 224.1208; Found: 224.1209.

### Preparation of 3-bromo-5-chloro-N-methoxy-N-methylbenzamide. (A-3)

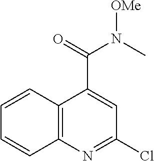 [SMILES: CON(C)C(=O)c1cc(Cl)nc2ccccc12]

To a solution of 2-chloroquinoline-4-carboxylic acid (1.0 equiv), N,O-dimethylhydroxylamine hydrochloride (1.2 equiv), HOBt (1.3 equiv) and EDCI (1.3 equiv) in DCM (0.1 M) was added dropwise of triethylamine (3.0 equiv) at 0° C. The mixture was stirred at rt for 24 hours. After the reaction was completed, the organic layer was washed with water and brine, dried over Na₂SO₄, filtered, and concentrated under vacuum to give the product A-3 for the next step without purification.

### Preparation of 1-(2-chloroquinolin-4-yl)ethan-1-one (A-4)

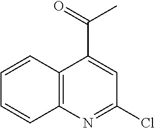

To a solution of 2-chloro-N-methoxy-N-methylquinoline-4-carboxamide (A-3, 1.0 equiv) in dry ether at −78° C. under N₂, the methyl magnesium bromide (3 M solution in diethyl ether, 2.0 equiv) was added slowly. After warming to room temperature and stirred for 2 hours, the reaction mixture was quenched with ice-cold saturated NH₄Cl and washed with water and brine, dried over Na₂SO₄, filtered, and concentrated under vacuum to give the crude residue, which was purified by silica gel column chromatography (Hexanes/EtOAc=5:1) to provide the desired product A-4. 1-(2-chloroquinolin-4-yl)ethan-1-one (A-4) White solid, 75% yield for two steps. ¹H NMR (400 MHz, CDCl₃) δ 8.48-8.33 (m, 1H), 8.07 (dd, J=8.5, 1.3 Hz, 1H), 7.79 (ddd, J=8.5, 6.9, 1.4 Hz, 1H), 7.65 (ddd, J=8.4, 6.9, 1.3 Hz, 1H), 7.60 (s, 1H), 2.75 (s, 3H). ¹³C NMR (101 MHz, CDCl₃) δ 199.72, 150.10, 148.96, 145.72, 131.03, 129.08, 128.49, 125.56, 122.55, 121.27, 30.07. LCMS calcd for C₁₁H₉ClNO (M+H⁺): 206.0294; Found: 206.0297.

### Preparation of (S)—N-(1-(2-chloroquinolin-4-yl)ethylidene)-2-methylpropane-2-sulfinamide (A-5)

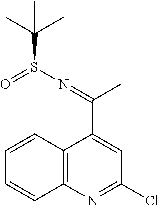 [SMILES: CC(=N[S@@](=O)C(C)(C)C)c1cc(Cl)nc2ccccc12]

To a solution of 1-(2-chloroquinolin-4-yl)ethan-1-one (20.0 g, 97.5 mmol) in toluene (500.0 mL) under N₂ were added (S)-2-methylpropane-2-sulfinamide (35.4 g, 292.5 mmol) and Ti(O-iPr)₄ (110.9 g, 390.0 mmol) at 20° C. The reaction mixture was stirred at 100° C. under N₂ for 15 h. LCMS showed a peak with a desired mass. The reaction mixture was then evaporated in vacuo to afford a crude, which was purified by silica gel chromatography using a Biotage (330 g column, petroleum ether/ethyl acetate=1/0 to 0/1) to afford (A-5, 27.3 g) as a yellow oil.

### Preparation of (S)—N—((R)-1-(2-chloroquinolin-4-yl)ethyl)-2-methylpropane-2-sulfinamide (A-6)

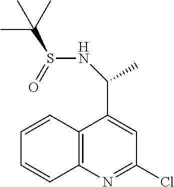 [SMILES: C[C@@H](N[S@@](=O)C(C)(C)C)c1cc(Cl)nc2ccccc12]

To a solution of (S)—N-(1-(2-chloroquinolin-4-yl)ethylidene)-2-methylpropane-2-sulfinamide (27.3 g, 88.61 mmol) in THF (500 mL) under N₂, was added L-selectride (159.5 mL, 1 M, 159.5 mmol) dropwise at −78° C. The reaction mixture was stirred at −78° C. under N₂ for 5 h before being quenched by 100 mL sat. aq. NH₄Cl at 0° C. The reaction mixture was then partitioned between ethyl acetate and water (500/500 mL). The organic layer was separated and the aqueous layer was re-extracted with ethyl acetate (500 mL). The combined organic layer was washed with brine, dried and evaporated in vacuo before being purified by silica gel chromatography using a Biotage (330 g column, petroleum ether/ethyl acetate=1/0 to 0/1, ethyl acetate/methanol=1/0 to 0/1) to afford (A-6, 20.6 g). White solid, 75% yield. ¹H NMR (400 MHz, CDCl₃) δ 8.10-7.97 (m, 2H), 7.74 (ddd, J=8.2, 6.8, 1.3 Hz, 1H), 7.58 (ddd, J=8.5, 6.9, 1.3 Hz, 1H), 7.47 (s, 1H), 5.29 (qd, J=6.6, 2.9 Hz, 1H), 3.80 (d, J=3.0 Hz, 1H), 1.66 (d, J=6.6 Hz, 3H), 1.28 (s, 9H). ¹³C NMR (101 MHz, CDCl₃) δ 152.39, 150.86, 148.29, 130.56, 129.56, 127.35, 124.21, 122.86, 119.19, 55.98, 48.51, 22.64, 21.89. LCMS calcd for C₁₅H₂₀ClN₂OS (M+H⁺): 311.0907; Found: 311.0909.

### Preparation of (R)-1-(2-chloroquinolin-4-yl)ethan-1-amine (A-7)

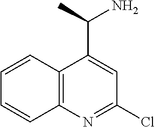

To a solution of (S)—N—((R)-1-(2-chloroquinolin-4-yl)ethyl)-2-methylpropane-2-sulfinamide (14.5 g, 46.7 mmol) in MeOH (100 mL) was added HCl/MeOH (58.4 mL, 233.5 mmol, 4M in MeOH). The reaction was stirred at 20° C. for 8 h. TLC (petroleum ether/ethyl acetate=3/1) showed that starting material was consumed and a new spot was detected. The mixture was filtered and the filter cake was dissolved in water (20 mL). The solution was basified with sat. aq. NaHCO₃ to pH=8 and extracted with dichloromethane/MeOH (v/v=10/1, 5 Å˜200 mL). The organic layers were combined, dried over Na₂SO₄, filtered, evaporated under reduced pressure to A-7 as a yellow liquid, which was used for the next step without further purification.

### Preparation of (R)—N-(1-(2-chloroquinolin-4-yl)ethyl)-5-(2-(dimethylamino)-ethoxy)-2-methylbenzamide

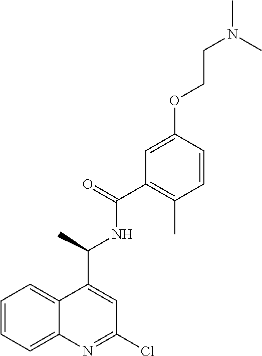 [SMILES: Cc1ccc(OCCN(C)C)cc1C(=O)N[C@H](C)c1cc(Cl)nc2ccccc12]

5-(2-(dimethylamino)ethoxy)-2-methylbenzoic acid (1.1 equiv), HATU (1.1 equiv) and DIPEA (3.0 equiv) were dissolved in DMF (0.2 M) and stirred for 15 min. After that ((R)-1-(2-chloroquinolin-4-yl)ethan-1-amine (A-9, 1.0 equiv) was added and stirred at room temperature overnight. When the starting material was consumed entirely as monitored by LCMS, the mixture was diluted with EtOAc and was then washed with saturated aq. NaHCO₃, water, and brine. The organic layer was dried over Na₂SO₄, filtered and concentrated under vacuum to give a residue which was purified by Prep-HPLC to give the final product. White solid, 61% yield. ¹H NMR (400 MHz, CD3OD) δ 8.33 (d, J=8.4 Hz, 1H), 7.99 (d, J=8.4 Hz, 1H), 7.91-7.78 (m, 1H), 7.71 (ddd, J=8.3, 6.9, 1.3 Hz, 1H), 7.56 (s, 1H), 7.19-7.13 (m, 1H), 6.97-6.90 (m, 2H), 5.96 (q, J=7.0 Hz, 1H), 4.11 (t, J=5.3 Hz, 2H), 2.81 (t, J=5.4 Hz, 2H), 2.37 (s, 6H), 2.27 (s, 3H), 1.68 (d, J=7.0 Hz, 3H). ¹³C NMR (101 MHz, CD3OD) δ 170.52, 156.52, 154.15, 150.60, 147.90, 136.81, 131.56, 130.56, 128.31, 127.50, 127.17, 124.77, 123.43, 118.25, 115.65, 113.06, 65.15, 57.57, 45.04, 44.32, 19.70, 17.44. LCMS calcd for C₂₃H₂₇ClN₃O₂ (M+H⁺): 412.1714; Found: 412.1717.

### General Procedures for the Suzuki Coupling II.

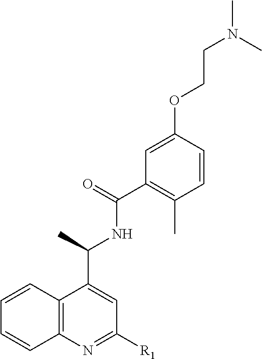

To a solution of compound (R)—N-(1-(2-chloroquinolin-4-yl)ethyl)-5-(2-(dimethylamino)-ethoxy)-2-methylbenzamide (1.0 equiv) in dioxane/H₂O (4:1) in a microwave reaction vial was added corresponding boronic acid or ester (1.2 equiv) and potassium carbonate (1.8 equiv). The mixture was purged with nitrogen for 5 min. Then Pd(dppf)Cl₂ (0.1 equiv) was added, and the solution was heated in the biotage microwave reactor at 130° C. for 90 min. The reaction mixture was diluted with EtOAc and extracted with aqueous NaHCO₃ solution and brine. The organic layer was separated, dried over anhydrous Na₂SO₄, filtered, and concentrated under vacuum to give a residue which was purified by Prep-HPLC to give the final product.

### Example 1. Synthesis of (R)-5-(2-(dimethylamino)ethoxy)-2-methyl-N-(1-(2-(thiophen-2-yl)quinolin-4-yl)ethyl)benzamide

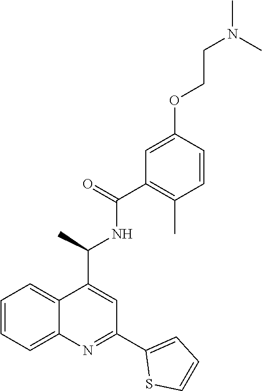 [SMILES: Cc1ccc(OCCN(C)C)cc1C(=O)N[C@H](C)c1cc(-c2cccs2)nc2ccccc12]

(R)-5-(2-(dimethylamino)ethoxy)-2-methyl-N-(1-(2-(thiophen-2-yl)quinolin-4-yl)ethyl) benzamide. White solid, 72% yield. ¹H NMR (400 MHz, CD₃OD) δ 8.25 (d, J=8.5 Hz, 1H), 8.14-8.00 (m, 2H), 7.85 (d, J=3.7 Hz, 1H), 7.76 (t, J=7.7 Hz, 1H), 7.61 (t, J=6.7 Hz, 2H), 7.23-7.12 (m, 2H), 6.96 (dt, J=8.1, 2.8 Hz, 2H), 6.02 (q, J=7.0 Hz, 1H), 4.11 (t, J=5.3 Hz, 2H), 2.86 (t, J=5.3 Hz, 2H), 2.41 (s, 6H), 2.29 (s, 3H), 1.72 (d, J=7.0 Hz, 3H). ¹³C NMR (101 MHz, CD₃OD) δ 170.56, 156.45, 152.28, 150.66, 148.21, 144.72, 137.15, 131.52, 129.62, 129.12, 128.56, 127.86, 127.49, 126.16, 126.09, 124.96, 122.98, 115.71, 113.78, 112.89, 64.89, 57.45, 45.04, 44.14, 19.91, 17.39. LCMS calcd for C₂₇H₃₀N₃O₂S (M+H⁺): 460.6080; Found: 460.6.

### Example 2. Synthesis of (S)-5-(2-(dimethylamino)ethoxy)-2-methyl-N-(1-(2-(thiophen-2-yl)quinolin-4-yl)ethyl)benzamide

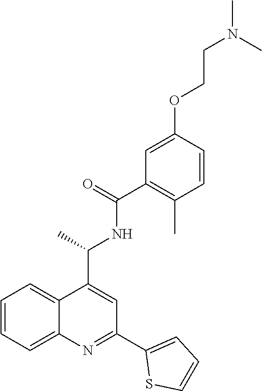 [SMILES: Cc1ccc(OCCN(C)C)cc1C(=O)N[C@@H](C)c1cc(-c2cccs2)nc2ccccc12]

White solid, δ ¹H NMR (400 MHz, CD₃OD) δ 8.24 (d, J=8.5 Hz, 1H), 8.10-7.98 (m, 2H), 7.85 (d, J=3.7 Hz, 1H), 7.80-7.71 (m, 1H), 7.61 (t, J=6.8 Hz, 2H), 7.27-7.09 (m, 2H), 7.01-6.90 (m, 2H), 6.01 (q, J=7.0 Hz, 1H), 4.10 (t, J=5.4 Hz, 2H), 2.82 (t, J=5.4 Hz, 2H), 2.38 (s, 6H), 2.28 (s, 3H), 1.72 (d, J=7.0 Hz, 3H). ¹³C NMR (101 MHz, CD₃OD) δ 170.58, 156.49, 152.29, 150.68, 148.21, 144.71, 137.12, 131.52, 129.62, 129.11, 128.56, 127.87, 127.46, 126.16, 126.11, 124.96, 122.98, 115.72, 113.80, 112.90, 65.07, 57.51, 45.05, 44.24, 19.92, 17.39. LCMS calcd for C₂₇H₃₀N₃O₂S (M+H⁺): 460.6080; Found: 460.1.

### Example 3. Synthesis of (R)—N-(1-(2-(1-(difluoromethyl)-1H-pyrazol-4-yl)quinolin-4-yl)ethyl)-5-(2-(dimethylamino)ethoxy)-2-methylbenzamide

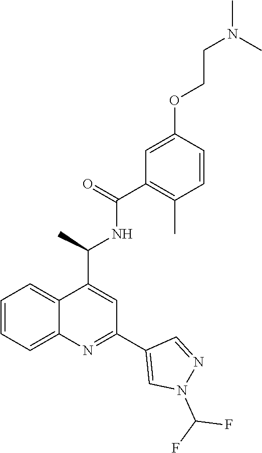 [SMILES: Cc1ccc(OCCN(C)C)cc1C(=O)N[C@H](C)c1cc(-c2cnn(C(F)F)c2)nc2ccccc12]

White solid ¹H NMR (400 MHz, CD₃OD) δ 8.79 (s, 1H), 8.41 (s, 1H), 8.29 (d, J=8.3 Hz, 1H), 8.09 (d, J=8.5 Hz, 1H), 7.94 (s, 1H), 7.81-7.72 (m, 1H), 7.66-7.61 (m, 1H), 7.19-7.12 (m, 1H), 6.95 (d, J=7.1 Hz, 2H), 6.03 (q, J=7.0 Hz, 1H), 4.13 (t, J=5.3 Hz, 2H), 2.94 (t, J=5.2 Hz, 2H), 2.47 (s, 6H), 2.26 (s, 3H), 1.73 (d, J=7.0 Hz, 3H). ¹³C NMR (101 MHz, CD₃OD) δ 170.50, 156.35, 151.03, 150.50, 148.18, 140.56, 137.12, 131.54, 131.49, 129.75, 128.86, 127.61, 126.78, 126.37, 125.70, 125.00, 123.11, 115.67, 115.59, 115.26, 113.02, 110.89, 108.40, 64.53, 57.31, 45.02, 43.93, 43.90, 19.83, 17.33. ¹⁹F NMR (376 MHz, CD₃OD) δ −96.32, −96.48. LCMS calcd for C₂₇H₃₀F₂N₅O₂ (M+H⁺): 494.5588; Found: 494.6.

### Example 4. Synthesis of (R)₅-(2-(dimethylamino)ethoxy)-N-(1-(2-(furan-2-yl)quinolin-4-yl)ethyl)-2-methylbenzamide

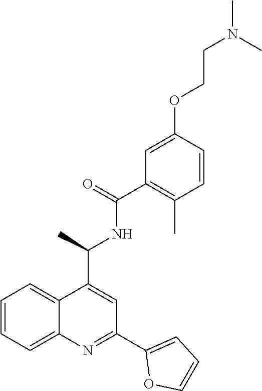 [SMILES: Cc1ccc(OCCN(C)C)cc1C(=O)N[C@H](C)c1cc(-c2ccco2)nc2ccccc12]

White solid ¹H NMR (400 MHz, CD₃OD) δ 8.26 (d, J=8.4 Hz, 1H), 8.10 (d, J=8.5 Hz, 1H), 8.04 (s, 1H), 7.77 (dd, J=8.3, 4.6 Hz, 2H), 7.62 (t, J=7.6 Hz, 1H), 7.32 (d, J=3.5 Hz, 1H), 7.15 (d, J=8.3 Hz, 1H), 6.98-6.88 (m, 2H), 6.67 (dd, J=3.5, 1.8 Hz, 1H), 6.00 (q, J=7.0 Hz, 1H), 4.11 (t, J=5.3 Hz, 2H), 2.85 (t, J=5.3 Hz, 2H), 2.40 (s, 6H), 2.28 (s, 3H), 1.71 (d, J=7.0 Hz, 3H). ¹³C NMR (101 MHz, CD₃OD) δ 170.57, 156.44, 153.18, 151.09, 148.80, 147.96, 144.44, 137.11, 131.51, 129.82, 128.71, 127.55, 126.36, 124.98, 123.10, 115.69, 113.38, 112.97, 112.16, 110.28, 64.94, 57.48, 47.42, 47.21, 46.99, 45.10, 44.18, 19.80, 17.40. LCMS calcd for C₂₇H₃₀N₃O₃ (M+H⁺): 444.5470; Found: 444.2.

### Example 5. Synthesis of (R)-5-(2-(dimethylamino)ethoxy)-2-methyl-N-(1-(2-(thiazol-5-yl)quinolin-4-yl)ethyl)benzamide

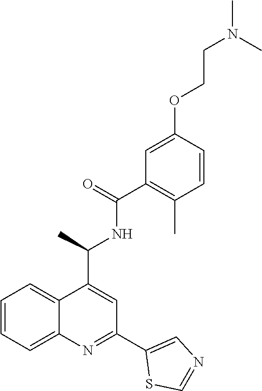 [SMILES: Cc1ccc(OCCN(C)C)cc1C(=O)N[C@H](C)c1cc(-c2cncs2)nc2ccccc12]

White solid ¹H NMR (400 MHz, CD₃OD) δ 9.10 (s, 1H), 8.58 (s, 1H), 8.29 (d, J=8.4 Hz, 1H), 8.07 (d, J=7.7 Hz, 2H), 7.78 (t, J=7.6 Hz, 1H), 7.65 (t, J=7.9 Hz, 1H), 7.16 (d, J=8.2 Hz, 1H), 7.01-6.92 (m, 2H), 6.03 (q, J=6.9 Hz, 1H), 4.15 (t, J=5.3 Hz, 2H), 2.95 (t, J=5.3 Hz, 2H), 2.48 (s, 6H), 2.27 (s, 3H), 1.74 (d, J=7.0 Hz, 3H). ¹³C NMR (101 MHz, CD₃OD) δ 170.56, 156.46, 156.39, 151.16, 149.94, 148.34, 141.20, 140.83, 137.10, 131.55, 129.86, 129.44, 127.60, 126.81, 125.24, 123.08, 115.77, 114.18, 113.06, 113.02, 64.67, 57.36, 57.35, 45.11, 44.00, 19.94, 17.36. LCMS calcd for C₂₆H₂₉N₄O₂S (M+H⁺): 461.5960; Found: 461.6.

### Example 6. Synthesis of (R)-5-(2-(dimethylamino)ethoxy)-N-(1-(2-(1-(2-methoxyethyl)-1H-pyrazol-4-yl)quinolin-4-yl)ethyl)-2-methylbenzamide

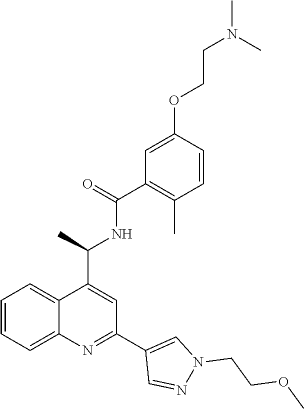 [SMILES: COCCn1cc(-c2cc([C@@H](C)NC(=O)c3cc(OCCN(C)C)ccc3C)c3ccccc3n2)cn1]

White solid ¹H NMR (400 MHz, CD₃OD) δ 8.37 (s, 1H), 8.27 (d, J=8.4 Hz, 1H), 8.19 (s, 1H), 8.06 (d, J=8.5 Hz, 1H), 7.86 (s, 1H), 7.76 (t, =7.7 Hz, 1H), 7.61 (t, J=7.6 Hz, 1H), 7.17 (d, J=9.0 Hz, 1H), 6.98-6.92 (m, 2H), 6.02 (q, J=7.0 Hz, 1H), 4.40 (t, J=5.1 Hz, 2H), 4.13 (t, J=5.3 Hz, 2H), 3.80 (t, J=5.1 Hz, 2H), 3.34 (s, 3H), 2.92 (t, J=5.3 Hz, 2H), 2.46 (s, 6H), 2.27 (s, 3H), 1.73 (d, J=7.0 Hz, 3H). ¹³C NMR (101 MHz, CD₃OD) δ 170.51, 156.36, 151.87, 150.78, 148.15, 137.89, 137.16, 131.53, 130.29, 129.60, 128.40, 127.62, 125.84, 124.66, 123.10, 123.05, 115.70, 115.11, 113.01, 70.56, 64.57, 57.71, 57.32, 51.90, 45.02, 43.96, 19.88, 17.36. LCMS calcd for C₂₉H₃₆N₅O₃ (M+H⁺): 502.6310; Found: 502.6.

### Example 7. Synthesis of (R)-5-(2-(dimethylamino)ethoxy)-2-methyl-N-(1-(2-(1-methyl-1H-pyrazol-4-yl)quinolin-4-yl)ethyl)benzamide

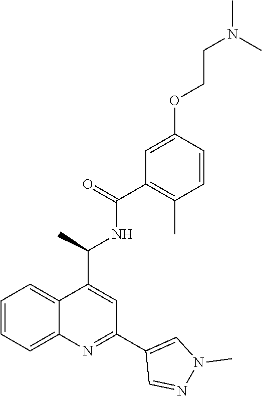 [SMILES: Cc1ccc(OCCN(C)C)cc1C(=O)N[C@H](C)c1cc(-c2cnn(C)c2)nc2ccccc12]

White solid. ¹H NMR (400 MHz, CD₃OD) δ 8.79 (s, 1H), 8.59 (d, J=8.4 Hz, 1H), 8.45 (s, 1H), 8.33-8.23 (m, 2H), 8.08 (ddd, J=8.4, 6.9, 1.1 Hz, 1H), 7.90 (ddd, J=8.3, 7.0, 1.1 Hz, 1H), 7.21 (d, J1=8.4 Hz, 1H), 7.09 (d, J=2.7 Hz, 1H), 7.03 (dd, J=8.4, 2.8 Hz, 1H), 6.07 (q, J=7.0 Hz, 1H), 4.36 (t, J=5.0 Hz, 2H), 4.06 (s, 3H), 3.63-3.56 (m, 2H), 2.99 (s, 6H), 2.26 (s, 3H), 1.79 (d, J=7.1 Hz, 3H). ¹³C NMR (101 MHz, CD₃OD) δ 170.55, 161.27, 155.60, 148.31, 139.42, 138.46, 136.55, 134.06, 133.46, 131.82, 128.70, 128.67, 124.41, 124.36, 120.76, 116.27, 116.02, 114.89, 113.36, 62.03, 56.29, 46.29, 42.50, 38.52, 19.52, 17.48.

LCMS calcd for C₂₇H₃₂N₅O₂ (M+H⁺): 458.5780; Found: 458.6.

### Example 8. Synthesis of (R)—N-(1-(2-(1-cyclopropyl-1H-pyrazol-4-yl)quinolin-4-yl)ethyl)-5-(2-(dimethylamino)ethoxy)-2-methylbenzamide

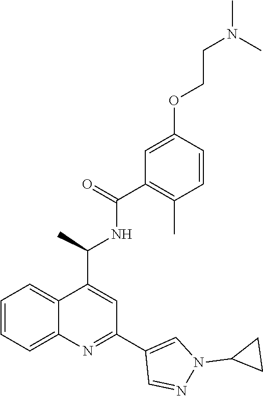 [SMILES: Cc1ccc(OCCN(C)C)cc1C(=O)N[C@H](C)c1cc(-c2cnn(C3CC3)c2)nc2ccccc12]

White solid. ¹H NMR (400 MHz, DMSO-d₆) δ 11.67 (s, 1H), 9.67 (s, 1H), 8.83 (d, J=7.6 Hz, 1H), 7.85 (d, J=8.2 Hz, 1H), 7.46 (t, J=7.6 Hz, 1H), 7.29 (d, J=8.2 Hz, 1H), 7.15 (s, 1H), 6.95-6.89 (m, 2H), 6.47 (s, 1H), 5.49 (p, J=7.1 Hz, 1H), 4.24 (t, J=4.9 Hz, 4H), 3.45 (q, J=5.1 Hz, 3H), 2.79 (d, J=3.9 Hz, 5H), 2.18 (s, 3H), 1.42 (d, J=6.8 Hz, 3H). ¹³C NMR (101 MHz, DMSO-d₆) δ 168.51, 158.83, 155.68, 151.07, 138.62, 138.17, 134.03, 132.06, 131.00, 130.68, 128.32, 126.82, 124.71, 124.11, 118.77, 116.08, 115.98, 114.14, 62.84, 55.93, 54.61, 44.98, 43.29, 21.44, 18.78, 6.83. LCMS calcd for C₂₉H₃₄N₅O₂ (M+H¹): 484.6160; Found: 484.2.

### Example 9. Synthesis of (R)—N-(1-(2-(6-aminopyridin-3-yl)quinolin-4-yl)ethyl)-5-(2-(dimethylamino)ethoxy)-2-methylbenzamide

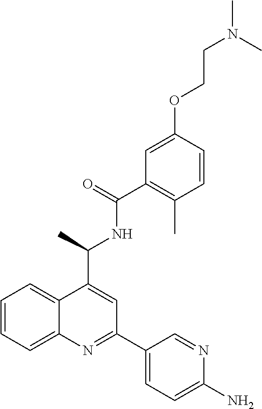 [SMILES: Cc1ccc(OCCN(C)C)cc1C(=O)N[C@H](C)c1cc(-c2ccc(N)nc2)nc2ccccc12]

White solid ¹H NMR (400 MHz, DMSO-d₆) δ 12.06 (s, 1H), 9.91 (s, 1H), 9.10 (d, J=6.9 Hz, 1H), 8.55 (d, J=5.5 Hz, 1H), 8.29 (dd, J=16.1, 8.4 Hz, 2H), 8.12 (t, J=8.0 Hz, 1H), 7.88 (t, J=7.7 Hz, 1H), 7.64 (q, J=7.2, 6.4 Hz, 2H), 7.52 (s, 1H), 7.34 (t, J=6.4 Hz, 1H), 7.21 (d, J=8.3 Hz, 1H), 7.09-6.73 (m, 2H), 5.81 (p, J=7.0 Hz, 1H), 4.33 (t, J=5.0 Hz, 2H), 3.56-3.51 (m, 2H), 2.87 (s, 6H), 2.24 (s, 3H), 1.62 (d, J=6.9 Hz, 3H). ¹³C NMR (101 MHz, DMSO-d₆) δ 168.58, 158.80, 158.47, 155.64, 152.29, 152.12, 137.72, 132.08, 131.71, 128.69, 124.43, 122.35, 118.91, 118.58, 116.03, 114.92, 114.51, 110.42, 62.91, 55.90, 45.48, 43.30, 20.83, 19.01. LCMS calcd for C₂₈H₃₂N₅O₂ (M+H⁺): 470.5890; Found: 470.2.

### Example 10. Synthesis of (R)-5-(2-(dimethylamino)ethoxy)-2-methyl-N-(1-(2-(1-(2,2,2-trifluoroethyl)-1H-pyrazol-4-yl)quinolin-4-yl)ethyl)benzamide

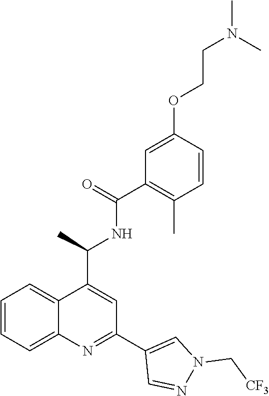 [SMILES: Cc1ccc(OCCN(C)C)cc1C(=O)N[C@H](C)c1cc(-c2cnn(CC(F)(F)F)c2)nc2ccccc12]

White solid. 1¹H NMR (400 MHz, CD₃OD) δ 8.67 (s, 1H), 8.39 (d, J=9.3 Hz, 2H), 8.20-8.08 (m, 1H), 8.04 (s, 1H), 7.88 (ddd, J=8.4, 6.8, 1.3 Hz, 1H), 7.73 (ddd, J=8.3, 6.9, 1.2 Hz, 1H), 7.22 (d, J=8.1 Hz, 1H), 7.04 (d, J=8.2 Hz, 2H), 6.06 (td, J=7.2, 4.7 Hz, 1H), 5.13 (q, J=8.7 Hz, 2H), 4.45-4.30 (m, 2H), 3.65-3.55 (m, 2H), 2.98 (s, 6H), 2.29 (s, 3H), 1.77 (d, J=7.0 Hz, 3H). ¹³C NMR (101 MHz, CD₃OD) δ 170.37, 155.56, 153.17, 150.48, 146.17, 139.48, 137.17, 131.97, 131.72, 130.64, 128.49, 127.05, 126.71, 124.78, 123.35, 122.84, 115.82, 115.42, 113.19, 61.99, 56.27, 45.37, 42.48, 19.77, 17.39. LCMS calcd for C₂₈H₃₁F₃N₅O₂ (M+H⁺): 526.5762; Found: 526.6.

### Example 11. Synthesis of (R)—N-(1-(2-chloroquinolin-4-yl)ethyl)-5-(2-(dimethylamino)-ethoxy)-2-methylbenzamide

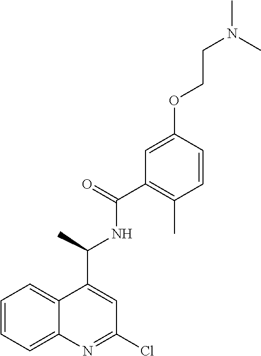 [SMILES: Cc1ccc(OCCN(C)C)cc1C(=O)N[C@H](C)c1cc(Cl)nc2ccccc12]

White solid ¹H NMR (400 MHz, CD₃OD) δ 8.33 (d, J=8.4 Hz, 1H), 7.99 (d, J=8.4 Hz, 1H), 7.87-7.78 (m, 1H), 7.71 (ddd, J=8.3, 6.9, 1.3 Hz, 1H), 7.56 (s, 1H), 7.20-7.11 (m, 1H), 6.98-6.91 (m, 2H), 5.96 (q, J=7.0 Hz, 1H), 4.11 (t, J=5.3 Hz, 2H), 2.81 (t, J=5.4 Hz, 2H), 2.37 (s, 6H), 2.27 (s, 3H), 1.68 (d, J=7.0 Hz, 3H). ¹³C NMR (101 MHz, CD₃OD) δ 170.52, 156.52, 154.15, 150.60, 147.90, 136.81, 131.56, 130.56, 128.31, 127.50, 127.17, 124.77, 123.43, 118.25, 115.65, 113.06, 65.15, 57.57, 45.04, 44.32, 19.70, 17.44. LCMS calcd for C₂₃H₂₇ClN₃O₂ (M+H⁺): 412.9300; Found: 413.0.

### Example 12. Synthesis of (R)-5-(2-(dimethylamino)ethoxy)-N-(1-(2-(1-(2-(dimethylamino)ethyl)-1H-pyrazol-4-yl)quinolin-4-yl)ethyl)-2-methylbenzamide

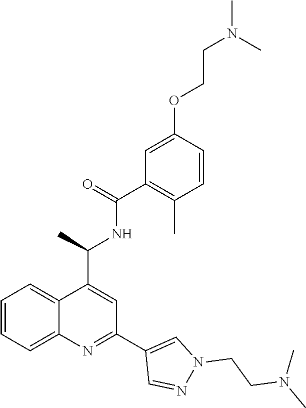 [SMILES: Cc1ccc(OCCN(C)C)cc1C(=O)N[C@H](C)c1cc(-c2cnn(CCN(C)C)c2)nc2ccccc12]

White solid ¹H NMR (400 MHz, CD₃OD) δ 8.40 (s, 1H), 8.28-8.22 (m, 1H), 8.05 (dd, J=8.5, 1.2 Hz, 1H), 7.86 (s, 1H), 7.75 (ddd, J=8.3, 6.8, 1.3 Hz, 1H), 7.59 (ddd, J=8.3, 6.8, 1.3 Hz, 1H), 7.19-7.11 (m, 1H), 6.93 (d, J=6.8 Hz, 2H), 6.01 (q, J=7.0 Hz, 1H), 4.35 (t, J=6.6 Hz, 2H), 4.07 (t, J=5.4 Hz, 2H), 2.86 (t, J=6.6 Hz, 2H), 2.74 (t, J=5.4 Hz, 2H), 2.32 (s, 6H), 2.30 (s, 6H), 2.26 (s, 3H), 1.72 (d, J=7.0 Hz, 3H). ¹³C NMR (101 MHz, CD₃OD) δ 170.56, 156.61, 151.83, 150.68, 148.16, 137.93, 137.10, 131.48, 130.00, 129.59, 128.42, 127.34, 125.84, 124.69, 123.19, 123.07, 115.60, 115.12, 112.99, 65.37, 58.33, 57.64, 49.67, 44.98, 44.41, 44.21, 19.82, 17.36. LCMS calcd for C₃₀H₃₉N₆O₂ (M+H⁺): 515.6740; Found: 515.3.

### Example 13. Synthesis of (R)-5-(2-(dimethylamino)ethoxy)-2-methyl-N-(1-(2-(1-methyl-1H-pyrazol-5-yl)quinolin-4-yl)ethyl)benzamide

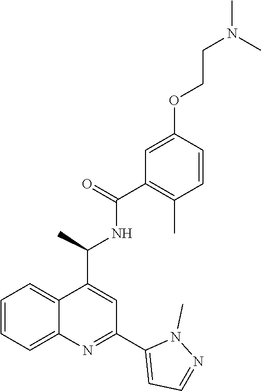 [SMILES: Cc1ccc(OCCN(C)C)cc1C(=O)N[C@H](C)c1cc(-c2ccnn2C)nc2ccccc12]

White solid ¹H NMR (400 MHz, CD₃OD) δ 8.33 (d, J=8.4 Hz, 1H), 8.15 (d, J=8.4 Hz, 1H), 7.90 (s, 1H), 7.82 (t, J=7.7 Hz, 1H), 7.70 (t, J=7.7 Hz, 1H), 7.57 (d, J=2.1 Hz, 1H), 7.17 (d, J=8.3 Hz, 1H), 6.95 (d, J=7.5 Hz, 2H), 6.86 (d, J=2.1 Hz, 1H), 6.04 (q, J=7.0 Hz, 1H), 4.34 (s, 3H), 4.12 (t, J=5.3 Hz, 2H), 2.88 (t, J=5.3 Hz, 2H), 2.43 (s, 6H), 2.27 (s, 3H), 1.72 (d, J=7.0 Hz, 3H). ¹³C NMR (101 MHz, CD₃OD) δ 174.46, 160.40, 154.97, 153.14, 151.73, 145.44, 141.75, 141.00, 135.48, 133.72, 133.69, 131.42, 130.97, 128.67, 126.94, 120.51, 119.61, 116.92, 110.98, 68.87, 61.40, 49.00, 48.12, 42.58, 23.89, 21.36. LCMS calcd for C₂₇H₃₂N₅O₂ (M+H⁺): 458.5780; Found: 458.5.

### Example 14. Synthesis of (R)-5-(2-(dimethylamino)ethoxy)-N-(1-(2-(1-isopropyl-1H-pyrazol-4-yl)quinolin-4-yl)ethyl)-2-methylbenzamide

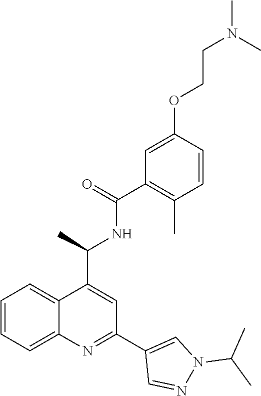 [SMILES: Cc1ccc(OCCN(C)C)cc1C(=O)N[C@H](C)c1cc(-c2cnn(C(C)C)c2)nc2ccccc12]

White solid. ¹H NMR (400 MHz, CD₃OD) δ 8.42 (s, 1H), 8.27 (d, J=8.3 Hz, 1H), 8.17 (s, 1H), 8.06 (d, J=8.4 Hz, 1H), 7.88 (s, 1H), 7.76 (dd, J=8.5, 7.0 Hz, 1H), 7.61 (t, J=7.6 Hz, 1H), 7.17 (d, J=8.2 Hz, 1H), 6.95 (d, J=7.4 Hz, 2H), 6.02 (q, J=7.0 Hz, 1H), 4.66-4.57 (m, 1H), 4.12 (t, J=5.3 Hz, 2H), 2.89 (t, J=5.3 Hz, 2H), 2.44 (s, 6H), 2.27 (s, 3H), 1.73 (d, J=7.0 Hz, 3H), 1.57 (d, J=6.7 Hz, 6H). ¹³C NMR (101 MHz, CD₃OD) δ 170.48, 156.43, 152.00, 150.67, 148.15, 137.43, 137.15, 131.53, 129.60, 128.37, 127.49, 127.05, 125.81, 124.66, 123.08, 122.71, 115.61, 115.17, 113.03, 64.83, 57.43, 54.21, 44.99, 44.12, 21.72, 19.88, 17.41. LCMS calcd for C₂₉H₃₆N₅O₂ (M+H⁺): 486.6320; Found: 486.6.

### Example 15. Synthesis of (R)—N-(1-(2-(1H-pyrazol-5-yl)quinolin-4-yl)ethyl)-5-(2-(dimethylamino)ethoxy)-2-methylbenzamide

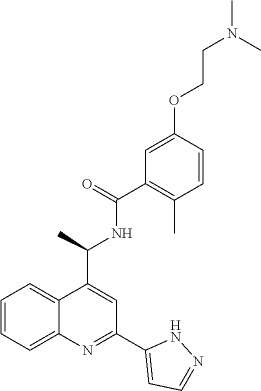 [SMILES: Cc1ccc(OCCN(C)C)cc1C(=O)N[C@H](C)c1cc(-c2ccn[nH]2)nc2ccccc12]

White solid. ¹H NMR (400 MHz, CD₃OD) δ 8.27 (d, J=8.5 Hz, 2H), 8.14 (d, J=8.4 Hz, 1H), 7.82-7.74 (m, 2H), 7.63 (t, J=7.6 Hz, 1H), 7.18-7.08 (m, 2H), 6.99 (d, J=2.8 Hz, 1H), 6.92 (dd, J=8.4, 2.8 Hz, 1H), 6.01 (q, J=7.0 Hz, 1H), 4.14 (t, J=5.5 Hz, 2H), 2.93 (t, J=5.4 Hz, 2H), 2.46 (s, 6H), 2.27 (s, 3H), 1.72 (d, J=7.0 Hz, 3H). ¹³C NMR (101 MHz, CD₃OD) δ 170.63, 156.42, 150.63, 148.03, 137.15, 131.45, 129.51, 129.02, 127.57, 126.37, 125.41, 123.07, 115.90, 114.72, 112.76, 103.84, 64.92, 57.46, 45.01, 44.13, 19.68, 17.32. LCMS calcd for C₂₆H₃₀N₅O₂ (M+H⁺): 444.5510; Found: 444.5.

### Example 16. Synthesis of (R)-5-(2-(dimethylamino)ethoxy)-2-methyl-N-(1-(2-(5-(morpholinomethyl)thiophen-2-yl)quinolin-4-yl)ethyl)benzamide

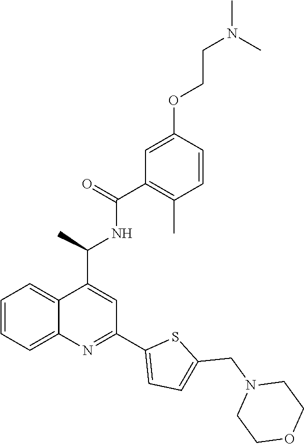 [SMILES: Cc1ccc(OCCN(C)C)cc1C(=O)N[C@H](C)c1cc(-c2ccc(CN3CCOCC3)s2)nc2ccccc12]

White solid. ¹H NMR (400 MHz, CD₃OD) δ 8.32 (d, J=8.5 Hz, 1H), 8.08 (d, J=9.1 Hz, 2H), 7.87 (dd, J=3.9, 1.5 Hz, 1H), 7.81 (ddd, J=8.3, 6.8, 1.3 Hz, 1H), 7.66 (ddd, J=8.3, 6.9, 1.3 Hz, 1H), 7.41 (dd, J=4.0, 1.8 Hz, 1H), 7.20 (d, J=8.3 Hz, 1H), 7.05-6.98 (m, 2H), 6.02 (q, 0.1=6.9 Hz, 1H), 4.65 (d, J=2.1 Hz, 2H), 4.33 (t, J=4.9 Hz, 2H), 3.91 (s, 2H), 3.60-3.55 (m, 2H), 3.35 (s, 2H), 2.96 (s, 6H), 2.28 (d, J=1.5 Hz, 3H), 1.74 (d, J=7.0 Hz, 3H). ¹³C NMR (101 MHz, CD₃OD) δ 170.38, 160.72, 160.36, 155.56, 152.19, 150.82, 147.52, 147.11, 137.20, 133.59, 132.56, 131.72, 130.39, 128.47, 126.95, 125.23, 123.23, 115.82, 113.80, 113.18, 63.58, 61.99, 56.26, 54.07, 51.17, 45.36, 42.48, 19.82, 17.49. LCMS calcd for C₃₂H₃₉N₄O₃S (M+H⁺): 559.7410; Found: 559.7.

### Example 17. Synthesis of (S)-5-(2-(dimethylamino)ethoxy)-N-(1-(2-(furan-2-yl)quinolin-4-yl)ethyl)-2-methylbenzamide

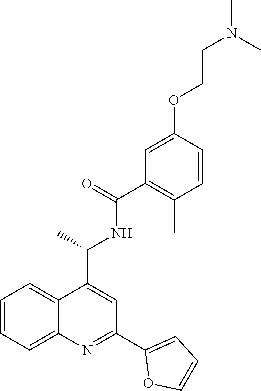 [SMILES: Cc1ccc(OCCN(C)C)cc1C(=O)N[C@@H](C)c1cc(-c2ccco2)nc2ccccc12]

White solid ¹H NMR (400 MHz, CD₃OD) δ 8.26 (d, J=8.4 Hz, 1H), 8.10 (d, J=8.5 Hz, 1H), 8.04 (s, 1H), 7.77 (dd, J=8.3, 4.6 Hz, 2H), 7.62 (t, J=7.6 Hz, 1H), 7.32 (d, J=3.5 Hz, 1H), 7.15 (d, J=8.3 Hz, 1H), 6.98-6.88 (m, 2H), 6.67 (dd, J=3.5, 1.8 Hz, 1H), 6.00 (q, J=7.0 Hz, 1H), 4.11 (t, J=5.3 Hz, 2H), 2.85 (t, J=5.3 Hz, 2H), 2.40 (s, 6H), 2.28 (s, 3H), 1.71 (d, J=7.0 Hz, 3H). ¹³C NMR (101 MHz, CD₃OD) δ 170.57, 156.44, 153.18, 151.09, 148.80, 147.96, 144.44, 137.11, 131.51, 129.82, 128.71, 127.55, 126.36, 124.98, 123.10, 115.69, 113.38, 112.97, 112.16, 110.28, 64.94, 57.48, 47.42, 47.21, 46.99, 45.10, 44.18, 19.80, 17.40. LCMS calcd for C₂₇H₃₀N₃O₃ (M+H⁺): 444.5470; Found: 444.2.

### Example 18. Synthesis of (S)—N-(1-(2-(1-(difluoromethyl)-1H-pyrrol-3-yl)quinolin-4-yl)ethyl)-5-(2-(dimethylamino)ethoxy)-2-methylbenzamide

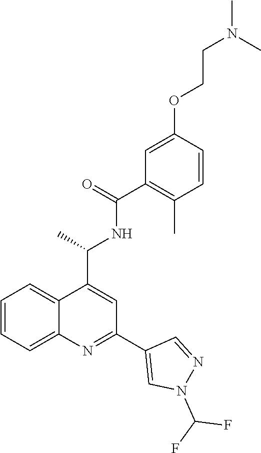 [SMILES: Cc1ccc(OCCN(C)C)cc1C(=O)N[C@@H](C)c1cc(-c2cnn(C(F)F)c2)nc2ccccc12]

White solid. ¹H NMR (400 MHz, CD₃OD) δ 8.78 (s, 1H), 8.41 (s, 1H), 8.34-8.24 (m, 1H), 8.09 (dd, J=8.5, 1.2 Hz, 1H), 7.93 (s, 1H), 7.82-7.73 (m, 1H), 7.69-7.55 (m, 2H), 7.19-7.09 (m, 1H), 6.94 (tt, J=4.2, 2.2 Hz, 2H), 6.03 (q, J=7.0 Hz, 1H), 4.12 (td, J=5.2, 2.1 Hz, 2H), 2.89 (t, J=5.3 Hz, 2H), 2.44 (d, J=4.6 Hz, 6H), 2.27 (d, J=3.4 Hz, 3H), 1.73 (d, J=7.0 Hz, 3H). ¹³C NMR (101 MHz, CD₃OD) δ 156.35, 151.03, 140.56, 131.54, 129.75, 128.86, 127.61, 126.78, 126.37, 125.70, 125.00, 123.11, 115.67, 115.26, 113.02, 64.53, 57.31, 47.39, 47.18, 46.97, 45.02, 43.93, 19.83, 17.33. LCMS calcd for C₂₇H₃₀F₂N₅O₂ (M+H⁺): 494.5588; Found: 494.5.

### Example 19. Synthesis of (S)-5-(2-(dimethylamino)ethoxy)-N-(1-(2-(1-(2-methoxyethyl)-1H-pyrazol-4-yl)quinolin-4-yl)ethyl)-2-methylbenzamide

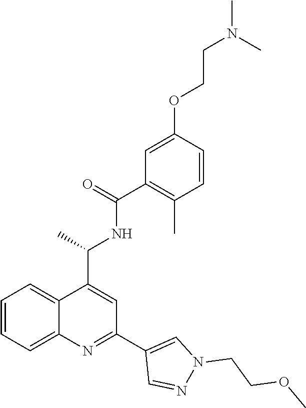 [SMILES: COCCn1cc(-c2cc([C@H](C)NC(=O)c3cc(OCCN(C)C)ccc3C)c3ccccc3n2)cn1]

White solid. ¹H NMR (400 MHz, CD₃OD) δ 8.37 (s, 1H), 8.27 (d, J=8.4 Hz, 1H), 8.19 (s, 1H), 8.06 (d, J=8.5 Hz, 1H), 7.86 (s, 1H), 7.76 (t, J=7.7 Hz, 1H), 7.61 (t, J=7.6 Hz, 1H), 7.17 (d, J=9.0 Hz, 1H), 6.98-6.92 (m, 2H), 6.02 (q, J=7.0 Hz, 1H), 4.40 (t, J=5.1 Hz, 2H), 4.13 (t, J=5.3 Hz, 2H), 3.80 (t, J=5.1 Hz, 2H), 3.34 (s, 3H), 2.92 (t, J=5.3 Hz, 2H), 2.46 (s, 6H), 2.27 (s, 3H), 1.73 (d, J=7.0 Hz, 3H). ¹³C NMR (101 MHz, CD₃OD) δ 170.51, 156.36, 151.87, 150.78, 148.15, 137.89, 137.16, 131.53, 130.29, 129.60, 128.40, 127.62, 125.84, 124.66, 123.10, 123.05, 115.70, 115.11, 113.01, 70.56, 64.57, 57.71, 57.32, 51.90, 45.02, 43.96, 19.88, 17.36. LCMS calcd for C₂₉H₃₆N₅O₃ (M+H⁺): 501.6310; Found: 501.6.

### Example 20. Synthesis of (S)—N-(1-(2-(1-cyclopropyl-1H-pyrazol-4-yl)quinolin-4-yl)ethyl)-5-(2-(dimethylamino)ethoxy)-2-methylbenzamide

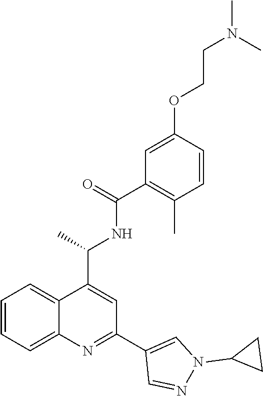 [SMILES: Cc1ccc(OCCN(C)C)cc1C(=O)N[C@@H](C)c1cc(-c2cnn(C3CC3)c2)nc2ccccc12]

White solid. ¹H NMR (400 MHz, DMSO-d₆) δ 11.67 (s, 1H), 9.67 (s, 1H), 8.83 (d, J=7.6 Hz, 1H), 7.85 (d, J=8.2 Hz, 1H), 7.46 (t, J=7.6 Hz, 1H), 7.29 (d, J=8.2 Hz, 1H), 7.15 (s, 1H), 6.95-6.89 (m, 2H), 6.47 (s, 1H), 5.49 (p, J=7.1 Hz, 1H), 4.24 (t, J=4.9 Hz, 4H), 3.45 (q, J=5.1 Hz, 3H), 2.79 (d, J=3.9 Hz, 5H), 2.18 (s, 3H), 1.42 (d, J=6.8 Hz, 3H). ¹³C NMR (101 MHz, DMSO-d₆) δ 168.51, 158.83, 155.68, 151.07, 138.62, 138.17, 134.03, 132.06, 131.00, 130.68, 128.32, 126.82, 124.71, 124.11, 118.77, 116.08, 115.98, 114.14, 62.84, 55.93, 54.61, 44.98, 43.29, 21.44, 18.78, 6.83. LCMS calcd for C₂₉H₃₄N₅O₂ (M+H⁺): 484.6160; Found: 484.6.

### Example 21. Synthesis of (S)-5-(2-(dimethylamino)ethoxy)-2-methyl-N-(1-(2-(1-methyl-1H-pyrazol-5-yl)quinolin-4-yl)ethyl)benzamide

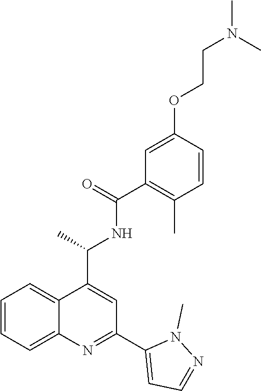 [SMILES: Cc1ccc(OCCN(C)C)cc1C(=O)N[C@@H](C)c1cc(-c2ccnn2C)nc2ccccc12]

White solid ¹H NMR (400 MHz, CD₃OD) δ 8.33 (d, J=8.4 Hz, 1H), 8.15 (d, J=8.4 Hz, 1H), 7.90 (s, 1H), 7.82 (t, J=7.7 Hz, 1H), 7.70 (t, J=7.7 Hz, 1H), 7.57 (d, J=2.1 Hz, 1H), 7.17 (d, J=8.3 Hz, 1H), 6.95 (d, J=7.5 Hz, 2H), 6.86 (d, J=2.1 Hz, 1H), 6.04 (q, J=7.0 Hz, 1H), 4.34 (s, 3H), 4.12 (t, J=5.3 Hz, 2H), 2.88 (t, J=5.3 Hz, 2H), 2.43 (s, 6H), 2.27 (s, 3H), 1.72 (d, J=7.0 Hz, 3H). ¹³C NMR (101 MHz, CD₃OD) δ 174.46, 160.40, 154.97, 153.14, 151.73, 145.44, 141.75, 141.00, 135.48, 133.72, 133.69, 131.42, 130.97, 128.67, 126.94, 120.51, 119.61, 116.92, 110.98, 68.87, 61.40, 49.00, 48.12, 42.58, 23.89, 21.36. LCMS calcd for C₂₇H₃₂N₅O₂ (M+H⁺): 458.5780, Found: 458.5.

### Example 22. Synthesis of (R)-5-(2-(dimethylamino)ethoxy)-N-(1-(2-(1-(2-hydroxyethyl)-1H-pyrazol-4-yl)quinolin-4-yl)ethyl)-2-methylbenzamide

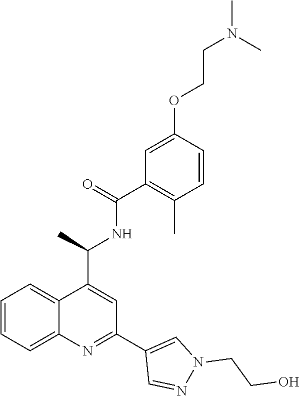 [SMILES: Cc1ccc(OCCN(C)C)cc1C(=O)N[C@H](C)c1cc(-c2cnn(CCO)c2)nc2ccccc12]

White solid. ¹H NMR (400 MHz, DMSO-d₆) δ 9.89 (s, 1H), 9.04 (d, J=7.6 Hz, 1H), 8.61 (s, 1H), 8.37 (d, J=8.4 Hz, 1H), 8.28 (s, 1H), 8.16-8.02 (m, 2H), 7.88 (p, J=7.1, 6.4 Hz, 1H), 7.70 (t, J=7.6 Hz, 1H), 7.20 (d, J=9.0 Hz, 1H), 7.00 (d, J=6.8 Hz, 2H), 5.94 (q, J=7.1 Hz, 1H), 4.30 (dt, J=16.6, 5.1 Hz, 4H), 3.82 (t, J=5.3 Hz, 2H), 3.51 (q, J=4.7 Hz, 2H), 2.87 (s, 6H), 2.22 (s, 3H), 1.64 (d, J=6.9 Hz, 3H). ¹³C NMR (101 MHz, DMSO-d₆) δ 168.54, 158.94, 158.59, 155.70, 138.71, 138.09, 132.06, 131.82, 131.53, 128.35, 127.10, 124.60, 124.26, 116.14, 116.09, 114.13, 62.88, 60.23, 55.90, 55.17, 45.12, 43.27, 21.47, 18.81. LCMS calcd for C₂₈H₃₄N₅O₃ (M+H⁺): 487.6040; Found: 487.6.

### Example 23. Synthesis of (R)—N-(1-(2-(1H-pyrazol-4-yl)quinolin-4-yl)ethyl)-5-(2-(dimethylamino)ethoxy)-2-methylbenzamide

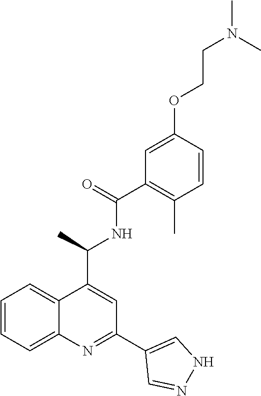 [SMILES: Cc1ccc(OCCN(C)C)cc1C(=O)N[C@H](C)c1cc(-c2cn[nH]c2)nc2ccccc12]

White solid. ¹H NMR (400 MHz, DMSO-d₆) δ 9.94 (s, 1H), 9.10 (d, J=7.6 Hz, 1H), 8.54 (s, 2H), 8.43 (d, J=8.5 Hz, 1H), 8.28-8.10 (m, 2H), 7.94 (t, J=7.7 Hz, 1H), 7.76 (t, J=7.6 Hz, 1H), 7.26 (d, J=8.9 Hz, 1H), 7.05 (d, J=6.8 Hz, 2H), 5.99 (p, J=7.0 Hz, 1H), 4.37 (t, J=4.9 Hz, 2H), 3.57 (q, J=4.6 Hz, 2H), 2.97 (s, 6H), 2.27 (s, 3H), 1.71 (d, J=6.9 Hz, 3H). ¹³C NMR (101 MHz, DMSO-d₆) δ 168.55, 158.94, 158.60, 155.69, 138.08, 132.05, 128.35, 127.13, 124.62, 124.28, 118.22, 116.40, 116.11, 114.17, 62.88, 55.89, 45.14, 43.27, 21.43, 18.80. LCMS calcd for C₂₆H₃₀N₅O₂ (M+H⁺): 444.5510; Found: 444.5.

### Example 24. Synthesis of (R)—N-(1-(2-(1-(2-amino-2-oxoethyl)-1H-pyrazol-4-yl)quinolin-4-yl)ethyl)-5-(2-(dimethylamino)ethoxy)-2-methylbenzamide

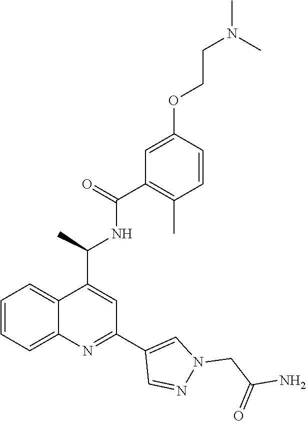 [SMILES: Cc1ccc(OCCN(C)C)cc1C(=O)N[C@H](C)c1cc(-c2cnn(CC(N)=O)c2)nc2ccccc12]

White solid. ¹H NMR (400 MHz, DMSO-d₆) δ 9.87 (s, 1H), 9.04 (d, J=7.7 Hz, 1H), 8.59 (s, 1H), 8.36 (d, J=8.5 Hz, 1H), 8.26 (s, 1H), 8.11 (d, J=8.4 Hz, 1H), 8.05 (s, 1H), 7.87 (t, J=7.7 Hz, 1H), 7.74-7.65 (m, 2H), 7.36 (s, 1H), 7.24-7.15 (m, 1H), 7.00 (q, J=3.2, 2.7 Hz, 2H), 5.95 (t, J=7.2 Hz, 1H), 4.93 (s, 4H), 4.31 (t, J=5.0 Hz, 2H), 3.51 (q, J=4.7 Hz, 2H), 2.22 (s, 3H), 1.64 (d, J=6.9 Hz, 3H. ¹³C NMR (101 MHz, DMSO-d₆) δ 168.59, 168.53, 158.93, 158.59, 155.69, 150.79, 138.80, 138.10, 132.78, 132.06, 131.37, 128.35, 127.04, 124.65, 124.21, 121.02, 118.09, 116.17, 116.03, 114.07, 62.86, 55.90, 54.61, 45.02, 43.27, 21.52, 18.81. LCMS calcd for C₂₈H₃₃N₆O₃ (−M+H⁺): 501.6030. Found: 501.2.

### Example 25. Synthesis of (R)-5-(2-(dimethylamino)ethoxy)-2-methyl-N-(1-(2-(pyrrolidin-1-yl)quinolin-4-yl)ethyl)benzamide

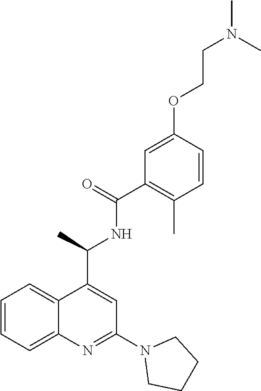 [SMILES: Cc1ccc(OCCN(C)C)cc1C(=O)N[C@H](C)c1cc(N2CCCC2)nc2ccccc12]

White solid, ¹H NMR (400 MHz, DMSO-d₆) δ 9.98 (s, 1H), 9.10 (d, J=7.4 Hz, 1H), 8.27 (d, J=8.4 Hz, 1H), 8.05 (d, J=8.5 Hz, 1H), 7.84 (t, J=7.7 Hz, 1H), 7.22 (d, J=9.3 Hz, 2H), 7.05-7.00 (m, 2H), 5.78 (p, J=7.1 Hz, 1H), 4.33 (t, J=5.1 Hz, 2H), 3.74 (s, 6H), 3.52 (s, 2H), 2.24 (s, 3H), 2.08 (s, 2H), 1.56 (d, J=7.0 Hz, 3H). ¹³C NMR (101 MHz, DMSO-d₆) δ 168.67, 158.68, 158.36, 155.74, 137.94, 132.12, 128.36, 124.91, 119.14, 116.32, 114.13, 109.04, 62.94, 55.91, 45.54, 43.29, 35.41, 25.17, 21.36, 18.94. LCMS calcd for C₂₇H₃₅N₄O₂ (M+H⁺): 447.5950; Found: 447.6.

### Example 26. Synthesis of (R)—N-(1-(2-(1-(azetidin-3-yl)-1H-pyrazol-4-yl)quinolin-4-yl)ethyl)-5-(2-(dimethylamino)ethoxy)-2-methylbenzamide

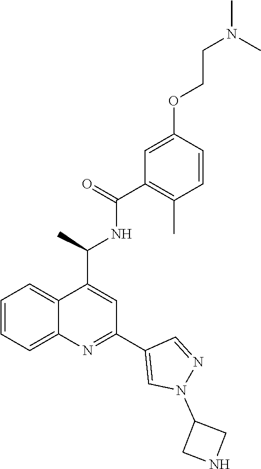 [SMILES: Cc1ccc(OCCN(C)C)cc1C(=O)N[C@H](C)c1cc(-c2cnn(C3CNC3)c2)nc2ccccc12]

White solid, ¹H NMR (400 MHz, CD₃OD) δ 9.12 (s, 1H), 8.65 (t, J=4.4 Hz, 2H), 8.55-8.39 (m, 2H), 8.14 (t, J=7.8 Hz, 1H), 7.96 (t, J=7.7 Hz, 1H), 7.22 (dd, J=5.6, 2.9 Hz, 2H), 7.06 (dd, J=8.4, 2.5 Hz, 1H), 6.12 (q, J=7.0 Hz, 1H), 4.69 (d, J=6.8 Hz, 2H), 4.44 (t, J=4.8 Hz, 2H), 4.21 (dt, J=27.8, 10.4 Hz, 3H), 3.65 (d, J=4.7 Hz, 2H), 3.03 (s, 6H), 2.29 (s, 3H), 1.84 (d, J=7.0 Hz, 3H). ¹³C NMR (101 MHz, CD₃OD) δ 140.31, 134.23, 133.85, 131.84, 128.87, 124.48, 120.61, 116.73, 116.28, 113.66, 62.17, 56.34, 53.08, 49.11, 47.41, 47.20, 46.99, 46.31, 42.68, 42.62, 32.24, 19.77, 17.63. LCMS calcd for C₂₉H₃₅N₆O₂ (M+H⁺): 499.6310; Found: 499.6.

### Example 27. Synthesis of (R)-5-(2-(dimethylamino)ethoxy)-2-methyl-N-(1-(2-(2-methyl-2H-tetrazol-5-yl)quinolin-4-yl)ethyl)benzamide

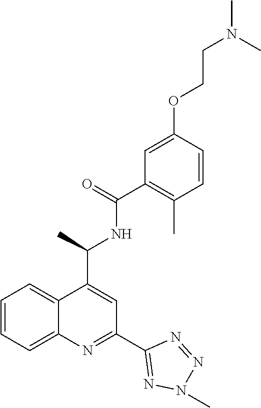 [SMILES: Cc1ccc(OCCN(C)C)cc1C(=O)N[C@H](C)c1cc(-c2nnn(C)n2)nc2ccccc12]

White solid, ¹H NMR (400 MHz, DMSO-d₆) δ 9.75 (s, 1H), 9.56 (d, J=2.2 Hz, 1H), 9.08 (d, J=7.9 Hz, 1H), 8.84 (dd, J=8.2, 2.2 Hz, 1H), 8.38 (d, J=5.1 Hz, 2H), 8.20 (d, J=8.5 Hz, 1H), 7.88 (t, J=7.7 Hz, 1H), 7.74 (t, J=7.7 Hz, 1H), 7.22 (d, J=8.2 Hz, 1H), 7.05-6.98 (m, 2H), 6.03 (p, J=7.2 Hz, 1H), 4.51 (s, 3H), 4.34 (t, J=5.0 Hz, 2H), 3.54 (d, J=15.0 Hz, 2H), 2.88 (d, J=4.6 Hz, 6H), 2.24 (s, 3H), 1.67 (d, J=6.9 Hz, 3H). ¹′C NMR (101 MHz, DMSO-d₆) δ 168.50, 164.30, 158.78, 158.44, 155.72, 153.38, 152.47, 149.08, 148.41, 147.26, 138.28, 136.31, 135.61, 132.08, 130.61, 128.31, 127.79, 125.47, 123.93, 122.99, 116.23, 115.41, 114.00, 62.92, 55.96, 44.89, 43.31, 21.94, 18.78. LCMS calcd for C₂₅H₃₀N₇O₂ (M+H⁺): 460.5540; Found: 460.6.

### Example 28. Synthesis of (R)-5-(2-(dimethylamino)ethoxy)-N-(1-(2-(1-(methoxymethyl)-1H-pyrazol-4-yl)quinolin-4-yl)ethyl)-2-methylbenzamide

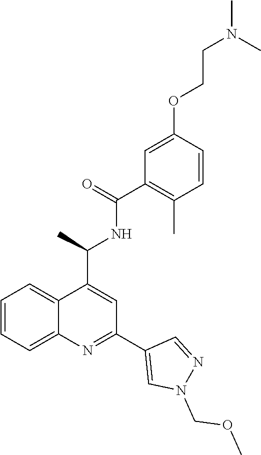 [SMILES: COCn1cc(-c2cc([C@@H](C)NC(=O)c3cc(OCCN(C)C)ccc3C)c3ccccc3n2)cn1]

White solid, ¹H NMR (400 MHz, DMSO-d₆) δ 9.86 (s, 1H), 9.02 (d, J=7.6 Hz, 1H), 8.77 (s, 1H), 8.35 (d, J=9.1 Hz, 2H), 8.22-8.05 (m, 2H), 7.86 (t, J=7.7 Hz, 1H), 7.69 (t, J=7.7 Hz, 1H), 7.20 (d, J=8.9 Hz, 1H), 6.99 (d, J=6.4 Hz, 2H), 5.93 (p, J=7.0 Hz, 1H), 5.51 (s, 2H), 4.32 (d, J=5.0 Hz, 2H), 3.51 (q, J=4.8 Hz, 2H), 2.86 (d, J=3.4 Hz, 6H), 2.21 (s, 3H), 1.65 (d, J=6.9 Hz, 3H). ¹³C NMR (101 MHz, DMSO-d₆) δ 168.50, 158.96, 158.61, 155.69, 150.85, 139.43, 138.15, 132.04, 131.70, 131.05, 128.36, 126.96, 124.84, 124.15, 117.89, 116.18, 116.10, 114.98, 114.17, 82.03, 62.88, 56.78, 55.93, 45.00, 43.28, 21.41, 18.79. LCMS calcd for C₂₈H₃₄N₅O₃ (M+H⁺): 488.6040; Found: 488.2.

### Example 29. Synthesis of (R)—N-(1-(2-(5-((cyclopentylamino)methyl)thiophen-2-yl)quinolin-4-yl)ethyl)-5-(2-(dimethylamino)ethoxy)-2-methylbenzamide

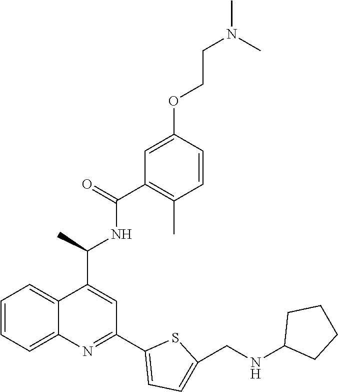 [SMILES: Cc1ccc(OCCN(C)C)cc1C(=O)N[C@H](C)c1cc(-c2ccc(CNC3CCCC3)s2)nc2ccccc12]

White solid, ¹H NMR (400 MHz, DMSO-d₆) δ 10.08 (s, 1H), 9.26 (s, 2H), 9.03 (d, J=7.7 Hz, 1H), 8.30 (d, J=8.4 Hz, 1H), 8.13 (s, 1H), 8.00 (d, J=8.2 Hz, 1H), 7.85-7.77 (m, 2H), 7.66 (ddd, J=8.3, 6.7, 1.3 Hz, 1H), 7.39 (d, J=3.7 Hz, 1H), 7.24-7.14 (m, 1H), 7.05-6.90 (m, 2H), 5.92 (p, J=7.1 Hz, 1H), 4.46 (t, J=5.3 Hz, 2H), 4.33 (t, J=5.1 Hz, 2H), 3.53 (dt, J=10.1, 5.6 Hz, 3H), 2.87 (s, 6H), 2.21 (s, 3H), 2.00 (td, J=10.0, 8.2, 3.8 Hz, 2H), 1.71 (dt, J=11.2, 4.9 Hz, 4H), 1.65 (d, J=6.9 Hz, 3H), 1.59-1.51 (m, 2H). ¹³C NMR (101 MHz, DMSO-d₆) δ 168.51, 158.95, 158.62, 155.71, 151.74, 151.61, 148.08, 147.14, 138.28, 136.83, 132.02, 131.94, 130.50, 129.81, 128.27, 127.09, 126.63, 125.37, 116.06, 114.15, 62.93, 58.38, 55.90, 44.99, 44.07, 43.27, 29.62, 24.13, 21.54, 18.76. LCMS calcd for C₃₃H₄₁N₄O₂S (M+H⁺): 557.7690; Found: 557.8.

### Example 30. Synthesis of (R)-5-(2-(dimethylamino)ethoxy)-N-(1-(2-(6-(4-hydroxypiperidin-1-yl)pyridin-3-yl)quinolin-4-yl)ethyl)-2-methylbenzamide

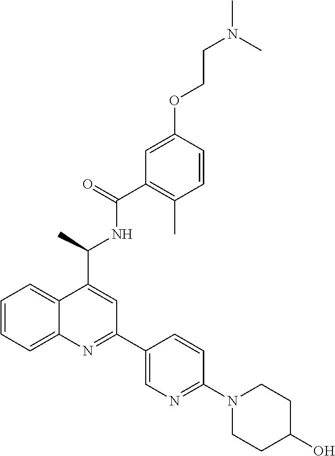 [SMILES: Cc1ccc(OCCN(C)C)cc1C(=O)N[C@H](C)c1cc(-c2ccc(N3CCC(O)CC3)nc2)nc2ccccc12]

White solid, ¹H NMR (400 MHz, DMSO-d₆) δ 9.07 (d, J=7.8 Hz, 1H), 8.89 (d, J=2.6 Hz, 1H), 8.50-8.26 (m, 2H), 8.25-8.04 (m, 2H), 7.90 (q, J=8.6, 7.7 Hz, 1H), 7.72 (t, J=7.7 Hz, 1H), 7.28-7.15 (m, 2H), 7.06-6.90 (m, 2H), 5.97 (p, J=7.1 Hz, 1H), 4.32 (t, J=5.1 Hz, 5H), 4.12 (dt, J=13.7, 4.7 Hz, 2H), 3.81 (tt, J=8.3, 4.0 Hz, 1H), 3.37 (ddd, J=13.3, 9.5, 3.3 Hz, 2H), 2.20 (s, 3H), 1.85 (dq, J=13.1, 3.9 Hz, 2H), 1.64 (d, J=7.0 Hz, 3H), 1.46 (dt, J=17.5, 5.4 Hz, 2H). ¹³C NMR (101 MHz, DMSO-d₆) δ 168.54, 162.28, 158.97, 158.61, 155.72, 139.53, 138.17, 132.07, 131.33, 130.79, 128.40, 128.28, 127.33, 124.76, 124.41, 124.10, 122.31, 116.12, 114.95, 114.14, 108.50, 65.97, 62.89, 55.91, 45.13, 43.28, 43.12, 34.12, 21.76, 18.80. LCMS calcd for C₃₃H₄₀N₅O₃ (M+H⁺): 554.7070; Found: 554.3.

### Example 31. Synthesis of (R)-5-(2-(dimethylamino)ethoxy)-2-methyl-N-(1-(2-(2-(pyrrolidin-1-yl)pyridin-3-yl)quinolin-4-yl)ethyl)benzamide

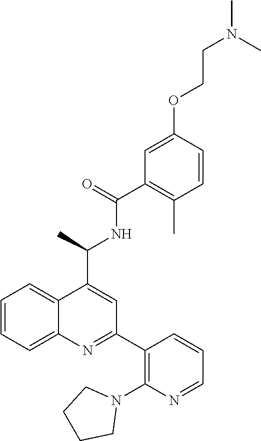 [SMILES: Cc1ccc(OCCN(C)C)cc1C(=O)N[C@H](C)c1cc(-c2cccnc2N2CCCC2)nc2ccccc12]

White solid, ¹H NMR (400 MHz, DMSO-d₆) δ 9.99 (s, 1H), 9.03 (d, J=7.2 Hz, 1H), 8.40 (d, J=8.5 Hz, 1H), 8.21 (d, J=5.7 Hz, 1H), 8.13 (d, J=8.4 Hz, 1H), 7.98 (d, J=7.4 Hz, 1H), 7.95-7.84 (m, 1H), 7.80-7.67 (m, 2H), 7.19 (d, J=8.8 Hz, 1H), 7.06-6.94 (m, 3H), 5.92 (p, J=7.0 Hz, 1H), 4.30 (t, J=5.2 Hz, 2H), 3.61-3.39 (m, 2H), 3.04 (s, 4H), 2.87 (s, 6H), 2.19 (s, 3H), 1.69 (p, J=6.9 Hz, 4H), 1.61 (d, J=6.9 Hz, 3H). ¹³C NMR (101 MHz, DMSO-d₆) δ 168.48, 162.27, 158.95, 158.60, 156.66, 155.69, 152.23, 144.04, 139.53, 137.90, 132.11, 130.90, 129.43, 128.46, 127.78, 124.84, 124.09, 119.07, 115.94, 114.42, 112.65, 62.91, 62.82, 55.87, 51.07, 45.18, 43.27, 25.40, 21.37, 18.87, 18.85. LCMS calcd for C₃₂H₃₈N₅O₂ (M+H⁺): 524.6810; Found: 524.7.

### Example 32. Synthesis of (R)-5-(2-(dimethylamino)ethoxy)-N-(1-(2-(1-(2-hydroxy-2-methylpropyl)-1H-pyrazol-4-yl)quinolin-4-yl)ethyl)-2-methylbenzamide

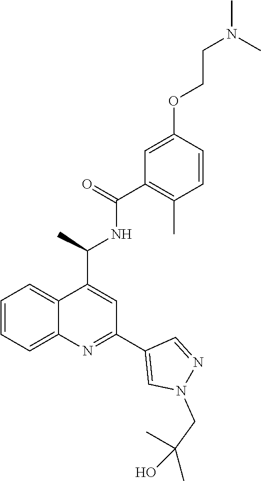 [SMILES: Cc1ccc(OCCN(C)C)cc1C(=O)N[C@H](C)c1cc(-c2cnn(CC(C)(C)O)c2)nc2ccccc12]

White solid, ¹H NMR (400 MHz, DMSO-d₆) δ 9.91 (s, 1H), 9.06 (d, J=7.6 Hz, 1H), 8.54 (s, 1H), 8.35 (d, J=8.5 Hz, 1H), 8.25 (s, 1H), 8.10 (d, J=8.5 Hz, 1H), 8.04 (s, 1H), 7.86 (t, J=7.7 Hz, 1H), 7.68 (t, J=7.7 Hz, 1H), 7.20 (d, J=8.3 Hz, 1H), 7.00 (d, J=7.0 Hz, 2H), 5.93 (p, J=7.0 Hz, 1H), 4.32 (t, J=5.0 Hz, 2H), 4.15 (s, 2H), 3.51 (q, J=4.8 Hz, 2H), 2.21 (s, 3H), 1.64 (d, J=7.0 Hz, 3H), 1.13 (s, 6H). ¹³C NMR (101 MHz, DMSO-d₆) δ 168.53, 158.91, 158.57, 155.70, 150.97, 138.14, 138.06, 132.06, 131.26, 128.33, 126.94, 124.62, 124.19, 118.22, 116.12, 116.00, 115.29, 114.11, 69.64, 62.92, 62.88, 55.90, 45.08, 43.27, 27.64, 21.50, 18.80.

LCMS calcd for C₃₀H₃₈N₅O₃ (M+H⁺): 516.2896; Found: 516.2899.

### Example 33. Synthesis of (R)-5-(2-(dimethylamino)ethoxy)-2-methyl-N-(1-(2-(2-morpholinopyrimidin-5-yl)quinolin-4-yl)ethyl)benzamide

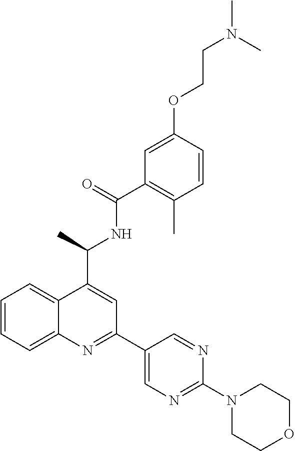 [SMILES: Cc1ccc(OCCN(C)C)cc1C(=O)N[C@H](C)c1cc(-c2cnc(N3CCOCC3)nc2)nc2ccccc12]

White solid, ¹H NMR (400 MHz, DMSO-d₆) δ 9.76 (s, 1H), 9.21 (s, 2H), 8.98 (d, J=7.9 Hz, 1H), 8.30 (d, J=8.5 Hz, 1H), 8.21-8.01 (m, 2H), 7.81 (t, J=7.7 Hz, 1H), 7.65 (t, J=7.7 Hz, 1H), 7.21 (d, J=8.8 Hz, 1H), 7.00 (d, J=6.1 Hz, 2H), 5.96 (p, J=7.0 Hz, 1H), 4.31 (d, J=5.0 Hz, 2H), 3.84 (t, J=4.8 Hz, 4H), 3.71 (t, J=4.8 Hz, 4H), 3.51 (q, J=4.9 Hz, 2H), 2.86 (d, J=4.0 Hz, 6H), 2.22 (s, 3H), 1.63 (d, J=6.9 Hz, 3H). ¹³C NMR (101 MHz, DMSO-d₆) δ 168.44, 161.70, 158.80, 158.46, 157.23, 155.69, 152.82, 151.94, 148.04, 138.31, 132.05, 130.39, 128.26, 126.86, 124.96, 123.85, 121.20, 116.07, 114.29, 114.14, 66.42, 62.87, 55.91, 44.81, 44.49, 43.28, 21.83, 18.80. LCMS calcd for C₃₁H₃₇N₆O₃ (M+H⁺): 541.6680; Found: 541.7.

### Example 34. Synthesis of (R)-5-(2-(dimethylamino)ethoxy)-2-methyl-N-(1-(2-(3-(4-methylpiperazine-1-carbonyl)phenyl)quinolin-4-yl)ethyl)benzamide

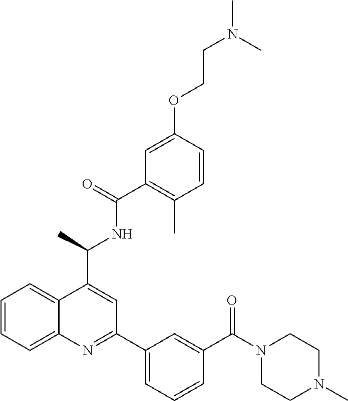 [SMILES: Cc1ccc(OCCN(C)C)cc1C(=O)N[C@H](C)c1cc(-c2cccc(C(=O)N3CCN(C)CC3)c2)nc2ccccc12]

White solid, ¹H NMR (400 MHz, DMSO-d₆) δ 9.95 (s, 1H), 9.10 (d, J=7.8 Hz, 1H), 5.99 (p, J=7.1 Hz, 1H), 4.31 (t, J=5.0 Hz, 2H), 3.51 (s, 3H), 2.85 (d, J=7.3 Hz, 9H), 2.20 (s, 3H), 1.65 (d, J=6.9 Hz, 3H). ¹³C NMR (101 MHz, DMSO-d₆) δ 169.47, 168.48, 159.04, 158.70, 155.72, 155.42, 152.14, 148.21, 139.62, 138.30, 135.96, 132.05, 130.41, 129.55, 128.23, 126.42, 125.29, 116.10, 115.49, 114.04, 62.85, 55.88, 52.60, 44.94, 43.24, 42.74, 21.81, 18.79. LCMS calcd for C₃₅H₄₂N₅O₃ (M+H⁺): 580.7450; Found: 580.3.

### Example 35. Synthesis of (R)-5-(2-(dimethylamino)ethoxy)-2-methyl-N-(1-(2-(5-(trifluoromethyl)thiophen-2-yl)quinolin-4-yl)ethyl)benzamide

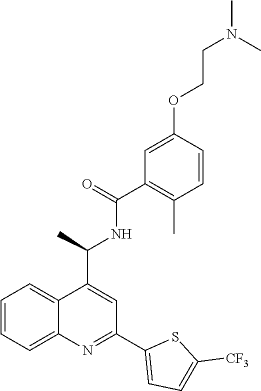 [SMILES: Cc1ccc(OCCN(C)C)cc1C(=O)N[C@H](C)c1cc(-c2ccc(C(F)(F)F)s2)nc2ccccc12]

White solid, ¹H NMR (400 MHz, DMSO-d₆) δ 9.71 (s, 1H), 9.01 (d, J=7.8 Hz, 1H), 8.32 (d, J=8.5 Hz, 1H), 8.23 (s, 1H), 8.06 (d, J=8.4 Hz, 1H), 7.98 (d, J=4.0 Hz, 1H), 7.83 (t, J=7.1 Hz, 2H), 7.70 (t, J=7.7 Hz, 1H), 7.20 (d, J=8.3 Hz, 1H), 6.99 (d, J=8.0 Hz, 2H), 5.95 (p, J=7.2 Hz, 1H), 4.31 (t, J=5.0 Hz, 2H), 2.86 (d, J=3.4 Hz, 6H), 2.21 (s, 3H), 1.65 (d, J=7.0 Hz, 3H). ¹³C NMR (101 MHz, DMSO-d₆) δ 168.48, 158.81, 158.46, 155.69, 152.22, 150.59, 149.94, 147.91, 138.22, 132.05, 131.68, 130.83, 129.97, 128.28, 127.71, 126.55, 125.73, 124.07, 116.12, 114.39, 114.08, 62.84, 55.91, 44.87, 43.27, 21.58, 18.79. LCMS calcd for C₂₈H₂₉F₃N₃O₂S (M+H⁺): 528.6062; Found: 528.6.

### Example 36. Synthesis of (R)-5-(2-(dimethylamino)ethoxy)-N-(1-(2-(1-ethyl-3-(trifluoromethyl)-1H-pyrazol-4-yl)quinolin-4-yl)ethyl)-2-methylbenzamide

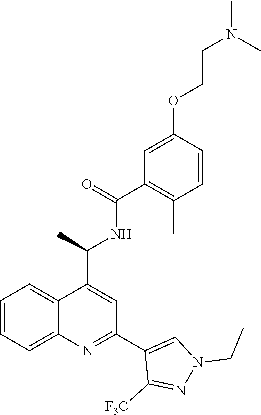 [SMILES: CCn1cc(-c2cc([C@@H](C)NC(=O)c3cc(OCCN(C)C)ccc3C)c3ccccc3n2)c(C(F)(F)F)n1]

White solid, ¹H NMR (400 MHz, DMSO-d₆) δ 9.85 (s, 1H), 8.99 (d, J=7.3 Hz, 1H), 8.61 (s, 1H), 8.30 (d, J=8.5 Hz, 1H), 8.01 (d, J=8.4 Hz, 1H), 7.94-7.75 (m, 2H), 7.66 (t, J=7.6 Hz, 1H), 7.19 (d, J=8.1 Hz, 1H), 7.11-6.80 (m, 2H), 5.90 (p, J=7.0 Hz, 1H), 4.39-4.20 (m, 4H), 3.51 (q, J=4.7 Hz, 2H), 2.86 (s, 6H), 2.22 (s, 3H), 1.63 (d, J=7.0 Hz, 3H), 1.48 (t, J=7.2 Hz, 3H). ¹³C NMR (101 MHz, DMSO-d₆) δ 168.39, 158.92, 158.57, 155.62, 151.07, 150.09, 148.12, 138.08, 133.13, 132.01, 130.20, 130.06, 128.45, 127.05, 124.87, 123.90, 121.76, 117.05, 115.96, 114.25, 62.86, 55.90, 47.83, 44.82, 43.26, 21.26, 18.82, 15.55. LCMS calcd for C₂₉H₃₃F₃N₅O₂ (M+H⁺): 540.6032; Found: 540.2.

### Example 37. Synthesis of (R)—N-(1-(2-(6-cyanopyridin-3-yl)quinolin-4-yl)ethyl)-5-(2-(dimethylamino)ethoxy)-2-methylbenzamide

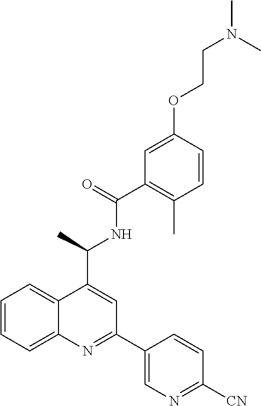 [SMILES: Cc1ccc(OCCN(C)C)cc1C(=O)N[C@H](C)c1cc(-c2ccc(C#N)nc2)nc2ccccc12]

White solid, ¹H NMR (400 MHz, DMSO-d₆) δ 9.72 (s, 1H), 9.57 (d, J=2.2 Hz, 1H), 9.01 (d, J=8.0 Hz, 1H), 8.83 (dd, J=8.1, 2.2 Hz, 1H), 8.46-8.35 (m, 2H), 8.28 (d, J=8.2 Hz, 1H), 8.20 (d, J=8.5 Hz, 1H), 7.89 (t, J=7.6 Hz, 1H), 7.76 (t, J=7.7 Hz, 1H), 7.20 (d, J=8.5 Hz, 1H), 7.00 (d, J=6.5 Hz, 2H), 6.01 (q, J=7.3 Hz, 1H), 4.31 (t, J=4.9 Hz, 2H), 3.51 (q, J=5.0 Hz, 2H), 2.86 (d, J=4.2 Hz, 6H), 2.21 (s, 3H), 1.66 (d, J=6.8 Hz, 3H). ¹³C NMR (101 MHz, DMSO-d₆) δ 168.48, 158.82, 158.47, 155.70, 152.57, 152.42, 150.04, 148.35, 138.22, 137.79, 136.31, 133.32, 132.06, 130.79, 129.78, 128.28, 128.23, 125.64, 123.98, 117.99, 116.15, 115.91, 114.09, 62.85, 55.92, 44.87, 43.28, 21.85, 18.79. LCMS calcd for C₂₉H₃₀N₅O₂ (M+H⁺): 480.5840; Found: 480.6.

### Example 38. Synthesis of (R)—N-(1-(2-(1,3-dimethyl-1H-pyrazol-5-yl)quinolin-4-yl)ethyl)-5-(2-(dimethylamino)ethoxy)-2-methylbenzamide

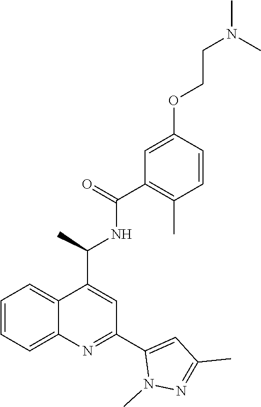 [SMILES: Cc1cc(-c2cc([C@@H](C)NC(=O)c3cc(OCCN(C)C)ccc3C)c3ccccc3n2)n(C)n1]

White solid, ¹H NMR (400 MHz, DMSO-d₆) δ 9.86 (s, 1H), 9.03 (d, J=7.8 Hz, 1H), 8.32 (d, J=8.4 Hz, 1H), 8.11 (d, J=8.4 Hz, 1H), 7.93 (s, 1H), 7.83 (t, J=7.6 Hz, 1H), 7.70 (t, J=7.6 Hz, 1H), 7.21 (d, J=8.2 Hz, 1H), 7.04-6.91 (m, 2H), 6.68 (s, 1H), 5.94 (p, J=7.1 Hz, 1H), 4.31 (t, J=4.9 Hz, 2H), 4.25 (s, 3H), 3.51 (q, J=4.7 Hz, 2H), 2.22 (d, J=4.4 Hz, 6H), 1.61 (d, J=6.9 Hz, 3H). ¹³C NMR (101 MHz, DMSO-d₆) δ 168.45, 158.88, 158.54, 155.70, 151.73, 149.56, 146.45, 141.60, 138.26, 132.05, 130.39, 128.27, 127.45, 124.75, 123.80, 117.26, 116.03, 114.13, 107.27, 62.85, 55.90, 44.67, 43.26, 21.70, 18.81, 13.61. LCMS calcd for C₂₈H₃₄N₅O₂ (M+H⁺): 472.6050; Found: 472.2.

### Example 39. Synthesis of (R)-5-(2-(dimethylamino)ethoxy)-2-methyl-N-(1-(2-(6-morpholinopyridin-3-yl)quinolin-4-yl)ethyl)benzamide

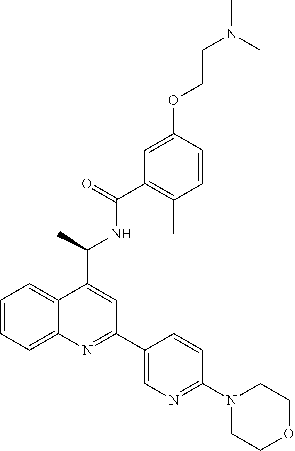 [SMILES: Cc1ccc(OCCN(C)C)cc1C(=O)N[C@H](C)c1cc(-c2ccc(N3CCOCC3)nc2)nc2ccccc12]

White solid, ¹H NMR (400 MHz, DMSO-d₆) δ 9.78 (s, 1H), 8.98 (d, J=2.3 Hz, 1H), 8.44 (dd, J=8.9, 2.6 Hz, 1H), 8.34 (d, J=8.5 Hz, 1H), 8.19 (s, 1H), 8.12 (d, J=8.5 Hz, 1H), 7.85 (t, J=7.7 Hz, 1H), 7.68 (t, J=7.6 Hz, 1H), 7.21 (d, J=8.5 Hz, 1H), 7.09 (d, J=9.1 Hz, 1H), 7.03-6.96 (m, 2H), 5.97 (t, J=7.3 Hz, 1H), 4.31 (t, J=5.1 Hz, 2H), 3.74 (t, J=4.8 Hz, 4H), 3.63 (t, J=4.8 Hz, 4H), 3.51 (q, J=4.7 Hz, 3H), 2.21 (s, 3H), 1.64 (d, J=6.9 Hz, 3H). ¹³C NMR (101 MHz, DMSO-d₆) δ 168.49, 159.59, 158.86, 158.51, 155.71, 154.10, 138.28, 136.96, 132.07, 130.84, 128.39, 128.26, 127.01, 124.79, 123.95, 122.31, 117.99, 117.67, 116.08, 114.80, 114.13, 107.51, 66.34, 62.88, 55.91, 45.37, 44.96, 43.28, 21.84, 18.80. LCMS calcd for C₃₂H₃₈N₅O₃ (M+H⁺): 540.6800; Found: 540.2.

### Example 40. Synthesis of (R)—N-(1-(2-(1-(3-(cyanomethyl)azetidin-3-yl)-1H-pyrazol-4-yl)quinolin-4-yl)ethyl)-5-(2-(dimethylamino)ethoxy)-2-methylbenzamide

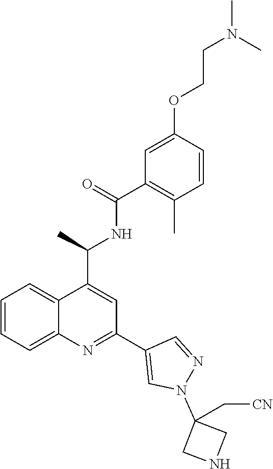 [SMILES: Cc1ccc(OCCN(C)C)cc1C(=O)N[C@H](C)c1cc(-c2cnn(C3(CC#N)CNC3)c2)nc2ccccc12]

White solid, ¹H NMR (400 MHz, DMSO-d₆) δ 10.12 (s, 1H), 9.85 (s, 1H), 9.17 (d, J=7.3 Hz, 1H), 8.78 (s, 1H), 8.45 (d, J=8.7 Hz, 2H), 7.96 (t, J=8.0 Hz, 1H), 7.78 (t, J=7.8 Hz, 1H), 7.17 (d, J=8.5 Hz, 1H), 7.10 (d, J=2.5 Hz, 1H), 6.99 (dd, J=8.2, 2.6 Hz, 2H), 5.97 (t, J=7.2 Hz, 1H), 4.66 (d, J=9.5 Hz, 2H), 4.40 (d, J=4.6 Hz, 2H), 3.94 (s, 2H), 3.72-3.67 (m, 2H), 3.49 (d, J=4.7 Hz, 2H), 2.83 (d, J=4.5 Hz, 6H), 2.21 (s, 3H), 1.69 (d, J=6.9 Hz, 3H). ¹³C NMR (101 MHz, DMSO-d₆) δ 168.54, 164.27, 155.65, 140.31, 139.90, 137.65, 132.07, 128.54, 124.90, 124.54, 116.39, 114.34, 60.65, 59.42, 55.66, 54.24, 44.07, 43.20, 26.75, 21.35, 18.96. LCMS calcd for C₃₁H₃₆N₇O₂ (M+H⁺): 538.6680; Found: 538.2.

### Example 41. Synthesis of (R)-5-(2-(dimethylamino)ethoxy)-2-methyl-N-(1-(2-(1-methyl-1H-pyrazol-3-yl)quinolin-4-yl)ethyl)benzamide

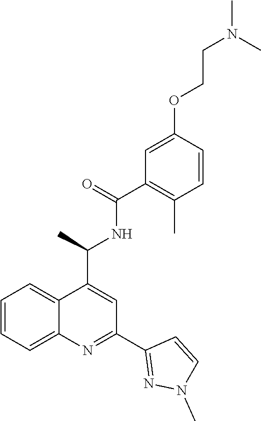 [SMILES: Cc1ccc(OCCN(C)C)cc1C(=O)N[C@H](C)c1cc(-c2ccn(C)n2)nc2ccccc12]

White solid, ¹H NMR (400 MHz, DMSO-d₆) δ 9.85 (s, 1H), 9.18 (d, J=7.4 Hz, 1H), 8.40-8.21 (m, 2H), 8.13 (d, J=8.4 Hz, 1H), 7.99-7.78 (m, 2H), 7.68 (t, J1=7.7 Hz, 1H), 7.21 (d, J=8.8 Hz, 1H), 7.13-6.94 (m, 3H), 5.90 (p, J=7.0 Hz, 1H), 4.34 (s, 2H), 3.97 (s, 3H), 3.52 (q, J=4.8 Hz, 2H), 2.87 (s, 6H), 2.23 (s, 3H), 1.61 (d, J=6.9 Hz, 3H). ¹³C NMR (101 MHz, DMSO-d₆) δ 168.54, 158.89, 158.54, 155.67, 152.70, 151.61, 150.26, 146.86, 138.23, 133.56, 132.05, 130.58, 128.91, 128.50, 127.06, 125.44, 124.02, 116.03, 114.84, 114.25, 105.36, 62.86, 55.91, 45.11, 43.28, 39.48, 21.45, 18.82. LCMS calcd for C₂₇H₃₂N₅O₂ (M+H⁺): 458.5780; Found: 458.2.

### Example 42. Synthesis of (R)-5-(2-(dimethylamino)ethoxy)-2-methyl-N-(1-(2-(4-methylthiazol-5-yl)quinolin-4-yl)ethyl)benzamide

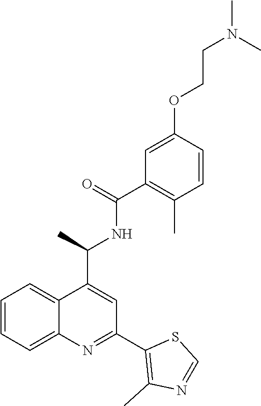 [SMILES: Cc1ccc(OCCN(C)C)cc1C(=O)N[C@H](C)c1cc(-c2scnc2C)nc2ccccc12]

‘White solid, ¹H NMR (400 MHz, DMSO-d₆) δ 9.81 (s, 1H), 9.18-9.05 (m, 1H), 8.33 (d, J=8.5 Hz, 1H), 8.06 (d, J=8.4 Hz, 1H), 7.83 (dd, J=8.6, 5.2 Hz, 1H), 7.69 (t, J=7.7 Hz, 1H), 7.50 (t, J=7.6 Hz, 1H), 7.21 (dd, J=8.8, 3.8 Hz, 1H), 7.03-6.99 (m, 2H), 5.94 (p, J=7.1 Hz, 1H), 4.31 (t, J=4.9 Hz, 2H), 3.98 (s, 3H), 2.23 (s, 3H), 1.62 (d, J=7.0 Hz, 3H). ¹³C NMR (101 MHz, DMSO-d₆) δ 168.50, 162.41, 155.69, 154.42, 152.11, 151.29, 148.13, 146.89, 138.13, 133.16, 132.09, 130.60, 128.37, 127.38, 124.64, 123.88, 116.55, 114.11, 109.20, 62.90, 55.94, 53.56, 44.93, 43.31, 21.58, 18.86. LCMS calcd for C₂₇H₃₁N₄O₂S (M+H⁺): 475.623; Found: 475.2.

### Example 43. Synthesis of (R)-5-(2-(dimethylamino)ethoxy)-2-methyl-N-(1-(2-(4-(methylsulfonyl)phenyl)quinolin-4-yl)ethyl)benzamide

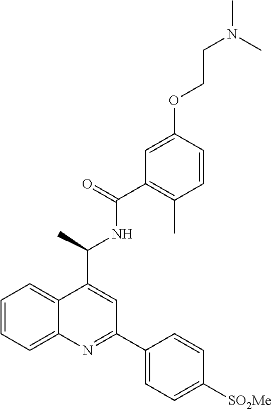 [SMILES: Cc1ccc(OCCN(C)C)cc1C(=O)N[C@H](C)c1cc(-c2ccc(S(C)(=O)=O)cc2)nc2ccccc12]

White solid, ¹H NMR (400 MHz, DMSO-d₆) δ 7.91 (d, J=2.1 Hz, 1H), 7.73 (d, J=7.9 Hz, 1H), 7.65 (d, J=8.5 Hz, 1H), 7.50-7.40 (m, 2H), 7.36 (d, J=3.6 Hz, 1H), 7.13-7.05 (m, 2H), 6.99 (t, J=7.7 Hz, 1H), 6.40 (d, J=5.5 Hz, 1H), 6.29-6.22 (m, 2H), 5.32 (q, J=7.0 Hz, 1H), 3.54 (d, J=8.8 Hz, 2H), 3.28 (s, 2H), 2.80 (d, J=4.1 Hz, 2H), 2.17 (s, 6H), 1.52 (s, 3H), 0.97 (d, J=7.0 Hz, 3H). ¹³C NMR (101 MHz, CD₃OD) δ 170.48, 162.51, 155.60, 154.64, 141.73, 137.15, 132.71, 131.77, 130.08, 128.52, 127.69, 126.16, 125.29, 124.28, 123.33, 123.12, 116.08, 115.70, 115.29, 113.17, 112.86, 108.39, 61.95, 61.93, 56.33, 56.29, 45.67, 42.96, 42.51, 20.10, 17.44. LCMS calcd for C₃₀H₃₄N₃O₄S (M+H⁺): 532.6170; Found: 532.2.

### Example 44. Synthesis of (R)-5-(2-(dimethylamino)ethoxy)-N-(1-(2-(1-ethyl-1H-pyrazol-3-yl)quinolin-4-yl)ethyl)-2-methylbenzamide

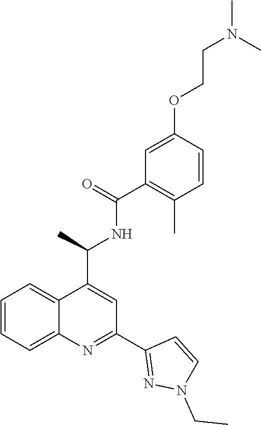 [SMILES: CCn1ccc(-c2cc([C@@H](C)NC(=O)c3cc(OCCN(C)C)ccc3C)c3ccccc3n2)n1]

White solid, 68% yield. ¹H NMR (400 MHz, DMSO-d₆) δ 9.25 (d, J=7.2 Hz, 1H), 8.43 (d, J=8.5 Hz, 1H), 8.38 (s, 1H), 8.24 (d, J=8.5 Hz, 1H), 8.02 (d, J=2.3 Hz, 1H), 7.93 (t, J=7.7 Hz, 1H), 7.29-7.24 (m, 1H), 7.17 (d, J=2.3 Hz, 1H), 7.13-6.98 (m, 2H), 5.95 (q, J=7.1 Hz, 1H), 4.38 (t, J=5.1 Hz, 2H), 3.57 (q, J=4.6 Hz, 1H), 2.93 (s, 3H), 2.29 (s, 2H), 1.68 (d, J=6.9 Hz, 2H). ¹³C NMR (101 MHz, DMSO-d₆) δ 168.64, 155.69, 151.18, 149.16, 138.21, 132.25, 132.04, 131.10, 128.48, 128.05, 127.36, 125.42, 124.17, 116.02, 114.92, 114.28, 105.62, 62.87, 55.91, 47.35, 45.41, 43.28, 21.39, 18.80, 15.91. LCMS calcd for C₂₈H₃₄N₅O₂ (M+H⁺): 472.2636; Found: 472.2639.

### General Synthetic Procedure Scheme 2.

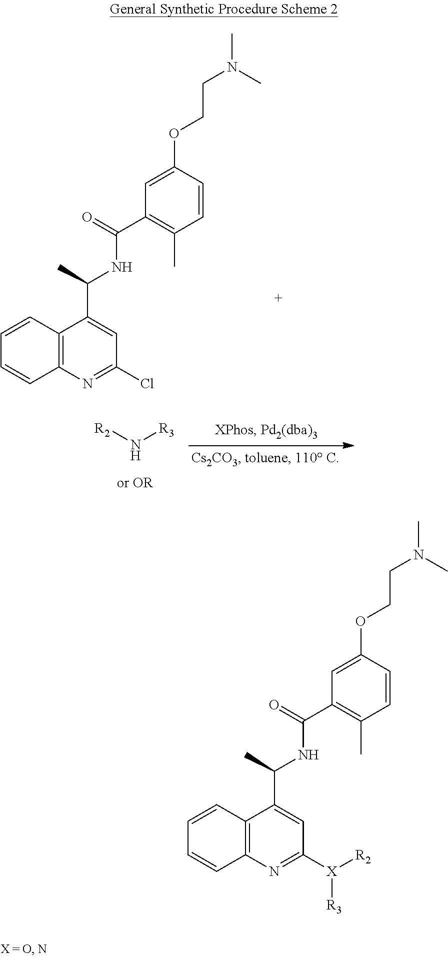

### General Coupling Procedure.

To a solution of methyl 5-bromo-2-methylbenzoate (1.0 equiv) in dry toluene in sealed tube, corresponding amine (1.5 equiv), XPhos (0.04 equiv), tris(dibenzylideneacetone)dipalladium (0.02 equiv) and Cs₂CO₃ (2.0 equiv) were added. The reaction mixture was degassed and purged with argon, and then the tube was sealed and heated to 110° C. overnight. The mixture was diluted with EtOAc and was then washed with water and brine. The organic layer was dried over Na₂SO₄, filtered, and concentrated. The residue was purified by silica gel column chromatography (Hexanes/EtOAc=1:1) to provide the desired product.

### Example 45. Synthesis of (R)-5-(2-(dimethylamino)ethoxy)-2-methyl-N-(1-(2-morpholinoquinoline-4-yl)ethyl)benzamide

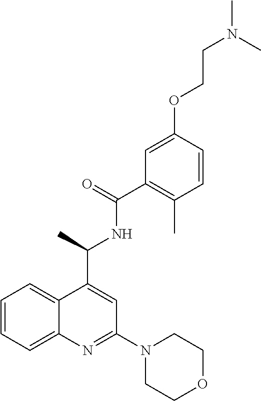 [SMILES: Cc1ccc(OCCN(C)C)cc1C(=O)N[C@H](C)c1cc(N2CCOCC2)nc2ccccc12]

White solid, ¹H NMR (400 MHz, DMSO-d₆) δ 10.01 (s, 1H), 9.00 (d, J=7.7 Hz, 1H), 8.22 (d, J=8.3 Hz, 1H), 7.94 (d, J=8.5 Hz, 1H), 7.79 (t, J=7.8 Hz, 1H), 7.54 (q, J=7.6, 6.2 Hz, 1H), 7.47 (s, 1H), 7.20 (d, J=7.8 Hz, 1H), 7.00 (d, J=9.3 Hz, 2H), 5.81 (d, J=7.2 Hz, 1H), 4.32 (t, J=4.9 Hz, 2H), 3.88-3.75 (m, 7H), 3.52 (q, J=4.8 Hz, 2H), 2.87 (d, J=3.7 Hz, 6H), 2.23 (d, J=6.6 Hz, 3H), 1.56 (d, J=7.0 Hz, 3H). ¹³C NMR (101 MHz, DMSO-d₆) δ 168.56, 159.25, 158.91, 158.56, 158.22, 155.72, 138.06, 132.06, 128.34, 124.96, 124.34, 120.11, 118.14, 116.27, 114.06, 107.78, 65.99, 62.92, 55.92, 46.61, 45.37, 43.29, 21.50, 18.82. LCMS calcd for C₂₇H₃₅N₄O₃ (M+H⁺): 463.5940; Found: 463.6.

### Example 46. Synthesis of (R)—N-(1-(2-(1H-imidazol-1-yl)quinolin-4-yl)ethyl)-5-(2-(dimethylamino)ethoxy)-2-methylbenzamide

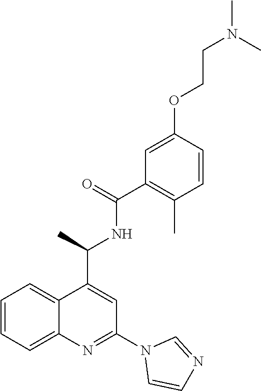 [SMILES: Cc1ccc(OCCN(C)C)cc1C(=O)N[C@H](C)c1cc(-n2ccnc2)nc2ccccc12]

White solid, 53% yield, ¹H NMR (400 MHz, DMSO) δ 9.81 (s, 1H), 9.10 (d, J=7.4 Hz, 1H), 8.28 (d, J=9.6 Hz, 2H), 8.08 (d, J=8.5 Hz, 1H), 7.85 (d, J=2.3 Hz, 1H), 7.77 (q, J=6.8, 6.2 Hz, 1H), 7.62 (q, J=8.1 Hz, 1H), 7.18-7.10 (m, 1H), 7.02 (d, J=2.4 Hz, 1H), 6.93 (d, J=6.6 Hz, 2H), 5.85 (q, J=7.2 Hz, 1H), 4.25 (d, J=5.4 Hz, 2H), 3.46 (q, J=5.0 Hz, 2H), 2.80 (d, J=3.8 Hz, 6H), 2.16 (d, J=6.2 Hz, 3H), 1.56 (d, J=6.9 Hz, 3H). ¹³C NMR (101 MHz, DMSO) δ 168.53, 155.66, 151.49, 146.63, 138.19, 132.04, 130.71, 128.75, 128.44, 127.15, 125.47, 124.08, 118.05, 116.10, 115.15, 114.13, 104.69, 93.94, 62.86, 55.89, 45.06, 43.26, 21.42, 18.84. LCMS calcd for C₂₆H₃₀NO₂ (M+H⁺): 444.2321; Found: 444.2323.

### Example 47. Synthesis of (R)—N-(1-(2-(1H-1,2,4-triazol-1-yl)quinoline 4-yl)ethyl)-5-(2-(dimethylamino)ethoxy)-2-methylbenzamide

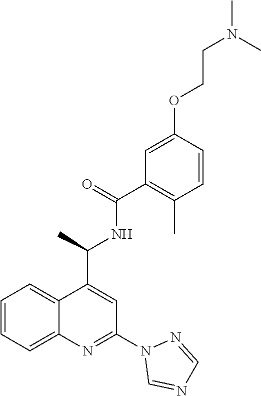 [SMILES: Cc1ccc(OCCN(C)C)cc1C(=O)N[C@H](C)c1cc(-n2cncn2)nc2ccccc12]

White solid, 58% yield, ¹H NMR (400 MHz, DMSO-d₆) δ 9.88 (s, 1H), 9.26 (d, J=17.0 Hz, 1H), 8.99-8.87 (m, 1H), 8.36 (d, J=17.1 Hz, 1H), 8.18-8.07 (m, 1H), 7.94-7.83 (m, 1H), 7.74 (t, J=10.6 Hz, 1H), 7.23 (d, J=17.6 Hz, 1H), 7.05 (dd, J=21.2, 10.4 Hz, 2H), 5.92 (s, 1H), 4.32 (d, J=16.2 Hz, 2H), 3.50 (d, J=19.2 Hz, 2H), 2.85 (d, J=16.4 Hz, 6H), 2.28 (dd, J=25.8, 11.9 Hz, 3H), 1.74-1.56 (m, 3H). ¹³C NMR (101 MHz, DMSO-d₆) δ 168.79, 158.53, 155.71, 152.02, 148.04, 138.46, 132.05, 130.43, 130.33, 128.47, 127.90, 126.40, 124.11, 115.93, 114.92, 114.34, 62.89, 55.91, 45.63, 43.28, 21.48, 18.91. LCMS calcd for C₂₆H₂₉N₆O₂ (M+H⁺): 445.2274; Found: 445.2278.

### Example 48. Synthesis of (R)-5-(2-(dimethylamino)ethoxy)-2-methyl-N-(1-(2-(5-(4-methylpiperazin-1-yl)thiophen-2-yl)quinolin-4-yl)ethyl)benzamide

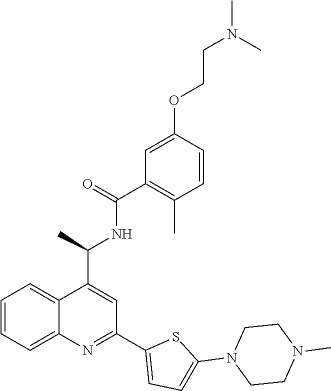 [SMILES: Cc1ccc(OCCN(C)C)cc1C(=O)N[C@H](C)c1cc(-c2ccc(N3CCN(C)CC3)s2)nc2ccccc12]

White solid, 55% yield, ¹H NMR (400 MHz, DMSO-d₆) δ9.92 (s, 2H), 9.04 (d, J=7.7 Hz, 1H), 8.30 (d, J=8.5 Hz, 1H), 8.14 (s, 1H), 8.00 (d, J=8.4 Hz, 1H), 7.87 (d, J=3.7 Hz, 1H), 7.81 (t, J=7.6 Hz, 1H), 7.66 (t, J=7.6 Hz, 1H), 7.40 (d, J=3.8 Hz, 1H), 7.20 (d, J=9.0 Hz, 1H), 7.03-6.96 (m, 2H), 5.92 (p, J=7.0 Hz, 1H), 4.59 (s, 2H), 3.51 (dt, J=7.6, 4.0 Hz, 2H), 3.44 (d, J=11.8 Hz, 2H), 2.94 (d, J=11.8 Hz, 2H), 2.86 (d, J=3.1 Hz, 5H), 2.50 (s, 3H), 2.20 (s, 3H), 1.85 (d, J=13.7 Hz, 2H), 1.70 (d, J=13.3 Hz, 2H), 1.65 (s, 3H). ¹³C NMR (101 MHz, DMSO-d₆) −168.49, 158.83, 155.71, 151.82, 151.45, 148.23, 148.05, 138.28, 133.90, 132.04, 130.58, 129.81, 128.24, 127.20, 126.77, 125.40, 124.04, 116.05, 114.13, 62.88, 55.89, 53.54, 52.06, 44.98, 43.26, 22.93, 21.59, 18.80. LCMS calcd for C₃₂H₄₀N₅O₂S (M+⁺): 557.2824; Found: 557.2826.

### Example 49. Synthesis of (R)—N-(1-(2-(1H-pyrazolo[3,4-b]pyridin-5-yl)quinolin-4-yl)-ethyl)-5-(2-(dimethylamino)-ethoxy)-2-methylbenzamide

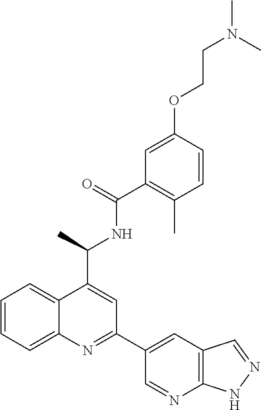 [SMILES: Cc1ccc(OCCN(C)C)cc1C(=O)N[C@H](C)c1cc(-c2cnc3[nH]ncc3c2)nc2ccccc12]

White solid, 51% yield, ¹H NMR (400 MHz, DMSO-d₆) δ 9.70 (s, 1H), 9.42 (s, 1H), 9.04 (d, J=4.7 Hz, 2H), 8.38-8.26 (m, 3H), 8.17 (d, J=8.4 Hz, 1H), 7.85 (t, J=7.7 Hz, 1H), 7.69 (t, J=7.7 Hz, 1H), 7.25-7.16 (m, 1H), 6.99 (d, J=6.0 Hz, 2H), 6.00 (q, J=7.3 Hz, 1H), 4.31 (t, J=5.0 Hz, 2H), 3.51 (t, J=5.2 Hz, 2H), 2.85 (d, J=4.3 Hz, 6H), 2.22 (s, 3H), 1.67 (d, J=6.9 Hz, 3H). ¹³C NMR (101 MHz, DMSO-d₆) δ 168.46, 158.45, 155.71, 152.03, 148.91, 138.33, 134.77, 132.06, 130.43, 130.26, 129.13, 128.27, 127.18, 125.07, 123.86, 116.11, 115.55, 115.00, 114.07, 62.84, 55.94, 43.29, 21.89, 18.79. LCMS calcd for C₂₉H₃₁N₆O₂ (M+H⁺): 495.2430; Found: 495.2433.

### Example 50. Synthesis of (R)—N-(1-(2-(1H-indazol-4-yl)quinolin-4-yl)ethyl)-5-(2-(dimethylamino)ethoxy)-2-methylbenzamide

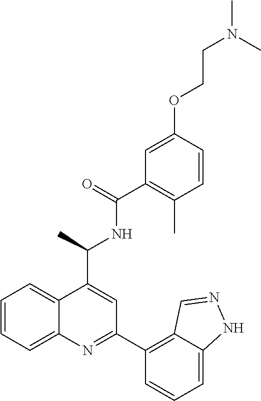 [SMILES: Cc1ccc(OCCN(C)C)cc1C(=O)N[C@H](C)c1cc(-c2cccc3[nH]ncc23)nc2ccccc12]

White solid, 53% yield, ¹H NMR (400 MHz, DMSO-d₆) δ 9.69 (s, 1H), 9.13 (d, J=7.8 Hz, 1H), 8.86 (s, 1H), 8.37 (d, J=8.5 Hz, 1H), 8.26 (s, 1H), 8.06 (s, 1H), 7.86 (s, 1H), 7.74 (s, 1H), 7.55 (t, J=8.2 Hz, 2H), 7.34 (t, J=7.7 Hz, 1H), 7.27-7.16 (m, 1H), 7.10 (t, J=7.5 Hz, 1H), 7.00 (s, 1H), 6.00 (q, J=7.2 Hz, 1H), 4.29 (t, J=5.1 Hz, 2H), 3.50 (q, J=4.9 Hz, 2H), 2.85 (d, J=4.4 Hz, 6H), 2.22 (s, 3H), 1.67 (d, J=6.9 Hz, 3H). ¹³C NMR (101 MHz, DMSO-d₆) δ 168.45, 158.81, 155.71, 151.63, 148.30, 141.39, 140.34, 138.31, 134.86, 133.78, 132.09, 130.32, 128.29, 127.32, 126.33, 125.11, 120.75, 120.62, 116.52, 114.02, 110.54, 62.81, 55.93, 44.83, 43.28, 21.80, 18.81. LCMS calcd for C₃₀H₃₂N₅O₂ (M+H⁺): 495.2430; Found: 495.2433.

### Example 51. Synthesis of (R)-5-(2-(dimethylamino)ethoxy)-2-methyl-N-(1-(2-(5-(pyrrolidin-1-ylmethyl)thiophen-2-yl)quinolin-4-yl)ethyl)benzamide

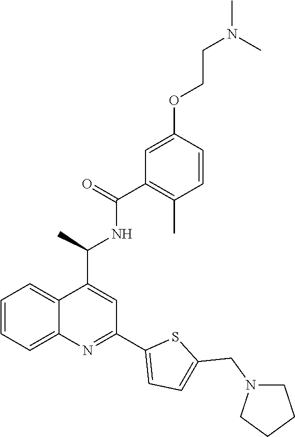 [SMILES: Cc1ccc(OCCN(C)C)cc1C(=O)N[C@H](C)c1cc(-c2ccc(CN3CCCC3)s2)nc2ccccc12]

White solid, 50% yield, ¹H NMR (400 MHz, DMSO-d₆) δ 9.05 (d, J=7.7 Hz, 1H), 8.30 (d, J=8.5 Hz, 1H), 8.14 (s, 1H), 8.01 (d, J=8.4 Hz, 1H), 7.86 (d, J=3.7 Hz, 1H), 7.80 (t, =7.6 Hz, 1H), 7.66 (t, J=7.6 Hz, 1H), 7.41 (d, J=3.7 Hz, 1H), 7.22-7.17 (m, 1H), 6.99 (dd, J=4.5, 2.1 Hz, 2H), 5.91 (q, J=7.2 Hz, 1H), 4.69-4.64 (m, 2H), 4.32 (t, J=5.0 Hz, 2H), 3.51 (dt, J=15.6, 7.1 Hz, 4H), 3.17 (d, J=9.3 Hz, 2H), 2.87 (s, 6H), 2.21 (s, 3H), 2.04 (q, J=7.8, 6.6 Hz, 2H), 1.96-1.82 (m, 2H), 1.64 (d, J=6.9 Hz, 3H). ¹³C NMR (101 MHz, DMSO-d₆) δ 168.49, 155.70, 151.79, 151.50, 148.04, 147.73, 138.26, 135.83, 132.86, 132.03, 130.56, 129.81, 128.25, 126.78, 125.39, 124.03, 118.23, 116.04, 114.19, 114.13, 62.88, 55.87, 53.31, 51.48, 44.96, 43.24, 23.08, 21.57, 18.79. LCMS calcd for C₃₂H₃₉N₄O₂S (M+H⁺): 543.2715; Found: 543.2718.

### Example 52. Synthesis of (R)-5-(2-(dimethylamino)ethoxy)-2-methyl-N-(1-(2-(1-(2-morpholinoethyl)-1H-pyrazol-4-yl)quinolin-4-yl)ethyl)benzamide

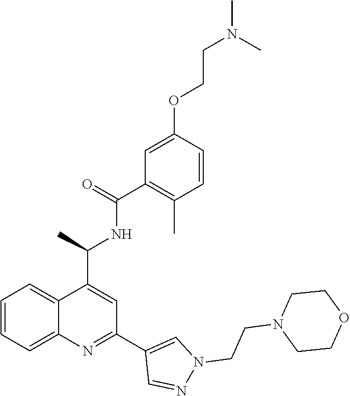 [SMILES: Cc1ccc(OCCN(C)C)cc1C(=O)N[C@H](C)c1cc(-c2cnn(CCN3CCOCC3)c2)nc2ccccc12]

White solid, 48% yield, ¹H NMR (400 MHz, DMSO-d₆) δ 9.03 (d, J=7.6 Hz, 1H), 8.68 (s, 1H), 8.32 (s, 2H), 8.05 (d, J=8.5 Hz, 1H), 7.98 (s, 1H), 7.81 (d, J=7.8 Hz, 1H), 7.65 (t, J=7.6 Hz, 1H), 7.19 (d, J=8.9 Hz, 1H), 6.99 (d, J=5.2 Hz, 2H), 5.90 (p, J=7.0 Hz, 1H), 4.69 (t, J=6.4 Hz, 2H), 4.31 (t, J=5.1 Hz, 2H), 3.96-3.76 (m, 2H), 3.72 (t, J=6.6 Hz, 2H), 3.51 (d, J=4.9 Hz, 2H), 3.28 (dd, J=64.9, 30.7 Hz, 4H), 2.87 (s, 6H), 2.50 (s, 2H), 2.20 (s, 3H), 1.65 (d, J=6.9 Hz, 3H). ¹³C NMR (101 MHz, DMSO-d₆) δ 168.49, 155.69, 151.11, 139.09, 138.19, 132.03, 131.44, 130.70, 128.29, 126.68, 124.83, 124.11, 122.90, 118.09, 116.02, 115.90, 115.17, 114.17, 63.74, 62.87, 55.87, 55.31, 51.90, 46.32, 45.05, 43.24, 21.37, 18.80. LCMS calcd for C₃₂H₄₁N₆O₃ (M+H⁺): 557.7110; Found: 557.7113.

### Example 53. Synthesis of (R)-2-(4-(4-(1-(5-(2-(dimethylamino)ethoxy)-2-methyl benzamido)ethyl)quinolin-2-yl)-1H-pyrazol-1-yl)acetic acid

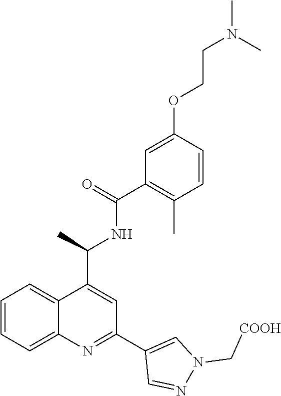 [SMILES: Cc1ccc(OCCN(C)C)cc1C(=O)N[C@H](C)c1cc(-c2cnn(CC(=O)O)c2)nc2ccccc12]

White solid, 50% yield, ¹H NMR (400 MHz, DMSO-d₆) δ 9.75 (s, 1H), 9.01 (d, J=7.7 Hz, 1H), 8.56 (s, 1H), 8.31 (d, J=8.5 Hz, 1H), 8.22 (s, 1H), 8.06 (d, J=8.5 Hz, 1H), 7.98 (s, 1H), 7.83 (t, J=7.7 Hz, 1H), 7.66 (t, J=7.7 Hz, 1H), 7.20 (d, J=8.2 Hz, 1H), 6.99 (d, J=9.3 Hz, 2H), 5.92 (q, J=7.2 Hz, 1H), 2.86 (s, 6H), 2.22 (s, 3H), 1.64 (d, J=6.9 Hz, 3H). ¹³C NMR (101 MHz, DMSO) δ 169.82, 168.48, 158.82, 158.48, 155.68, 138.66, 138.20, 132.18, 132.05, 130.86, 128.33, 126.76, 124.74, 124.07, 116.09, 115.87, 114.11, 62.84, 55.92, 53.51, 44.89, 43.28, 21.49, 18.80. LCMS calcd for C₂₉H₃₂N₅O₄ (M+H⁺): 502.2376; Found: 502.2376.

### Example 54. Synthesis of (R)-5-(2-(dimethylamino)ethoxy)-2-methyl-N-(1-(2-(2-oxo-1,2-dihydropyridin-4-yl)quinolin-4-yl)ethyl)benzamide

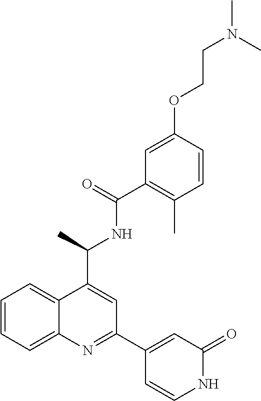 [SMILES: Cc1ccc(OCCN(C)C)cc1C(=O)N[C@H](C)c1cc(-c2cc[nH]c(=O)c2)nc2ccccc12]

White solid, 47% yield, ¹H NMR (400 MHz, DMSO-d₆) δ 9.59 (s, 1H), 9.06 (d, J=8.6 Hz, 1H), 8.36 (d, J=8.8 Hz, 1H), 8.28-8.02 (m, 2H), 7.85 (q, J=10.0, 8.8 Hz, 1H), 7.75 (t, J=7.9 Hz, 1H), 7.57 (d, J=7.1 Hz, 1H), 7.22 (d, J=8.6 Hz, 1H), 7.12 (d, J=7.9 Hz, 2H), 7.01 (d, J=7.6 Hz, 2H), 6.00 (t, J=8.4 Hz, 1H), 4.41-4.20 (m, 2H), 2.87 (d, J=4.9 Hz, 2H), 2.21 (d, J=7.6 Hz, 3H), 1.60 (d, J=7.5 Hz, 3H). LCMS calcd for C₂₈H₃₁N₄O₃ (M+H⁺): 471.2318; Found: 471.2320.

### Example 55. Synthesis of (R)—N-(1-(2-(5-(aminomethyl)thiophen-2-yl)quinolin-4-yl)-ethyl)-5-(2-(dimethylamino)ethoxy)-2-methylbenzamide

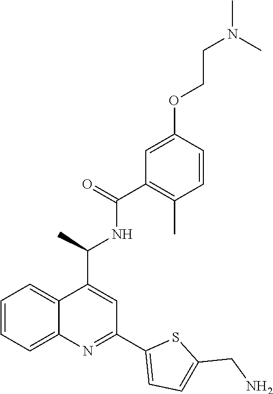 [SMILES: Cc1ccc(OCCN(C)C)cc1C(=O)N[C@H](C)c1cc(-c2ccc(CN)s2)nc2ccccc12]

White solid, 51% yield, ¹H NMR (400 MHz, DMSO-d₆) δ 9.10 (d, J=7.7 Hz, 1H), 8.49 (s, 3H), 8.35 (d, J=8.5 Hz, 1H), 8.17 (s, 1H), 8.06 (d, J=8.4 Hz, 1H), 7.86 (dd, J=9.5, 5.8 Hz, 2H), 7.71 (t, J=7.6 Hz, 1H), 7.37 (d, J=3.8 Hz, 1H), 7.25 (d, J=9.2 Hz, 1H), 7.06 (dd, J=5.9, 2.9 Hz, 2H), 5.95 (q, J=7.2 Hz, 1H), 4.38 (q, J=5.4 Hz, 4H), 3.57 (d, J=10.3 Hz, 4H), 2.92 (s, 6H), 2.26 (s, 3H), 1.70 (d, J=6.9 Hz, 3H). ¹³C NMR (101 MHz, DMSO-d₆) δ 168.54, 158.95, 158.62, 155.69, 151.70, 151.67, 148.06, 146.39, 139.17, 138.25, 132.03, 130.52, 129.78, 128.24, 127.06, 125.32, 123.99, 116.04, 114.09, 62.86, 55.88, 44.99, 43.25, 37.76, 21.52, 18.76. LCMS calcd for C₂₈H₃₁N₄O₃ (M+H⁺): 489.2246; Found: 489.2248.

### Example 56. Synthesis of (R)—N-(1-(2-(2,2-difluorobenzo[d][1,3]dioxol-5-yl)quinolin-4-yl) ethyl)-5-(2-(dimethylamino)ethoxy)-2-methylbenzamid

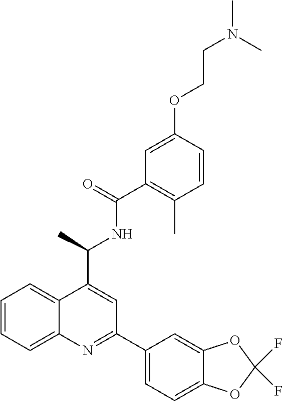 [SMILES: Cc1ccc(OCCN(C)C)cc1C(=O)N[C@H](C)c1cc(-c2ccc3c(c2)OC(F)(F)O3)nc2ccccc12]

White solid, 51% yield, ¹H NMR (400 MHz, DMSO-d₆) δ 9.86 (s, 1H), 9.02 (d, J=8.0 Hz, 1H), 8.34 (d, J=8.4 Hz, 1H), 8.23 (d, J=9.8 Hz, 2H), 8.13 (dd, J=8.3, 6.2 Hz, 2H), 7.83 (t, J=7.6 Hz, 1H), 7.77-7.57 (m, 2H), 7.28-7.13 (m, 1H), 7.07-6.90 (m, 2H), 5.98 (q, J=7.2 Hz, 1H), 4.31 (t, J=4.9 Hz, 2H), 3.51 (q, J=4.7 Hz, 2H), 2.85 (s, 6H), 2.21 (s, 3H), 1.65 (d, J=6.9 Hz, 3H). ¹³C NMR (101 MHz, DMSO-d₆) δ 168.43, 155.70, 154.69, 152.00, 148.12, 144.26, 144.07, 138.29, 136.34, 132.04, 130.41, 128.26, 127.36, 125.22, 124.09, 123.83, 116.08, 115.41, 114.09, 110.88, 109.08, 62.85, 55.90, 44.78, 43.25, 21.84, 18.78. LCMS calcd for C₃₀H₃₀F₂N₃O₄ (M+H⁺): 534.2126; Found: 534.2128.

### Example 57. Synthesis of (R)-5-(2-(dimethylamino)ethoxy)-2-methyl-N-(1-(2-(pyrimidin-5-yl)quinolin-4-yl)ethyl)benzamide

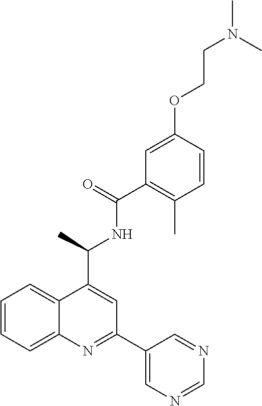 [SMILES: Cc1ccc(OCCN(C)C)cc1C(=O)N[C@H](C)c1cc(-c2cncnc2)nc2ccccc12]

White solid, 56% yield, ¹H NMR (400 MHz, DMSO-d₆) δ 9.64 (s, 2H), 9.39 (s, 1H), 9.06 (d, J=8.0 Hz, 1H), 8.44 (d, J=8.4 Hz, 1H), 8.38 (s, 1H), 8.27-8.21 (m, 1H), 7.93 (ddd, J=8.4, 6.7, 1.2 Hz, 1H), 7.80 (ddd, J=8.3, 6.7, 1.3 Hz, 1H), 7.26 (d, J=8.2 Hz, 1H), 7.09-7.04 (m, 2H), 6.07 (t, J=7.3 Hz, 1H), 4.37 (d, J=5.1 Hz, 2H), 3.57 (q, J=4.7 Hz, 2H), 2.92 (d, J=3.5 Hz, 6H), 2.27 (s, 3H), 1.72 (d, J=6.9 Hz, 3H). ¹³C NMR (101 MHz, DMSO) δ 168.48, 159.28, 158.93, 158.58, 155.78, 155.70, 152.49, 151.86, 148.35, 138.23, 132.38, 132.04, 130.66, 130.60, 128.29, 127.92, 125.54, 123.96, 117.86, 116.13, 115.53, 114.13, 62.87, 55.91, 44.86, 43.28, 21.84, 18.78. LCMS calcd for C₂₇H₃₀N₅O₂ (M+H⁺): 456.2321; Found: 456.2323.

### Example 58. Synthesis of (R)-5-(2-(dimethylamino)ethoxy)-2-methyl-N-(1-(2-((perfluorophenyl)methoxy)quinolin-4-yl)ethyl)benzamide

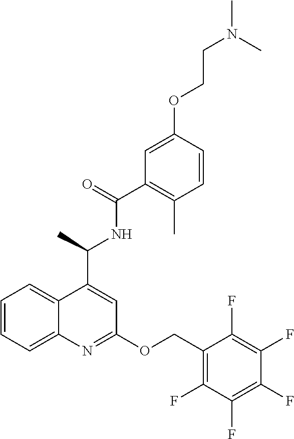 [SMILES: Cc1ccc(OCCN(C)C)cc1C(=O)N[C@H](C)c1cc(OCc2c(F)c(F)c(F)c(F)c2F)nc2ccccc12]

White solid, 46% yield, ¹H NMR (400 MHz, MeOD-d₄) δ 8.17 (d, J=8.4 Hz, 1H), 7.86 (d, J=8.3 Hz, 1H), 7.67 (d, J=7.7 Hz, 1H), 7.51 (q, J=7.5 Hz, 1H), 7.21 (d, J=8.3 Hz, 1H), 7.08 (d, J=6.4 Hz, 1H), 7.06-6.94 (m, 2H), 5.92 (q, J=7.0 Hz, 1H), 4.34 (q, J=5.1 Hz, 2H), 4.07 (d, J=5.7 Hz, 2H), 3.59 (q, J=5.5, 4.8 Hz, 2H), 2.97 (d, J=4.4 Hz, 6H), 2.31 (d, J=8.9 Hz, 3H), 1.64 (d, J=10.6 Hz, 3H). ¹³C NMR (101 MHz, MeOD-d₄) δ 174.19, 166.45, 159.45, 140.02, 142.80, 140.96, 135.60, 133.52, 132.47, 130.82, 128.15, 127.01, 126.79, 65.84, 60.24, 56.77, 54.68, 46.38, 23.52, 21.28. LCMS calcd for C₃₀H₂₉F₅N₃O₃ (M+H⁺): 574.2051; Found: 574.2055.

### Example 59. Synthesis of (R)—N-(1-(2-(1-(2-cyanoethyl)-1H-pyrazol-4-yl) quinolin-4-yl)ethyl)-5-(2-(dimethylamino)ethoxy)-2-methylbenzamide

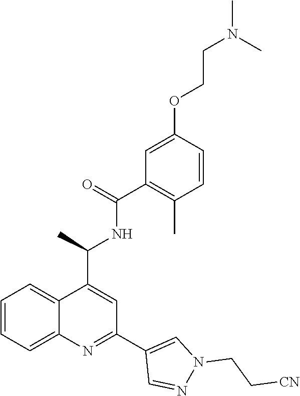 [SMILES: Cc1ccc(OCCN(C)C)cc1C(=O)N[C@H](C)c1cc(-c2cnn(CCC#N)c2)nc2ccccc12]

White solid, 52% yield, ¹H NMR (400 MHz, DMSO-d₆) δ 8.72 (d, J=13.9 Hz, 1H), 8.42 (d, J=11.6 Hz, 1H), 8.05 (d, J=7.2 Hz, 1H), 7.95 (t, J=10.3 Hz, 1H), 7.82 (t, J=7.7 Hz, 1H), 7.70 (t, J=7.7 Hz, 1H), 7.37 (t, J=7.4 Hz, 1H), 7.16 (t, J=8.7 Hz, 1H), 7.03-6.86 (m, 1H), 6.58 (s, 1H), 4.52 (t, J=6.4 Hz, 2H), 3.88-3.75 (m, 2H), 3.18 (t, J=6.2 Hz, 2H), 2.96 (s, 3H), 2.77 (d, J=10.3 Hz, 3H), 2.24 (s, 3H), 2.00 (d, J=7.5 Hz, 1H), 1.90 (s, 1H), 1.83 (d, J=7.4 Hz, 3H). LCMS calcd for C₂₉H₃₃N₆O₂ (M+H⁺): 497.2587; Found: 497.2589.

### Example 60. Synthesis of (R)—N-(1-(2-(1H-pyrrolo[2,3-b]pyridin-5-yl)-quinolin-4-yl)ethyl)-5-(2-(dimethylamino)ethoxy)-2-methylbenzamide

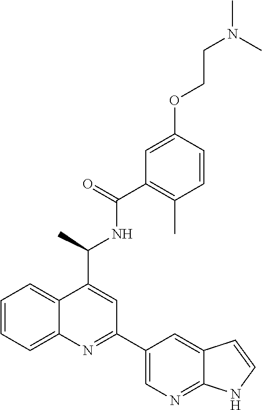 [SMILES: Cc1ccc(OCCN(C)C)cc1C(=O)N[C@H](C)c1cc(-c2cnc3[nH]ccc3c2)nc2ccccc12]

White solid, 50% yield, ¹H NMR (400 MHz, MeOD-d₄) δ 8.68 (s, 1H), 8.15 (d, J=8.3 Hz, 1H), 7.87 (d, J=8.3 Hz, 1H), 7.64 (d, J=7.9 Hz, 3H), 7.53 (d, J=7.9 Hz, 2H), 7.22 (t, J=7.6 Hz, 3H), 7.06-6.98 (m, 2H), 5.92 (q, J=6.9 Hz, 1H), 4.33 (t, J=5.1 Hz, 2H), 3.57 (t, J=5.0 Hz, 2H), 2.97 (d, J=5.6 Hz, 6H), 2.30 (s, 3H), 1.66 (d, J=7.0 Hz, 3H). ¹³C NMR (101 MHz, MeOD-d₄) δ 155.53, 148.79, 133.40, 131.71, 130.45, 128.45, 120.63, 120.39, 115.83, 113.00, 111.80, 109.01, 61.93, 56.28, 42.45, 19.84, 17.33. LCMS calcd for C₃₀H₃₂N₅O₂ (M+H⁺): 494.2478; Found: 494.2475.

### Example 61. Synthesis of (R)—N-(1-(2-(1H-indazol-6-yl)quinolin-4-yl)ethyl)-5-(2-(dimethylamino)ethoxy)-2-methylbenzamide

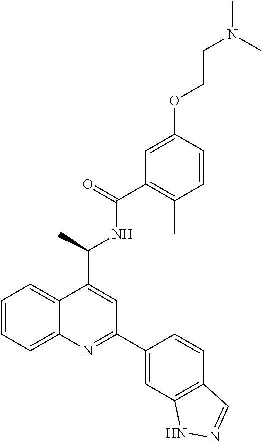 [SMILES: Cc1ccc(OCCN(C)C)cc1C(=O)N[C@H](C)c1cc(-c2ccc3cn[nH]c3c2)nc2ccccc12]

White solid, 53% yield, ¹H NMR (400 MHz, MeOD-d₄) δ 8.27 (s, 2H), 8.15 (s, 1H), 7.98 (s, 2H), 7.75 (s, 1H), 7.72 (s, 1H), 7.49 (s, 1H), 7.22 (s, 1H), 7.02-6.98 (m, 2H), 5.94 (d, J=6.8 Hz, 1H), 4.34 (d, J=5.0 Hz, 2H), 3.59 (d, J=4.9 Hz, 2H), 2.97 (s, 6H), 2.30 (s, 3H), 1.67 (s, 3H). ¹³C NMR (101 MHz, MeOD-d₄) δ 170.62, 155.55, 154.72, 151.57, 150.97, 149.23, 148.73, 137.32, 134.01, 131.74, 130.49, 129.45, 128.51, 128.47, 115.89, 115.00, 113.04, 112.05, 111.06, 62.01, 56.30, 42.50, 19.93, 17.39. LCMS calcd for C₃₀H₃₂N₅O₂ (M+H⁺): 494.2478; Found: 494.2479.

### Example 62. Synthesis of (R)-5-(2-(dimethylamino)ethoxy)-N-(1-(2-(2-methoxy-pyrimidin-5-yl)quinolin-4-yl)ethyl)-2-methylbenzamide

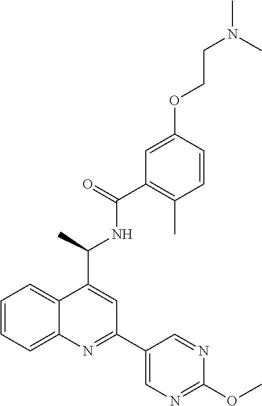 [SMILES: COc1ncc(-c2cc([C@@H](C)NC(=O)c3cc(OCCN(C)C)ccc3C)c3ccccc3n2)cn1]

White solid, 51% yield, ¹H NMR (400 MHz, MeOD-d₄) δ 8.12 (d, J=5.4 Hz, 2H), 7.86 (s, 1H), 7.67 (t, J=7.6 Hz, 1H), 7.48 (t, J=7.6 Hz, 1H), 7.34 (s, 1H), 7.20 (d, J=8.1 Hz, 1H), 7.06 (s, 1H), 7.00 (d, J=8.8 Hz, 2H), 5.91 (q, J=7.0 Hz, 1H), 4.34 (q, J=6.8, 4.9 Hz, 2H), 3.99 (s, 3H), 3.58 (t, J=4.9 Hz, 2H), 2.96 (s, 6H), 2.29 (s, 3H), 1.66 (d, J=7.0 Hz, 3H). ¹³C NMR (101 MHz, CDCl₃) δ 166.57, 159.41, 156.22, 149.95, 146.55, 142.38, 141.54, 135.58, 133.38, 132.68, 131.02, 128.03, 126.95, 124.94, 119.59, 118.42, 117.06, 112.44, 65.84, 60.18, 57.91, 46.37, 23.52, 21.28. LCMS calcd for C₂₈H₃₂N₅O₃ (M+H⁺): 486.2427; Found: 486.2429.

### Example 63. Synthesis of (R)-5-(2-(dimethylamino)ethoxy)-2-methyl-N-(1-(2-(1-(trifluoro-methyl)-1H-pyrazol-4-yl)quinolin-4-yl)ethyl)benzamide

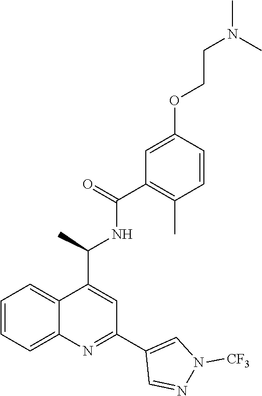 [SMILES: Cc1ccc(OCCN(C)C)cc1C(=O)N[C@H](C)c1cc(-c2cnn(C(F)(F)F)c2)nc2ccccc12]

White solid, 54% yield, ¹H NMR (400 MHz, DMSO-d₆) δ 9.25 (s, 1H), 8.92 (d, J=7.8 Hz, 1H), 8.64 (s, 1H), 8.30 (d, J=8.4 Hz, 1H), 8.08 (d, J=9.1 Hz, 2H), 7.81 (t, J=7.7 Hz, 1H), 7.66 (t, J=7.6 Hz, 1H), 7.26-7.14 (m, 1H), 6.97 (d, J=6.6 Hz, 2H), 5.93 (p, J=6.9 Hz, 1H), 4.29 (t, J=5.1 Hz, 2H), 3.49 (q, J=4.3 Hz, 2H), 2.85 (s, 6H), 2.21 (s, 3H), 1.67 (d, J=6.9 Hz, 3H). ¹³C NMR (101 MHz, DMSO-d₆) δ 168.42, 158.94, 155.64, 151.40, 149.78, 148.08, 143.21, 138.13, 132.01, 130.41, 129.82, 128.35, 127.09, 126.44, 125.38, 124.04, 116.45, 116.05, 114.13, 62.79, 55.91, 44.77, 43.24, 21.21, 18.79. LCMS calcd for C₂₇H₂₉F₃N₅O₂ (M+H⁺): 512.2195; Found: 512.2199.

### Example 64. Synthesis of (R)—N-(1-(2-(1-cyclobutyl-1H-pyrazol-4-yl)quinolin-4-yl)ethyl)-5-(2-(dimethylamino)ethoxy)-2-methylbenzamide

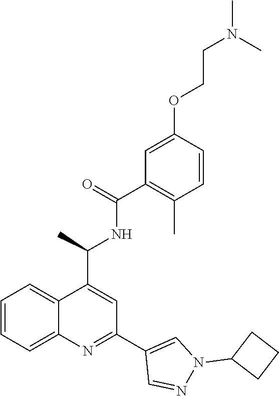 [SMILES: Cc1ccc(OCCN(C)C)cc1C(=O)N[C@H](C)c1cc(-c2cnn(C3CCC3)c2)nc2ccccc12]

White solid, 52% yield, ¹H NMR (400 MHz, DMSO-d₆) δ 5.92 (p, J=7.1 Hz, 1H), 5.00-4.93 (m, 2H), 4.81 (d, J=8.4 Hz, 1H), 4.31 (t, J=5.0 Hz, 2H), 3.51 (q, J=4.9 Hz, 2H), 2.87 (s, 6H), 2.56 (d, J=9.4 Hz, 2H), 2.47-2.41 (m, 2H), 2.22 (s, 3H), 1.85 (tt, J=10.7, 6.6 Hz, 2H), 1.65 (d, J=6.9 Hz, 3H). ¹³C NMR (101 MHz, DMSO-d₆) δ 168.48, 158.83, 158.49, 155.68, 138.86, 138.50, 138.17, 132.05, 131.11, 128.33, 124.72, 124.17, 116.14, 116.07, 114.18, 105.38, 62.86, 55.93, 55.57, 43.29, 30.65, 30.42, 21.40, 18.81, 14.64. LCMS calcd for C₃₀H₃₆N₅O₂ (M+H⁺): 498.2791; Found: 498.2794.

### Example 65. Synthesis of (R)—N-(1-(2-(1-(tert-butyl)-1H-pyrazol-4-yl)quinolin-4-yl)ethyl)-5-(2-(dimethyl-amino)ethoxy)-2-methylbenzamide

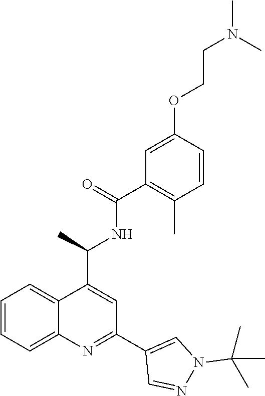 [SMILES: Cc1ccc(OCCN(C)C)cc1C(=O)N[C@H](C)c1cc(-c2cnn(C(C)(C)C)c2)nc2ccccc12]

White solid, 53% yield, ¹H NMR (400 MHz, DMSO-d₆) δ 9.08 (d, J=7.6 Hz, 1H), 8.77 (s, 1H), 8.40 (d, J=8.5 Hz, 1H), 8.34 (s, 1H), 8.17 (d, J=9.1 Hz, 2H), 7.92 (t, J=7.7 Hz, 1H), 7.73 (t, J=7.7 Hz, 1H), 7.20 (d, J=8.2 Hz, 1H), 7.03-6.98 (m, 2H), 5.96-5.91 (m, 1H), 4.32 (t, J=5.0 Hz, 2H), 3.51 (d, J=5.6 Hz, 2H), 2.87 (d, J=3.0 Hz, 6H), 2.21 (s, 3H), 1.67 (s, 3H), 1.62 (s, 9H). ¹³C NMR (101 MHz, DMSO-d₆) δ 168.58, 158.97, 158.62, 155.70, 150.56, 138.49, 138.03, 132.06, 131.87, 128.36, 127.30, 124.58, 124.41, 116.44, 116.13, 114.19, 62.88, 59.74, 55.91, 45.33, 43.27, 29.73, 21.35, 18.80. LCMS calcd for C₃₀H₃₈N₅O₂ (M+H⁺): 500.2947; Found: 500.2949.

### Example 66. Synthesis of (R)-5-(2-(dimethylamino)ethoxy)-2-methyl-N-(1-(2-(1-methyl-1H-imidazol-5-yl)quinolin-4-yl)ethyl)benzamide

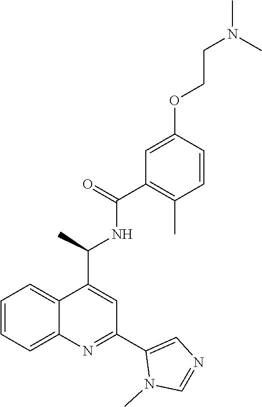 [SMILES: Cc1ccc(OCCN(C)C)cc1C(=O)N[C@H](C)c1cc(-c2cncn2C)nc2ccccc12]

White solid, 57% yield, ¹H NMR (400 MHz, DMSO-d₆) δ 9.15 (s, 1H), 9.00 (d, J=7.6 Hz, 1H), 8.34 (d, J=5.4 Hz, 2H), 8.15 (d, J=8.5 Hz, 1H), 8.04 (s, 1H), 7.87 (s, 1H), 7.74 (s, 1H), 7.18 (d, J=4.7 Hz, 1H), 6.98 (d, J=5.7 Hz, 2H), 5.93 (t, J=7.2 Hz, 1H), 4.28 (s, 2H), 2.85 (s, 6H), 2.50 (s, 3H), 2.19 (s, 3H), 1.64 (d, J=6.9 Hz, 3H). ¹³C NMR (101 MHz, DMSO-d₆) δ 168.56, 159.01, 158.68, 155.65, 152.12, 147.52, 139.43, 138.01, 132.56, 132.06, 130.78, 130.40, 128.36, 128.09, 125.15, 124.02, 116.93, 116.12, 114.10, 62.79, 55.91, 44.99, 43.25, 37.22, 21.37, 18.80. LCMS calcd for C₂₈H₃₂NO₂ (M+H⁺): 458.2478; Found: 458.2479.

### Example 67. Synthesis of (R)-5-(2-(dimethylamino)ethoxy)-N-(1-(2-(3-methoxyphenyl)-quinolin-4-yl)ethyl)-2-methylbenzamide

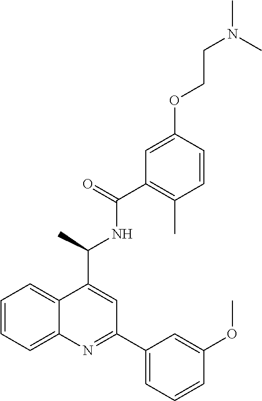 [SMILES: COc1cccc(-c2cc([C@@H](C)NC(=O)c3cc(OCCN(C)C)ccc3C)c3ccccc3n2)c1]

White solid, 56% yield, ¹H NMR (400 MHz, DMSO-d₆) δ 9.15 (d, J=7.9 Hz, 1H), 8.41 (d, J=8.4 Hz, 1H), 8.27-8.18 (m, 2H), 7.91 (d, J=7.5 Hz, 1H), 7.84 (d, J=1.9 Hz, 2H), 7.76 (t, J=7.6 Hz, 1H), 7.57 (t, J=7.9 Hz, 1H), 7.30-7.22 (m, 1H), 7.22-7.15 (m, 1H), 7.09-7.01 (m, 2H), 6.04 (q, J=7.2 Hz, 1H), 4.37 (t, J=5.0 Hz, 2H), 3.93 (s, 3H), 3.56 (q, J=4.6 Hz, 2H), 2.92 (s, 6H), 2.27 (s, 3H), 1.69 (d, J=6.9 Hz, 3H). ¹³C NMR (101 MHz, DMSO-d₆) δ 168.50, 160.26, 158.59, 155.98, 155.71, 152.44, 147.70, 140.32, 138.31, 132.07, 130.53, 130.49, 128.25, 127.33, 125.21, 123.83, 119.98, 115.70, 114.08, 113.23, 62.84, 55.91, 55.76, 44.89, 43.27, 21.89, 18.78. LCMS calcd for C₃₀H₃₄N₃O₃ (M+H⁺): 484.2522; Found: 484.2524.

### Example 68. Synthesis of (R)-5-(2-(dimethylamino)ethoxy)-N-(1-(2-(5-methoxythiophene-3-yl)quinolin-4-yl)ethyl)-2-methylbenzamide

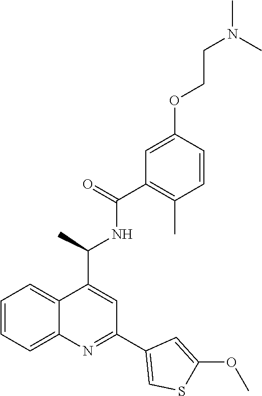 [SMILES: COc1cc(-c2cc([C@@H](C)NC(=O)c3cc(OCCN(C)C)ccc3C)c3ccccc3n2)cs1]

White solid, 50% yield, ¹H NMR (400 MHz, DMSO-d₆) δ 9.08-9.01 (m, 1H), 8.26 (d, J=8.3 Hz, 1H), 8.03 (s, 1H), 7.99 (d, J=8.3 Hz, 1H), 7.79 (d, J=7.7 Hz, 1H), 7.65 (d, J=4.0 Hz, 1H), 7.54 (d, J=8.2 Hz, 1H), 7.26 (s, 1H), 7.07 (s, 1H), 7.03 (d, J=5.3 Hz, 1H), 6.50 (d, J=4.1 Hz, 1H), 5.91 (t, J=7.2 Hz, 1H), 4.35 (d, J=4.9 Hz, 2H), 4.00 (s, 3H), 3.54 (s, 2H), 2.90 (s, 6H), 2.25 (s, 3H), 1.66 (d, J=6.9 Hz, 3H). ¹³C NMR (101 MHz, DMSO-d₆) δ 169.62, 168.71, 155.62, 151.44, 147.18, 132.11, 128.40, 127.98, 126.46, 124.76, 123.83, 114.12, 113.96, 105.94, 62.68, 60.64, 55.99, 53.61, 43.27, 21.44, 18.70. LCMS calcd for C₂₈H₃₁N₃O₃S (M+H⁺): 490.2086; Found: 490.2089.

### Example 69. Synthesis of (R)-5-(2-(dimethylamino)ethoxy)-2-methyl-N-(1-(2-(1-(2-oxo-2-(pyrrolidin-1-yl)ethyl)-1H-pyrazol-4-yl)quinolin-4-yl)ethyl)benzamide

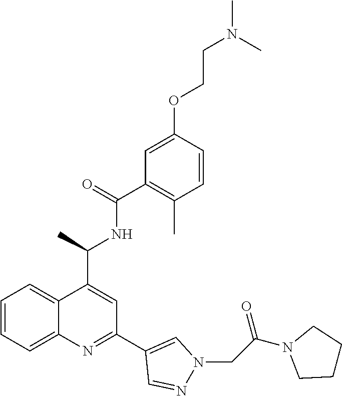 [SMILES: Cc1ccc(OCCN(C)C)cc1C(=O)N[C@H](C)c1cc(-c2cnn(CC(=O)N3CCCC3)c2)nc2ccccc12]

White solid, 45% yield, ¹H NMR (400 MHz, MeOD-d₄) δ 8.73 (s, 1H), 8.58 (d, J=8.5 Hz, 1H), 8.44 (s, 1H), 8.26 (d, J=8.5 Hz, 1H), 8.21 (s, 1H), 8.09 (t, =7.8 Hz, 1H), 7.91 (t, J=7.7 Hz, 1H), 7.25 (d, J=8.3 Hz, 1H), 7.06 (d, J=7.3 Hz, 2H), 6.11 (d, J=7.0 Hz, 1H), 5.28 (s, 2H), 4.37 (t, J=5.0 Hz, 3H), 3.67 (d, J=6.8 Hz, 2H), 3.62 (d, J=4.9 Hz, 2H), 3.50 (d, J=6.7 Hz, 2H), 3.00 (s, 6H), 2.30 (s, 3H), 2.09 (d, J=6.9 Hz, 2H), 1.96 (d, J=7.1 Hz, 2H), 1.80 (d, J=7.0 Hz, 3H). ¹³C NMR (101 MHz, MeOD-d₄) δ 170.52, 165.15, 155.59, 148.97, 139.20, 136.88, 133.87, 133.33, 131.82, 128.60, 128.24, 124.50, 124.19, 122.24, 116.06, 113.14, 61.99, 56.31, 53.73, 45.80, 42.49, 25.60, 23.69, 19.60, 17.41. LCMS calcd for C₃₂H₃₉N₆O₃ (M+H⁺): 555.3005; Found: 555.3009.

### Example 70. Synthesis of (R)-5-(2-(dimethylamino)ethoxy)-2-methyl-N-(1-(2-(4-(methylthio)phenyl)quinolin-4-yl)ethyl)benzamide

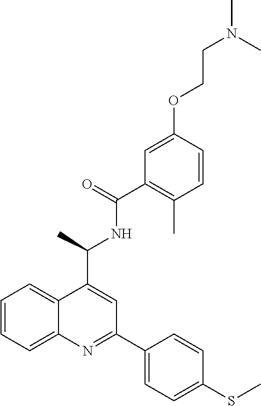 [SMILES: CSc1ccc(-c2cc([C@@H](C)NC(=O)c3cc(OCCN(C)C)ccc3C)c3ccccc3n2)cc1]

White solid, 48% yield, ¹H NMR (400 MHz, DMSO-d₆) δ 9.16 (d, J=7.8 Hz, 1H), 8.39 (d, J=8.4 Hz, 1H), 8.20 (d, J=8.5 Hz, 3H), 7.90 (t, J=1.7 Hz, 1H), 7.75 (ddd, J=8.2, 6.8, 1.2 Hz, 1H), 7.51 (d, J=8.3 Hz, 2H), 7.26 (s, 1H), 7.03 (q, J=2.7 Hz, 2H), 6.00 (q, J=7.2 Hz, 1H), 4.35 (d, J=4.9 Hz, 2H), 3.55 (s, 2H), 2.60 (s, 3H), 2.24 (s, 3H), 1.69 (d, J=7.0 Hz, 3H). ¹³C NMR (101 MHz, DMSO-d₆) δ 168.70, 159.03, 158.69, 155.64, 152.70, 141.58, 138.10, 134.72, 132.12, 130.75, 128.30, 128.11, 127.31, 126.26, 125.03, 123.84, 116.11, 115.27, 113.99, 62.71, 55.97, 45.05, 43.27, 21.66, 18.72, 14.72. LCMS calcd for C₃₀H₃₄N₃O₂S (M+H⁺): 500.2293; Found: 500.2298.

### Example 71. Synthesis of (R)—N-(1-(2-(1-(2-(dimethylamino)-2-oxoethyl)-1H-pyrazol-4-yl) quinolin-4-yl)ethyl)-5-(2-(dimethylamino)ethoxy)-2-methylbenzamide

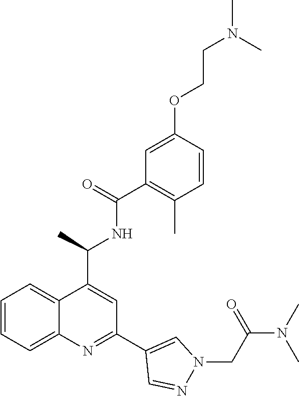 [SMILES: Cc1ccc(OCCN(C)C)cc1C(=O)N[C@H](C)c1cc(-c2cnn(CC(=O)N(C)C)c2)nc2ccccc12]

White solid, 49% yield, ¹H NMR (400 MHz, MeOD) δ 8.80 (d, J=5.3 Hz, 1H), 8.59 (d, J=8.6 Hz, 1H), 8.27 (d, J=5.9 Hz, 2H), 8.12-8.06 (m, 1H), 7.95-7.88 (m, 1H), 7.24-7.18 (m, 1H), 7.07 (d, J=2.7 Hz, 1H), 7.02 (dd, J=8.4, 2.8 Hz, 1H), 6.12-6.06 (m, 1H), 5.37 (d, J=6.9 Hz, 2H), 4.36 (s, 2H), 3.60 (s, 2H), 3.02 (d, J=9.8 Hz, 6H), 2.98 (d, J=4.4 Hz, 6H), 2.27 (d, J=7.7 Hz, 3H), 1.78 (d, J=7.3 Hz, 3H). ¹³C NMR (101 MHz, MeOD) δ 170.57, 166.75, 155.59, 148.27, 139.44, 136.60, 134.67, 134.11, 131.83, 128.73, 124.41, 120.79, 116.28, 116.11, 115.30, 113.23, 62.03, 56.28, 53.23, 47.40, 47.18, 46.97, 42.51, 35.45, 34.81, 19.56, 17.47. LCMS calcd for C₃₀H₃₇N₆O₃ (M+H⁺): 529.2849; Found: 529.2853.

### Example 72. Synthesis of (R)-5-(2-(dimethylamino)ethoxy)-2-methyl-N-(1-(2-(5-(morpholinomethyl)furan-2-yl)quinolin-4-yl)ethyl)benzamide

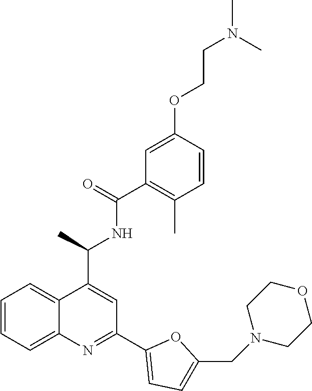 [SMILES: Cc1ccc(OCCN(C)C)cc1C(=O)N[C@H](C)c1cc(-c2ccc(CN3CCOCC3)o2)nc2ccccc12]

White solid, 48% yield, ¹H NMR (400 MHz, DMSO) δ 8.13 (s, 1H), 8.00 (d, J=8.4 Hz, 1H), 7.86 (d, J=3.7 Hz, 1H), 7.80 (dd, J=8.4, 6.9 Hz, 1H), 7.66 (t, J=7.6 Hz, 1H), 7.53 (t, J=7.7 Hz, 1H), 7.38 (d, J=5.4 Hz, 1H), 7.20 (dd, J=8.8, 4.6 Hz, 2H), 7.01-6.97 (m, 2H), 5.90 (q, J=7.2 Hz, 1H), 4.64 (s, 2H), 4.31 (t, J=4.9 Hz, 2H), 3.89-3.74 (m, 4H), 3.40 (d, J=4.1 Hz, 2H), 3.25 (dd, J=21.0, 8.9 Hz, 4H), 2.86 (s, 6H), 2.20 (s, 3H), 1.64 (d, J=6.9 Hz, 3H). LCMS calcd for C₃₂H₃₉N₄O₄ (M+H⁺): 543.2893; Found: 543.2898.

### Example 74. Synthesis of (R)—N-(1-(2-(furan-2-yl)quinolin-4-yl)ethyl)-2-methyl-5-(4-methylpiperazin-1-yl)benzamide

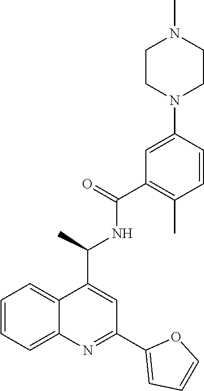 [SMILES: Cc1ccc(N2CCN(C)CC2)cc1C(=O)N[C@H](C)c1cc(-c2ccco2)nc2ccccc12]

White solid, 62% yield, ¹H NMR (400 MHz, DMSO-d₆) δ 9.13 (d, J=7.5 Hz, 1H), 8.36 (d, J=8.4 Hz, 1H), 8.13 (d, J=11.0 Hz, 2H), 8.02 (d, J=1.7 Hz, 1H), 7.87 (t, J=7.7 Hz, 1H), 7.71 (t, J=7.6 Hz, 1H), 7.41 (d, J=3.5 Hz, 1H), 7.20 (d, J=8.3 Hz, 1H), 7.07 (dd, J=8.1, 2.7 Hz, 1H), 7.05 (d, J=2.6 Hz, 1H), 6.82 (dd, J=3.5, 1.8 Hz, 1H), 5.96 (d, J=7.1 Hz, 1H), 3.95-3.82 (m, 2H), 3.61 (d, J=12.0 Hz, 2H), 3.23 (s, 2H), 3.02 (t, J=12.6 Hz, 2H), 2.93 (s, 3H), 2.25 (s, 3H), 1.67 (d, J=7.0 Hz, 3H). ¹³C NMR (101 MHz, DMSO-d₆) δ 169.03, 153.30, 152.66, 148.39, 147.74, 145.63, 137.74, 131.69, 130.61, 129.47, 127.14, 127.04, 125.09, 124.00, 117.84, 115.26, 113.67, 113.28, 111.24, 52.77, 46.35, 45.07, 42.58, 21.54, 18.73. LCMS calcd for C₂₈H₃₁N₄O₂ (M+H¹): 455.2369; Found: 455.2372.

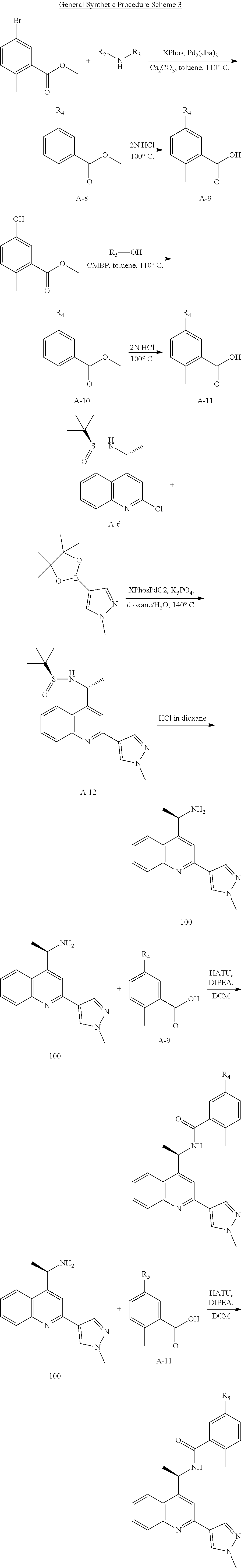

### Synthetic Procedures for Scheme 3

### General Procedures for the Buchwald Coupling I.

To a solution of methyl 5-bromo-2-methylbenzoate (1.0 equiv) in dry toluene in sealed tube, corresponding amine (1.5 equiv), XPhos (0.04 equiv), tris(dibenzylideneacetone)dipalladium (0.02 equiv) and Cs₂CO₃ (2.0 equiv) were added. The reaction mixture was degassed and purged with argon, and then the tube was sealed and heated to 110° C. overnight. The mixture was diluted with EtOAc and was then washed with water and brine. The organic layer was dried over Na₂SO₄, filtered, and concentrated. The residue was purified by silica gel column chromatography (Hexanes/EtOAc=1:1) to provide the desired intermediate. The intermediate was dissolved in DCM and HCl (4M in dioxane, 10.0 equiv) was added. When the starting material was consumed entirely as monitored by LCMS, the mixture was diluted with DCM, adjusted to pH=10, and then washed with water and brine. The organic layer was dried over Na₂SO₄, filtered, and concentrated under vacuum to give a residue which was purified by Prep-HPLC to give the final product A-8 as a white solid.

### General Procedures for the Ester Hydrolysis.

A solution of (A-8, 1.0 equiv) in HCl (2 M aqueous, 8 mL/gm) was stirred at 110° C. for 16 h. LCMS indicated that the starting material completely consumed, and the desired product was detected. The reaction mixture was allowed to cool to room temperature, basified to pH 6 using NaOH (2 M aqueous) the mixture was concentrated under vacuum to give the desired product A-9, 90% yield as a white solid.

### General Procedures for the Mitsonobu Reaction.

To a solution of compound methyl 5-hydroxy-2-methylbenzoate (1.0 equiv) in dry toluene in a sealed tube corresponding alcohol (1.5 equiv) and cyanomethylene trimethylphosphorane (CMMP) (2.0 equiv) were added. The reaction mixture was degassed and purged with argon, and then the tube was sealed and heated to 110° C. overnight. When the starting material was consumed entirely as monitored by LCMS, the mixture was diluted with EtOAc and washed with water and brine. The organic layer was dried over Na₂SO₄, filtered, and concentrated. The residue was purified by silica gel column chromatography (DCM/MeOH=20:1) to provide the desired product A-10 as a white solid.

### General Procedures for the Ester Hydrolysis.

A solution of (A-10, 1.0 equiv) in HCl (2 M aqueous, 8 mL/gm) was stirred at 110° C. for 16 h. LCMS indicated that the starting material completely consumed, and the desired product was detected. The reaction mixture was allowed to cool to room temperature, basified to pH 6 using NaOH (2 M aqueous) the mixture was concentrated under vacuum to give the desired product A-11, 90% yield as a white solid.

### Preparation of 2-methyl-5-(1-methyl-1,2,3,6-tetrahydropyridin-4-yl)benzoic acid

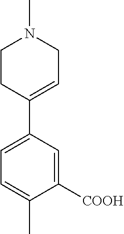 [SMILES: Cc1ccc(C2=CCN(C)CC2)cc1C(=O)O]

White solid, 89% yield. ¹H NMR (400 MHz, CD3OD) δ 7.95 (s, 1H), 7.54 (s, 1H), 7.29 (d, J=7.9 Hz, 1H), 6.16 (s, 1H), 3.91 (d, J=18.0 Hz, 2H), 3.56 (d, J=11.6 Hz, 2H), 3.01 (s, 3H), 2.90 (s, 2H), 2.56 (s, 3H). ¹³C NMR (101 MHz, CD3OD) δ 171.61, 140.60, 137.39, 135.72, 132.95, 129.09, 127.92, 116.85, 55.85, 53.42, 42.96, 35.38, 25.58, 21.37. LCMS calcd for C₁₄H₁₈NO₂ (M+H⁺): 232.1259; Found: 232.1259.

### Preparation of 5-(4-(dimethylamino)piperidin-1-yl)-2-methylbenzoic acid

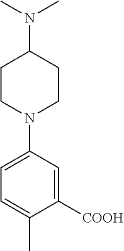 [SMILES: Cc1ccc(N2CCC(N(C)C)CC2)cc1C(=O)O]

White solid, 89% yield. ¹H NMR (400 MHz, CD3OD) δ 7.38 (d, J=2.7 Hz, 1H), 7.12 (d, J1=8.4 Hz, 1H), 7.01 (dd, J=8.4, 2.8 Hz, 1H), 3.80 (dp, J=12.7, 2.1 Hz, 2H), 3.28-3.17 (m, 1H), 2.87 (s, 6H), 2.80-2.62 (m, 2H), 2.15 (dt, J=12.5, 2.9 Hz, 2H), 1.82 (qd, J=12.2, 4.1 Hz, 2H). ¹³C NMR (101 MHz, CD3OD) δ 174.64, 150.70, 136.31, 133.99, 131.56, 121.53, 119.64, 65.68, 50.36, 41.32, 28.25, 21.48. LCMS calcd for C₁₅H₂₃N₂O₂ (M+H⁺): 262.1681; Found: 262.1685.

### Preparation of 2-methyl-5-(2-methyl-2,7-diazaspiro[3.5]nonan-7-yl)benzoic acid

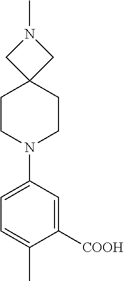 [SMILES: Cc1ccc(N2CCC3(CC2)CN(C)C3)cc1C(=O)O]

White solid, 89% yield. ¹H NMR (400 MHz, CD3OD) δ 7.76 (d, J=2.8 Hz, 1H), 7.34 (dd, J=8.5, 2.8 Hz, 1H), 7.27 (d, J=8.4 Hz, 1H), 4.22 (d, J=10.6 Hz, 2H), 3.95 (d, J=10.7 Hz, 2H), 3.38-3.32 (m, 2H), 3.27 (t, J=5.5 Hz, 2H), 2.98 (s, 3H), 2.52 (s, 3H), 2.20 (t, J=5.7 Hz, 2H), 2.17-2.07 (m, 2H). ¹³C NMR (101 MHz, CD3OD) δ 170.48, 147.20, 136.26, 133.88, 132.37, 123.21, 121.44, 65.67, 49.95, 42.68, 35.30, 34.34, 21.07. LCMS calcd for C₁₆H₂₃N₂O₂ (M+H⁺): 275.1681; Found: 275.1684.

### Preparation of 2-methyl-5-(8-methyl-3,8-diazabicyclo[3.2.1]octan-3-yl)benzoic acid

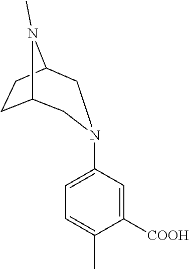 [SMILES: Cc1ccc(N2CC3CCC(C2)N3C)cc1C(=O)O]

White solid, 86% yield. ¹H NMR (400 MHz, CD3OD) δ 8.51 (s, 1H), 6.98 (d, J=12.3 Hz, 2H), 6.74 (d, J=8.3 Hz, 1H), 3.86-3.76 (m, 2H), 3.52-3.41 (m, 2H), 3.18-2.99 (m, 2H), 2.69 (s, 3H), 2.34 (d, J=16.6 Hz, 3H), 2.21-2.16 (m, 1H), 2.05-1.99 (m, 1H), 1.92-1.83 (m, 2H). ¹³C NMR (101 MHz, CD3OD) δ 148.88, 141.94, 132.13, 127.52, 116.42, 115.35, 63.62, 53.33, 39.19, 25.35, 24.10, 19.78. LCMS calcd for C₁₅H₂₁N₂O₂ (M+H⁺): 261.1525; Found: 260.1527.

### Preparation of 2-methyl-5-((1R,4R)-5-methyl-2,5-diazabicyclo[2.2.1]heptan-2-yl)benzoic acid

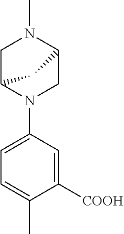

White solid, 86% yield. ¹H NMR (400 MHz, CD3OD) δ 6.99 (d, J=8.2 Hz, 1H), 6.79-6.68 (m, 1H), 6.50 (dd, J=8.3, 2.5 Hz, 1H), 4.41 (s, 1H), 4.10 (s, 1H), 3.54 (d, J=10.6 Hz, 1H), 3.44 (d, J=10.9 Hz, 1H), 3.04 (d, J=11.1 Hz, 1H), 2.69 (s, 3H), 2.30 (s, 3H), 2.21 (d, J=11.1 Hz, 1H), 2.10 (d, J=11.3 Hz, 1H), 1.88 (s, 1H). ¹³C NMR (101 MHz, CD3OD) δ 178.07, 144.97, 141.73, 132.54, 125.46, 114.59, 113.50, 66.81, 60.96, 57.73, 52.51, 41.25, 23.92, 19.81. LCMS calcd for C₁₄H₁₉N₂O₂ (M+H⁺): 247.1368; Found: 247.1369.

### Preparation of 2-methyl-5-(4-methylpiperazin-1-yl)benzoic acid

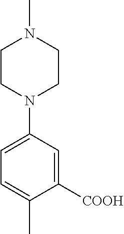 [SMILES: Cc1ccc(N2CCN(C)CC2)cc1C(=O)O]

White solid, 91% yield. ¹H NMR (400 MHz, CD3OD) δ 7.17 (t, J=4.4 Hz, 1H), 7.12-6.98 (m, 1H), 6.92 (dq, J=9.5, 3.0 Hz, 1H), 3.26 (q, J=3.7 Hz, 4H), 3.10 (t, J=5.1 Hz, 4H), 2.72 (s, 3H), 2.40 (d, J=4.5 Hz, 3H). ¹³C NMR (101 MHz, CD3OD) δ 177.20, 149.26, 140.47, 132.53, 129.34, 118.90, 117.30, 54.69, 48.36, 44.09, 20.05. LCMS calcd for C₁₃H₁₉N₂O₂ (M+H⁺): 235.1368; Found: 235.1369.

### Preparation of (R)-5-(3,4-dimethylpiperazin-1-yl)-2-methylbenzoic acid

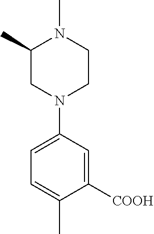 [SMILES: Cc1ccc(N2CCN(C)[C@H](C)C2)cc1C(=O)O]

White solid, 76% yield. ¹H NMR (400 MHz, CD3OD) δ 7.11 (d, J=2.7 Hz, 1H), 7.04 (d, J=8.3 Hz, 1H), 6.85 (dd, J=8.3, 2.7 Hz, 1H), 3.57-3.47 (m, 2H), 3.32-3.20 (m, 1H), 3.03 (ddd, J=12.3, 6.1, 2.6 Hz, 1H), 2.99-2.88 (m, 2H), 2.71 (s, 3H), 2.70-2.64 (m, 1H), 2.35 (s, 3H), 1.29 (d, J=6.4 Hz, 3H). ¹³C NMR (101 MHz, CD3OD) δ 177.12, 149.11, 140.35, 132.56, 129.44, 118.98, 117.32, 60.29, 55.40, 55.01, 48.49, 40.93, 20.07, 14.84. LCMS calcd for C₁₄H₂₁N₂O₂ (M+H⁺): 249.1525; Found: 249.1528.

### Preparation of -methyl-5-(4-methyl-4,7-diazaspiro[2.5]octan-7-yl)benzoic acid

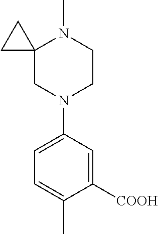 [SMILES: Cc1ccc(N2CCN(C)C3(CC3)C2)cc1C(=O)O]

White solid, 70% yield. ¹H NMR (400 MHz, CD3OD) δ 7.12 (q, J=3.6 Hz, 1H), 7.04 (dd, J=8.6, 2.6 Hz, 1H), 6.86-6.81 (m, 1H), 3.41 (t, J=5.8 Hz, 1H), 3.35 (d, J=3.3 Hz, 1H), 3.19 (d, J=3.1 Hz, 1H), 3.13-3.10 (m, 1H), 2.48 (d, J=3.8 Hz, 1H), 2.43 (s, 1H), 2.37 (d, J=2.8 Hz, 3H), 1.91 (d, J=2.7 Hz, 3H), 0.82 (d, J=6.4 Hz, 2H), 0.67-0.54 (m, 2H). ¹³C NMR (101 MHz, CD3OD) δ 178.37, 150.26, 141.57, 132.05, 128.06, 117.60, 116.56, 52.74, 44.29, 42.97, 36.95, 23.91, 19.88, 11.91. LCMS calcd for C₁₅H₂₁N₂O₂ (M+H⁺): 261.1525; Found: 260.1528.

### Preparation of 2-methyl-5-((1R,4R)-5-methyl-2,5-diazabicyclo[2.2.1]heptan-2-yl)benzoic acid

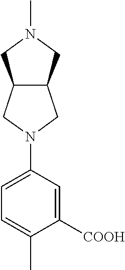 [SMILES: Cc1ccc(N2C[C@H]3CN(C)C[C@H]3C2)cc1C(=O)O]

White solid, 80% yield. ¹H NMR (400 MHz, CD3OD) δ 6.99 (s, 1H), 6.93 (s, 1H), 6.68-6.57 (m, 1H), 3.56-3.46 (m, 2H), 3.37 (d, J=10.0 Hz, 2H), 3.18-3.10 (m, 2H), 3.04 (d, J=9.7 Hz, 4H), 2.76 (s, 3H), 2.33 (s, 3H). ¹³C NMR (101 MHz, CD3OD) δ 177.63, 147.84, 140.33, 132.21, 126.65, 116.69, 115.53, 62.14, 54.73, 42.35, 40.61, 19.98. LCMS calcd for C₁₄H₁₉N₂O₂ (M+H⁺): 246.1368; Found: 246.1368.

### Preparation of 5-(3-(dimethylamino)azetidin-1-yl)-2-methylbenzoic acid

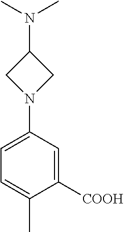 [SMILES: Cc1ccc(N2CC(N(C)C)C2)cc1C(=O)O]

White solid, 48% yield. ¹H NMR (400 MHz, CD3OD) δ 7.34 (s, 1H), 7.06 (d, J=8.2 Hz, 1H), 6.55 (dd, J=8.3, 2.6 Hz, 1H), 4.05 (t, J=7.4 Hz, 2H), 3.76 (dd, J=8.0, 5.5 Hz, 2H), 3.65 (p, J=6.2 Hz, 1H), 2.52 (s, 6H), 2.40 (s, 3H). ¹³C NMR (101 MHz, CD3OD) δ 173.91, 150.29, 132.79, 129.58, 129.46, 115.42, 113.94, 57.49, 56.00, 41.36, 20.53. LCMS calcd for C₁₃H₁₉N₂O₂ (M+H⁺): 235.1368; Found: 250.1369.

### Preparation of (R)-5-(3-(dimethylamino)pyrrolidin-1-yl)-2-methylbenzoic acid

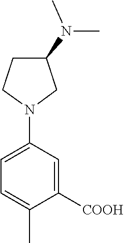 [SMILES: Cc1ccc(N2CC[C@@H](N(C)C)C2)cc1C(=O)O]

White solid, 87% yield. ¹H NMR (400 MHz, CD3OD) δ 7.29 (d, J=3.5 Hz, 1H), 7.06 (d, J=8.3 Hz, 1H), 6.85 (dd, J=8.5, 2.7 Hz, 1H), 3.07 (dt, J=13.2, 3.1 Hz, 2H), 2.53 (ddd, J=14.4, 12.6, 2.2 Hz, 2H), 2.46 (s, 3H), 2.25 (d, J=1.4 Hz, 6H), 1.84-1.80 (m, 1H), 1.36 (qd, J=12.2, 4.1 Hz, 2H). ¹³C NMR (101 MHz, CD3OD) δ 169.55, 156.84, 133.64, 131.40, 120.51, 118.06, 63.34, 52.21, 46.31, 41.40, 29.71, 20.80. LCMS calcd for C₁₄H₂₁N₂O₂ (M+H⁺): 249.1525; Found: 249.1529.

### Preparation of (S)-2-methyl-5-((1-methylazetidin-2-yl)methoxy)benzoic acid

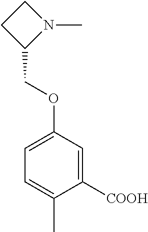

White solid, 75% yield. ¹H NMR (400 MHz, CD3OD) δ 7.30 (d, J=2.9 Hz, 1H), 7.15 (d, J=8.4 Hz, 1H), 6.97 (dd, J=8.4, 2.8 Hz, 1H), 4.69 (dd, J=9.1, 4.9 Hz, 1H), 4.36-4.30 (m, 2H), 4.18 (td, J=9.5, 5.5 Hz, 1H), 3.97 (q, J=9.4 Hz, 1H), 2.95 (s, 3H), 2.58 (dd, J=7.3, 4.9 Hz, 1H), 2.44 (s, 3H), 2.40-2.35 (m, 1H). ¹³C NMR (101 MHz, CD3OD) δ 174.90, 156.85, 138.63, 133.04, 131.26, 117.22, 115.77, 69.49, 67.42, 54.17, 41.38, 20.29, 19.58. LCMS calcd for C₁₃H₁₈NO₃ (M+H⁺): 236.1208; Found: 236.1209.

### Preparation of (S)-2-methyl-5-((1-methylpyrrolidin-3-yl)oxy)benzoic acid

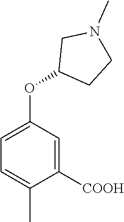

White solid, 89% yield. ¹H NMR (400 MHz, CD3OD) δ 6.88 (dq, J=6.5, 2.5 Hz, 2H), 6.59 (dt, J=7.7, 3.6 Hz, 1H), 4.85 (t, J=5.4 Hz, 1H), 4.78-4.69 (m, 2H), 3.24 (dt, J=12.5, 5.2 Hz, 1H), 3.10-3.00 (m, 1H), 2.70-2.63 (m, 3H), 2.26 (dt, J=14.2, 7.1 Hz, 1H), 2.20-2.16 (m, 3H), 2.00 (dt, J=13.9, 7.0 Hz, 1H). ¹³C NMR (101 MHz, CD3OD) δ 177.06, 155.52, 141.74, 132.78, 129.92, 116.95, 116.30, 61.47, 55.15, 41.86, 32.03, 19.95. LCMS calcd for C₁₃H₁₈NO₃ (M+H⁺): 236.1208; Found: 236.1209.

### Preparation of (R)-2-methyl-5-((1-methylpyrrolidin-2-yl)methoxy)benzoic acid

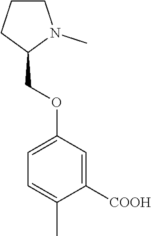 [SMILES: Cc1ccc(OC[C@H]2CCCN2C)cc1C(=O)O]

White solid, 85% yield. ¹H NMR (400 MHz, CD3OD) δ 6.73 (d, J=8.4 Hz, 1H), 6.52 (td, J=8.5, 7.2, 2.7 Hz, 1H), 3.89 (t, J=5.5 Hz, 1H), 3.43-3.33 (m, 1H), 3.31 (td, J=7.0, 3.6 Hz, 1H), 2.96-2.68 (m, 2H), 2.60 (s, 3H), 2.02 (d, J=4.2 Hz, 3H), 1.98-1.79 (m, 1H), 1.73 (dq, J=14.4, 7.2 Hz, 2H), 1.66 (d, J=6.6 Hz, 1H). ¹³C NMR (101 MHz, CD3OD) δ 176.33, 156.93, 155.38, 132.76, 130.35, 116.48, 115.16, 68.58, 67.42, 58.14, 41.34, 27.75, 23.36, 20.05. LCMS calcd for C₁₄H₂₀NO₃ (M+H⁺): 250.1365; Found: 250.1369.

### Preparation of (S)-2-methyl-5-((1-methylpyrrolidin-2-yl)methoxy)benzoic acid

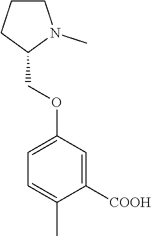 [SMILES: Cc1ccc(OC[C@@H]2CCCN2C)cc1C(=O)O]

White solid, 87% yield. ¹H NMR (400 MHz, CD3OD) δ 6.78-6.72 (m, 1H), 6.66 (s, 1H), 6.48 (td, J=8.1, 2.6 Hz, 1H), 3.83 (d, J=6.5 Hz, 1H), 3.37-3.29 (m, 1H), 3.25 (dt, J=11.9, 6.1 Hz, 1H), 2.96-2.78 (m, 1H), 2.79-2.64 (m, 1H), 2.54 (s, 3H), 1.97 (d, J=4.5 Hz, 3H), 1.88 (dt, J=15.1, 7.3 Hz, 1H), 1.66 (dp, J=14.0, 7.0 Hz, 2H), 1.52 (dd, J=13.3, 7.1 Hz, 1H). ¹³C NMR (101 MHz, CD3OD) δ 175.81, 156.92, 155.36, 132.86, 130.67, 116.72, 115.38, 68.57, 67.36, 58.13, 41.30, 27.72, 23.34, 20.15. LCMS calcd for C₁₄H₂₀NO₃ (M+H⁺): 250.1365; Found: 250.1366.

### Preparation of 2-methyl-5-((1-methylazetidin-3-yl)oxy)benzoic acid

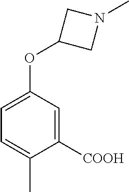 [SMILES: Cc1ccc(OC2CN(C)C2)cc1C(=O)O]

White solid, 80% yield. ¹H NMR (400 MHz, CD3OD) δ 7.09 (d, J=8.4 Hz, 1H), 6.96 (d, J=2.8 Hz, 1H), 6.71 (dd, J=8.3, 2.8 Hz, 1H), 4.35 (dd, J=10.7, 6.2 Hz, 1H), 2.80 (s, 3H), 2.41 (s, 3H), 1.77 (td, J=11.6, 7.6 Hz, 2H), 1.56-1.52 (m, 1H), 1.46 (d, J=7.3 Hz, 1H). ¹³C NMR (101 MHz, CD3OD) δ 177.36, 155.27, 142.38, 132.72, 129.96, 116.08, 114.92, 63.42, 27.53, 25.07, 13.93. LCMS calcd for C₁₂H₁₃NO₃ (M+H⁺): 222.1052; Found: 222.1053.

### Preparation of 5-(((2S,4R)-4-fluoro-1-methylpyrrolidin-2-yl)methoxy)-2-methylbenzoic acid

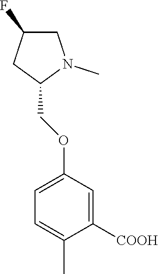 [SMILES: Cc1ccc(OC[C@@H]2C[C@@H](F)CN2C)cc1C(=O)O]

White solid, 72% yield. ¹H NMR (400 MHz, CD3OD) δ 7.21 (s, 1H), 6.91 (d, J=18.9 Hz, 1H), 6.85-6.73 (m, 1H), 5.38-5.17 (m, 1H), 5.12 (d, J=19.3 Hz, 1H), 4.15 (d, J=11.0 Hz, 1H), 4.04 (s, 2H), 3.85 (d, J=47.6 Hz, 2H), 2.82 (s, 3H), 2.29 (d, J=17.0 Hz, 1H), 2.15 (s, 3H). ¹³C NMR (101 MHz, CD3OD) δ 170.48, 156.79, 134.29, 133.88, 132.36, 119.57, 117.43, 66.40, 63.47, 63.23, 35.90, 35.68, 20.92. LCMS calcd for C₁₄H₁₉FNO₃ (M+H₇): 268.1271; Found: 268.1275.

### Preparation of (R)-1-(2-(1-methyl-1H-pyrazol-4-yl)quinolin-4-yl)ethan-1-amine (100)

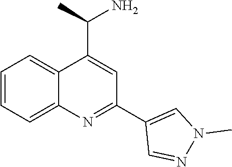 [SMILES: C[C@@H](N)c1cc(-c2cnn(C)c2)nc2ccccc12]

To a solution of (S)—N—((R)-1-(2-chloroquinolin-4-yl)ethyl)-2-methylpropane-2-sulfinamide (A-6) (1.0 equiv) in dioxane/H₂O (4:1) in a microwave reaction vial was added 1-methyl-4-(4,4,5,5-tetramethyl-1,3,2-dioxaborolan-2-yl)-1H-pyrazole (1.5 equiv) and tripotassium phosphate (1.8 equiv). The mixture was purged with nitrogen for 5 min, then XPhosPdG2 (0.1 equiv) was added. The resulting mixture was heated in the biotage microwave reactor at 140° C. for 90 min. LCMS indicated that the starting material was completely consumed. The reaction mixture was diluted with EtOAc and extracted with water and brine. The organic layers were dried over anhydrous Na₂SO₄, filtered, and concentrated under reduced pressure to give a residue for the next step without purification. The crude was dissolved in 1,4-dioxane (0.1 M) and then concentrated HCl was added. The reactions were stirred at room temperature. When the starting material was consumed entirely as monitored by LCMS, solvent was removed under vacuum to give 100, which was used in the next step without purification. Yellow solid, 67% yield. ¹H NMR (400 MHz, CD3OD) δ 8.24 (s, 1H), 8.16 (s, 1H), 7.97 (d, J=8.5 Hz, 2H), 7.84 (s, 1H), 7.71-7.61 (m, 1H), 7.48 (t, J=7.7 Hz, 1H), 3.93 (s, 3H), 1.51 (d, J=6.7 Hz, 3H). ¹³C NMR (101 MHz, CD3OD) δ 154.15, 153.22, 149.22, 139.25, 131.74, 130.74, 129.71, 127.05, 125.64, 124.69, 123.91, 115.55, 47.10, 39.24, 24.40. LCMS calcd for C₁₅H₁₇N₄ (M+H⁺): 253.1375; Found: 253.1376.

### General Procedures for Amide Coupling

The corresponding acid (A-9) or (A-11) (1.1 equiv), HATU (1.1 equiv) and DIPEA (3.0 equiv) were dissolved in DMF (0.2 M) and stirred for 15 min. After that (R)-1-(2-(1-methyl-1H-pyrazol-4-yl)quinolin-4-yl)ethan-1-amine (100) (1.0 equiv) was added and stirred at room temperature overnight. When the starting material was consumed entirely as monitored by LCMS, the mixture was diluted with EtOAc and was then washed with saturated aq. NaHCO₃, water, and brine. The organic layer was dried over Na₂SO₄, filtered, and concentrated under vacuum to give a residue which was purified by Prep-HPLC to give the final product.

### Example 75. Synthesis of 2-methyl-N—((R)-1-(2-(1-methyl-1H-pyrazol-4-yl)quinolin-4-yl)ethyl)-5-(((S)-1-methylazetidin-2-yl)methoxy)benzamide

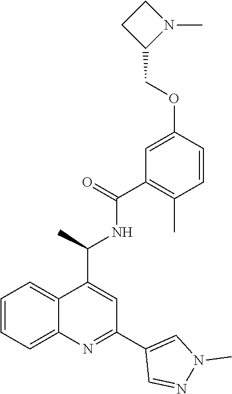

White solid, 67% yield. ¹H NMR (400 MHz, DMSO-d₆) δ 9.02 (d, J=7.4 Hz, 1H), 8.62 (s, 1H), 8.39 (d, J=8.5 Hz, 1H), 8.27 (s, 1H), 8.12 (d, J=8.5 Hz, 1H), 8.07 (s, 1H), 7.91 (t, J=7.7 Hz, 1H), 7.73 (t, J=7.7 Hz, 1H), 7.21 (d, J=8.0 Hz, 1H), 7.01 (d, J=7.9 Hz, 2H), 5.92 (q, J=7.1 Hz, 1H), 4.67 (dt, J=9.4, 5.0 Hz, 1H), 4.35-4.27 (m, 2H), 4.10-4.02 (m, 2H), 3.98 (s, 3H), 3.93 (s, 1H), 2.89 (d, J=4.1 Hz, 3H), 2.45-2.33 (m, 2H), 2.21 (s, 3H), 1.65 (d, J=6.9 Hz, 3H). ¹³C NMR (101 MHz, DMSO-d₆) δ 168.57, 155.78, 150.34, 138.86, 138.01, 132.17, 132.09, 131.91, 128.35, 127.37, 124.59, 124.36, 116.17, 116.06, 114.21, 68.29, 67.06, 53.12, 45.26, 41.48, 21.38, 18.82. LCMS calcd for C₂₈H₃₂N₅O₂ (M+H⁺): 470.2478; Found: 470.2479.

### Example 76. Synthesis of 2-methyl-N—((R)-1-(2-(1-methyl-1H-pyrazol-4-yl)quinolin-4-yl)ethyl)-5-((1R,5S)-8-methyl-3,8-diazabicyclo[3.2.1]octan-3-yl)benzamide

White solid, 64% yield. ¹H NMR (400 MHz, CD3OD) δ 8.69 (d, J=4.1 Hz, 1H), 8.57 (d, J=8.5 Hz, 1H), 8.40 (s, 1H), 8.25 (d, J=8.5 Hz, 1H), 8.20 (d, J=3.1 Hz, 1H), 8.07 (ddd, J=8.5, 7.0, 1.1 Hz, 1H), 7.89 (dd, J=8.6, 7.3 Hz, 1H), 7.15 (d, J=8.2 Hz, 1H), 6.97 (d, J=8.0 Hz, 2H), 6.04 (q, J=7.1 Hz, 1H), 4.05 (s, 5H), 3.73 (dd, J=13.5, 2.7 Hz, 2H), 3.22-3.10 (m, 2H), 2.88 (s, 3H), 2.37-2.27 (m, 2H), 2.21 (s, 3H), 2.19-2.13 (m, 2H), 1.77 (d, J=7.1 Hz, 3H). ¹³C NMR (101 MHz, DMSO-d₆) δ 169.18, 150.60, 147.68, 138.71, 137.60, 131.97, 131.58, 127.12, 126.11, 124.61, 124.31, 116.16, 116.12, 114.03, 62.57, 52.02, 51.94, 45.27, 38.75, 23.83, 21.45, 18.67. LCMS calcd for C₃₀H₃N₆O (M+H⁺): 495.2794; Found: 495.2798.

### Example 77. Synthesis of 2-methyl-N—((R)-1-(2-(1-methyl-1H-pyrazol-4-yl)quinolin-4-yl)ethyl)-5-((1R,4R)-5-methyl-2,5-diazabicyclo[2.2.1]heptan-2-yl)benzamide

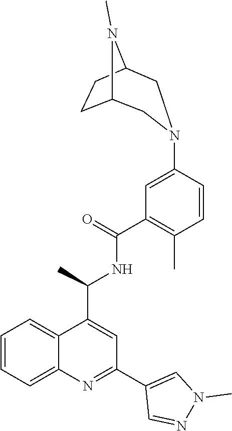 [SMILES: Cc1ccc(N2CC3CCC(C2)N3C)cc1C(=O)N[C@H](C)c1cc(-c2cnn(C)c2)nc2ccccc12]

White solid, 60% yield. ¹H NMR (400 MHz, DMSO-d₆) δ 9.05 (d, J=7.2 Hz, 1H), 8.72 (d, J=5.7 Hz, 1H), 8.47 (d, J=8.5 Hz, 1H), 8.36 (d, J=4.6 Hz, 1H), 8.26-8.15 (m, 2H), 7.98 (t, J=7.7 Hz, 1H), 7.79 (t, J=7.6 Hz, 1H), 7.13 (d, J=3.4 Hz, 1H), 6.77-6.66 (m, 2H), 5.97 (p, J=7.0 Hz, 1H), 4.67 (d, J=20.7 Hz, 1H), 4.41 (s, 1H), 4.04 (s, 3H), 3.75-3.59 (m, 2H), 3.36 (d, J=10.8 Hz, 1H), 3.20-3.06 (m, 1H), 2.93 (d, J=4.8 Hz, 3H), 2.80 (s, 1H), 2.43 (d, J=11.3 Hz, 1H), 2.21 (d, J=6.0 Hz, 4H), 1.70 (d, J=7.0 Hz, 3H). ¹³C NMR (101 MHz, DMSO-d₆) δ 169.30, 158.76, 150.19, 144.15, 138.96, 137.77, 132.33, 132.04, 131.80, 127.43, 124.57, 124.44, 123.73, 118.06, 116.35, 115.14, 114.58, 112.12, 66.26, 61.28, 56.37, 52.99, 45.40, 42.35, 33.37, 21.37, 18.68. LCMS calcd for C₂₉H₁₉N₆O (M+H⁺): 481.2638; Found: 481.2639.

### Example 78. Synthesis of (R)-2-methyl-N-(1-(2-(1-methyl-1H-pyrazol-4-yl)quinolin-4-yl)ethyl)-5-(4-methylpiperazin-1-yl)benzamide

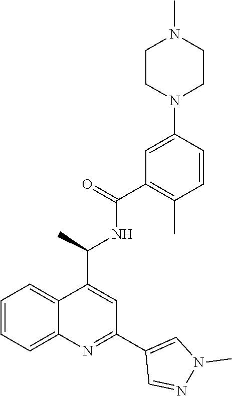 [SMILES: Cc1ccc(N2CCN(C)CC2)cc1C(=O)N[C@H](C)c1cc(-c2cnn(C)c2)nc2ccccc12]

White solid, 64% yield. ¹H NMR (400 MHz, DMSO-d₆) δ 8.96 (d, J=7.7 Hz, 1H), 8.56 (s, 1H), 8.33 (s, 1H), 8.21 (s, 1H), 8.07 (d, J=8.4 Hz, 1H), 8.01 (s, 1H), 7.89-7.77 (m, 1H), 7.66 (d, J=7.7 Hz, 1H), 7.09 (d, J=8.7 Hz, 1H), 6.99-6.95 (m, 2H), 5.96-5.80 (m, 1H), 3.94 (s, 3H), 3.79 (d, J=13.6 Hz, 2H), 3.50 (d, J=11.6 Hz, 2H), 3.21-3.13 (m, 2H), 2.99-2.92 (m, 2H), 2.86 (d, J=17.0 Hz, 3H), 2.15 (s, 3H), 1.61 (d, J=7.3 Hz, 3H). ¹³C NMR (101 MHz, DMSO-d₆) δ 169.06, 150.75, 147.73, 145.21, 138.66, 137.60, 131.86, 131.70, 131.39, 127.12, 127.01, 124.64, 124.26, 120.85, 118.27, 117.75, 116.10, 115.36, 52.77, 46.33, 45.22, 42.58, 39.47, 21.43, 18.77. LCMS calcd for C₂₈H₃₃N₆O (M+H⁺): 469.2638; Found: 469.2639.

### Example 79. Synthesis of 5-((R)-3,4-dimethylpiperazin-1-yl)-2-methyl-N—((R)-1-(2-(1-methyl-1H-pyrazol-4-yl)quinolin-4-yl)ethyl)benzamide

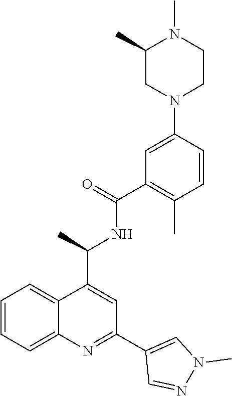 [SMILES: Cc1ccc(N2CCN(C)[C@H](C)C2)cc1C(=O)N[C@H](C)c1cc(-c2cnn(C)c2)nc2ccccc12]

White solid, 62% yield. ¹H NMR (400 MHz, DMSO-d₆) δ 9.10 (d, J=7.2 Hz, 1H), 8.72 (s, 1H), 8.46 (d, J=8.4 Hz, 1H), 8.36 (s, 1H), 8.21 (d, J=8.5 Hz, 1H), 8.18 (s, 1H), 7.97 (t, J=7.7 Hz, 1H), 7.79 (t, J=7.7 Hz, 1H), 7.19 (d, J=9.1 Hz, 1H), 7.09-7.02 (m, 2H), 5.98 (p, J=7.0 Hz, 1H), 4.04 (s, 3H), 3.92 (dd, J=17.3, 13.6 Hz, 2H), 3.63 (d, J=12.4 Hz, 1H), 3.41 (s, 1H), 3.29 (d, J=18.4 Hz, 1H), 3.01 (td, J=13.0, 2.6 Hz, 1H), 2.94 (s, 3H), 2.87-2.75 (m, 1H), 2.23 (s, 3H), 1.70 (d, J=6.9 Hz, 3H), 1.39 (t, J=5.7 Hz, 3H). ¹C NMR (101 MHz, DMSO-d₆) δ 169.13, 158.77, 150.27, 147.56, 138.94, 137.57, 132.30, 131.96, 131.67, 127.38, 127.02, 125.99, 124.57, 124.42, 117.69, 116.27, 115.21, 59.30, 53.66, 52.98, 46.46, 45.37, 21.43, 18.76, 14.47. LCMS calcd for C₂₉H₅N₆₀ (M+H⁺): 483.2794; Found: 483.2798.

### Example 80. Synthesis of (R)-2-methyl-N-(1-(2-(1-methyl-1H-pyrazol-4-yl)quinolin-4-yl)ethyl)-5-(4-methyl-4,7-diazaspiro[2.5]octan-7-yl)benzamide

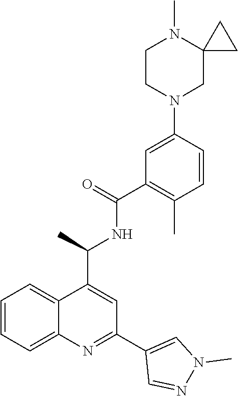 [SMILES: Cc1ccc(N2CCN(C)C3(CC3)C2)cc1C(=O)N[C@H](C)c1cc(-c2cnn(C)c2)nc2ccccc12]

White solid, 60% yield. ¹H NMR (400 MHz, DMSO-d₆) δ 8.97 (d, J=7.4 Hz, 1H), 8.58 (s, 1H), 8.32 (d, J=8.5 Hz, 1H), 8.21 (s, 1H), 8.10-8.00 (m, 2H), 7.83 (t, J=7.7 Hz, 1H), 7.65 (t, J=7.7 Hz, 1H), 7.06 (d, J=9.1 Hz, 1H), 6.99-6.87 (m, 2H), 5.86 (p, J=7.0 Hz, 1H), 3.91 (s, 3H), 3.52 (d, J=43.8 Hz, 2H), 3.44-3.21 (m, 3H), 3.12-3.00 (m, 1H), 2.84 (s, 3H), 2.11 (s, 3H), 1.57 (d, J=6.9 Hz, 3H), 1.18 (d, J=25.1 Hz, 2H), 0.96-0.73 (m, 2H). ¹³C NMR (101 MHz, DMSO-d₆) δ 169.14, 150.42, 147.98, 138.86, 137.60, 132.18, 131.80, 131.54, 127.27, 126.83, 124.58, 124.38, 118.08, 117.60, 116.24, 115.18, 51.74, 49.96, 45.26, 44.40, 42.12, 36.25, 21.48, 18.78. LCMS calcd for C₃₀H₃₅N₆O (M+H⁺): 494.2794; Found: 494.2798.

### Example 81. Synthesis of 2-methyl-N—((R)-1-(2-(1-methyl-1H-pyrazol-4-yl)quinolin-4-yl)ethyl)-5-((3aR,6aS)-5-methylhexahydropyrrolo[3,4-c]pyrrol-2(1H)-yl)benzamide

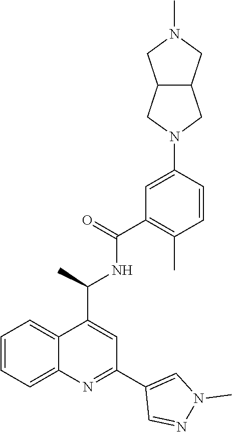 [SMILES: Cc1ccc(N2CC3CN(C)CC3C2)cc1C(=O)N[C@H](C)c1cc(-c2cnn(C)c2)nc2ccccc12]

White solid, 63% yield. ¹H NMR (400 MHz, DMSO-d₆) δ 8.88 (dd, J=7.7, 3.4 Hz, 1H), 8.50 (s, 1H), 8.28 (d, J=8.4 Hz, 1H), 8.15 (s, 1H), 8.02 (d, J=8.5 Hz, 1H), 7.96 (s, 1H), 7.79 (t, J=7.7 Hz, 1H), 7.61 (t, J=7.6 Hz, 1H), 7.02-6.98 (m, 1H), 6.69-6.53 (m, 2H), 5.85 (p, J=7.0 Hz, 1H), 3.90 (s, 3H), 3.88 (t, J=3.7 Hz, 1H), 3.81 (s, 1H), 3.40 (t, J=9.1 Hz, 2H), 3.25 (d, J=17.6 Hz, 2H), 3.17 (d, J=6.2 Hz, 2H), 3.04-2.93 (m, 2H), 2.77 (dd, J=20.9, 4.4 Hz, 3H), 2.10 (d, J=1.9 Hz, 2H), 1.56 (d, J=7.0 Hz, 3H). ¹³C NMR (101 MHz, DMSO-d₆) δ 169.29, 158.58, 146.76, 138.55, 137.64, 131.68, 131.49, 126.91, 124.67, 124.23, 124.18, 116.00, 61.12, 60.40, 54.15, 52.78, 45.05, 40.96, 21.48, 18.73. LCMS calcd for C₃₀H₃₅N₆O (M+H⁺): 495.2794; Found: 495.2797.

### Example 82. Synthesis of 2-methyl-N—((R)-1-(2-(1-methyl-1H-pyrazol-4-yl)quinolin-4-yl)ethyl)-5-(((S)-1-methyl-pyrrolidin-3-yl)oxy)benzamide

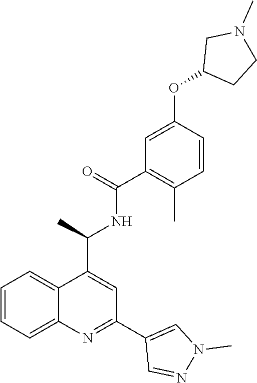

White solid, 59% yield. ¹H NMR (400 MHz, CD3OD) δ 9.03-8.96 (m, 1H), 8.87 (d, J=8.6 Hz, 1H), 8.70 (dt, J=7.8, 3.5 Hz, 1H), 8.57 (d, J=8.6 Hz, 1H), 8.49 (dt, J=9.7, 4.2 Hz, 1H), 8.39 (t, J=8.3 Hz, 1H), 8.26-8.13 (m, 11H), 7.53 (d, J=8.2 Hz, 11H), 7.30 (d, J=8.1 Hz, 2H), 6.37 (q, J=7.0 Hz, 1H), 5.50 (d, J=14.7 Hz, 1H), 4.37 (s, 3H), 4.16 (s, 2H), 3.78-3.65 (m, 1H), 3.62 (t, J=1.8 Hz, 3H), 3.51 (d, J=16.4 Hz, 1H), 2.91 (d, J=20.2 Hz, 1H), 2.67 (s, 1H), 2.56 (s, 3H), 2.09 (d, J=7.0 Hz, 3H). ¹³C NMR (101 MHz, DMSO-d₆) δ 168.47, 158.60, 154.38, 150.82, 138.64, 138.25, 132.18, 131.82, 131.38, 127.02, 124.64, 124.22, 116.07, 115.22, 75.99, 60.22, 53.98, 45.10, 40.96, 30.62, 21.41, 18.78. LCMS calcd for C₂₈H₃₂N₅O₂ (M+H⁺): 470.2478; Found: 470.2479.

### Example 83. Synthesis of 2-methyl-N—((R)-1-(2-(1-methyl-1H-pyrazol-4-yl)quinolin-4-yl)ethyl)-5-(((R)-1-methylpyrrolidin-2-yl)methoxy)benzamide

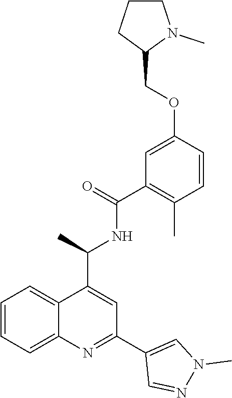

White solid, 60% yield. ¹H NMR (400 MHz, DMSO-d₆) δ 9.13 (t, J=6.9 Hz, 1H), 8.72 (d, J=2.5 Hz, 1H), 8.52-8.43 (m, 1H), 8.36 (s, 1H), 8.25-8.14 (m, 2H), 7.97 (ddd, J=8.3, 6.9, 1.2 Hz, 1H), 7.79 (ddd, J=8.3, 6.9, 1.3 Hz, 1H), 7.25 (dd, J=8.4, 3.4 Hz, 1H), 7.16-7.09 (m, 1H), 6.00 (t, J=7.2 Hz, 1H), 4.40 (dd, J=11.1, 3.5 Hz, 1H), 4.36-4.24 (m, 1H), 3.86 (td, J=7.5, 3.4 Hz, 1H), 3.75-3.62 (m, 1H), 3.27-3.13 (m, 1H), 3.03 (d, J=3.9 Hz, 3H), 2.37-2.30 (m, 1H), 2.27 (s, 3H), 2.17-2.08 (m, 1H), 1.98 (qd, J=7.5, 5.2 Hz, 1H), 1.88 (dt, J=11.4, 3.9 Hz, 1H), 1.70 (d, J=7.0 Hz, 3H). ¹³C NMR (101 MHz, DMSO-d₆) δ 168.58, 159.06, 155.78, 150.28, 138.95, 137.96, 132.32, 132.06, 128.38, 127.42, 124.58, 124.40, 116.25, 116.01, 114.15, 67.22, 66.73, 57.07, 45.27, 41.14, 26.75, 22.49, 21.37, 18.82. LCMS calcd for C₂₉H₃₄N₅O₂ (M+H⁺): 484.2634; Found: 484.2636.

### Example 84. Synthesis of 2-methyl-N—((R)-1-(2-(1-methyl-1H-pyrazol-4-yl)quinolin-4-yl)ethyl)-5-(((S)-1-methylpyrrolidin-2-yl)methoxy)benzamide

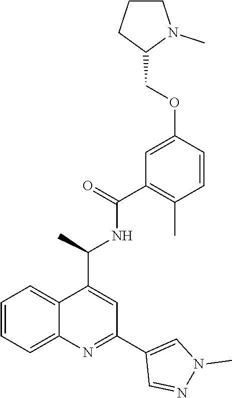

White solid, 66% yield. ¹H NMR (400 MHz, DMSO-d₆) δ 9.15 (d, J=7.4 Hz, 1H), 8.74 (s, 1H), 8.55-8.40 (m, 1H), 8.38 (s, 1H), 8.26-8.12 (m, 2H), 7.99 (ddd, J=8.4, 6.9, 1.2 Hz, 1H), 7.80 (ddd, J=8.3, 6.9, 1.3 Hz, 1H), 7.25 (d, J=8.4 Hz, 1H), 7.15-7.01 (m, 2H), 6.01 (d, J=7.1 Hz, 1H), 4.40 (dd, J=11.2, 3.5 Hz, 1H), 4.34-4.24 (m, 1H), 4.04 (s, 3H), 3.93-3.80 (m, 1H), 3.67 (td, J=13.6, 12.7, 7.7 Hz, 1H), 3.21 (dd, =11.2, 7.2 Hz, 1H), 3.03 (d, J=4.3 Hz, 3H), 2.35-2.29 (m, 1H), 2.27 (s, 3H), 2.13 (qq, J=7.6, 4.6 Hz, 1H), 2.06-1.95 (m, 1H), 1.89 (dtd, J=14.1, 8.3, 5.8 Hz, 1H), 1.71 (d, J=6.9 Hz, 3H). ¹³C NMR (101 MHz, DMSO-d₆) δ 168.58, 158.73, 155.78, 150.14, 139.04, 137.91, 132.45, 132.15, 132.07, 128.40, 127.52, 124.57, 124.45, 116.30, 116.02, 114.17, 67.22, 66.74, 57.07, 45.32, 41.13, 26.75, 22.49, 21.35, 18.84. LCMS calcd for C₂₉H₃₄N₅O₂ (M+H⁺): 484.2634; Found: 484.2638.

### Example 85. Synthesis of 5-(((2S,4R)-4-fluoro-1-methylpyrrolidin-2-yl)methoxy)-2-methyl-N—((R)-1-(2-(1-methyl-1H-pyrazol-4-yl)quinolin-4-yl)ethyl)benzamide

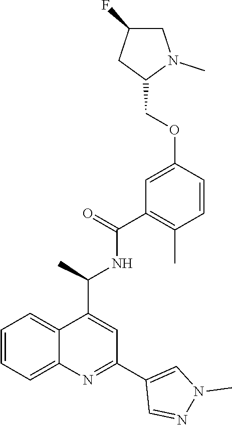

White solid, 56% yield, ¹H NMR (400 MHz, DMSO-d₆) δ 9.08 (d, J=7.4 Hz, 1H), 8.69 (s, 1H), 8.42 (d, J=8.5 Hz, 1H), 8.33 (s, 1H), 8.17 (d, J=8.5 Hz, 1H), 8.13 (s, 1H), 7.94 (t, J=7.7 Hz, 1H), 7.74 (d, J=7.6 Hz, 1H), 7.20 (d, J=8.3 Hz, 1H), 7.05 (d, J=2.8 Hz, 1H), 7.01 (dd, J=8.4, 2.8 Hz, 1H), 5.94 (d, J=7.1 Hz, 1H), 4.42 (d, J=11.0 Hz, 1H), 4.28 (dd, J=11.1, 6.7 Hz, 1H), 4.10 (s, 1H), 3.99 (s, 3H), 3.55 (dd, J=23.0, 14.0 Hz, 1H), 3.05 (s, 3H), 2.58 (d, J=5.8 Hz, 1H), 2.32 (d, J=33.4 Hz, 1H), 2.21 (s, 3H), 1.65 (d, J=7.0 Hz, 3H). ^{H}C NMR (101 MHz, DMSO-d₆) δ 168.57, 159.13, 155.69, 150.07, 139.07, 137.94, 132.51, 132.23, 132.05, 128.48, 127.57, 124.56, 124.47, 117.84, 116.34, 116.01, 114.93, 114.26, 67.16, 66.01, 45.34, 43.70, 34.81, 21.34, 18.82. LCMS calcd for C₂₉H₃₃FN₅O₂ (M+H⁺): 502.2540; Found: 502.2544.

### Example 86. Synthesis of (R)-2-methyl-5-(1-methyl-1,2,3,6-tetrahydropyridin-4-yl)-N-(1-(2-(1-methyl-1H-pyrazol-4-yl)quinolin-4-yl)ethyl)benzamide

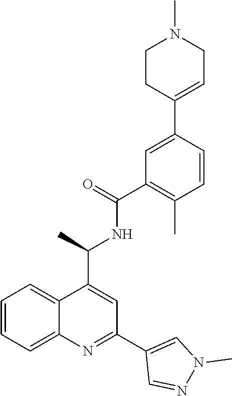 [SMILES: Cc1ccc(C2=CCN(C)CC2)cc1C(=O)N[C@H](C)c1cc(-c2cnn(C)c2)nc2ccccc12]

White solid, 73% yield. ¹H NMR (400 MHz, DMSO-d₆) δ 9.00 (d, J=7.4 Hz, 1H), 8.67 (s, 1H), 8.41 (d, J=8.5 Hz, 1H), 8.30 (s, 1H), 8.25-8.04 (m, 2H), 7.92 (t, J=7.6 Hz, 1H), 7.74 (t, J=7.7 Hz, 1H), 7.07 (d, =8.3 Hz, 1H), 6.84-6.61 (m, 2H), 5.93 (p, J=7.0 Hz, 1H), 3.64 (t, J=7.4 Hz, 2H), 3.29-3.15 (m, 2H), 2.90 (s, 3H), 2.50 (d, J=3.2 Hz, 2H), 2.15 (s, 3H), 1.65 (d, J=6.9 Hz, 3H). ¹³C NMR (101 MHz, DMSO-d₆) δ 169.43, 158.74, 150.14, 146.89, 138.97, 137.68, 132.36, 132.09, 131.68, 127.45, 124.59, 124.46, 123.67, 116.34, 114.48, 112.06, 53.07, 47.27, 45.31, 43.05, 38.55, 21.40, 18.56, 14.39. LCMS calcd for C₂₉H₃₂N₅O (M+H⁺): 466.2529; Found: 466.2528.

### Example 87. Synthesis of (R)-5-(4-(dimethylamino)piperidin-1-yl)-2-methyl-N-(1-(2-(1-methyl-1H-pyrazol-4-yl)quinolin-4-yl)ethyl)benzamide

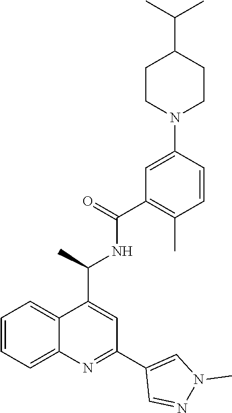 [SMILES: Cc1ccc(N2CCC(C(C)C)CC2)cc1C(=O)N[C@H](C)c1cc(-c2cnn(C)c2)nc2ccccc12]

White solid, 71% yield. ¹H NMR (400 MHz, DMSO-d₆) δ 9.96 (s, 1H), 9.04 (d, J=7.2 Hz, 1H), 8.74 (s, 1H), 8.47 (d, J=8.5 Hz, 1H), 8.37 (s, 1H), 8.25-8.13 (m, 2H), 7.98 (t, J=7.8 Hz, 2H), 7.79 (t, J=7.8 Hz, 2H), 7.10 (d, J=8.4 Hz, 1H), 6.99 (d, J=7.1 Hz, 2H), 5.94 (p, J=7.1 Hz, 1H), 4.00 (s, 3H), 3.82 (d, J=12.3 Hz, 2H), 3.33 (d, J=10.4 Hz, 1H), 2.79 (d, J=4.6 Hz, 6H), 2.72 (d, J=12.5 Hz, 2H), 2.50 (d, J=3.0 Hz, 2H), 2.17 (s, 3H), 2.11-2.03 (m, 2H), 1.72 (td, J=13.3, 10.8, 4.8 Hz, 2H), 1.65 (d, J=6.8 Hz, 3H). ¹³C NMR (101 MHz, DMSO-d₆) δ 169.26, 158.74, 149.59, 148.21, 139.31, 137.31, 132.87, 131.67, 127.88, 126.53, 124.65, 124.53, 118.07, 117.65, 116.52, 115.65, 114.75, 62.82, 48.23, 48.16, 45.56, 25.93, 21.32, 18.76. LCMS calcd for C₃₀H₇N₆O (M+H⁺): 497.2951; Found: 497.2956.

### Example 88. Synthesis of (R)-2-methyl-5-(methyl(1-methylazetidin-3-yl)amino)-N-(1-(2-(1-methyl-1H-pyrazol-4-yl)quinolin-4-yl)ethyl)benzamide

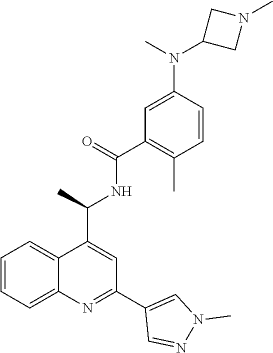 [SMILES: Cc1ccc(N(C)C2CN(C)C2)cc1C(=O)N[C@H](C)c1cc(-c2cnn(C)c2)nc2ccccc12]

White solid, 55% yield. ¹H NMR (400 MHz, DMSO-d₆) δ 9.07-8.96 (m, 1H), 8.64 (s, 1H), 8.39 (d, J=8.5 Hz, 1H), 8.28 (s, 1H), 8.18-8.04 (m, 2H), 7.90 (t, J=7.8 Hz, 1H), 7.72 (t, J=7.8 Hz, 1H), 7.10 (d, J=8.2 Hz, 1H), 6.79 (d, J=4.4 Hz, 2H), 5.92 (p, J=7.2 Hz, 1H), 4.69 (t, J=7.3 Hz, 1H), 4.42 (s, 2H), 4.34-4.20 (m, 1H), 4.15-4.03 (m, 1H), 3.98 (d, J=2.0 Hz, 3H), 2.87 (s, 3H), 2.85 (s, 3H), 2.16 (s, 3H), 1.64 (d, J=6.9 Hz, 3H). ¹³C NMR (101 MHz, DMSO-d₆) δ169.11, 158.76, 150.42, 147.25, 138.83, 137.74, 132.15, 131.78, 131.65, 127.26, 126.27, 124.60, 124.36, 117.20, 116.24, 115.41, 115.17, 60.32, 59.20, 48.93, 45.28, 42.14, 35.81, 21.40, 18.69.

LCMS calcd for C₂₈H₃₃N₆O (M+H⁺): 469.2638; Found: 469.2639.

### Example 89. Synthesis of (R)-2-methyl-5-(methyl((1-methylazetidin-3-yl)methyl)amino)-N-(1-(2-(1-methyl-1H-pyrazol-4-yl)quinolin-4-yl)ethyl)benzamide

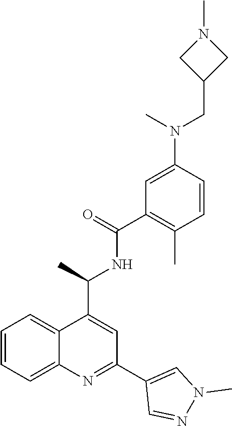 [SMILES: Cc1ccc(N(C)CC2CN(C)C2)cc1C(=O)N[C@H](C)c1cc(-c2cnn(C)c2)nc2ccccc12]

White solid, 56% yield. ¹H NMR (400 MHz, DMSO-d₆) δ 9.00 (d, J=7.4 Hz, 1H), 8.67 (s, 1H), 8.43 (s, 1H), 8.31 (s, 1H), 8.19-8.11 (m, 2H), 7.93 (t, J=7.8 Hz, 1H), 7.74 (t, J=7.7 Hz, 1H), 7.05 (d, J=8.5 Hz, 1H), 6.81 (ddd, J=13.6, 8.4, 2.7 Hz, 1H), 6.72 (dd, J=4.8, 2.7 Hz, 1H), 5.92 (p, J=7.0 Hz, 1H), 4.16-4.06 (m, 1H), 3.99 (s, 3H), 3.85 (dd, J=42.1, 7.0 Hz, 2H), 3.58 (dd, J=15.3, 7.2 Hz, 2H), 2.87 (d, J=9.3 Hz, 3H), 2.82 (d, J=5.3 Hz, 1H), 2.78 (d, J=4.8 Hz, 1H), 2.14 (s, 3H), 1.64 (d, J=6.9 Hz, 3H). ¹³C NMR (101 MHz, DMSO-d₆) δ 169.51, 159.11, 150.11, 147.34, 147.13, 138.97, 137.74, 132.36, 132.10, 131.58, 127.45, 124.57, 124.46, 123.31, 116.36, 114.51, 111.92, 59.74, 58.56, 53.99, 45.35, 41.64, 28.19, 27.36, 21.44, 18.52. LCMS calcd for C₂₉H₃₅N₆O (M+H⁺): 483.2794; Found: 483.2795.

### Example 90. Synthesis of (R)-2-methyl-N-(1-(2-(1-methyl-1H-pyrazol-4-yl)quinolin-4-yl)ethyl)-5-((1-methylazetidin-3-yl)oxy)benzamide

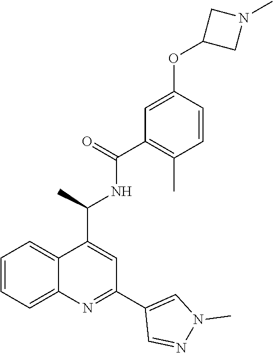 [SMILES: Cc1ccc(OC2CN(C)C2)cc1C(=O)N[C@H](C)c1cc(-c2cnn(C)c2)nc2ccccc12]

White solid, 59% yield. ¹H NMR (400 MHz, DMSO-d₆) δ 9.00 (d, J=7.4 Hz, 1H), 8.58 (s, 1H), 8.31 (d, J=8.5 Hz, 1H), 8.22 (s, 1H), 8.07 (d, J=8.5 Hz, 1H), 8.00 (s, 1H), 7.83 (t, J=7.7 Hz, 1H), 7.74-7.57 (m, 1H), 7.13 (d, J=8.7 Hz, 1H), 6.87-6.71 (m, 2H), 5.84 (d, J=7.2 Hz, 1H), 5.11 (d, J=17.5 Hz, 1H), 4.95 (t, J=13.1 Hz, 1H), 4.66 (t, J=13.7 Hz, 1H), 4.31 (s, 1H), 4.26-4.01 (m, 1H), 3.91 (s, 3H), 2.86 (s, 3H), 2.14 (s, 3H), 1.58 (d, J=6.9 Hz, 3H). ¹³C NMR (101 MHz, DMSO-d₆) δ 168.36, 158.79, 153.94, 150.45, 138.86, 138.29, 132.32, 132.17, 131.78, 127.28, 126.36, 124.60, 124.32, 118.05, 116.25, 115.13, 65.53, 62.25, 61.74, 45.26, 21.35, 18.77. LCMS calcd for C₂₇H₃₀N₅O₂ (M+H⁺): 456.2321; Found: 456.2324.

### Example 91. Synthesis of (R)-2-methyl-N-(1-(2-(1-methyl-1H-pyrazol-4-yl)quinolin-4-yl)ethyl)-5-((1-methylazetidin-3-yl)methoxy)benzamide

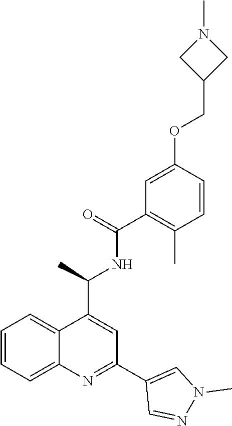 [SMILES: Cc1ccc(OCC2CN(C)C2)cc1C(=O)N[C@H](C)c1cc(-c2cnn(C)c2)nc2ccccc12]

White solid, 63% yield. ¹H NMR (400 MHz, DMSO-d₆) δ 9.13-8.96 (m, 1H), 8.63 (s, 1H), 8.37 (d, J=8.6 Hz, 1H), 8.27 (s, 1H), 8.17-7.99 (m, 2H), 7.89 (d, J=7.3 Hz, 1H), 7.71 (d, J=7.6 Hz, 1H), 7.17 (d, J=8.0 Hz, 1H), 7.03-6.96 (m, 2H), 5.92 (p, J=6.9 Hz, 1H), 4.31 (t, J=8.4 Hz, 1H), 4.21-4.03 (m, 4H), 3.97 (d, J=8.6 Hz, 3H), 3.85 (t, J=8.1 Hz, 1H), 3.18 (dd, J=14.5, 7.5 Hz, 1H), 2.91-2.81 (m, 3H), 2.21 (d, J=12.9 Hz, 3H), 1.63 (d, J=7.0 Hz, 3H). ¹³C NMR (101 MHz, DMSO-d₆) δ 168.64, 158.75, 156.44, 156.33, 150.45, 138.84, 137.97, 132.14, 132.04, 131.76, 127.99, 127.26, 124.61, 124.34, 116.19, 115.84, 114.22, 114.09, 67.56, 57.95, 45.20, 41.80, 28.81, 28.53, 21.39, 18.80. LCMS calcd for C₂₈H₃₂N₅O₂ (M+H⁺): 470.2478; Found: 470.2478.

### Example 92. Synthesis of (R)-2-methyl-N-(1-(2-(1-methyl-1H-pyrazol-4-yl)quinolin-4-yl)ethyl)-5-(2-methyl-2,7-diazaspiro[3.5]nonan-7-yl)benzamide

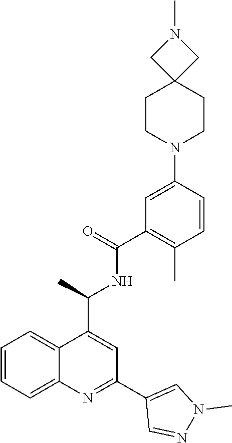 [SMILES: Cc1ccc(N2CCC3(CC2)CN(C)C3)cc1C(=O)N[C@H](C)c1cc(-c2cnn(C)c2)nc2ccccc12]

White solid, 62% yield. ¹H NMR (400 MHz, DMSO-d₆) δ 9.04 (d, J=7.3 Hz, 1H), 8.70 (s, 1H), 8.43 (d, J=8.5 Hz, 1H), 8.33 (s, 1H), 8.17 (s, 1H), 7.93 (d, J=7.8 Hz, 1H), 7.74 (d, J=7.6 Hz, 1H), 7.11 (s, 1H), 7.05 (d, J=9.5 Hz, 2H), 5.93 (p, J=7.0 Hz, 1H), 4.04 (dd, J=10.5, 6.6 Hz, 2H), 3.99 (s, 3H), 3.81 (dd, J=10.5, 6.4 Hz, 2H), 3.20 (t, J=5.4 Hz, 2H), 3.11 (t, J=5.3 Hz, 2H), 2.87 (d, J=4.8 Hz, 3H), 2.18 (s, 3H), 1.93 (d, J=5.9 Hz, 4H), 1.65 (d, J=6.9 Hz, 3H). ¹³C NMR (101 MHz, DMSO-d₆) δ 169.11, 158.78, 149.97, 147.92, 139.09, 137.41, 132.55, 132.28, 131.66, 127.58, 124.57, 124.52, 118.47, 116.38, 116.12, 64.33, 46.82, 45.43, 42.06, 34.46, 33.60, 32.89, 21.34, 18.81. LCMS calcd for C₃₁H₃₇N₆O (M+H⁺): 509.2951; Found: 509.2952.

### Example 93. Synthesis of (R)-5-((1,3-dimethylazetidin-3-yl)(methyl)amino)-2-methyl-N-(1-(2-(1-methyl-1H-pyrazol-4-yl)quinolin-4-yl)ethyl)benzamide

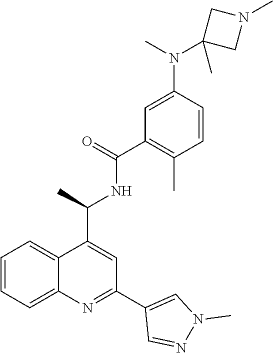 [SMILES: Cc1ccc(N(C)C2(C)CN(C)C2)cc1C(=O)N[C@H](C)c1cc(-c2cnn(C)c2)nc2ccccc12]

White solid, 50% yield. ¹H NMR (400 MHz, DMSO-d₆) δ 8.30 (d, J=8.7 Hz, 1H), 7.93 (d, J=12.3 Hz, 1H), 7.82 (t, J=7.9 Hz, 1H), 6.91-6.74 (m, 2H), 3.96 (s, 3H), 3.77 (s, 8H), 3.33 (d, J=34.5 Hz, 4H), 2.83 (s, 3H), 2.50 (s, 3H), 2.23-2.08 (m, 3H), 1.62 (d, J=7.1 Hz, 3H), 1.22 (s, 3H). ¹³C NMR (101 MHz, CD3OD) δ 172.57, 172.48, 149.11, 143.92, 139.82, 137.55, 135.49, 135.45, 132.88, 130.35, 129.28, 126.51, 125.68, 122.67, 119.83, 116.89, 116.67, 108.87, 61.39, 55.44, 54.92, 47.91, 40.90, 40.22, 21.01, 18.95, 14.68. LCMS calcd for C₂₉H₃₅N₆O (M+H⁺): 483.2794; Found: 483.2797.

### Example 94. Synthesis of (R)-5-((1,3-dimethylazetidin-3-yl)amino)-2-methyl-N-(1-(2-(1-methyl-1H-pyrazol-4-yl)quinolin-4-yl)ethyl)benzamide

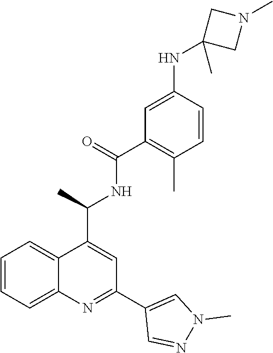 [SMILES: Cc1ccc(NC2(C)CN(C)C2)cc1C(=O)N[C@H](C)c1cc(-c2cnn(C)c2)nc2ccccc12]

White solid, 49% yield. ¹H NMR (400 MHz, DMSO-d₆) δ 8.91 (d, J=7.2 Hz, 1H), 8.52 (s, 1H), 8.30 (d, J=8.7 Hz, 1H), 8.17 (d, J=3.7 Hz, 1H), 8.04 (d, J=8.7 Hz, 1H), 7.93 (d, J=12.3 Hz, 1H), 7.82 (t, J=7.9 Hz, 1H), 7.64 (t, J=7.8 Hz, 1H), 7.03 (d, J=8.3 Hz, 1H), 6.91-6.74 (m, 2H), 5.91 (t, J=7.9 Hz, 1H), 3.96 (s, 3H), 3.38-3.25 (m, 4H), 2.83 (s, 3H), 2.22-2.08 (m, 3H), 1.62 (d, J=7.1 Hz, 3H), 1.22 (s, 3H). ¹³C NMR (101 MHz, CD3OD) δ 172.60, 163.05, 161.81, 161.45, 149.61, 148.86, 140.82, 139.65, 137.58, 135.56, 134.84, 132.83, 130.11, 128.35, 125.86, 125.76, 121.97, 118.93, 118.39, 117.72, 116.11, 115.27, 64.68, 53.68, 47.80, 39.93, 39.58, 26.78, 24.73, 20.89, 18.76. LCMS calcd for C₂₈H₃₃N₆O (M+H⁺): 469.2638; Found: 469.2638.

### Example 95. Synthesis of (R)-5-(3-(dimethylamino)azetidin-1-yl)-2-methyl-N-(1-(2-(1-methyl-1H-pyrazol-4-yl)quinolin-4-yl)ethyl)benzamide

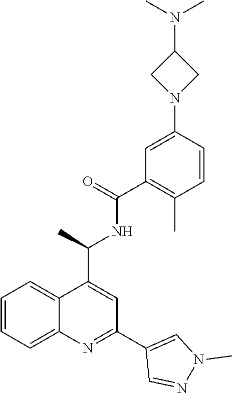 [SMILES: Cc1ccc(N2CC(N(C)C)C2)cc1C(=O)N[C@H](C)c1cc(-c2cnn(C)c2)nc2ccccc12]

White solid, 48% yield. ¹H NMR (400 MHz, CD3OD) δ 8.74 (d, J=2.6 Hz, 1H), 8.59 (d, J=8.5 Hz, 1H), 8.43 (d, J=1.5 Hz, 1H), 8.29 (d, J=8.5 Hz, 1H), 8.23 (d, J=5.5 Hz, 2H), 8.17-8.07 (m, 1H), 7.92 (ddd, J=8.5, 7.0, 1.3 Hz, 1H), 7.07 (d, J=8.1 Hz, 1H), 6.86-6.74 (m, 2H), 6.06 (q, J=7.0 Hz, 1H), 4.07 (s, 3H), 3.98 (tt, J=5.8, 3.2 Hz, 1H), 3.66 (ddd, J=11.2, 3.3, 1.5 Hz, 1H), 3.62-3.54 (m, 1H), 3.35 (d, J=2.8 Hz, 1H), 3.23 (dq, J=7.4, 2.6, 1.9 Hz, 1H), 2.97 (s, 3H), 2.90 (s, 3H), 2.20 (d, J=2.9 Hz, 3H), 1.78 (dd, J=7.1, 1.8 Hz, 3H). ¹³C NMR (101 MHz, CD3OD) δ 172.85, 149.88, 146.04, 140.70, 137.86, 135.33, 134.60, 132.95, 129.99, 126.19, 125.81, 122.47, 119.26, 117.60, 117.09, 117.01, 114.06, 113.96, 62.11, 60.38, 51.68, 47.67, 39.91, 20.92, 18.70. LCMS calcd for C₂₈H₃₃N₆O (M+H⁺): 469.2638; Found: 469.2638.

### Example 96. Synthesis of 5-((R)-3-(dimethylamino)pyrrolidin-1-yl)-2-methyl-N—((R)-1-(2-(1-methyl-1H-pyrazol-4-yl)quinolin-4-yl)ethyl)benzamide

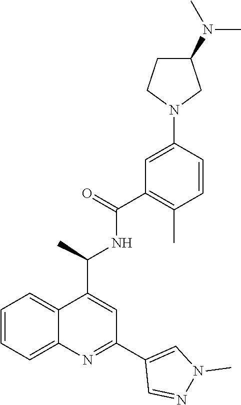 [SMILES: Cc1ccc(N2CC[C@@H](N(C)C)C2)cc1C(=O)N[C@H](C)c1cc(-c2cnn(C)c2)nc2ccccc12]

White solid, 68% yield. ¹H NMR (400 MHz, DMSO-d₆) δ 8.98 (d, J=7.7 Hz, 1H), 8.62 (t, J=4.5 Hz, 1H), 8.40 (d, J=8.8 Hz, 1H), 8.31-8.21 (m, 1H), 8.21-8.03 (m, 3H), 7.90 (t, J=7.8 Hz, 2H), 7.72 (t, J=7.9 Hz, 1H), 7.14-7.02 (m, 1H), 6.63 (dd, J=7.1, 3.0 Hz, 2H), 5.92 (q, J=7.4 Hz, 1H), 3.97 (t, =4.6 Hz, 4H), 3.58 (d, J=8.6 Hz, 1H), 3.53-3.42 (m, 2H), 3.24 (t, J=8.3 Hz, 1H), 2.86 (s, 6H), 2.46-2.36 (m, 1H), 2.26-2.17 (m, 1H), 2.16 (d, J=3.5 Hz, 3H), 1.64 (t, J=5.1 Hz, 3H). ¹³C NMR (101 MHz, DMSO-d₆) δ 169.43, 150.37, 145.41, 138.83, 137.70, 132.14, 131.80, 131.55, 127.26, 124.59, 124.39, 123.06, 118.03, 116.23, 115.11, 113.86, 111.46, 64.59, 49.46, 46.89, 45.27, 41.74, 41.53, 27.05, 21.47, 18.63. LCMS calcd for C₂₉H₃₅N₆O (M+H⁺): 483.2794; Found: 483.2796.

### General Synthetic Procedure Scheme 4

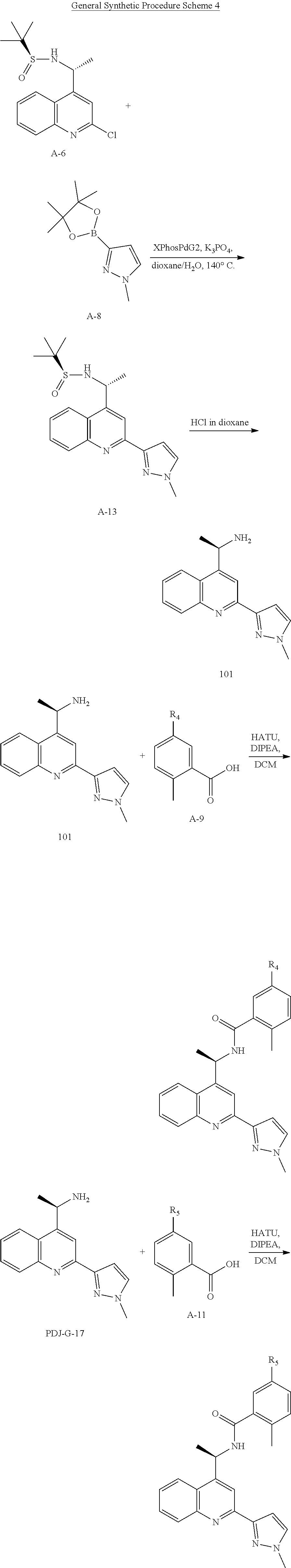

### Synthetic Procedures for Scheme 4

### Preparation of (R)-1-(2-(1-methyl-1H-pyrazol-3-yl)quinolin-4-yl)ethan-1-amine (101)

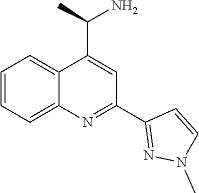 [SMILES: C[C@@H](N)c1cc(-c2ccn(C)n2)nc2ccccc12]

To a solution of (S)—N—((R)-1-(2-chloroquinolin-4-yl)ethyl)-2-methylpropane-2-sulfinamide (A-6) (1.0 equiv) in dioxane/H₂O (4:1) in a microwave reaction vial was added 1-methyl-3-(4,4,5,5-tetramethyl-1,3,2-dioxaborolan-2-yl)-1H-pyrazole (1.5 equiv) and tripotassium phosphate (1.8 equiv). The mixture was purged with nitrogen for 5 min, then XPhosPdG2 (0.1 equiv) was added. The resulting mixture was heated in the biotage microwave reactor at 140° C. for 90 min. LCMS indicated that the starting material was completely consumed. The reaction mixture was diluted with EtOAc and extracted with water and brine. The organic layers were dried over anhydrous Na₂SO₄, filtered, and concentrated under reduced pressure to give a residue for the next step without purification. The crude was dissolved in 1,4-dioxane (0.1 M) and then concentrated HCl was added. The reactions were stirred at room temperature. When the starting material was consumed entirely as monitored by LCMS, solvent was removed under vacuum to give 101, which was used in the next step without purification. Yellow solid, 65% yield. ¹H NMR (400 MHz, CD30) δ 8.86 (s, 1H), 8.55 (dd, J=12.9, 8.4 Hz, 2H), 8.18 (t, J=7.7 Hz, 1H), 8.04-7.95 (m, 2H), 7.64 (d, J=2.1 Hz, 1H), 5.69 (q, J=6.6 Hz, 1H), 4.16 (s, 3H), 1.90 (d, J=6.4 Hz, 3H). ¹³C NMR (101 MHz, CD3OD) δ 157.09, 149.16, 143.28, 139.43, 136.27, 135.71, 131.24, 125.90, 125.57, 122.52, 117.71, 110.22, 48.33, 40.46, 25.74. LCMS calcd for C₁₅H₁₇N₄ (M+H⁺): 253.1375; Found: 253.1378.

### General Procedures for Amide Coupling

The corresponding acid (A-9) or (A-11) (1.1 equiv), HATU (1.1 equiv) and DIPEA (3.0 equiv) were dissolved in DMF (0.2 M) and stirred for 15 min. After that (R)-1-(2-(1-methyl-1H-pyrazol-3-yl)quinolin-4-yl)ethan-1-amine (101, 1.0 equiv) was added and stirred at room temperature overnight. When the starting material was consumed entirely as monitored by LCMS, the mixture was diluted with EtOAc and was then washed with saturated aq. NaHCO₃, water, and brine. The organic layer was dried over Na₂SO₄, filtered, and concentrated under vacuum to give a residue which was purified by Prep-HPLC to give the final product.

### Example 97. Synthesis of (R)-2-methyl-N-(1-(2-(1-methyl-1H-pyrazol-3-yl)quinolin-4-yl)ethyl)-5-(4-methylpiperazin-1-yl)benzamide

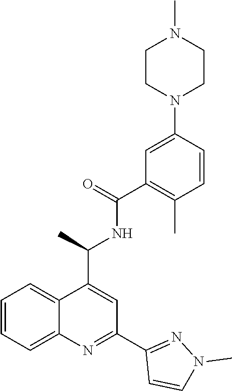 [SMILES: Cc1ccc(N2CCN(C)CC2)cc1C(=O)N[C@H](C)c1cc(-c2ccn(C)n2)nc2ccccc12]

White solid, 69% yield. ¹H NMR (400 MHz, DMSO-d₆) δ 9.07 (d, J=7.3 Hz, 1H), 8.29 (dd, J=8.6, 1.3 Hz, 1H), 8.24 (s, 1H), 8.09 (dd, J=8.5, 1.3 Hz, 1H), 7.83 (d, J=2.3 Hz, 1H), 7.78 (ddd, J=8.3, 6.8, 1.3 Hz, 1H), 7.68-7.61 (m, 1H), 7.08 (d, J=8.3 Hz, 1H), 7.02 (d, J=2.3 Hz, 1H), 6.99-6.87 (m, 2H), 5.83 (p, J=7.0 Hz, 1H), 3.91 (s, 3H), 3.81-3.71 (m, 2H), 3.48 (d, J=12.0 Hz, 2H), 3.08 (d, J=14.7 Hz, 2H), 2.92 (d, J=12.6 Hz, 2H), 2.80 (s, 3H), 2.13 (s, 3H), 1.55 (d, J=7.0 Hz, 3H). ¹³C NMR (101 MHz, DMSO-d₆) δ 169.07, 151.26, 149.67, 147.72, 137.66, 133.68, 131.71, 130.91, 128.34, 127.28, 127.24, 125.42, 124.13, 117.81, 115.35, 114.88, 105.61, 52.74, 46.35, 45.30, 42.57, 21.49, 18.80. LCMS calcd for C₂₈H₃₃N₆₀ (M+H¹): 469.2638; Found: 469.2639.

### Example 98. Synthesis of 2-methyl-N—((R)-1-(2-(1-methyl-1H-pyrazol-3-yl)quinolin-4-yl)ethyl)-5-(((S)-1-methylpyrrolidin-2-yl)methoxy)benzamide

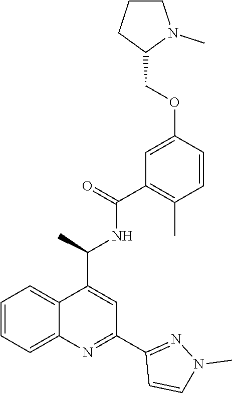

White solid, 67% yield. ¹H NMR (400 MHz, DMSO-d₆) δ 9.19 (d, J=7.4 Hz, 1H), 8.36 (dt, J=8.7, 1.9 Hz, 1H), 8.31 (d, J=6.9 Hz, 1H), 8.16 (dd, J=8.5, 1.3 Hz, 1H), 7.95-7.80 (m, 2H), 7.70 (ddd, J=8.3, 6.9, 1.3 Hz, 1H), 7.21 (dd, J=9.1, 2.7 Hz, 1H), 7.07-7.00 (m, 2H), 5.91 (td, J=7.1, 2.8 Hz, 1H), 4.41-4.32 (m, 1H), 4.24 (dt, J=11.1, 6.9 Hz, 1H), 3.98 (d, J=2.0 Hz, 3H), 3.80 (tt, J=11.0, 5.4 Hz, 1H), 3.74-3.51 (m, 1H), 3.48-3.27 (m, 1H), 3.15 (dq, J=11.2, 7.9 Hz, 1H), 3.08-2.81 (m, 3H), 2.79 (d, J=4.7 Hz, 1H), 2.23 (d, J=5.8 Hz, 3H), 2.10-1.97 (m, 1H), 1.96-1.85 (m, 1H), 1.62 (d, J=7.1 Hz, 3H). ¹³C NMR (101 MHz, DMSO-d₆) δ 168.53, 158.67, 155.78, 151.34, 138.20, 133.66, 132.04, 130.86, 128.47, 127.22, 125.43, 124.10, 117.94, 115.94, 115.02, 114.95, 114.18, 105.57, 67.19, 66.70, 57.05, 53.79, 45.20, 41.07, 26.77, 22.46, 21.45, 18.86. LCMS calcd for C₂₉H₃₄N₅O₂ (M+H⁺): 484.2634; Found: 484.2638.

### Example 99. Synthesis of 5-((R)-3,4-dimethylpiperazin-1-yl)-2-methyl-N—((R)-1-(2-(1-methyl-1H-pyrazol-3-yl)quinolin-4-yl)ethyl)benzamide

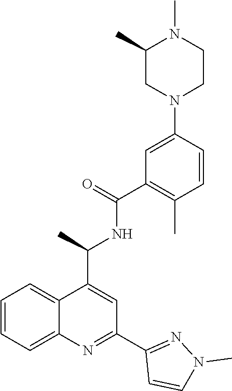 [SMILES: Cc1ccc(N2CCN(C)[C@H](C)C2)cc1C(=O)N[C@H](C)c1cc(-c2ccn(C)n2)nc2ccccc12]

White solid, 67% yield. ¹H NMR (400 MHz, DMSO-d₆) δ 9.07 (d, J=7.3 Hz, 1H), 8.35-8.26 (m, 1H), 8.24 (s, 1H), 8.09 (dd, J=8.5, 1.2 Hz, 1H), 7.86-7.74 (m, 2H), 7.63 (ddd, J=8.3, 6.9, 1.3 Hz, 1H), 7.09-6.99 (m, 2H), 6.99-6.88 (m, 2H), 5.82 (p, J=7.0 Hz, 1H), 3.90 (s, 3H), 3.85-3.69 (m, 2H), 3.50 (d, J=12.4 Hz, 1H), 3.31-3.24 (m, 1H), 3.14 (d, J=11.1 Hz, 1H), 2.89 (td, J=12.8, 2.7 Hz, 1H), 2.79 (d, J=3.3 Hz, 3H), 2.73-2.61 (m, 1H), 2.11 (s, 3H), 1.54 (d, J=7.0 Hz, 3H), 1.24 (dd, J=6.4, 4.4 Hz, 3H). ¹³C NMR (101 MHz, DMSO-d₆) δ 169.14, 159.06, 158.56, 133.80, 131.67, 131.22, 127.83, 127.43, 127.14, 125.40, 124.20, 115.21, 115.06, 105.85, 63.24, 59.30, 53.63, 53.06, 46.44, 45.37, 21.47, 18.76, 14.47. LCMS calcd for C₂₉H₃₅N₆O (M+H⁺): 483.2794; Found: 483.2796.

### Example 100. Synthesis of 5-((R)-3-(dimethylamino)pyrrolidin-1-yl)-2-methyl-N—((R)-1-(2-(1-methyl-1H-pyrazol-3-yl)quinolin-4-yl)ethyl)benzamide

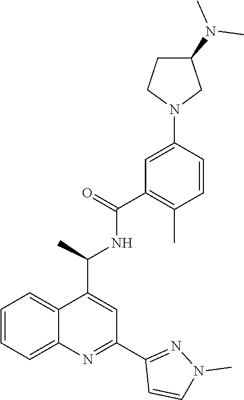 [SMILES: Cc1ccc(N2CC[C@@H](N(C)C)C2)cc1C(=O)N[C@H](C)c1cc(-c2ccn(C)n2)nc2ccccc12]

White solid, 65% yield. ¹H NMR (400 MHz, DMSO-d₆) δ 9.05 (dd, J=7.4, 3.1 Hz, 1H), 8.31-8.23 (m, 2H), 8.09 (d, J=8.4 Hz, 1H), 7.83 (d, J=2.3 Hz, 1H), 7.78 (t, J=7.7 Hz, 1H), 7.63 (ddd, J=8.3, 6.7, 1.2 Hz, 1H), 7.02 (t, J=2.2 Hz, 1H), 6.61-6.55 (m, 2H), 5.84 (q, J=7.1 Hz, 1H), 3.95 (d, J=6.4 Hz, 1H), 3.91 (s, 3H), 3.53 (td, J=7.6, 3.7 Hz, 1H), 3.46-3.37 (m, 2H), 3.24-3.15 (m, 1H), 2.79 (s, 6H), 2.37 (ddt, J=15.5, 7.6, 3.7 Hz, 1H), 2.15 (dd, J=8.4, 4.5 Hz, 1H), 2.11 (d, J=1.9 Hz, 3H), 1.54 (d, J=7.0 Hz, 3H). ¹³C NMR (101 MHz, DMSO-d₆) δ 169.41, 151.29, 145.39, 137.81, 133.66, 131.55, 130.88, 128.36, 127.21, 125.43, 124.14, 123.28, 123.21, 117.97, 115.05, 114.92, 113.88, 111.62, 105.60, 64.60, 49.49, 46.93, 45.25, 41.75, 41.47, 39.52, 27.02, 21.54, 18.67. LCMS calcd for C₂₉H₃₅N₆O (M+H⁺): 483.2794; Found: 483.2796.

### Example 101. Synthesis of (R)-2-methyl-N-(1-(2-(1-methyl-1H-pyrazol-3-yl)quinolin-4-yl)ethyl)-5-(4-methyl-4,7-diazaspiro[2.5]octan-7-yl)benzamide

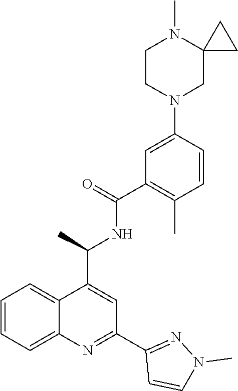 [SMILES: Cc1ccc(N2CCN(C)C3(CC3)C2)cc1C(=O)N[C@H](C)c1cc(-c2ccn(C)n2)nc2ccccc12]

White solid, 60% yield. ¹H NMR (400 MHz, DMSO-d₆) δ 9.07 (d, J=7.3 Hz, 1H), 8.33-8.21 (m, 2H), 8.09 (d, J=8.5 Hz, 1H), 7.86-7.76 (m, 2H), 7.63 (t, J=7.7 Hz, 1H), 7.11-6.99 (m, 2H), 6.95-6.88 (m, 2H), 5.84 (p, J=7.1 Hz, 1H), 3.91 (s, 3H), 3.58-3.45 (m, 2H), 3.45-3.20 (m, 3H), 3.05 (d, J=18.7 Hz, 1H), 2.84 (s, 3H), 2.13 (s, 3H), 1.55 (d, J=7.0 Hz, 3H), 1.18 (d, J=26.8 Hz, 2H), 0.89 (d, J=15.9 Hz, 2H). ¹³C NMR (101 MHz, DMSO-d₆) δ 169.11, 151.31, 149.75, 147.94, 137.75, 133.65, 131.54, 130.85, 128.42, 127.20, 126.97, 125.42, 124.12, 118.00, 117.64, 115.20, 115.08, 115.00, 105.58, 51.74, 50.02, 45.23, 44.41, 42.15, 39.52, 36.26, 21.53, 18.82. LCMS calcd for C₃₀H₃₅N₆₀ (M+H⁺): 495.2794; Found: 495.2795.

### Example 102. Synthesis of 2-methyl-N—((R)-1-(2-(1-methyl-1H-pyrazol-3-yl)quinolin-4-yl)ethyl)-5-(8-methyl-3,8-diazabicyclo[3.2.1]octan-3-yl)benzamide

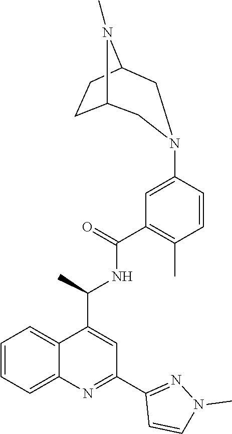 [SMILES: Cc1ccc(N2CC3CCC(C2)N3C)cc1C(=O)N[C@H](C)c1cc(-c2ccn(C)n2)nc2ccccc12]

White solid, 71% yield. ¹H NMR (400 MHz, DMSO-d₆) δ 9.07 (d, J=7.3 Hz, 1H), 8.35-8.28 (m, 1H), 8.24 (s, 1H), 8.09 (dd, J=8.5, 1.3 Hz, 1H), 7.89-7.81 (m, 1H), 7.81-7.73 (m, 1H), 7.63 (ddd, J=8.3, 6.9, 1.3 Hz, 1H), 7.08-6.98 (m, 2H), 6.87-6.80 (m, 2H), 5.83 (q, J=7.0 Hz, 1H), 4.00 (t, J=3.5 Hz, 2H), 3.91 (s, 3H), 3.64 (dt, J=13.5, 3.5 Hz, 2H), 3.05 (d, J=12.4 Hz, 2H), 2.72 (d, J=4.8 Hz, 3H), 2.17-2.12 (m, 2H), 2.11 (s, 3H), 1.96-1.86 (m, 2H), 1.54 (d, J=7.0 Hz, 3H). ¹³C NMR (101 MHz, DMSO-d₆) δ 169.17, 151.24, 147.66, 137.68, 133.68, 131.58, 130.91, 128.32, 127.23, 126.28, 125.42, 124.14, 117.96, 116.20, 115.04, 114.92, 113.99, 105.62, 68.71, 62.92, 62.54, 53.31, 51.96, 45.30, 39.53, 38.74, 23.86, 21.51, 18.71. LCMS calcd for C₃₀H₃₅N₆O (M+H⁺): 495.2794; Found: 495.2798.

### Example 103. Synthesis of 2-methyl-N—((R)-1-(2-(1-methyl-1H-pyrazol-3-yl)quinolin-4-yl)ethyl)-5-((3aR,6aS)-5-methylhexahydropyrrolo[3,4-c]pyrrol-2(1H)-yl)benzamide

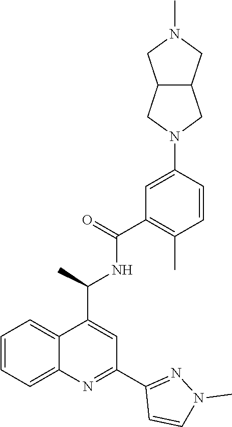 [SMILES: Cc1ccc(N2CC3CN(C)CC3C2)cc1C(=O)N[C@H](C)c1cc(-c2ccn(C)n2)nc2ccccc12]

White solid, 63% yield. ¹H NMR (400 MHz, DMSO-d₆) δ 9.19 (d, J=7.3 Hz, 1H), 8.47-8.37 (m, 2H), 8.26 (d, J=8.4 Hz, 1H), 8.02-7.89 (m, 2H), 7.77 (ddd, J=8.3, 6.8, 1.2 Hz, 1H), 7.20 (d, J=2.3 Hz, 1H), 7.13 (d, J=8.2 Hz, 1H), 6.81-6.71 (m, 2H), 5.97 (p, J=7.0 Hz, 1H), 4.04 (s, 3H), 3.97-3.88 (m, 1H), 3.54 (dd, J=13.6, 9.2 Hz, 2H), 3.33 (dd, J=18.0, 9.5 Hz, 2H), 3.12 (td, J=11.3, 9.7, 5.0 Hz, 2H), 2.91-2.86 (m, 3H), 2.24 (s, 3H), 1.66 (d, J=7.0 Hz, 3H). ¹³C NMR (101 MHz, DMSO-d₆) δ 169.36, 151.01, 146.75, 137.63, 133.76, 131.50, 131.12, 127.93, 127.37, 125.43, 124.37, 124.20, 117.88, 115.92, 114.96, 113.45, 105.86, 60.83, 60.26, 53.97, 52.78, 45.29, 40.99, 21.50, 18.77. LCMS calcd for C₃₀H₃₅N₆O (M+H⁺): 495.2794; Found: 495.2798.

### General Synthetic Procedure Scheme 5

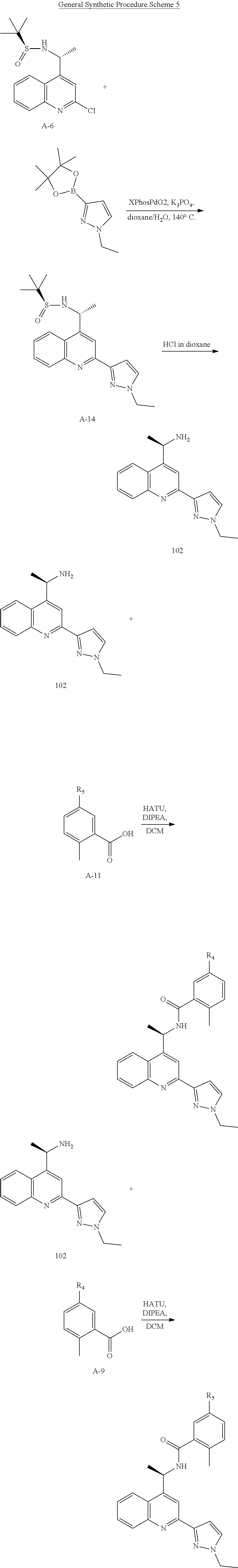

### Synthetic Procedures for Scheme 5

### Preparation of (R)-1-(2-(1-ethyl-1H-pyrazol-3-yl)quinolin-4-yl)ethan-1-amine (102)

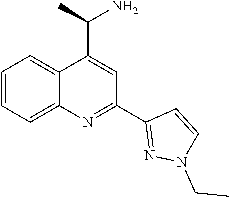 [SMILES: CCn1ccc(-c2cc([C@@H](C)N)c3ccccc3n2)n1]

To a solution of (S)—N—((R)-1-(2-chloroquinolin-4-yl)ethyl)-2-methylpropane-2-sulfinamide (A-6) (1.0 equiv) in dioxane/H₂O (4:1) in a microwave reaction vial was added 1-ethyl-3-(4,4,5,5-tetramethyl-1,3,2-dioxaborolan-2-yl)-1H-pyrazole (1.5 equiv) and tripotassium phosphate (1.8 equiv). The mixture was purged with nitrogen for 5 min, then XPhosPdG2 (0.1 equiv) was added. The resulting mixture was heated in the biotage microwave reactor at 140° C. for 90 min. LCMS indicated that the starting material was completely consumed. The reaction mixture was diluted with EtOAc and extracted with water and brine. The organic layers were dried over anhydrous Na₂SO₄, filtered, and concentrated under reduced pressure to give a residue for the next step without purification. The crude was dissolved in 1,4-dioxane (0.1 M) and then concentrated HCl was added. The reactions were stirred at room temperature. When the starting material was consumed entirely as monitored by LCMS, solvent was removed under vacuum to give 102, which was used in the next step without purification. Yellow solid, 63% yield. ¹H NMR (400 MHz, CD3OD) δ 8.76 (s, 1H), 8.55-8.48 (m, 2H), 8.18 (d, J=8.7 Hz, 1H), 8.02 (d, J=10.1 Hz, 2H), 7.55 (d, J=13.6 Hz, 1H), 5.71-5.61 (m, 1H), 4.45 (d, J=8.6 Hz, 2H), 1.91-1.84 (m, 3H), 1.60 (t, J=7.6 Hz, 3H). ¹³C NMR (101 MHz, CD3OD) δ 156.88, 149.40, 143.38, 139.63, 136.18, 134.24, 131.17, 125.91, 125.52, 122.68, 117.59, 109.93, 48.28, 25.75, 21.07, 15.87. LCMS calcd for C₁₅H₁₉N₄ (M+H⁺): 267.1531; Found: 267.1533.

### General Procedures for Amide Coupling

The corresponding acid (A-11) or (A-9) (1.1 equiv), HATU (1.1 equiv) and DIPEA (3.0 equiv) were dissolved in DMF (0.2 M) and stirred for 15 min. After that (R)-1-(2-(1-ethyl-1H-pyrazol-3-yl)quinolin-4-yl)ethan-1-amine (102, 1.0 equiv) was added and stirred at room temperature overnight. When the starting material was consumed entirely as monitored by LCMS, the mixture was diluted with EtOAc and was then washed with saturated aq. NaHCO₃, water, and brine. The organic layer was dried over Na₂SO₄, filtered, and concentrated under vacuum to give a residue which was purified by Prep-HPLC to give the final product.

### Example 104. Synthesis of 5-((R)-3,4-dimethylpiperazin-1-yl)-N—((R)-1-(2-(1-ethyl-1H-pyrazol-3-yl)quinolin-4-yl)ethyl)-2-methylbenzamide

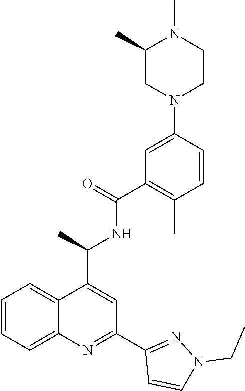 [SMILES: CCn1ccc(-c2cc([C@@H](C)NC(=O)c3cc(N4CCN(C)[C@H](C)C4)ccc3C)c3ccccc3n2)n1]

White solid. 63% yield. ¹H NMR (400 MHz, DMSO-d₆) δ 9.15 (t, J=4.5 Hz, 1H), 8.34 (dd, J=19.9, 5.3 Hz, 2H), 8.17 (d, J=8.3 Hz, 1H), 7.95 (q, J=2.8 Hz, 1H), 7.85 (d, J=7.7 Hz, 1H), 7.71 (t, J=7.6 Hz, 1H), 7.25-7.09 (m, 2H), 7.01 (dt, J=13.1, 3.1 Hz, 2H), 5.92-5.85 (m, 1H), 4.26 (tp, J=6.5, 3.3, 2.7 Hz, 2H), 3.86 (t, J=16.0 Hz, 2H), 3.58 (d, J=12.7 Hz, 1H), 3.37 (d, J=20.1 Hz, 1H), 3.20 (t, J=10.7 Hz, 1H), 2.98 (t, J=12.6 Hz, 1H), 2.78 (dd, J=15.7, 8.2 Hz, 1H), 2.50 (d, J=3.4 Hz, 1H), 2.20 (t, J=2.8 Hz, 3H), 1.62 (dt, J=5.8, 2.8 Hz, 3H), 1.45 (tt, J=6.0, 2.7 Hz, 3H), 1.34 (dt, J=6.2, 2.9 Hz, 3H). ¹³C NMR (101 MHz, DMSO-d₆) δ 169.15, 151.29, 149.40, 147.54, 146.03, 137.85, 132.17, 131.64, 130.94, 128.24, 127.24, 127.10, 125.41, 124.15, 117.69, 115.21, 114.94, 105.53, 59.28, 53.63, 53.04, 47.32, 46.46, 45.45, 21.46, 18.78, 15.94, 14.47. LCMS calcd for C₃H₃₇N₆O (M+H⁺): 497.2951; Found: 497.2956.

### Example 105. Synthesis of N—((R)-1-(2-(1-ethyl-1H-pyrazol-3-yl)quinolin-4-yl)ethyl)-2-methyl-5-(((S)-1-methyl-azetidin-2-yl)methoxy)benzamide

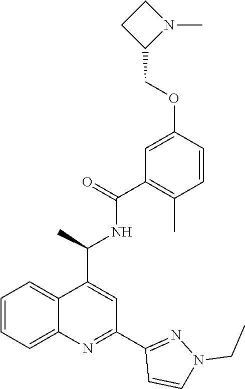

White solid, 63% yield. ¹H NMR (400 MHz, DMSO-d₆) δ 9.13 (d, J=7.2 Hz, 1H), 8.29 (d, J=8.3 Hz, 1H), 8.25 (s, 1H), 8.09 (d, J=8.4 Hz, 1H), 7.88 (d, J=2.4 Hz, 1H), 7.81-7.73 (m, 1H), 7.67-7.55 (m, 1H), 7.20-7.05 (m, 1H), 7.03 (d, J=2.3 Hz, 1H), 6.95 (d, J=7.0 Hz, 2H), 5.83 (p, J=7.0 Hz, 1H), 4.61 (t, J=6.2 Hz, 1H), 4.27 (t, J=5.1 Hz, 2H), 4.20 (q, J=7.3 Hz, 2H), 4.02 (tt, J=10.2, 5.6 Hz, 1H), 3.85 (tt, J=9.5, 4.5 Hz, 1H), 2.82 (d, J=4.1 Hz, 3H), 2.33 (qd, J=8.4, 7.7, 3.6 Hz, 2H), 2.17 (s, 3H), 1.55 (d, J=6.9 Hz, 3H), 1.39 (t, J=7.2 Hz, 3H). ¹³C NMR (101 MHz, DMSO-d₆) δ 168.59, 155.80, 151.42, 138.24, 132.15, 132.02, 130.83, 128.44, 127.20, 125.42, 124.11, 116.00, 114.86, 114.25, 105.44, 68.21, 67.09, 53.04, 47.29, 45.37, 41.40, 21.41, 18.93, 18.85, 15.93. LCMS calcd for C₂₉H₃₄N₅O₂ (M+H⁺): 484.2634; Found: 484.2638.

### Example 106. Synthesis of N—((R)-1-(2-(1-ethyl-1H-pyrazol-3-yl)quinolin-4-yl)ethyl)-2-methyl-5-(8-methyl-3,8-diazabicyclo[3.2.1]octan-3-yl)benzamide

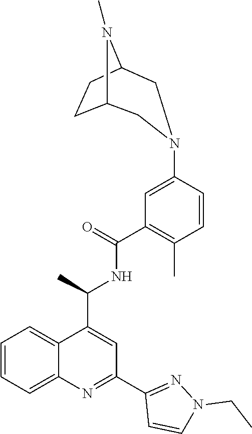 [SMILES: CCn1ccc(-c2cc([C@@H](C)NC(=O)c3cc(N4CC5CCC(C4)N5C)ccc3C)c3ccccc3n2)n1]

White solid, 60% yield. ¹H NMR (400 MHz, DMSO-d₆) δ 9.11 (d, J=7.0 Hz, 1H), 8.80 (dd, J=8.7, 4.2 Hz, 1H), 8.31 (d, J=18.4 Hz, 1H), 8.14 (d, J=8.2 Hz, 1H), 7.94 (d, J=2.7 Hz, 1H), 7.84 (q, J=7.6, 5.9 Hz, 1H), 7.69 (t, J=5.9 Hz, 1H), 7.15-7.06 (m, 2H), 6.96-6.85 (m, 2H), 5.87 (p, J=6.3 Hz, 1H), 4.27 (dd, J=9.7, 5.3 Hz, 2H), 4.06 (s, 2H), 3.70 (d, J=12.6 Hz, 2H), 3.11 (d, J=12.5 Hz, 2H), 2.78 (d, J=4.5 Hz, 2H), 2.50 (s, 3H), 2.26-2.12 (m, 4H), 2.00-1.95 (m, 1H), 1.69 (d, J=6.3 Hz, 1H), 1.61 (d, J=6.6 Hz, 3H), 1.50-1.36 (m, 3H). ¹³C NMR (101 MHz, DMSO-d₆) δ 169.21, 152.91, 151.52, 147.66, 145.31, 140.68, 137.77, 132.06, 131.90, 131.55, 130.69, 130.33, 128.64, 127.48, 127.08, 126.26, 125.41, 124.26, 124.09, 122.09, 117.94, 116.18, 115.02, 114.82, 114.00, 105.75, 105.33, 62.57, 52.00, 47.28, 46.13, 45.39, 38.75, 23.85, 21.47, 20.99, 18.70, 15.95. LCMS calcd for C₃₁H₃₇N₆O (M+H⁺): 509.2951; Found: 509.2953.

### Example 107. Synthesis of N—((R)-1-(2-(1-ethyl-1H-pyrazol-3-yl)quinolin-4-yl)ethyl)-2-methyl-5-((1R,4R)-5-methyl-2,5-diazabicyclo[2.2.1]heptan-2-yl)benzamide

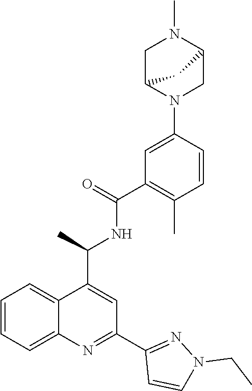

White solid. 61% yield. ¹H NMR (400 MHz, DMSO-d₆) δ 9.15 (d, J=7.1 Hz, 1H), 8.43-8.27 (m, 2H), 8.18 (d, J=8.4 Hz, 1H), 7.99 (d, J=2.3 Hz, 1H), 7.89 (t, J=7.7 Hz, 1H), 7.73 (t, J=7.6 Hz, 1H), 7.13 (dt, J=11.9, 3.4 Hz, 2H), 6.77-6.62 (m, 2H), 5.93 (p, J=7.1 Hz, 1H), 4.31 (q, J=7.3 Hz, 2H), 3.64 (s, 2H), 3.35 (dd, J=10.9, 7.0 Hz, 1H), 3.13 (d, J=10.8 Hz, 1H), 2.93 (d, J=4.9 Hz, 3H), 2.43 (t, J=5.9 Hz, 1H), 2.25 (d, J=7.2 Hz, 3H), 2.14 (d, J=12.3 Hz, 1H), 1.67 (d, J=7.0 Hz, 3H), 1.51 (t, J=7.3 Hz, 3H). ¹³C NMR (101 MHz, DMSO-d₆) δ 169.53, 151.70, 144.14, 132.01, 131.72, 130.55, 128.89, 127.01, 125.42, 124.03, 114.88, 112.21, 105.23, 66.28, 56.38, 47.26, 45.33, 42.37, 33.37, 21.46, 18.68, 15.94. LCMS calcd for C₃₀H₃₅N₆O (M+H⁺): 495.2794; Found: 495.2797.

### Example 108. Synthesis of (R)—N-(1-(2-(1-ethyl-1H-pyrazol-3-yl)quinolin-4-yl)ethyl)-2-methyl-5-(4-methylpiperazin-1-yl)benzamide

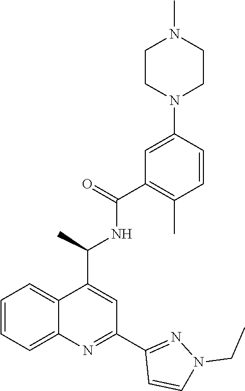 [SMILES: CCn1ccc(-c2cc([C@@H](C)NC(=O)c3cc(N4CCN(C)CC4)ccc3C)c3ccccc3n2)n1]

White solid, 64% yield. ¹H NMR (400 MHz, DMSO-d₆) δ 9.15 (d, J=7.0 Hz, 1H), 8.44 (d, J=8.5 Hz, 1H), 8.36 (s, 1H), 8.26 (d, J=8.5 Hz, 1H), 8.01 (d, J=2.4 Hz, 1H), 7.95 (t, J=7.7 Hz, 1H), 7.78 (t, J=7.7 Hz, 1H), 7.22-7.12 (m, 2H), 7.05-6.95 (m, 2H), 5.91 (p, J=7.0 Hz, 1H), 4.30 (q, J=7.3 Hz, 2H), 3.84 (s, 2H), 3.53 (d, J=12.3 Hz, 2H), 3.17 (dt, J=18.3, 9.7 Hz, 2H), 2.94 (t, J=12.5 Hz, 2H), 2.88 (s, 3H), 2.19 (s, 3H), 1.63 (d, J=7.0 Hz, 3H), 1.46 (t, J=7.3 Hz, 3H). ¹³C NMR (101 MHz, DMSO-d₆) δ 169.24, 150.27, 147.72, 137.61, 132.62, 132.01, 131.73, 127.91, 127.27, 126.64, 125.38, 124.42, 117.87, 115.39, 115.17, 106.29, 52.84, 47.53, 46.40, 45.72, 42.61, 21.40, 18.77, 15.91. LCMS calcd for C₂₉H₃₅N₆O (M+H⁺): 483.2794; Found: 483.2795.

### Example 109. Synthesis of N—((R)-1-(2-(1-ethyl-1H-pyrazol-3-yl)quinolin-4-yl)ethyl)-2-methyl-5-(((S)-1-methylpyrrolidin-2-yl)methoxy)benzamide

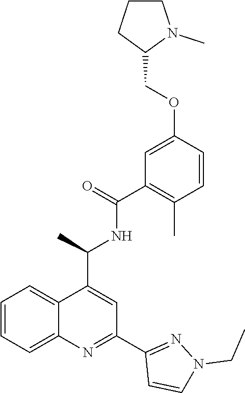

White solid, 64% yield. ¹H NMR (400 MHz, DMSO-d₆) δ 9.11 (d, J=7.2 Hz, 1H), 8.31-8.21 (m, 2H), 8.08 (d, J=8.4 Hz, 1H), 7.87 (d, J=2.3 Hz, 1H), 7.77 (ddd, J=8.4, 6.8, 1.2 Hz, 1H), 7.62 (td, J=7.5, 6.9, 1.2 Hz, 1H), 7.17-7.10 (m, 1H), 7.01 (d, J=2.3 Hz, 1H), 6.95 (dt, J=5.1, 2.1 Hz, 1H), 5.82 (dd, J=8.5, 5.6 Hz, 1H), 4.29-4.12 (m, 4H), 3.76-3.69 (m, 1H), 3.58-3.48 (m, 1H), 3.39-3.21 (m, 1H), 3.08 (dq, J=11.2, 7.8 Hz, 1H), 2.89 (d, J=4.3 Hz, 2H), 2.72 (d, J=4.7 Hz, 1H), 2.16 (d, J=5.7 Hz, 3H), 2.03-1.94 (m, 1H), 1.86 (tt, J=12.5, 6.3 Hz, 1H), 1.55 (d, J=7.0 Hz, 3H), 1.39 (t, J=7.3 Hz, 3H). ¹³C NMR (101 MHz, DMSO-d₆) δ 168.58, 158.67, 155.79, 151.55, 138.28, 132.09, 132.01, 130.69, 128.66, 128.44, 127.11, 125.43, 124.07, 118.04, 115.92, 115.12, 114.84, 114.18, 105.33, 67.19, 66.72, 57.04, 47.27, 45.33, 41.06, 26.79, 22.45, 21.41, 18.85, 15.94. LCMS calcd for C₃₀H₆N₅O₂ (M+H⁺): 498.2791; Found: 498.2795.

### Example 110. Synthesis of N—((R)-1-(2-(1-ethyl-1H-pyrazol-3-yl)quinolin-4-yl)ethyl)-2-methyl-5-(((R)-1-methylpyrrolidin-2-yl)methoxy)benzamide

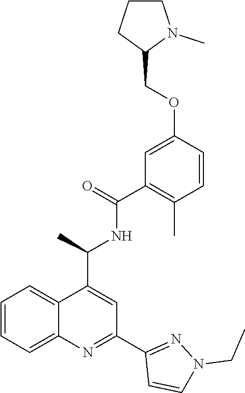

White solid, 69% yield. ¹H NMR (400 MHz, DMSO-d₆) δ 9.13 (d, J=7.2 Hz, 1H), 8.30 (d, J=8.3 Hz, 1H), 8.25 (d, J=3.7 Hz, 1H), 8.11 (d, J=8.4 Hz, 1H), 7.89 (d, J=2.3 Hz, 1H), 7.80 (dd, J=8.4, 6.9 Hz, 1H), 7.64 (ddd, J=8.3, 6.8, 1.4 Hz, 1H), 7.17-7.11 (m, 1H), 7.04 (d, J=2.3 Hz, 1H), 6.99-6.92 (m, 2H), 5.83 (p, J=7.0 Hz, 1H), 4.25-4.14 (m, 4H), 3.74 (dq, J=7.8, 5.2, 3.6 Hz, 1H), 3.62-3.47 (m, 1H), 3.28 (dt, J=30.8, 12.0 Hz, 1H), 3.08 (dq, J=11.3, 7.9 Hz, 1H), 2.90 (s, 3H), 2.72 (d, J=4.7 Hz, 1H), 2.17 (d, J=2.5 Hz, 3H), 2.00 (qd, J=7.6, 2.7 Hz, 1H), 1.84 (ddt, J=18.8, 12.2, 7.4 Hz, 2H), 1.55 (d, J 7.5 Hz, 3H), 1.38 (d, J=7.3 Hz, 3H). ¹³C NMR (101 MHz, DMSO-d₆) δ 168.61, 155.79, 154.07, 151.27, 138.26, 132.21, 132.02, 130.99, 128.43, 127.29, 125.41, 124.15, 117.87, 115.86, 114.92, 114.26, 105.55, 67.20, 66.72, 57.05, 47.32, 45.40, 41.08, 26.76, 22.45, 21.41, 18.83, 15.93. LCMS calcd for C₃₀H₃₆NO₂ (M+H⁺): 498.2791; Found: 498.2794.

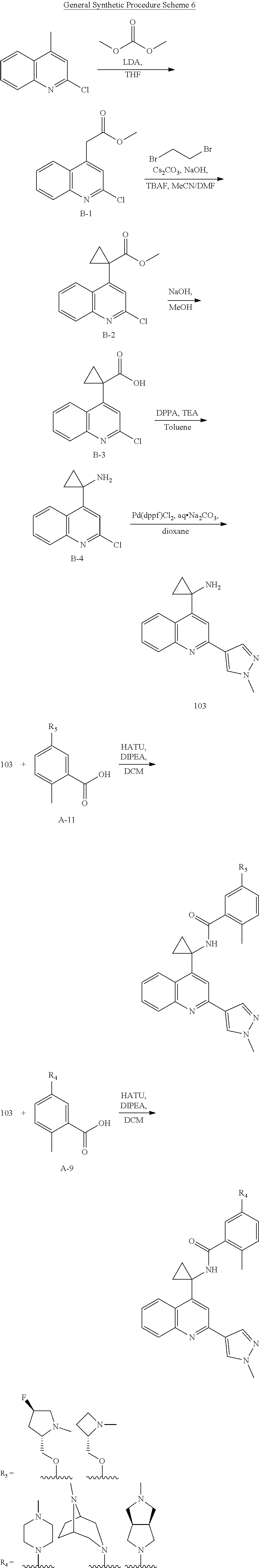

### Synthetic Procedures for Scheme 6

### Preparation of methyl 2-(2-chloroquinolin-4-yl)acetate (B-1)

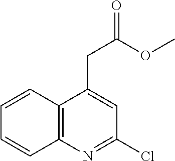 [SMILES: COC(=O)Cc1cc(Cl)nc2ccccc12]

To a solution of 2-chloro-4-methylquinoline (1.0 g, 6.984 mmol) in THF (30 mL) under N₂ was added LDA (6.0 mL, 2 M, 12 mmol) at −78° C. The reaction mixture was stirred at −78° C. under N₂ for 30 min, followed by the addition of dimethyl carbonate (1.27 g, 14.1 mmol) at 0° C. The reaction mixture was stirred at 0° C. under N₂ for 1 h, then quenched with 3 mL sat. NH₄Cl at 0° C. The reaction mixture was partitioned between ethyl acetate and H₂O (200/50 mL), the organic layer was separated, and the aqueous layer was re-extracted with ethyl acetate (50 mL). The combined organic layer was washed with brine, dried with anhydrous Na₂SO₄, and evaporated in vacuo. The residue was purified by silica gel chromatography using a Biotage (20 g silica gel column, PE:EA=1:0 to 0:1) to afford B-1(744 mg) as a yellow oil.

### Preparation of methyl 2-(2-chloroquinolin-4-yl)acetate (B-1)

Yellow oil, 56% yield. ¹H NMR (400 MHz, CDCl₃) δ 8.04 (d, J=8.4 Hz, 1H), 7.94 (d, J=8.4 Hz, 1H), 7.78-7.70 (m, 1H), 7.63-7.55 (m, 1H), 7.34 (s, 1H), 4.04 (s, 2H), 3.72 (s, 3H). ¹³C NMR (101 MHz, CDCl₃) δ 169.84, 150.35, 147.97, 143.17, 130.49, 129.28, 127.27, 126.12, 123.56, 123.43, 52.53, 37.79. LCMS calcd for C₁₂H₁₁ClNO₂ (M+H⁺): 236.0400; Found: 236.0400.

### Preparation of methyl 1-(2-chloroquinolin-4-yl)cyclopropane-1-carboxylate (B-2)

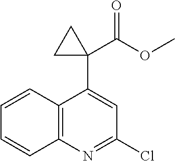 [SMILES: COC(=O)C1(c2cc(Cl)nc3ccccc23)CC1]

To a solution of methyl 2-(2-chloroquinolin-4-yl)acetate (3.0 g, 12.73 mmol) in DMF (60.0 mL) were added 1,2-dibromoethane (3.59 g, 19.1 mmol), Cs₂CO₃ (8.3 g, 25.5 mmol), tetra-n-butylammonium fluoride (13 mL, 1 M, 13 mmol), and NaOH (1.020 g, 25.5 mmol) at 20° C. The reaction mixture was stirred at 20° C. for 15 h. LCMS showed a mass peak of the desired product. The reaction mixture was then partitioned between ethyl acetate and H₂O (250/100 mL). The organic layer was separated, and the aqueous layer was reextracted with ethyl acetate (150 mL). The combined organic layer was washed with brine, dried with anhydrous Na₂SO₄, filtered, and concentrated in vacuo. The residue was then purified by silica gel chromatography using a Biotage (80 g silica gel column, PE:EA=1:0 to 0:1) to afford B-2.

### Preparation of methyl 1-(2-chloroquinolin-4-yl)cyclopropane-1-carboxylate (B-2)

Yellow solid, 87% yield. ¹H NMR (400 MHz, CDCl₃) δ 8.04 (t, J=9.1 Hz, 2H), 7.73 (t, J=7.7 Hz, 1H), 7.58 (t, J=7.8 Hz, 1H), 7.35 (s, 1H), 3.58 (s, 3H), 1.97-1.77 (m, 3H), 1.32 (s, 4H). ¹³C NMR (101 MHz, CDCl₃) δ 173.20, 150.37, 148.64, 148.06, 130.24, 129.23, 127.30, 127.06, 124.46, 123.40, 52.66, 26.31, 17.01. LCMS calcd for C₁₄H₁₂ClNO₂ (M+H⁺): 262.0557; Found: 262.0559.

### Preparation of 1-(2-chloroquinolin-4-yl)cyclopropane-1-carboxylic acid (B-3)

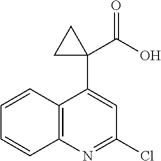 [SMILES: O=C(O)C1(c2cc(Cl)nc3ccccc23)CC1]

To a solution of methyl 1-(2-chloroquinolin-4-yl)cyclopropane-1-carboxylate (2.88 g, 11.02 mmol) in MeOH (60 mL) was added 10% aq. NaOH (15 mL) at 20° C. The reaction mixture was stirred at 20° C. for 15 h. LCMS showed a mass peak of the desired product. The reaction mixture was then acidified with 1N HCl to pH=1 at 0° C. and partitioned between ethyl acetate and H₂O (250/50 mL). The organic layer was separated, and the aqueous layer was re-extracted with ethyl acetate (150 mL). The combined organic layer was washed with brine, dried with anhydrous Na₂SO₄, and evaporated in vacuo to afford B-3 (2.68 g) as a yellow solid.

### Preparation of 1-(2-chloroquinolin-4-yl)cyclopropane-1-carboxylic acid (B-3)

Yellow solid, 99% yield. ¹H NMR (400 MHz, CD3OD) δ 8.12 (d, J=7.9 Hz, 1H), 7.87 (d, J=8.0 Hz, 1H), 7.70 (t, J=7.3 Hz, 1H), 7.58 (t, J=7.3 Hz, 1H), 7.45 (s, 1H), 1.81-1.75 (m, 2H), 1.29 (s, 2H). ¹³C NMR (101 MHz, CD3OD) δ 175.84, 151.95, 151.38, 148.53, 131.80, 128.88, 128.67, 128.36, 126.19, 124.47, 27.24, 17.45. LCMS calcd for C₁₃H₁₁ClNO₂ (M+H⁺): 248.0400; Found: 247.0403.

### Preparation of 1-(2-chloroquinolin-4-yl)cyclopropan-1-amine (B-4)

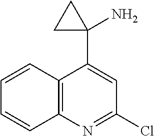 [SMILES: NC1(c2cc(Cl)nc3ccccc23)CC1]

To a solution of 1-(2-chloroquinolin-4-yl)cyclopropane-1-carboxylic acid (2.50 g, 10.09 mmol) in toluene (60.0 mL) under N₂, was added diphenylphosphoryl azide (3060 mg, 11.1 mmol) and TEA (2.25 g, 22.2 mmol) at 20° C. The reaction mixture was stirred at 90° C. under N₂ for 2 hours, then evaporated in vacuo to afford a crude. The crude was dissolved in 1,4-dioxane (40 mL), followed by adding aq. NaOH (10%, 20 mL) at 20° C. under N₂. The reaction mixture was stirred at 90° C. for 15 hours. LCMS showed a mass peak of the desired product. The reaction mixture was then partitioned between ethyl acetate and H₂O (250/150 mL). The organic layer was separated, and the aqueous layer was re-extracted with ethyl acetate (50 mL). The combined organic layer was washed with brine, dried with anhydrous Na₂SO₄, filtered, and concentrated in vacuo. The residue was dissolved in 30 mL MeOH, then 10 mL 1 N HCl was added to the solution at 20° C. The reaction mixture was stirred at 20° C. for 1 h, then partitioned between ethyl acetate and H₂O (250/150 mL). The organic layer was discarded, and the aqueous layer was basified with NH₃·H₂O to pH=9 at 0° C., followed by extraction with DCM (200 mL). The organic layer was washed with brine, dried with anhydrous Na₂SO₄ and concentrated in vacuo to afford B-4 (1.44 mg, 65%) as a white solid.

### Preparation of 1-(2-(1-methyl-1H-pyrazol-4-yl)quinolin-4-yl)cyclopropan-1-amine (103)

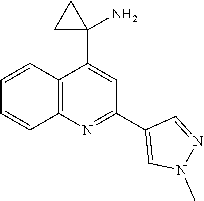 [SMILES: Cn1cc(-c2cc(C3(N)CC3)c3ccccc3n2)cn1]

To a solution of 1-(2-chloroquinolin-4-yl)cyclopropan-1-amine (1.0 g, 4.573 mmol) in 1,4-dioxane (40.0 mL) under N₂ were added sat. aq. Na₂CO₃ (8.0 mL), Pd(dppf)Cl₂ (335 mg, 0.457 mmol), and 1-methyl-4-(4,4,5,5-tetramethyl-1,3,2-dioxaborolan-2-yl)-1H-pyrazole (1.90 mg, 9.15 mmol) at 20° C. The reaction mixture was stirred at 100° C. under N₂ for 15 h. LCMS showed a mass peak of the desired product. The reaction mixture was then filtered, and the filtrate was concentrated in vacuo and purified by silica gel chromatography using a Biotage (20 g silica gel column, PE:EA=1:0 to 0:1) to afford 103. Yellow oil, 87% yield. ¹H NMR (400 MHz, CD3OD) δ 8.81 (s, 1H), 8.61-8.57 (m, 1H), 8.50 (s, 1H), 8.41 (s, 1H), 8.32 (d, J=8.5 Hz, 1H), 8.08 (ddd, J=8.5, 7.0, 1.3 Hz, 1H), 7.93 (ddd, J=8.4, 7.0, 1.1 Hz, 1H), 4.05 (s, 3H), 1.86-1.73 (m, 2H), 1.63-1.50 (m, 2H). ¹³C NMR (101 MHz, CD3OD) δ 150.81, 150.71, 141.68, 140.92, 135.13, 135.03, 130.17, 126.57, 126.14, 124.41, 123.52, 117.30, 39.82, 35.34, 12.64. LCMS calcd for C₁₆H₁₇N₄ (M+H⁺): 265.1375; Found: 265.1377.

### General Procedures for Amide Coupling

The corresponding acid (A-11) or (A-9) (1.1 equiv), HATU (1.1 equiv) and DIPEA (3.0 equiv) were dissolved in DMF (0.2 M) and stirred for 15 min. After that (R)-1-(2-(1-ethyl-1H-pyrazol-3-yl)quinolin-4-yl)ethan-1-amine (PDJ-G-10, 1.0 equiv) was added and stirred at room temperature overnight. When the starting material was consumed entirely as monitored by LCMS, the mixture was diluted with EtOAc and was then washed with saturated aq. NaHCO₃, water, and brine. The organic layer was dried over Na₂SO₄, filtered, and concentrated under vacuum to give a residue which was purified by Prep-HPLC to give the final product.

### Example 111. Synthesis of 2-methyl-N-(1-(2-(1-methyl-1H-pyrazol-4-yl)quinolin-4-yl)cyclopropyl)-5-(4-methylpiperazin-1-yl)benzamide

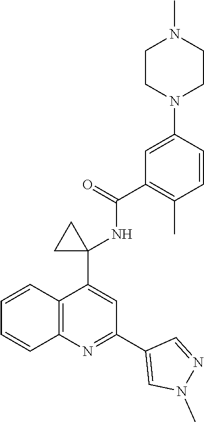 [SMILES: Cc1ccc(N2CCN(C)CC2)cc1C(=O)NC1(c2cc(-c3cnn(C)c3)nc3ccccc23)CC1]

White solid, 62% yield. ¹H NMR (400 MHz, CD3OD) δ 8.83 (s, 1H), 8.75 (d, J=8.3 Hz, 1H), 8.53 (s, 1H), 8.44 (d, J=1.9 Hz, 1H), 8.26 (d, J=8.5 Hz, 1H), 8.15-7.99 (m, 1H), 7.88 (t, J=7.6 Hz, 1H), 7.09 (d, J=8.5 Hz, 1H), 6.98 (dd, J=8.5, 2.7 Hz, 1H), 6.79 (d, J=2.7 Hz, 1H), 4.08 (s, 3H), 3.73 (d, J=13.4 Hz, 2H), 3.57 (d, J=11.8 Hz, 2H), 3.27-3.13 (m, 2H), 2.94 (s, 5H), 1.99 (s, 3H), 1.61 (d, J=3.7 Hz, 4H). ¹³C NMR (101 MHz, DMSO-d₆) δ 170.41, 149.99, 147.64, 139.27, 137.55, 132.90, 131.77, 131.57, 129.34, 128.65, 126.98, 126.83, 126.16, 125.89, 120.80, 119.35, 117.75, 115.10, 52.70, 46.26, 42.54, 34.60, 18.37, 14.28. CMS calcd for C₂₉H₃₃N₆O (M+H⁺): 481.2638; Found: 481.2639.

### Example 112. Synthesis of 2-methyl-N-(1-(2-(1-methyl-1H-pyrazol-4-yl)quinolin-4-yl)cyclopropyl)-5-(8-methyl-3,8-diazabicyclo[3.2.1]octan-3-yl)benzamide

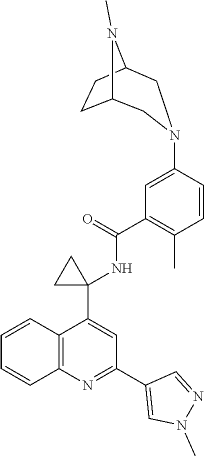 [SMILES: Cc1ccc(N2CC3CCC(C2)N3C)cc1C(=O)NC1(c2cc(-c3cnn(C)c3)nc3ccccc23)CC1]

White solid, 58% yield. ¹H NMR (400 MHz, CD3OD) δ 8.78 (s, 1H), 8.73 (d, J=8.4 Hz, 1H), 8.50 (s, 1H), 8.40 (s, 1H), 8.24 (d, J=8.5 Hz, 1H), 8.04 (t, J=7.8 Hz, 1H), 7.85 (t, J=7.8 Hz, 1H), 7.07 (d, J=8.4 Hz, 1H), 6.90 (dd, J=8.4, 2.7 Hz, 1H), 6.71 (d, J=2.7 Hz, 1H), 4.08 (s, 3H), 4.06-3.93 (m, 2H), 3.64 (d, J=12.8 Hz, 2H), 3.09 (d, J=12.9 Hz, 2H), 2.87 (s, 3H), 2.37-2.21 (m, 2H), 2.14 (t, J=6.7 Hz, 2H), 1.98 (s, 3H), 1.59 (d, J=4.7 Hz, 4H). ¹³C NMR (101 MHz, DMSO-d₆) δ 170.52, 159.06, 150.07, 147.57, 139.23, 137.61, 132.83, 131.67, 131.42, 126.90, 126.16, 125.90, 120.75, 118.12, 116.12, 115.20, 113.70, 62.48, 51.87, 39.50, 38.70, 34.59, 23.80, 18.24, 14.25. LCMS calcd for C₃₁H₃₅N₆O (M+H⁺): 507.2794; Found: 507.2796.

### Example 113. Synthesis of 2-methyl-N-(1-(2-(1-methyl-1H-pyrazol-4-yl)quinolin-4-yl)cyclopropyl)-5-((3aR,6aS)-5-methylhexahydropyrrolo[3,4-c]pyrrol-2(1H)-yl)benzamide

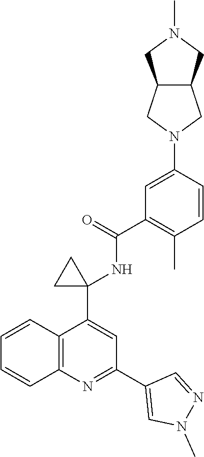 [SMILES: Cc1ccc(N2C[C@H]3CN(C)C[C@H]3C2)cc1C(=O)NC1(c2cc(-c3cnn(C)c3)nc3ccccc23)CC1]

White solid, 60% yield. ¹H NMR (400 MHz, CD3OD) δ 8.80 (q, J=4.7, 3.5 Hz, 1H), 8.74 (d, J=8.4 Hz, 1H), 8.52 (d, J=2.5 Hz, 1H), 8.42 (q, J=3.8, 2.7 Hz, 1H), 8.25 (d, J=8.4 Hz, 1H), 8.05 (t, J=7.5 Hz, 1H), 7.87 (t, J=7.2 Hz, 1H), 7.02 (d, J=8.4 Hz, 1H), 6.76 (dd, J=8.3, 2.6 Hz, 1H), 6.56 (d, J=2.6 Hz, 1H), 4.08 (s, 3H), 3.93 (s, 1H), 3.58 (d, J=11.6 Hz, 1H), 3.43 (dt, J=21.2, 10.4 Hz, 3H), 3.13 (s, 2H), 2.90 (d, J=20.4 Hz, 4H), 1.96 (s, 3H), 1.60 (s, 4H). ¹³C NMR (101 MHz, DMSO-d₆) δ 170.68, 149.96, 146.65, 146.46, 139.30, 137.54, 132.94, 131.82, 131.35, 127.02, 126.20, 125.90, 123.90, 120.81, 118.07, 115.81, 115.15, 113.29, 61.02, 60.34, 54.01, 52.66, 40.91, 34.60, 18.36, 14.30. LCMS calcd for C₃₁H₃₅N₆O (M+H⁺): 507.6540; Found: 507.6543.

### Example 114. Synthesis of 5-(((2S,4R)-4-fluoro-1-methylpyrrolidin-2-yl)methoxy)-2-methyl-N-(1-(2-(1-methyl-1H-pyrazol-4-yl)quinolin-4-yl)cyclopropyl)benzamide

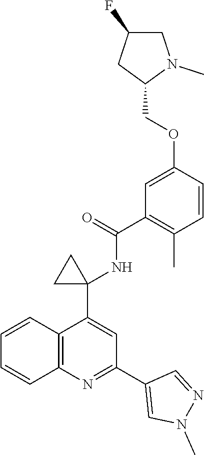 [SMILES: Cc1ccc(OC[C@@H]2C[C@@H](F)CN2C)cc1C(=O)NC1(c2cc(-c3cnn(C)c3)nc3ccccc23)CC1]

White solid, 64% yield. ¹H NMR (400 MHz, CD3OD) δ 8.81 (s, 1H), 8.74 (d, J=8.5 Hz, 1H), 8.52 (s, 1H), 8.42 (d, J=1.7 Hz, 1H), 8.25 (d, J=8.6 Hz, 1H), 8.02 (t, J=7.8 Hz, 1H), 7.85 (t, J=7.7 Hz, 1H), 7.04 (d, J=7.3 Hz, 1H), 6.84 (d, J=2.6 Hz, 1H), 5.49 (s, 1H), 5.36 (s, 1H), 4.39 (d, J=9.9 Hz, 1H), 4.26-4.10 (m, 2H), 4.06 (s, 3H), 3.66-3.47 (m, 1H), 3.07 (s, 3H), 2.59 (d, J=27.3 Hz, 1H), 2.43-2.18 (m, 1H), 2.03 (s, 3H), 1.59 (d, J=5.4 Hz, 4H). ¹³C NMR (101 MHz, DMSO-d₆) δ 169.81, 155.57, 152.64, 150.46, 139.06, 132.43, 131.91, 131.18, 129.82, 128.16, 126.65, 126.03, 125.91, 123.95, 120.73, 120.41, 115.89, 114.02, 67.00, 65.99, 62.14, 61.95, 43.60, 34.44, 18.49, 14.25. LCMS calcd for C₃₀H₃₃FN₅O₂ (M+H⁺): 514.2540; Found: 514.2544.

### Example 115. Synthesis of(S)-2-methyl-N-(1-(2-(1-methyl-1H-pyrazol-4-yl)quinolin-4-yl)cyclopropyl)-5-((1-methylazetidin-2-yl)methoxy)benzamide

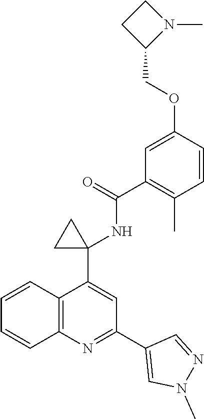 [SMILES: Cc1ccc(OC[C@@H]2CCN2C)cc1C(=O)NC1(c2cc(-c3cnn(C)c3)nc3ccccc23)CC1]

White solid, 64% yield. ¹H NMR (400 MHz, CD3OD) δ 8.80 (s, 1H), 8.74 (d, J=8.4 Hz, 1H), 8.51 (s, 1H), 8.43 (s, 1H), 8.25 (d, J=8.6 Hz, 1H), 8.05 (dd, J=8.6, 7.0 Hz, 1H), 7.90-7.83 (m, 1H), 7.13 (d, J=8.5 Hz, 1H), 6.98 (dd, J=8.4, 2.5 Hz, 1H), 6.82 (d, J=2.7 Hz, 1H), 4.69 (d, J=8.3 Hz, 1H), 4.30 (dd, J=11.5, 3.2 Hz, 1H), 4.26-4.16 (m, 2H), 4.08 (s, 3H), 3.96 (q, J=9.6 Hz, 1H), 2.95 (s, 3H), 2.56 (q, J=8.9 Hz, 2H), 2.02 (s, 3H), 1.61 (d, J=4.2 Hz, 4H). ¹³C NMR (101 MHz, DMSO-d₆) δ 169.42, 158.61, 155.25, 149.72, 138.73, 137.63, 132.30, 131.47, 131.16, 127.56, 126.47, 125.65, 125.45, 120.41, 119.25, 117.67, 115.48, 114.74, 113.52, 67.71, 66.59, 52.54, 40.93, 34.05, 18.40, 18.01, 13.84. LCMS calcd for C₂₉H₃₂N₅O₂ (M+H⁺): 482.2478; Found: 482.2479.

### Example 116. Biological Assays

### FRET-Based PL^{pro} Enzymatic Assay

The SARS-CoV-2 PL^{pro} enzymatic assays were carried out as follows. The assay was assembled in 96-well plates with 100 μl of 200 nM PL^{Pro} protein in PL^{Pro} reaction buffer [50 mM Hepes (pH 7.5), 0.01% Triton X-100, and 5 mM DTT]. Then, 1 μl of testing compound at various concentrations was added to each well and incubated at 30° C. for 30 minutes. The enzymatic reaction was initiated by adding 1 μl of 1 mM FRET substrate (the final substrate concentration is 10 μM). The reaction was monitored in a Cytation 5 image reader with filters for excitation at 360/40 nm and emission at 460/40 nm at 30° C. for 1 hour. The initial velocity of the enzymatic reaction with and without testing compounds was calculated by linear regression for the first 15 minutes of the kinetic progress curve. The IC₅₀ values were calculated by plotting the initial velocity against various concentrations of testing compounds with a dose-response function in Prism 8 software. Data for representative compounds is provided in the following table.

### Cytotoxicity Assay in Vero E6 Cells

Evaluation of the cytotoxicity of compounds was carried out using the neutral red uptake assay (Repetto, G., et al., Nat Protoc 2008, 3, 1125-31). Briefly, 80.000 cells/ml of the tested cell lines were dispensed into 96-well cell culture plates at 100 μl per well Twenty-four hours later, the growth medium was removed and washed with 150 μl of PBS buffer. Two hundred microliters of fresh serum-free medium containing serial diluted compounds was added to each well. After incubating for 2 days at 37° C., the medium was removed and replaced with 100 μl of DMEM medium containing neutral red (40 μg/ml) and incubated for 2 to 4 hours at 37° C. The amount of neutral red taken up was determined by measuring the absorbance at 540 nm using a Multiskan FC Microplate Photometer (Thermo Fisher Scientific) The CC₅₀ values were calculated from best-fit dose-response curves with variable slope in Prism 8. Data for representative compounds is provided in the following table.

TABLE

| | | | Vero |
|---|---|---|---|
| Example | Compound | IC₅₀ | CC₅₀ |
| 1 | 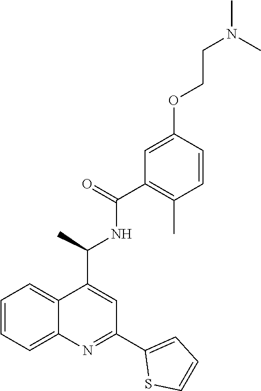 [SMILES: Cc1ccc(OCCN(C)C)cc1C(=O)N[C@H](C)c1cc(-c2cccs2)nc2ccccc12] | A | 12.22 ± 0.85 μM |
| 2 | 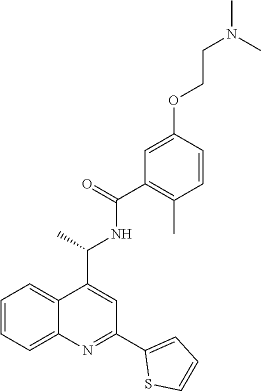 [SMILES: Cc1ccc(OCCN(C)C)cc1C(=O)N[C@@H](C)c1cc(-c2cccs2)nc2ccccc12] | C | 11.38 ± 0.79 μM |
| 3 | 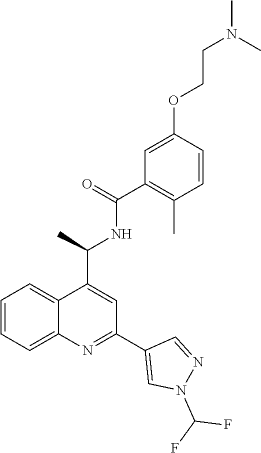 [SMILES: Cc1ccc(OCCN(C)C)cc1C(=O)N[C@H](C)c1cc(-c2cnn(C(F)F)c2)nc2ccccc12] | A | 15.18 ± 2.54 μM |
| 4 | 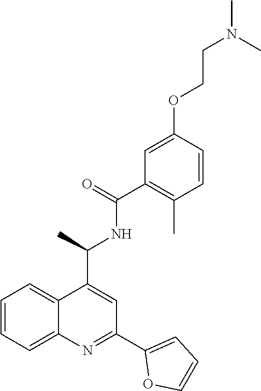 [SMILES: Cc1ccc(OCCN(C)C)cc1C(=O)N[C@H](C)c1cc(-c2ccco2)nc2ccccc12] | A | 57.28 ± 20.41 μM |
| 5 | 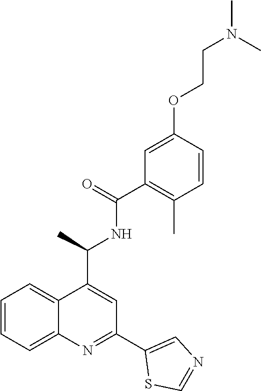 [SMILES: Cc1ccc(OCCN(C)C)cc1C(=O)N[C@H](C)c1cc(-c2cncs2)nc2ccccc12] | A | 44.72 ± 11.66 μM |
| 6 | 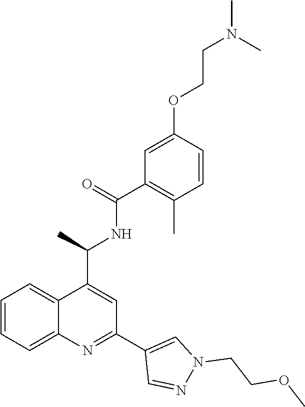 [SMILES: COCCn1cc(-c2cc([C@@H](C)NC(=O)c3cc(OCCN(C)C)ccc3C)c3ccccc3n2)cn1] | A | 200.16 ± 86.37 μM |
| 7 | 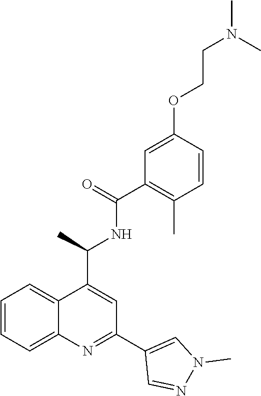 [SMILES: Cc1ccc(OCCN(C)C)cc1C(=O)N[C@H](C)c1cc(-c2cnn(C)c2)nc2ccccc12] | A | |
| 8 | 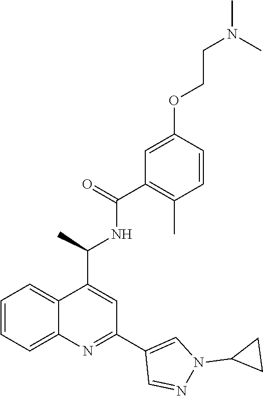 [SMILES: Cc1ccc(OCCN(C)C)cc1C(=O)N[C@H](C)c1cc(-c2cnn(C3CC3)c2)nc2ccccc12] | A | 63.08 ± 8.7 μM |
| 9 | 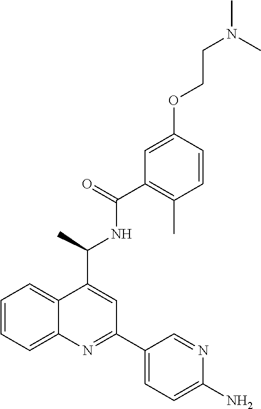 [SMILES: Cc1ccc(OCCN(C)C)cc1C(=O)N[C@H](C)c1cc(-c2ccc(N)nc2)nc2ccccc12] | A | 36.19 ± 6.83 μM |
| 10 | 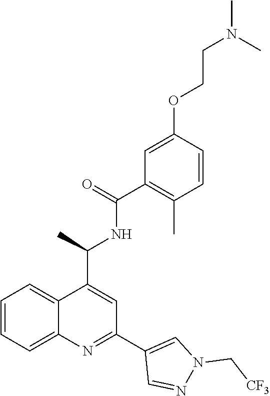 [SMILES: Cc1ccc(OCCN(C)C)cc1C(=O)N[C@H](C)c1cc(-c2cnn(CC(F)(F)F)c2)nc2ccccc12] | A | 60.75 ± 14.13 μM |

| | | | |
|---|---|---|---|
| 11 | 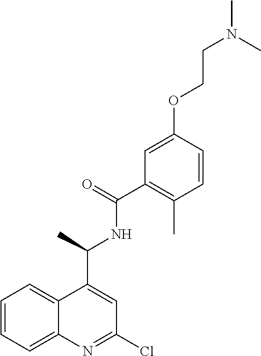 [SMILES: Cc1ccc(OCCN(C)C)cc1C(=O)N[C@H](C)c1cc(Cl)nc2ccccc12] | B | 100.72 ± 11.33 μM |

| | | | |
|---|---|---|---|
| 12 | 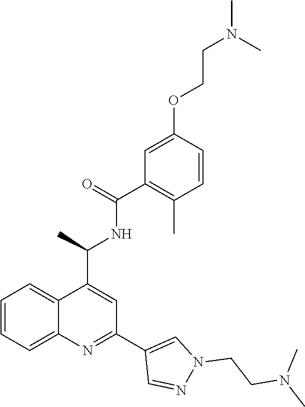 [SMILES: Cc1ccc(OCCN(C)C)cc1C(=O)N[C@H](C)c1cc(-c2cnn(CCN(C)C)c2)nc2ccccc12] | A | 113.78 ± 18.72 μM |

| | | | |
|---|---|---|---|
| 13 | 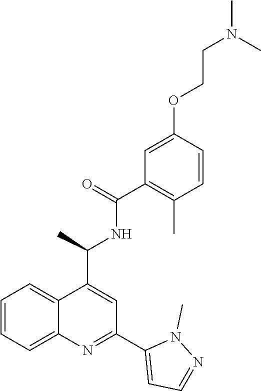 [SMILES: Cc1ccc(OCCN(C)C)cc1C(=O)N[C@H](C)c1cc(-c2ccnn2C)nc2ccccc12] | A | 60.01 ± 8.43 μM |

| | | | |
|---|---|---|---|
| 14 | 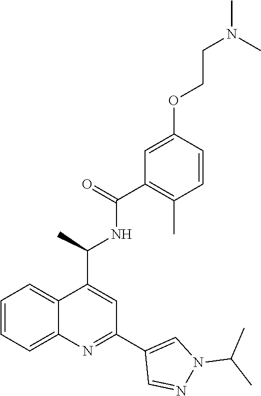 [SMILES: Cc1ccc(OCCN(C)C)cc1C(=O)N[C@H](C)c1cc(-c2cnn(C(C)C)c2)nc2ccccc12] | A | 37.69 ± 5.87 μM |

| | | | |
|---|---|---|---|
| 15 | 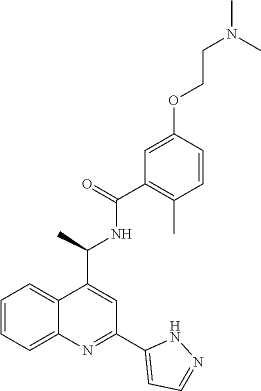 [SMILES: Cc1ccc(OCCN(C)C)cc1C(=O)N[C@H](C)c1cc(-c2ccn[nH]2)nc2ccccc12] | A | 63.18 ± 9.51 μM |

| | | | |
|---|---|---|---|
| 16 | 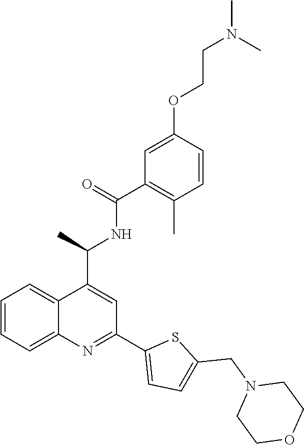 [SMILES: Cc1ccc(OCCN(C)C)cc1C(=O)N[C@H](C)c1cc(-c2ccc(CN3CCOCC3)s2)nc2ccccc12] | A | 30.64 ± 10.60 μM |

| | | |
|---|---|---|
| 17 | 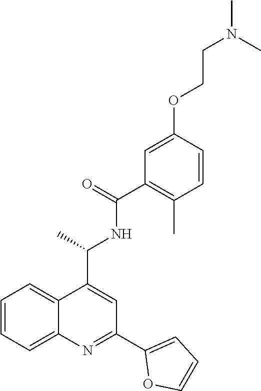 [SMILES: Cc1ccc(OCCN(C)C)cc1C(=O)N[C@@H](C)c1cc(-c2ccco2)nc2ccccc12] | A |

| | | | |
|---|---|---|---|
| 18 | 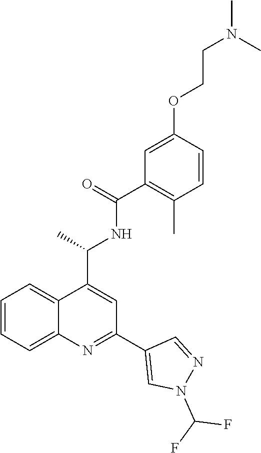 [SMILES: Cc1ccc(OCCN(C)C)cc1C(=O)N[C@@H](C)c1cc(-c2cnn(C(F)F)c2)nc2ccccc12] | B | 47.11 ± 6.43 μM |

| | | | |
|---|---|---|---|
| 19 | 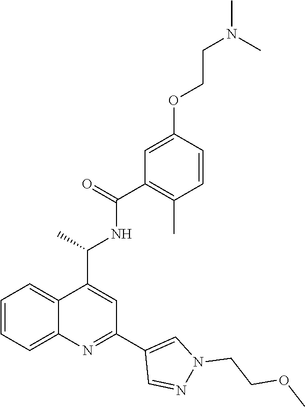 [SMILES: COCCn1cc(-c2cc([C@H](C)NC(=O)c3cc(OCCN(C)C)ccc3C)c3ccccc3n2)cn1] | C | >125 μM |

| | | | |
|---|---|---|---|
| 20 | 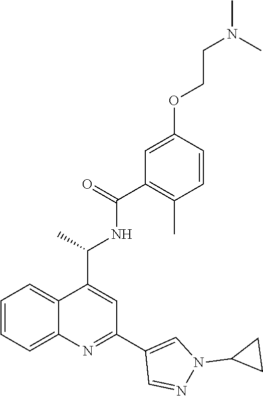 [SMILES: Cc1ccc(OCCN(C)C)cc1C(=O)N[C@@H](C)c1cc(-c2cnn(C3CC3)c2)nc2ccccc12] | A | 59.59 ± 8.56 μM |

| | | | |
|---|---|---|---|
| 21 | 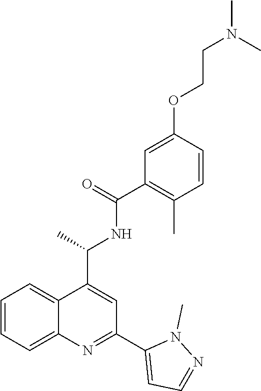 [SMILES: Cc1ccc(OCCN(C)C)cc1C(=O)N[C@@H](C)c1cc(-c2ccnn2C)nc2ccccc12] | B | 64.70 ± 8.94 μM |

| | | | |
|---|---|---|---|
| 22 | 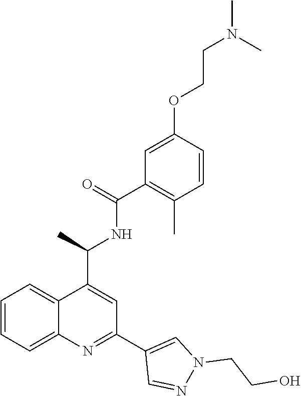 [SMILES: Cc1ccc(OCCN(C)C)cc1C(=O)N[C@H](C)c1cc(-c2cnn(CCO)c2)nc2ccccc12] | A | 152.86 ± 43.85 μM |

| | | |
|---|---|---|
| 23 | 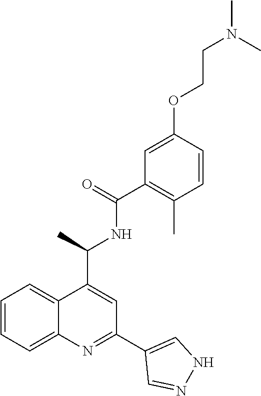 [SMILES: Cc1ccc(OCCN(C)C)cc1C(=O)N[C@H](C)c1cc(-c2cn[nH]c2)nc2ccccc12] | A |

| | | | |
|---|---|---|---|
| 24 | 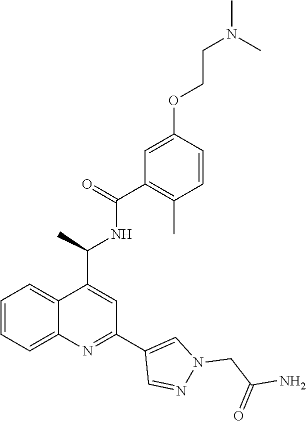 [SMILES: Cc1ccc(OCCN(C)C)cc1C(=O)N[C@H](C)c1cc(-c2cnn(CC(N)=O)c2)nc2ccccc12] | A | >250 μM |

| | | | |
|---|---|---|---|
| 25 | 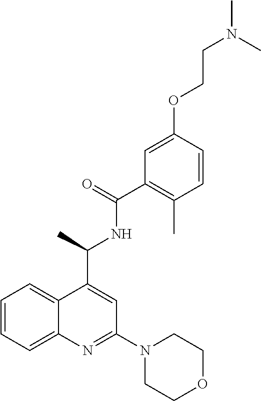 [SMILES: Cc1ccc(OCCN(C)C)cc1C(=O)N[C@H](C)c1cc(N2CCOCC2)nc2ccccc12] | R | 166.27 ± 33.52 μM |

| | | | |
|---|---|---|---|
| 26 | 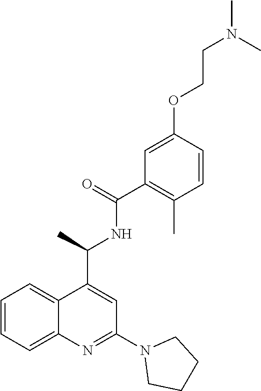 [SMILES: Cc1ccc(OCCN(C)C)cc1C(=O)N[C@H](C)c1cc(N2CCCC2)nc2ccccc12] | C | 118.39 ± 72.57 μM |

| | | | |
|---|---|---|---|
| 27 | 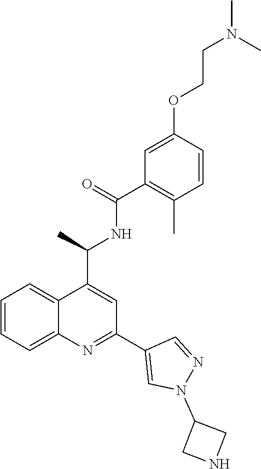 [SMILES: Cc1ccc(OCCN(C)C)cc1C(=O)N[C@H](C)c1cc(-c2cnn(C3CNC3)c2)nc2ccccc12] | A | >250 μM |

| | | | |
|---|---|---|---|
| 28 | 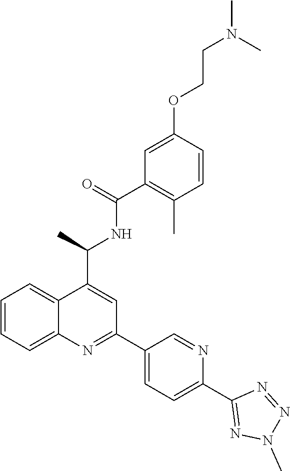 [SMILES: Cc1ccc(OCCN(C)C)cc1C(=O)N[C@H](C)c1cc(-c2ccc(-c3nnn(C)n3)nc2)nc2ccccc12] | A | 36.19 ± 9.62 μM |

| | | | |
|---|---|---|---|
| 29 | 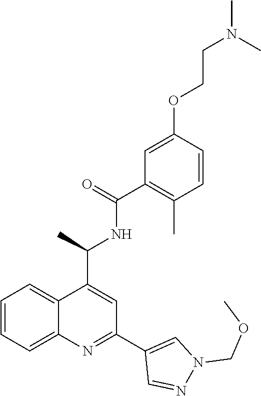 [SMILES: COCn1cc(-c2cc([C@@H](C)NC(=O)c3cc(OCCN(C)C)ccc3C)c3ccccc3n2)cn1] | A | 159.88 ± 14.45 μM |

| | | | |
|---|---|---|---|
| 30 | 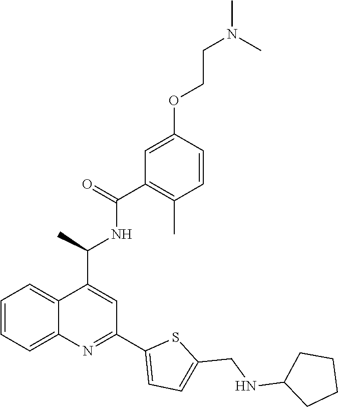 [SMILES: Cc1ccc(OCCN(C)C)cc1C(=O)N[C@H](C)c1cc(-c2ccc(CNC3CCCC3)s2)nc2ccccc12] | A | 13.80 ± 9.93 μM |

| | | | |
|---|---|---|---|
| 31 | 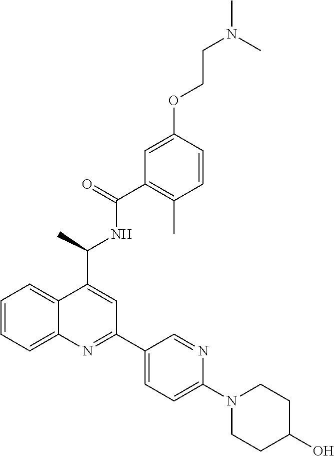 [SMILES: Cc1ccc(OCCN(C)C)cc1C(=O)N[C@H](C)c1cc(-c2ccc(N3CCC(O)CC3)nc2)nc2ccccc12] | A | 38.60 ± 3.56 μM |

| | | | |
|---|---|---|---|
| 32 | 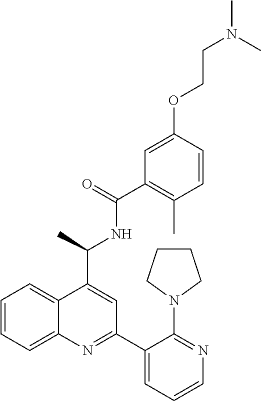 [SMILES: Cc1ccc(OCCN(C)C)cc1C(=O)N[C@H](C)c1cc(-c2cccnc2N2CCCC2)nc2ccccc12] | A | 47.21 ± 7.45 μM |

| | | | |
|---|---|---|---|
| 33 | 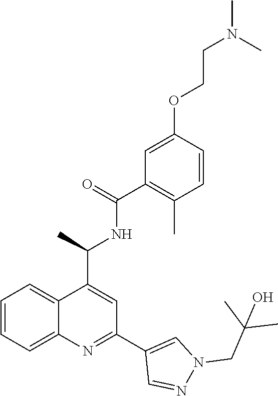 [SMILES: Cc1ccc(OCCN(C)C)cc1C(=O)N[C@H](C)c1cc(-c2cnn(CC(C)(C)O)c2)nc2ccccc12] | A | 174.45 ± 44.65 μM |

| | | | |
|---|---|---|---|
| 34 | 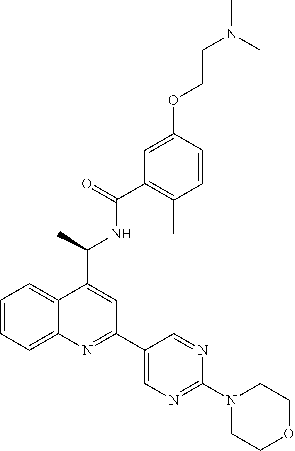 [SMILES: Cc1ccc(OCCN(C)C)cc1C(=O)N[C@H](C)c1cc(-c2cnc(N3CCOCC3)nc2)nc2ccccc12] | A | 24.76 ± 6.84 μM |

| | | | |
|---|---|---|---|
| 35 | 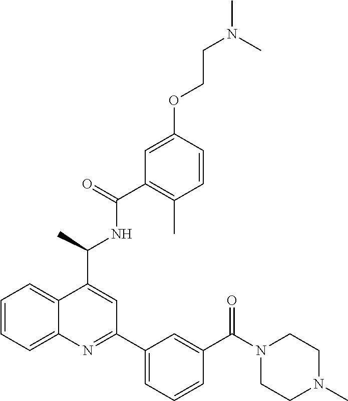 [SMILES: Cc1ccc(OCCN(C)C)cc1C(=O)N[C@H](C)c1cc(-c2cccc(C(=O)N3CCN(C)CC3)c2)nc2ccccc12] | A | 113.2 μM |

| | | | |
|---|---|---|---|
| 36 | 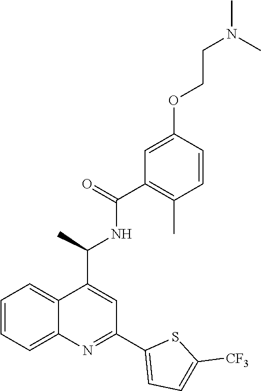 [SMILES: Cc1ccc(OCCN(C)C)cc1C(=O)N[C@H](C)c1cc(-c2ccc(C(F)(F)F)s2)nc2ccccc12] | A | 14.89 ± 10.37 μM |

| | | | |
|---|---|---|---|
| 37 | 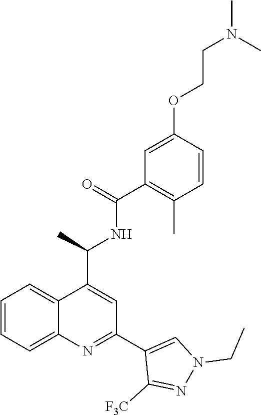 [SMILES: CCn1cc(-c2cc([C@@H](C)NC(=O)c3cc(OCCN(C)C)ccc3C)c3ccccc3n2)c(C(F)(F)F)n1] | A | 16.10 ± 1.39 μM |

| | | | |
|---|---|---|---|
| 38 | 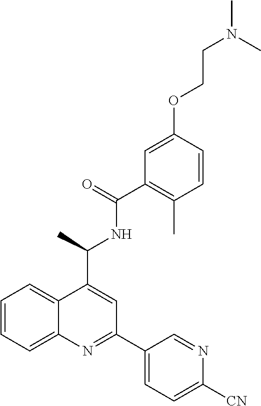 [SMILES: Cc1ccc(OCCN(C)C)cc1C(=O)N[C@H](C)c1cc(-c2ccc(C#N)nc2)nc2ccccc12] | A | 27.50 ± 2.18 μM |

| | | | |
|---|---|---|---|
| 39 | 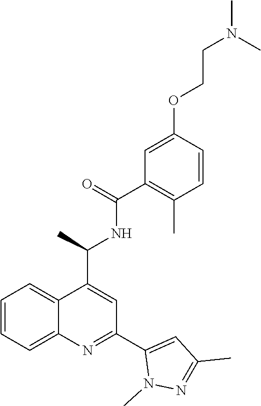 [SMILES: Cc1cc(-c2cc([C@@H](C)NC(=O)c3cc(OCCN(C)C)ccc3C)c3ccccc3n2)n(C)n1] | A | 47.18 ± 4 74 μM |

| | | | |
|---|---|---|---|
| 40 | 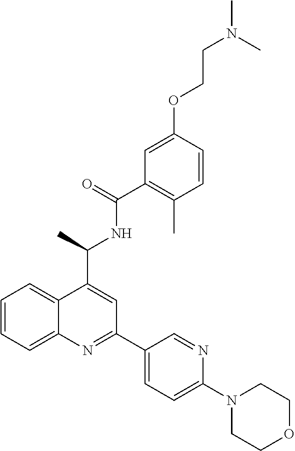 [SMILES: Cc1ccc(OCCN(C)C)cc1C(=O)N[C@H](C)c1cc(-c2ccc(N3CCOCC3)nc2)nc2ccccc12] | A | 16.66 ± 3.39 μM |

| | | | |
|---|---|---|---|
| 41 | 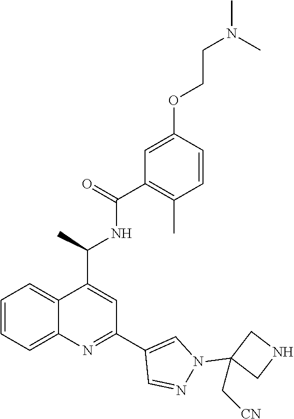 [SMILES: Cc1ccc(OCCN(C)C)cc1C(=O)N[C@H](C)c1cc(-c2cnn(C3(CC#N)CNC3)c2)nc2ccccc12] | A | >100 μM |

| | | | |
|---|---|---|---|
| 42 | 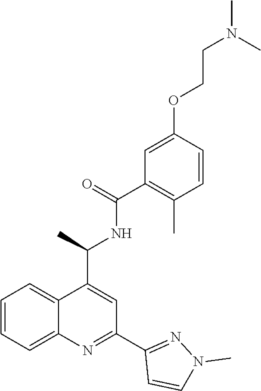 [SMILES: Cc1ccc(OCCN(C)C)cc1C(=O)N[C@H](C)c1cc(-c2ccn(C)n2)nc2ccccc12] | A | 86.06 ± 11.68 μM |

| | | | |
|---|---|---|---|
| 43 | 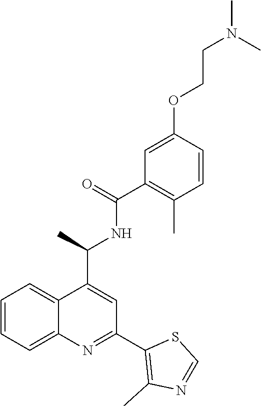 [SMILES: Cc1ccc(OCCN(C)C)cc1C(=O)N[C@H](C)c1cc(-c2scnc2C)nc2ccccc12] | B | 37.30 ± 2.63 μM |

| | | | |
|---|---|---|---|
| 44 | 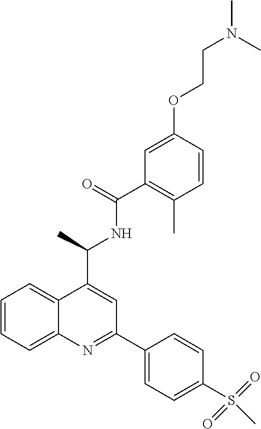 [SMILES: Cc1ccc(OCCN(C)C)cc1C(=O)N[C@H](C)c1cc(-c2ccc(S(C)(=O)=O)cc2)nc2ccccc12] | A | 34 ± 3.07 μM |

| | | | |
|---|---|---|---|
| 45 | 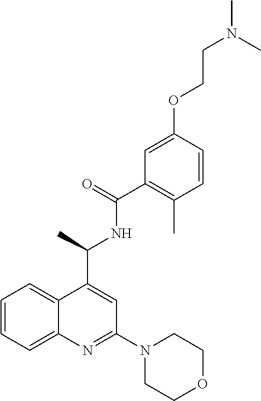 [SMILES: Cc1ccc(OCCN(C)C)cc1C(=O)N[C@H](C)c1cc(N2CCOCC2)nc2ccccc12] | B | 166.27 ± 33.25 μM |

| | | |
|---|---|---|
| 46 | 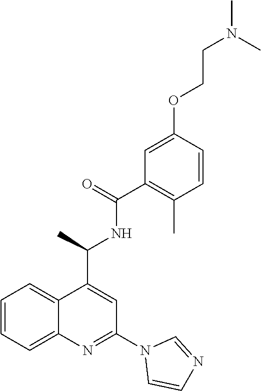 [SMILES: Cc1ccc(OCCN(C)C)cc1C(=O)N[C@H](C)c1cc(-n2ccnc2)nc2ccccc12] | A |

| | | | |
|---|---|---|---|
| 47 | 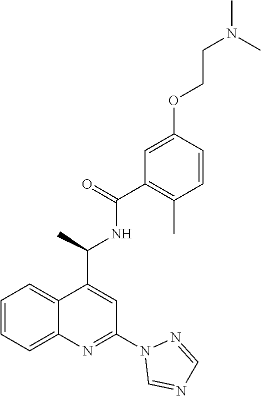 [SMILES: Cc1ccc(OCCN(C)C)cc1C(=O)N[C@H](C)c1cc(-n2cncn2)nc2ccccc12] | B | 17.46 ± 5.23 μM |

| | | | |
|---|---|---|---|
| 48 | 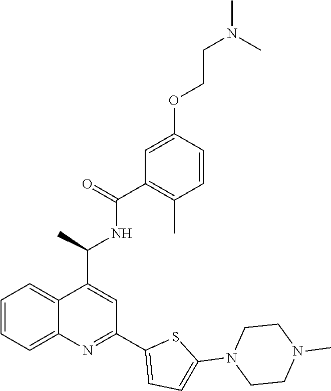 [SMILES: Cc1ccc(OCCN(C)C)cc1C(=O)N[C@H](C)c1cc(-c2ccc(N3CCN(C)CC3)s2)nc2ccccc12] | A | 15.41 ± 7.94 μM |

| | | |
|---|---|---|
| 49 | 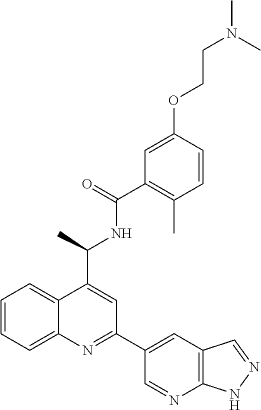 [SMILES: Cc1ccc(OCCN(C)C)cc1C(=O)N[C@H](C)c1cc(-c2cnc3[nH]ncc3c2)nc2ccccc12] | >250 μM |

| | | | |
|---|---|---|---|
| 50 | 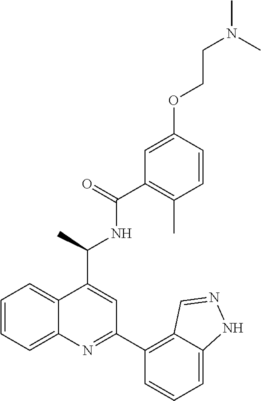 [SMILES: Cc1ccc(OCCN(C)C)cc1C(=O)N[C@H](C)c1cc(-c2cccc3[nH]ncc23)nc2ccccc12] | A | 59.46 ± 25.45 μM |

| | | | |
|---|---|---|---|
| 51 | 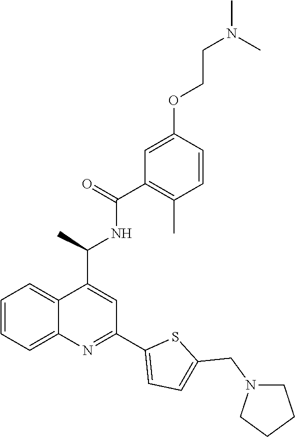 [SMILES: Cc1ccc(OCCN(C)C)cc1C(=O)N[C@H](C)c1cc(-c2ccc(CN3CCCC3)s2)nc2ccccc12] | A | 27.06 ± 14 μM |

| | | | |
|---|---|---|---|
| 53 | 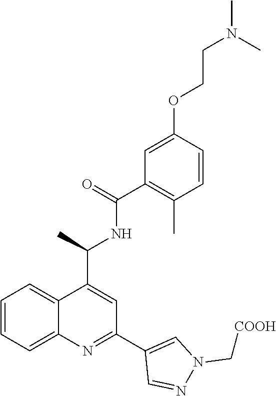 [SMILES: Cc1ccc(OCCN(C)C)cc1C(=O)N[C@H](C)c1cc(-c2cnn(CC(=O)O)c2)nc2ccccc12] | A | >250 μM |

| | | | |
|---|---|---|---|
| 54 | 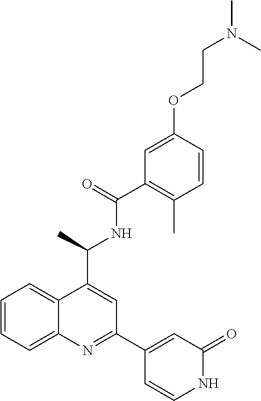 [SMILES: Cc1ccc(OCCN(C)C)cc1C(=O)N[C@H](C)c1cc(-c2cc[nH]c(=O)c2)nc2ccccc12] | B | >125 μM |

| | | | |
|---|---|---|---|
| 55 | 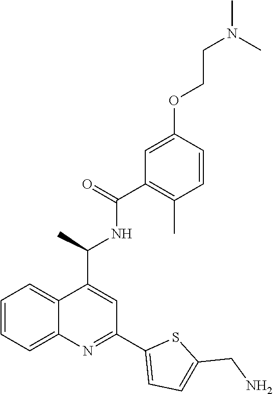 [SMILES: Cc1ccc(OCCN(C)C)cc1C(=O)N[C@H](C)c1cc(-c2ccc(CN)s2)nc2ccccc12] | A | 34.64 ± 8.18 μM |

| | | | |
|---|---|---|---|
| 56 | 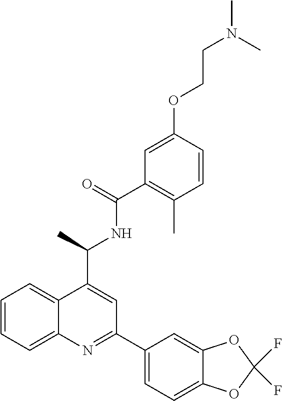 [SMILES: Cc1ccc(OCCN(C)C)cc1C(=O)N[C@H](C)c1cc(-c2ccc3c(c2)OC(F)(F)O3)nc2ccccc12] | A | 15.15 ± 7.09 μM |

| | | | |
|---|---|---|---|
| 57 | 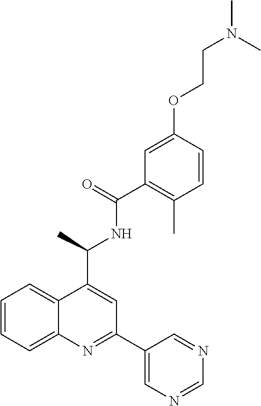 [SMILES: Cc1ccc(OCCN(C)C)cc1C(=O)N[C@H](C)c1cc(-c2cncnc2)nc2ccccc12] | B | 74.62 ± 17.35 μM |

| | | | |
|---|---|---|---|
| 58 | 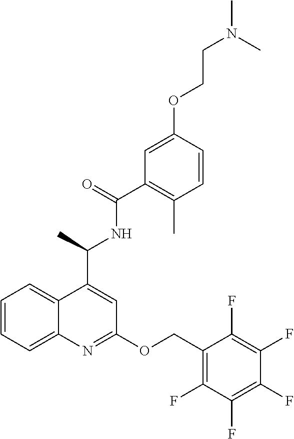 [SMILES: Cc1ccc(OCCN(C)C)cc1C(=O)N[C@H](C)c1cc(OCc2c(F)c(F)c(F)c(F)c2F)nc2ccccc12] | D | >50 μM |

| | | | |
|---|---|---|---|
| 59 | 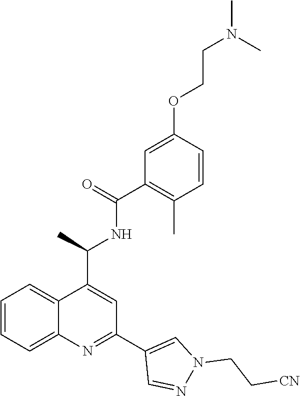 [SMILES: Cc1ccc(OCCN(C)C)cc1C(=O)N[C@H](C)c1cc(-c2cnn(CCC#N)c2)nc2ccccc12] | D | 101.31 ± 15.2 μM |

| | | | |
|---|---|---|---|
| 60 | 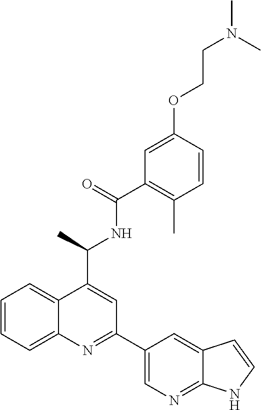 [SMILES: Cc1ccc(OCCN(C)C)cc1C(=O)N[C@H](C)c1cc(-c2cnc3[nH]ccc3c2)nc2ccccc12] | D | 25.75 ± 2.25 μM |

| | | | |
|---|---|---|---|
| 61 | 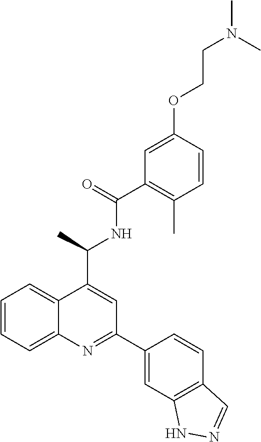 [SMILES: Cc1ccc(OCCN(C)C)cc1C(=O)N[C@H](C)c1cc(-c2ccc3cn[nH]c3c2)nc2ccccc12] | D | 25.66 ± 2.09 μM |

| | | | |
|---|---|---|---|
| 62 | 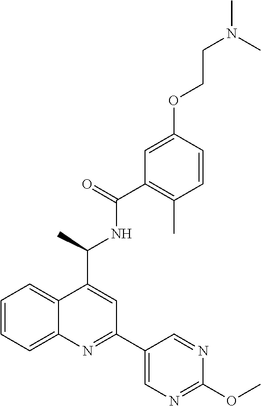 [SMILES: COc1ncc(-c2cc([C@@H](C)NC(=O)c3cc(OCCN(C)C)ccc3C)c3ccccc3n2)cn1] | D | 34.79 ± 4,47 μM |

| | | | |
|---|---|---|---|
| 63 | 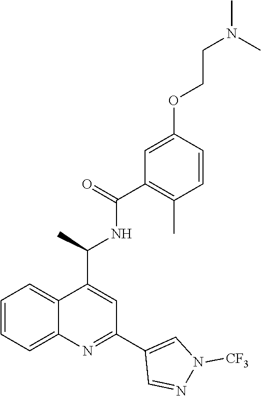 [SMILES: Cc1ccc(OCCN(C)C)cc1C(=O)N[C@H](C)c1cc(-c2cnn(C(F)(F)F)c2)nc2ccccc12] | A | 17.69 ± 2.04 μM |

| | | | |
|---|---|---|---|
| 64 | 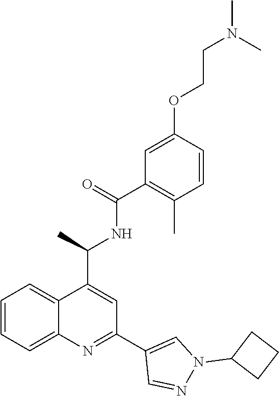 [SMILES: Cc1ccc(OCCN(C)C)cc1C(=O)N[C@H](C)c1cc(-c2cnn(C3CCC3)c2)nc2ccccc12] | A | 25.31 ± 5.82 μM |

| | | | |
|---|---|---|---|
| 65 | 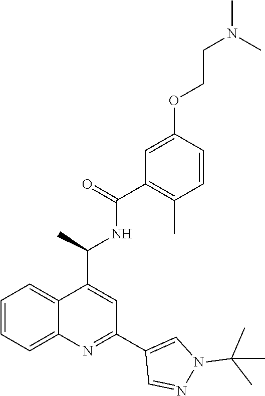 [SMILES: Cc1ccc(OCCN(C)C)cc1C(=O)N[C@H](C)c1cc(-c2cnn(C(C)(C)C)c2)nc2ccccc12] | A | 19.11 ± 2.75 μM |

| | | | |
|---|---|---|---|
| 66 | 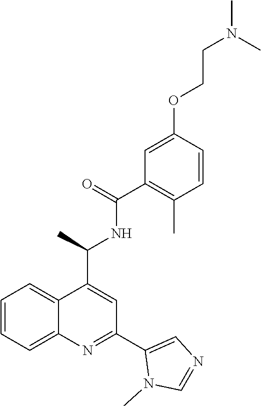 [SMILES: Cc1ccc(OCCN(C)C)cc1C(=O)N[C@H](C)c1cc(-c2cncn2C)nc2ccccc12] | A | 79.46 ± 8.62 μM |

| | | | |
|---|---|---|---|
| 67 | 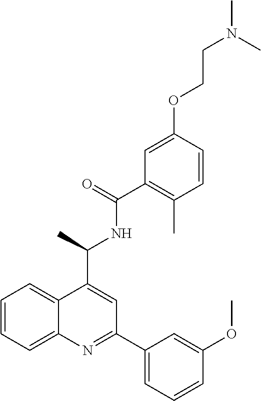 [SMILES: COc1cccc(-c2cc([C@@H](C)NC(=O)c3cc(OCCN(C)C)ccc3C)c3ccccc3n2)c1] | A | 17.15 ± 3.25 μM |

| | | | |
|---|---|---|---|
| 68 | 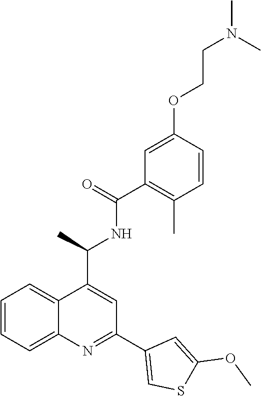 [SMILES: COc1cc(-c2cc([C@@H](C)NC(=O)c3cc(OCCN(C)C)ccc3C)c3ccccc3n2)cs1] | A | 13.86 ± 4.32 μM |

| | | | |
|---|---|---|---|
| 69 | 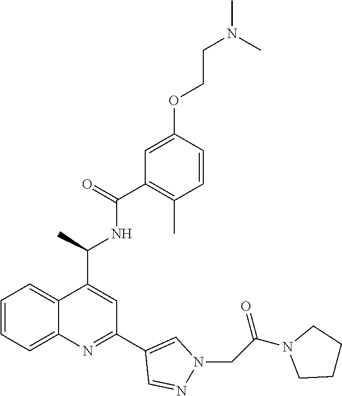 [SMILES: Cc1ccc(OCCN(C)C)cc1C(=O)N[C@H](C)c1cc(-c2cnn(CC(=O)N3CCCC3)c2)nc2ccccc12] | A | 26.4 ± 4.5 μM |

| | | | |
|---|---|---|---|
| 70 | 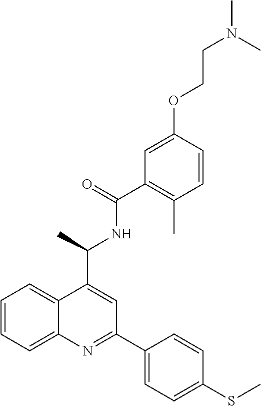 [SMILES: CSc1ccc(-c2cc([C@@H](C)NC(=O)c3cc(OCCN(C)C)ccc3C)c3ccccc3n2)cc1] | A | 8.50 ± 2.01 μM |

| | | | |
|---|---|---|---|
| 71 | 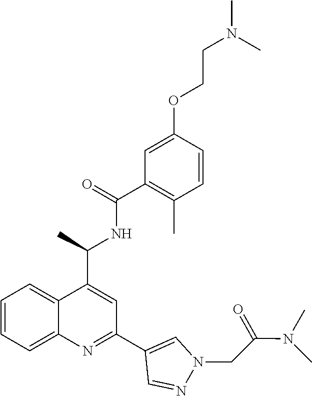 [SMILES: Cc1ccc(OCCN(C)C)cc1C(=O)N[C@H](C)c1cc(-c2cnn(CC(=O)N(C)C)c2)nc2ccccc12] | A | >250 μM |

| | | |
|---|---|---|
| 72 | 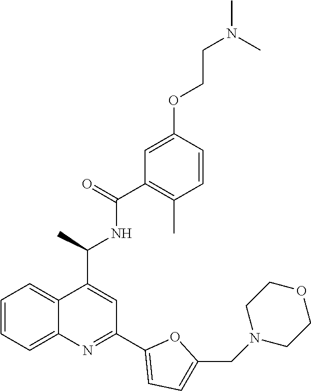 [SMILES: Cc1ccc(OCCN(C)C)cc1C(=O)N[C@H](C)c1cc(-c2ccc(CN3CCOCC3)o2)nc2ccccc12] | A |

| | | | |
|---|---|---|---|
| 74 | 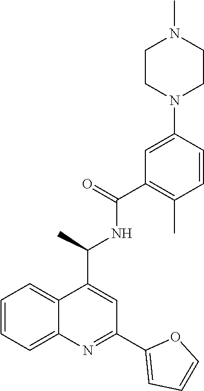 [SMILES: Cc1ccc(N2CCN(C)CC2)cc1C(=O)N[C@H](C)c1cc(-c2ccco2)nc2ccccc12] | A | 19.48 ± 1.13 μM |

| | | | |
|---|---|---|---|
| 75 | 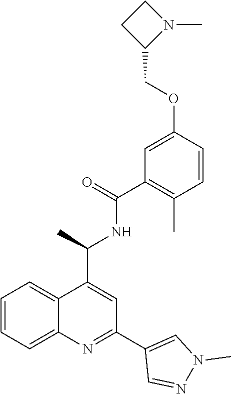 | A | 112.974 ± 7.902 μM |

| | | | |
|---|---|---|---|
| 76 | 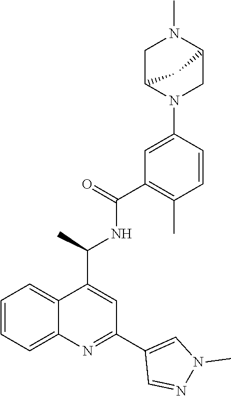 | A | 93.44 ± 5.06 μM |

| | | | |
|---|---|---|---|
| 77 | 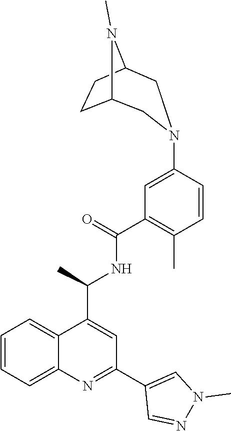 [SMILES: Cc1ccc(N2CC3CCC(C2)N3C)cc1C(=O)N[C@H](C)c1cc(-c2cnn(C)c2)nc2ccccc12] | A | 44.316 ± 2.462 μM |

| | | | |
|---|---|---|---|
| 78 | 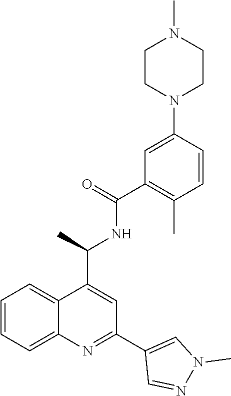 [SMILES: Cc1ccc(N2CCN(C)CC2)cc1C(=O)N[C@H](C)c1cc(-c2cnn(C)c2)nc2ccccc12] | A | 79.31 ± 5.79 μM |

| | | | |
|---|---|---|---|
| 79 | 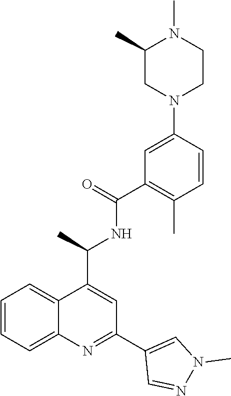 [SMILES: Cc1ccc(N2CCN(C)[C@H](C)C2)cc1C(=O)N[C@H](C)c1cc(-c2cnn(C)c2)nc2ccccc12] | A | 72.09 ± 5.88 μM |

| | | | |
|---|---|---|---|
| 80 | 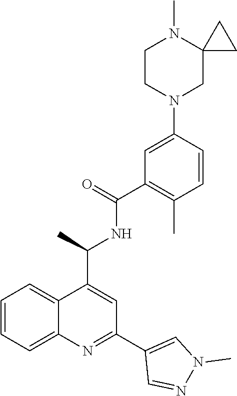 [SMILES: Cc1ccc(N2CCN(C)C3(CC3)C2)cc1C(=O)N[C@H](C)c1cc(-c2cnn(C)c2)nc2ccccc12] | A | 49.72 ± 4.65 μM |

| | | | |
|---|---|---|---|
| 81 | 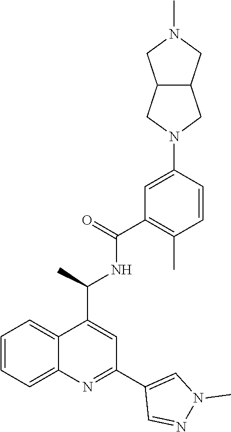 [SMILES: Cc1ccc(N2CC3CN(C)CC3C2)cc1C(=O)N[C@H](C)c1cc(-c2cnn(C)c2)nc2ccccc12] | A | 32.58 ± 1.34 μM |

| | | | |
|---|---|---|---|
| 82 | 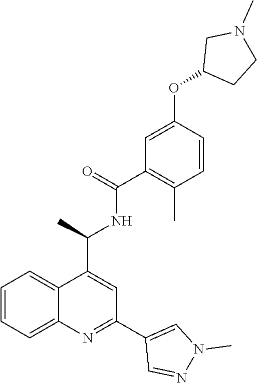 | A | 48.27 ± 2.90 μM |

| | | | |
|---|---|---|---|
| 83 | 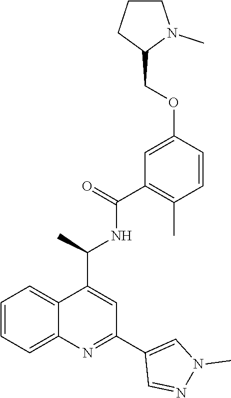 | A | 33.94 ± 3.09 μM |

| | | | |
|---|---|---|---|
| 84 | 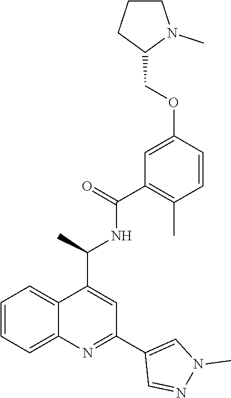 | A | 103.48 ± 24.4 μM |

| | | | |
|---|---|---|---|
| 85 | 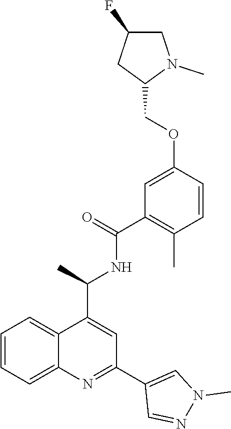 | A | 33.90 ± 3.74 μM |

| | | | |
|---|---|---|---|
| 86 | 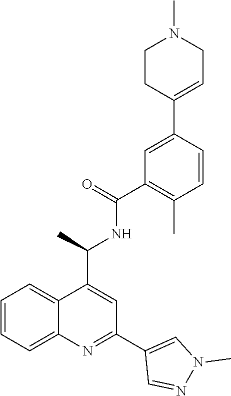 [SMILES: Cc1ccc(C2=CCN(C)CC2)cc1C(=O)N[C@H](C)c1cc(-c2cnn(C)c2)nc2ccccc12] | C | 92.74 ± 4.59 μM |

| | | | |
|---|---|---|---|
| 87 | 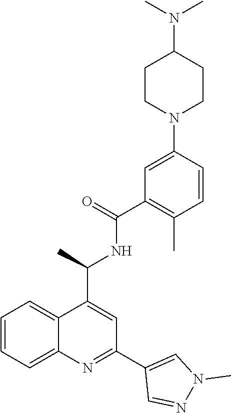 [SMILES: Cc1ccc(N2CCC(N(C)C)CC2)cc1C(=O)N[C@H](C)c1cc(-c2cnn(C)c2)nc2ccccc12] | C | 73.78 ± 5.88 μM |

| | | | |
|---|---|---|---|
| 88 | 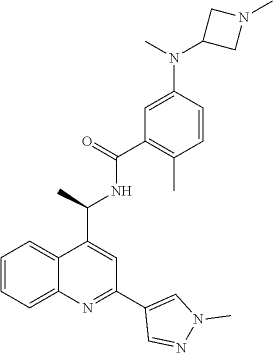 [SMILES: Cc1ccc(N(C)C2CN(C)C2)cc1C(=O)N[C@H](C)c1cc(-c2cnn(C)c2)nc2ccccc12] | C | 97.36 ± 6.51 μM |

| | | | |
|---|---|---|---|
| 89 | 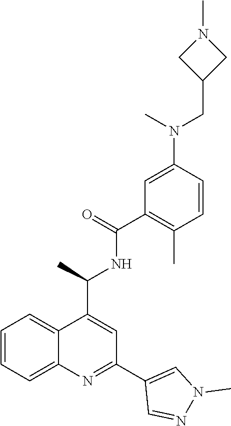 [SMILES: Cc1ccc(N(C)CC2CN(C)C2)cc1C(=O)N[C@H](C)c1cc(-c2cnn(C)c2)nc2ccccc12] | C | 89.34 μM |

| | | | |
|---|---|---|---|
| 90 | 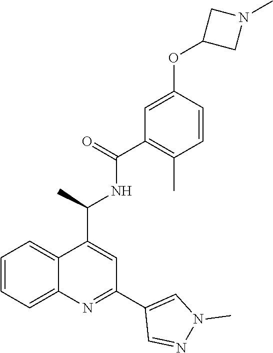 [SMILES: Cc1ccc(OC2CN(C)C2)cc1C(=O)N[C@H](C)c1cc(-c2cnn(C)c2)nc2ccccc12] | A | 72.54 ± 2.08 μM |

| | | | |
|---|---|---|---|
| 91 | 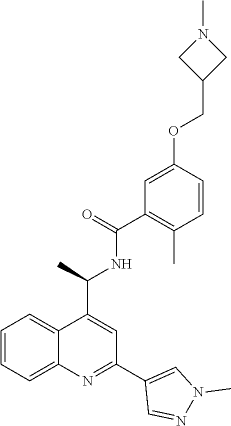 [SMILES: Cc1ccc(OCC2CN(C)C2)cc1C(=O)N[C@H](C)c1cc(-c2cnn(C)c2)nc2ccccc12] | A | 96.06 ± 5.36 μM |

| | | | |
|---|---|---|---|
| 92 | 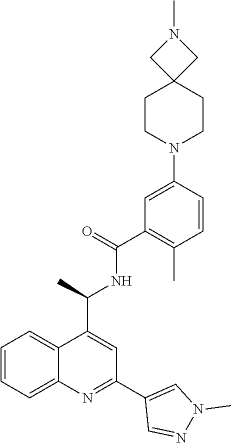 [SMILES: Cc1ccc(N2CCC3(CC2)CN(C)C3)cc1C(=O)N[C@H](C)c1cc(-c2cnn(C)c2)nc2ccccc12] | B | 76.54 ± 2.18 μM |

| | | | |
|---|---|---|---|
| 93 | 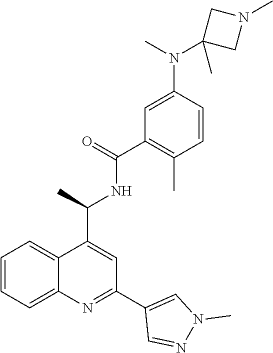 [SMILES: Cc1ccc(N(C)C2(C)CN(C)C2)cc1C(=O)N[C@H](C)c1cc(-c2cnn(C)c2)nc2ccccc12] | C | 128.81 ± 2.32 μM |

| | | | |
|---|---|---|---|
| 94 | 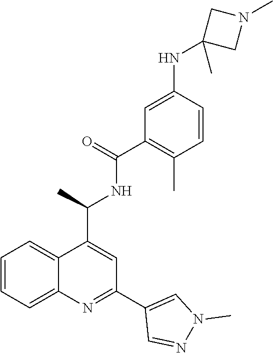 [SMILES: Cc1ccc(NC2(C)CN(C)C2)cc1C(=O)N[C@H](C)c1cc(-c2cnn(C)c2)nc2ccccc12] | C | 153.71 μM |

| | | | |
|---|---|---|---|
| 95 | 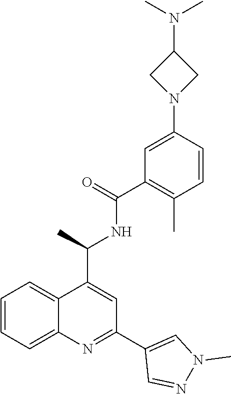 [SMILES: Cc1ccc(N2CC(N(C)C)C2)cc1C(=O)N[C@H](C)c1cc(-c2cnn(C)c2)nc2ccccc12] | B | 310.53 ± 21.14 μM |

| | | | |
|---|---|---|---|
| 96 | 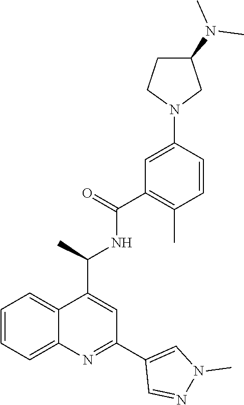 [SMILES: Cc1ccc(N2CC[C@@H](N(C)C)C2)cc1C(=O)N[C@H](C)c1cc(-c2cnn(C)c2)nc2ccccc12] | A | 28.82 μM |

| | | | |
|---|---|---|---|
| 97 | 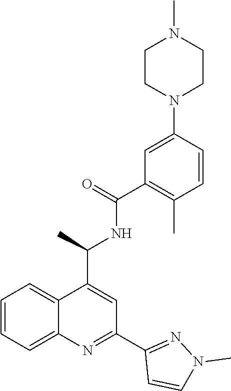 [SMILES: Cc1ccc(N2CCN(C)CC2)cc1C(=O)N[C@H](C)c1cc(-c2ccn(C)n2)nc2ccccc12] | A | 30.73 ± 2.45 μM |

| | | | |
|---|---|---|---|
| 98 | 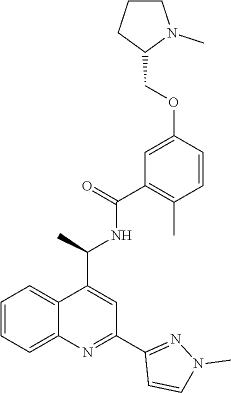 | A | 18.82 ± 1.49 μM |

| | | | |
|---|---|---|---|
| 99 | 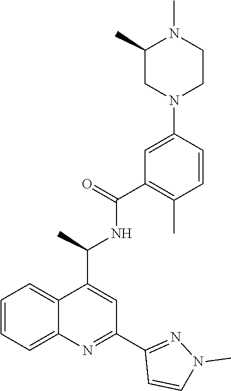 [SMILES: Cc1ccc(N2CCN(C)[C@H](C)C2)cc1C(=O)N[C@H](C)c1cc(-c2ccn(C)n2)nc2ccccc12] | A | 24.34 ± 2.62 μM |
| 100 | 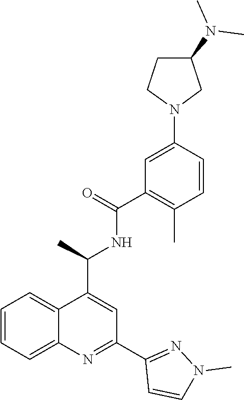 [SMILES: Cc1ccc(N2CC[C@@H](N(C)C)C2)cc1C(=O)N[C@H](C)c1cc(-c2ccn(C)n2)nc2ccccc12] | A | 30.62 ± 1.72 μM |
| 101 | 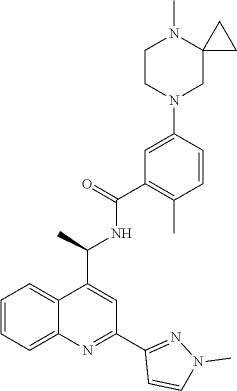 [SMILES: Cc1ccc(N2CCN(C)C3(CC3)C2)cc1C(=O)N[C@H](C)c1cc(-c2ccn(C)n2)nc2ccccc12] | B | 40.82 ± 4.27 μM |
| 102 | 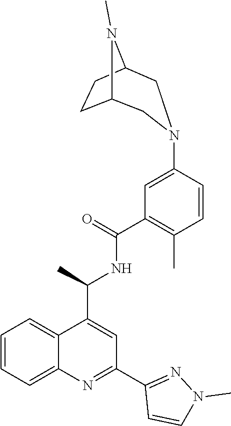 [SMILES: Cc1ccc(N2CC3CCC(C2)N3C)cc1C(=O)N[C@H](C)c1cc(-c2ccn(C)n2)nc2ccccc12] | A | 15.16 ± 1.09 μM |
| 103 | 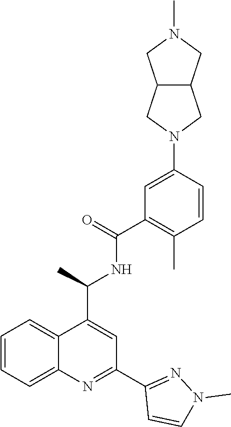 [SMILES: Cc1ccc(N2CC3CN(C)CC3C2)cc1C(=O)N[C@H](C)c1cc(-c2ccn(C)n2)nc2ccccc12] | A | 27.26 ± 1.74 μM |
| 104 | 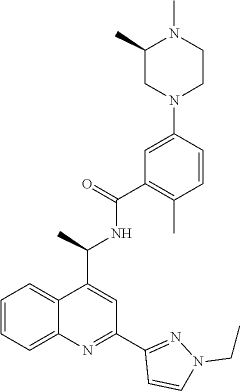 [SMILES: CCn1ccc(-c2cc([C@@H](C)NC(=O)c3cc(N4CCN(C)[C@H](C)C4)ccc3C)c3ccccc3n2)n1] | A | 30.47 ± 2.21 μM |
| 105 | 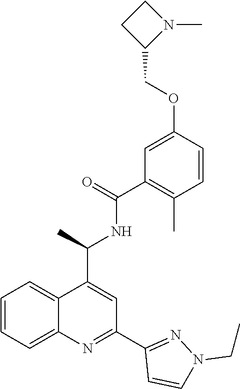 | A | 41.83 ± 2.29 μM |
| 106 | 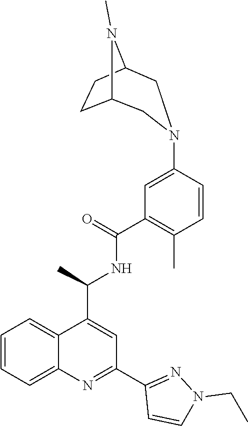 [SMILES: CCn1ccc(-c2cc([C@@H](C)NC(=O)c3cc(N4CC5CCC(C4)N5C)ccc3C)c3ccccc3n2)n1] | A | 31.33 ± 7.45 μM |
| 107 | 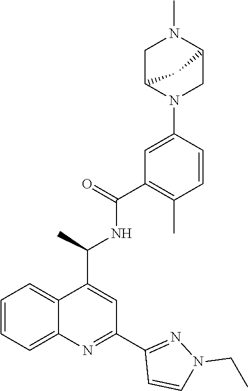 | A | 22.31 ± 1.39 μM |
| 108 | 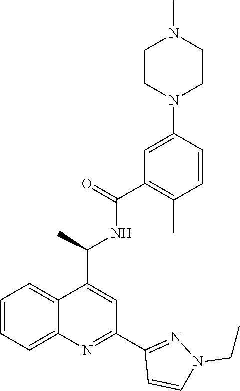 [SMILES: CCn1ccc(-c2cc([C@@H](C)NC(=O)c3cc(N4CCN(C)CC4)ccc3C)c3ccccc3n2)n1] | A | 18.52 ± 1.40 μM |
| 109 | 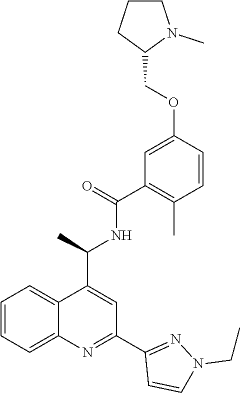 | | 26.96 ± 1.66 μM |
| 110 | 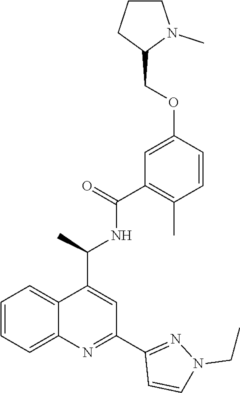 | A | 46.34 ± 13.95 μM |
| 111 | 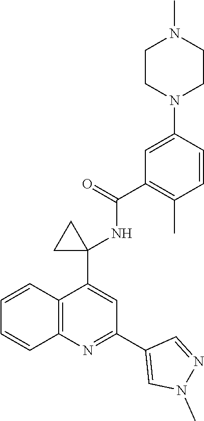 [SMILES: Cc1ccc(N2CCN(C)CC2)cc1C(=O)NC1(c2cc(-c3cnn(C)c3)nc3ccccc23)CC1] | A | >150 μM |
| 112 | 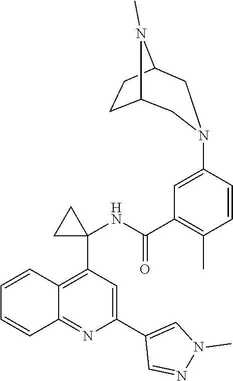 [SMILES: Cc1ccc(N2CC3CCC(C2)N3C)cc1C(=O)NC1(c2cc(-c3cnn(C)c3)nc3ccccc23)CC1] | A | 53.77 ± 2.52 μM |
| 113 | 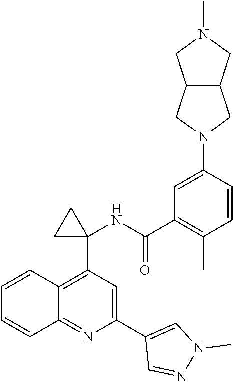 [SMILES: Cc1ccc(N2CC3CN(C)CC3C2)cc1C(=O)NC1(c2cc(-c3cnn(C)c3)nc3ccccc23)CC1] | A | 56.11 μM |
| 114 | 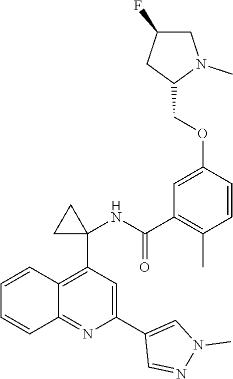 | A | 51.33 ± 6.52 μM |
| 115 | 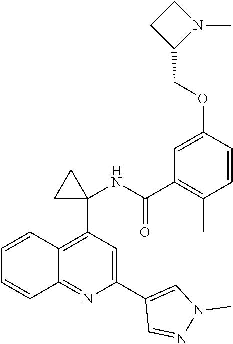 | A | 126.68 ± 7.89 μM |

A: IC₅₀ < 0.5 μM;
B: 0.5 μM ≤ IC₅₀ < 1 μM;
C: 1 μM ≤ IC₅₀ < 5 μM;
D: 5 μM ≤ IC₅₀ 7.89 μM

Example 124. The following illustrate representative pharmaceutical dosage forms, containing a compound of formula I (‘Compound X’), for therapeutic or prophylactic use in humans

| (i) Tablet 1 | mg/tablet |
|---|---|
| Compound X= | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| Compound X= | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound X= | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml |
|---|---|
| Compound X= (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | |
| (pH adjustment to 7.0-7.5) | q.s. |
| Water for injection | q.s. ad 1 mL |

| (v) Injection 2 (10 mg/ml) | mg/ml |
|---|---|
| Compound X= (free acid form) | 10.0 |
| Monobasic sodium phosphate | 0.3 |
| Dibasic sodium phosphate | 1.1 |
| Polyethylene glycol 400 | 200.0 |
| 1.0N Sodium hydroxide solution | |
| (pH adjustment to 7.0-7.5) | q.s. |
| Water for injection | q.s. ad 1 mL |

| (vi) Aerosol | mg/can |
|---|---|
| Compound X= | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A compound of formula (I): 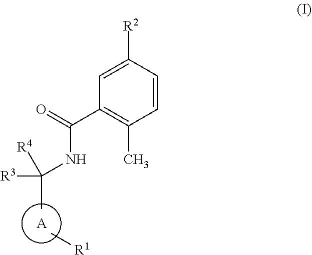
or a salt thereof, wherein:
R¹ is halo, NR^{a}R^{b}, phenyl, benzyloxy, a 3-10 membered heterocycle, or a 5-10 membered heteroaryl, which phenyl, benzyloxy, 3-10 membered heterocycle, and a 5-10 membered heteroaryl is optionally substituted with one or more groups R^{x} independently selected from the group consisting of halo, hydroxy, cyano, (C₁-C₆)alkyl, (C₁-C₆)alkoxy, (C₁-C₆)alkylthio, (C₃-C₆)cycloalkyl, NR^{a}R^{b}, oxo, 3-10 membered heterocycle, a 5-10 membered heteroaryl, —C(═O)NR^{a}R^{b}, —C(═O)3-10 membered heterocycle, —C(═O)5-10 membered heteroaryl, methylenedioxy, and (C₁-C₆)alkylsulfonyl, wherein any R^{x} is optionally substituted with one or more groups R^{y} independently selected from the group consisting of halo, hydroxy, cyano, (C₁-C₆)alkyl, (C₁-C₆)alkoxy, NR^{c}R^{d}—C(═O)NR^{c}R^{d} (C₃-C₆)cycloalkyl, carboxy, 3-10 membered heterocycle, —C(═O)3-10 membered heterocycle, and phenyl, wherein any R^{y} is optionally substituted with one or more groups independently selected from the group consisting of hydroxy, (C₁-C₆)alkyl, (C₁-C₆)alkoxy, cyano, and halo;
A is a 9-membered bicyclic heteroaryl or a 10-membered bicyclic heteroaryl, which 9-membered bicyclic heteroaryl and 10-membered bicyclic heteroaryl is optionally substituted with one or more groups independently selected from the group consisting of halo, cyano, (C₁-C₆)alkyl, (C₁-C₆)alkoxy, (C₃-C₆)cycloalkyl, (C₁-C₆)alkoxycarbonyl, and (C₁-C₆)alkanoyloxy, wherein any (C₁-C₆)alkyl, (C₁-C₆)alkoxy, (C₃-C₆)cycloalkyl, (C₁-C₆)alkoxycarbonyl, and (C₁-C₆)alkanoyloxy is optionally substituted with one or more groups independently selected from halo;
R² is R^{e}, —OR^{e}, —SR^{e}, or —NR^{g}R^{h};
R³ is H or (C₁-C₃)alkyl and R⁴ is H or (C₁-C₆)alkyl; or R³ and R⁴ taken together with the carbon to which they are attached form a (C₃-C₆)cycloalkyl;
each R^{a} and R^{b} is independently selected from the group consisting of H, (C₁-C₆)alkyl, (C₃-C₆)cycloalkyl, (C₃-C₆)cycloalkyl(C₁-C₆)alkyl, and (C₁-C₆)alkanolyl;
each R^{c} and R^{d} is independently selected from the group consisting of H, (C₁-C₆)alkyl, (C₃-C₆)cycloalkyl, (C₃-C₆)cycloalkyl(C₁-C₆)alkyl, and (C₁-C₆)alkanolyl;
R^{e} is a 3-10 membered heterocycle that is optionally substituted with one or more groups independently selected from the group consisting of halo and (C₁-C₆)alkyl; or R^{e} is (C₁-C₆)alkyl, that is substituted with NRⁱR^{k} or with a 3-10 membered heterocycle that is optionally substituted with one or more groups independently selected from the group consisting of halo and (C₁-C₆)alkyl;
each R^{g} and R^{h} is independently selected from the group consisting of H, (C₁-C₆)alkyl, (C₃-C₆)cycloalkyl, (C₃-C₆)cycloalkyl(C₁-C₆)alkyl, (C₁-C₆)alkanolyl, a 3-10 membered heterocycle, —C(═NH)—NH₂, and (C₁-C₆)alkylsulfonyl, wherein any (C₁-C₆)alkyl, (C₃-C₆)cycloalkyl, (C₃-C₆)cycloalkyl(C₁-C₆)alkyl, (C₁-C₆)alkanolyl, 3-10 membered heterocycle, and (C₁-C₆)alkylsulfonyl is optionally substituted with one or more groups independently selected from halo, (C₁-C₆)alkyl, a 3-10 membered heterocycle and NRⁿR^{m}; and wherein any a 3-10 membered heterocycle is optionally substituted with one or more groups independently selected from the group consisting of halo and (C₁-C₆)alkyl; or R^{g} and R^{h} together with the nitrogen to which they are attached form a 3-10 membered heterocycle that is optionally substituted with one or more groups independently selected from the group consisting of halo, (C₁-C₆)alkyl, 3-10 membered heterocycle, and NRⁿR^{m};
each Rⁱ and R^{k} is independently selected from the group consisting of H, (C₁-C₆)alkyl, (C₃-C₆)cycloalkyl, (C₃-C₆)cycloalkyl(C₁-C₆)alkyl, —C(═NH)—NH₂, and (C₁-C₆)alkanolyl; and
each R^{m} and Rⁿ is independently selected from the group consisting of H, (C₁-C₆)alkyl, (C₃-C₆)cycloalkyl, (C₃-C₆)cycloalkyl(C₁-C₆)alkyl, —C(═NH)—NH₂, and (C₁-C₆)alkanolyl.

2. The compound or salt of claim 1, wherein:
R¹ is halo, NR^{a}R^{b}, phenyl, benzyloxy, a 5-membered heteroaryl, a 6-membered heteroaryl, a 5-membered heterocycle, a 6-membered heterocycle, a 9-membered bicyclic heterocycle, or a 9-membered bicyclic heteroaryl, which phenyl, benzyloxy, 5-membered heteroaryl, 6-membered heteroaryl, 5-membered heterocycle, 6-membered heterocycle, 9-membered bicyclic heterocycle, and 9-membered bicyclic heteroaryl is optionally substituted with one or more groups R^{x} independently selected from the group consisting of halo, hydroxy, cyano, (C₁-C₆)alkyl, (C₁-C₆)alkoxy, (C₃-C₆)cycloalkyl, NR^{a}R^{b}, oxo, 5-membered heteroaryl, 6-membered heterocycle, —C(═O)NR^{a}R^{b}, 4-membered heterocycle, 5-membered heterocycle, methylenedioxy, and (C₁-C₆)alkyl sulfonyl, wherein any R^{x} is optionally substituted with one or more groups R^{y} independently selected from the group consisting of halo, hydroxy, cyano, (C₁-C₆)alkyl, (C₁-C₆)alkoxy, NR^{c}R^{d}—C(═O)NR^{c}R^{d} (C₃-C₆)cycloalkyl, carboxy, 6-membered heterocycle, and phenyl, wherein any R^{y} is optionally substituted with one or more groups independently selected from the group consisting of hydroxy, (C₁-C₆)alkyl, (C₁-C₆)alkoxy, cyano, and halo;
A is a 9-membered bicyclic heteroaryl or a 10-membered bicyclic heteroaryl, which 9-membered bicyclic heteroaryl and 10-membered bicyclic heteroaryl is optionally substituted with one or more groups independently selected from the group consisting of halo, cyano, (C₁-C₆)alkyl, (C₁-C₆)alkoxy, (C₃-C₆)cycloalkyl, (C₁-C₆)alkoxycarbonyl, and (C₁-C₆)alkanoyloxy, wherein any (C₁-C₆)alkyl, (C₁-C₆)alkoxy, (C₃-C₆)cycloalkyl, (C₁-C₆)alkoxycarbonyl, and (C₁-C₆)alkanoyloxy is optionally substituted with one or more groups independently selected from halo;
R² is —OR^{e}, —SR^{e}, or —NR^{g}R^{h};
R³ is H;\
R⁴ is methyl;
each R^{a} and R^{b} is independently selected from the group consisting of H, (C₁-C₆)alkyl, (C₃-C₆)cycloalkyl, (C₃-C₆)cycloalkyl(C₁-C₆)alkyl, and (C₁-C₆)alkanolyl; or R^{a} and R^{b} together with the nitrogen to which they are attached form a aziridino, azetidino, morpholino, piperazino, pyrrolidino or piperidino, which a aziridino, azetidino, morpholino, piperazino, pyrrolidino and piperidino, is optionally substituted with (C₁-C₆)alkyl;
each R^{c} and R^{d} is independently selected from the group consisting of H, (C₁-C₆)alkyl, (C₃-C₆)cycloalkyl, (C₃-C₆)cycloalkyl(C₁-C₆)alkyl, and (C₁-C₆)alkanolyl; or R^{c} and R^{d} together with the nitrogen to which they are attached form a aziridino, azetidino, morpholino, piperazino, pyrrolidino or piperidino, which a aziridino, azetidino, morpholino, piperazino, pyrrolidino and piperidino, is optionally substituted with (C₁-C₆)alkyl;
R^{e} is (C₁-C₆)alkyl, that is substituted with a 4-membered heterocycle or NRⁱR^{k};
each R^{g} and R^{h} is independently selected from the group consisting of H, (C₁-C₆)alkyl, (C₃-C₆)cycloalkyl, (C₁-C₆)cycloalkyl(C₁-C₆)alkyl, (C₁-C₆)alkanolyl, 4-membered heterocycle, —C(═NH)—NH₂, and (C₁-C₆)alkylsulfonyl, wherein any (C₁-C₆)alkyl, (C₃-C₆)cycloalkyl, (C₃-C₆)cycloalkyl(C₁-C₆)alkyl, (C₁-C₆)alkanolyl, and (C₁-C₆)alkylsulfonyl is optionally substituted with one or more groups independently selected from 4-membered heterocycle and NRⁿR^{m}; or R^{g} and R^{h} together with the nitrogen to which they are attached form a aziridino, azetidino, morpholino, piperazino, pyrrolidino or piperidino, which a aziridino, azetidino, morpholino, piperazino, pyrrolidino and piperidino, is optionally substituted with (C₁-C₆)alkyl;
each Rⁱ and R^{k} is independently selected from the group consisting of H, (C₁-C₆)alkyl, (C₃-C₆)cycloalkyl, (C₃-C₆)cycloalkyl(C₁-C₆)alkyl, —C(═NH)—NH₂, and (C₁-C₆)alkanolyl; or Rⁱ and R^{k} together with the nitrogen to which they are attached form a aziridino, azetidino, morpholino, piperazino, pyrrolidino or piperidino, which a aziridino, azetidino, morpholino, piperazino, pyrrolidino and piperidino, is optionally substituted with (C₁-C₆)alkyl; and
each R^{m} and Rⁿ is independently selected from the group consisting of H, (C₁-C₆)alkyl, (C₃-C₆)cycloalkyl, (C₃-C₆)cycloalkyl(C₁-C₆)alkyl, —C(═NH)—NH₂, and (C₁-C₆)alkanolyl; or R^{m} and Rⁿ together with the nitrogen to which they are attached form a aziridino, azetidino, morpholino, piperazino, pyrrolidino or piperidino, which a aziridino, azetidino, morpholino, piperazino, pyrrolidino and piperidino, is optionally substituted with (C₁-C₆)alkyl.

3. The compound or salt of claim 1, which is a compound of formula (Ib): 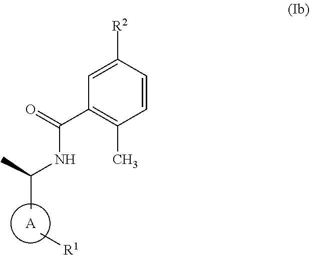
or a salt thereof.

4. The compound or salt of claim 1, wherein R¹ is a 5-membered heteroaryl that is optionally substituted with one or more groups R^{x} independently selected from the group consisting of halo, hydroxy, cyano, (C₁-C₆)alkyl, (C₁-C₆)alkoxy, (C₃-C₆)cycloalkyl, NR^{a}R^{b}, oxo, 5-membered heteroaryl, 6-membered heterocycle, —C(═O)NR^{a}R^{b}, 4-membered heterocycle, 5-membered heterocycle, methylenedioxy, and (C₁-C₆)alkyl sulfonyl, wherein any R^{x} is optionally substituted with one or more groups R^{y} independently selected from the group consisting of halo, hydroxy, cyano, (C₁-C₆)alkyl, (C₁-C₆)alkoxy, NR^{c}R^{d}—C(═O)NR^{c}R^{d} (C₃-C₆)cycloalkyl, carboxy, 6-membered heterocycle, and phenyl, wherein any R^{y} is optionally substituted with one or more groups independently selected from the group consisting of hydroxy, (C₁-C₆)alkyl, (C₁-C₆)alkoxy, cyano, and halo.

5. The compound or salt of claim 1, wherein R¹ is selected from the group consisting of chloro,

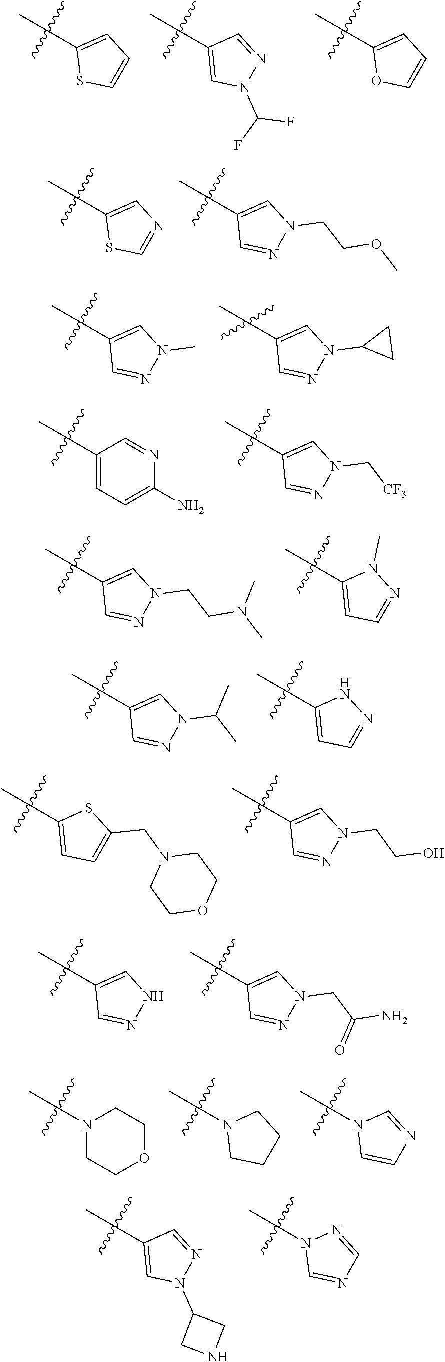

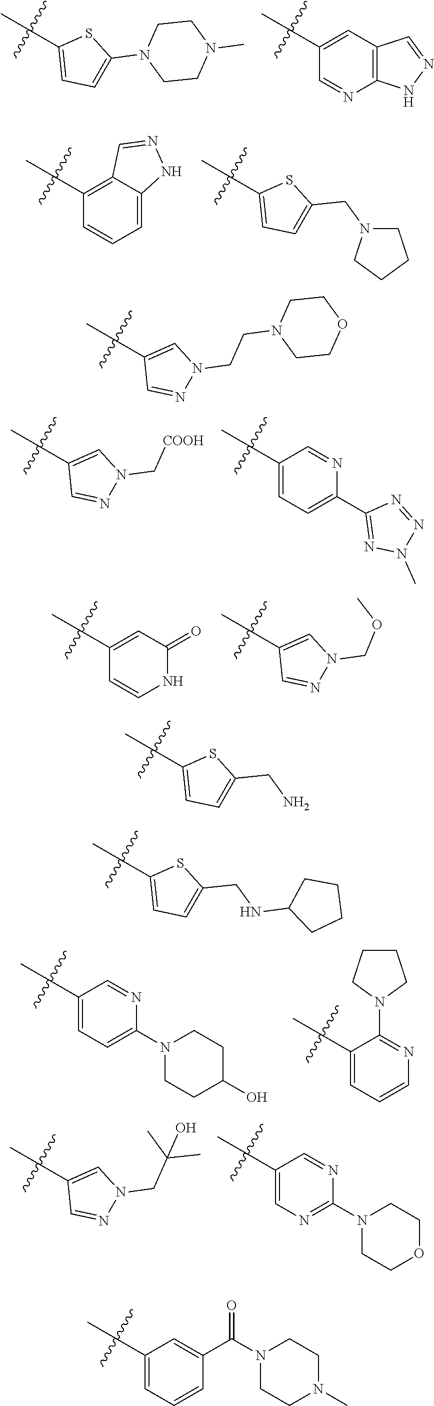

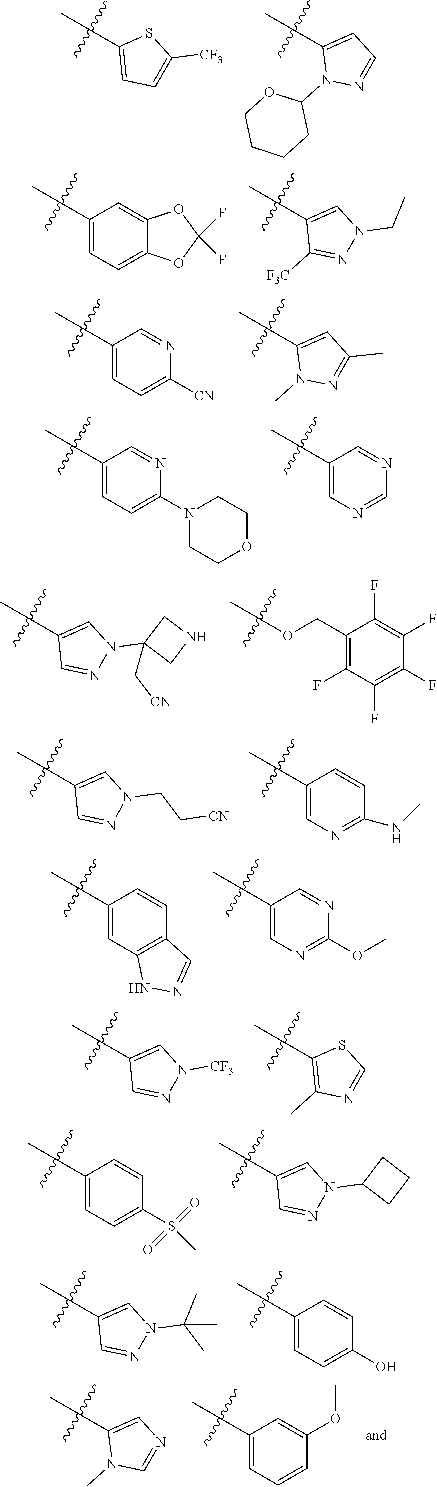

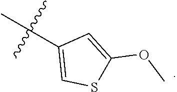 [SMILES: *c1csc(OC)c1]

6. The compound or salt of claim 1, wherein R¹ is selected from the group consisting of:

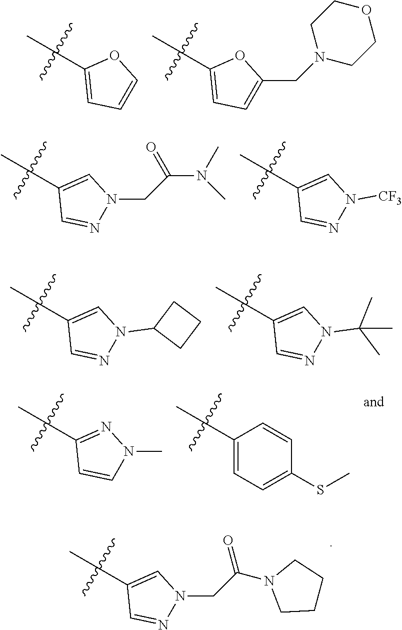

7. The compound or salt of claim 1, wherein R² is —OR^{e}.

8. The compound or salt of claim 1, wherein R² is —SR^{e}.

9. The compound or salt of claim 1, wherein R² is —NR^{g}R^{h}.

10. The compound or salt of claim 1, wherein R² is —OCH₂CH₂N(CH)₂.

11. The compound or salt of claim 1, wherein R² is selected from the group consisting of amino, methylamino, dimethylamino, 2-aminoethoxy,

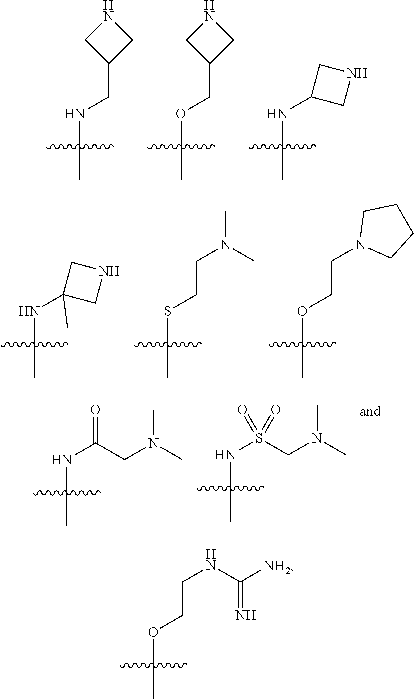

12. The compound or salt of claim 1, wherein R² is selected from the group consisting of

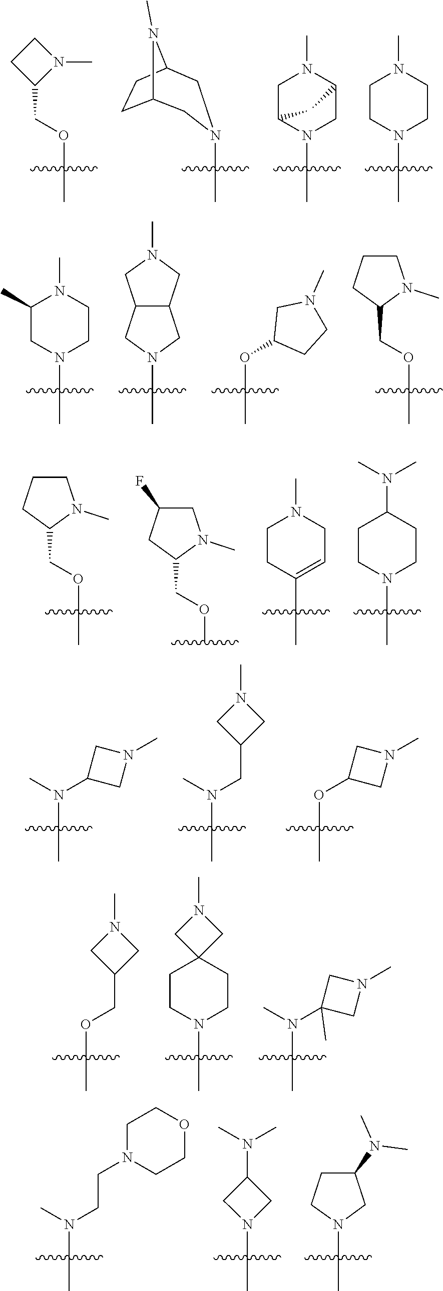

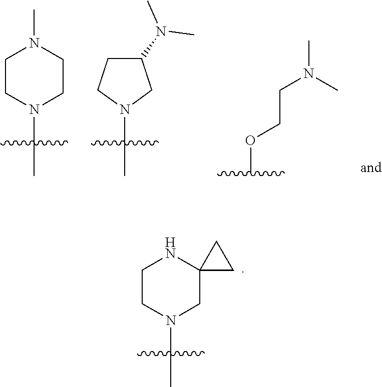

13. The compound or salt of claim 1, wherein A is a 9-membered bicyclic heteroaryl that is optionally substituted with one or more groups independently selected from the group consisting of halo, cyano, (C₁-C₆)alkyl, (C₁-C₆)alkoxy, (C₃-C₆)cycloalkyl, (C₁-C₆)alkoxycarbonyl, and (C₁-C₆)alkanoyloxy, wherein any (C₁-C₆)alkyl, (C₁-C₆)alkoxy, (C₃-C₆)cycloalkyl, (C₁-C₆)alkoxycarbonyl, and (C₁-C₆)alkanoyloxy is optionally substituted with one or more groups independently selected from halo.

14. The compound or salt of claim 1, wherein A is a 10-membered bicyclic heteroaryl that is optionally substituted with one or more groups independently selected from the group consisting of halo, cyano, (C₁-C₆)alkyl, (C₁-C₆)alkoxy, (C₃-C₆)cycloalkyl, (C₁-C₆)alkoxycarbonyl, and (C₁-C₆)alkanoyloxy, wherein any (C₁-C₆)alkyl, (C₁-C₆)alkoxy, (C₃-C₆)cycloalkyl, (C₁-C₆)alkoxycarbonyl, and (C₁-C₆)alkanoyloxy is optionally substituted with one or more groups independently selected from halo.

15. The compound or salt of claim 1, wherein A is a 9-membered bicyclic heteroaryl.

16. The compound or salt of claim 1, wherein A is a 10-membered bicyclic heteroaryl.

17. The compound or salt of claim 1, wherein A is: 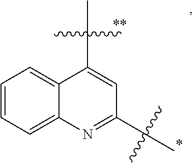 [SMILES: *c1cc(*)c2ccccc2n1]
wherein * designates the point of attachment to R¹ and ** designates the point of attachment to:

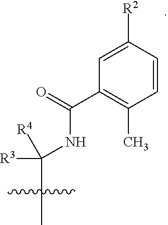

18. A pharmaceutical composition comprising a compound as described in claim 1 or a pharmaceutically acceptable salt thereof and a pharmaceutically acceptable excipient.

19. A method for promoting an antiviral effect in an animal, comprising administering a compound as described in claim 1 or a pharmaceutically acceptable salt thereof to the animal.

20. A method for inhibiting a papain-like protease in an animal in need thereof, comprising administering a compound as described in claim 1 or a pharmaceutically acceptable salt thereof to the animal.
